SEQUENCE LISTING (189 sequences)

SEQ ID NO: 1 - protein, 133 aa, unidentified [sequence omitted]

SEQ ID NO: 2 - protein, 132 aa, synthetic construct
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 3 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 4 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 5 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEELKPLEEVLXYAQSKNFHLRPRDLISNINVIVLELKGSETTFXCEYADETATIVEFLNRWITFSQSIISTLT

SEQ ID NO: 6 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEELKPLEEVLXYAQSKNFHLRPRDLISNINVIVLELKGSETTFXCEYADETATIVEFLNRWITFSQSIISTLT

SEQ ID NO: 7 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEELKPLEEVLXGAQSKNFHLRPRDLISNINVIVLELKGSETTFXCEYADETATIVEFLNRWITFSQSIISTLT

SEQ ID NO: 8 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEELKYLEEVLXLAQSKNFHLRPRDLISNINVIVLELKGSETTFXCEYADETATIVEFLNRWITFSQSIISTLT

SEQ ID NO: 9 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEELKYLEEVLXLAQSKNFHLRPRDLISNINVIVLELKGSETTFXCEYADETATIVEFLNRWITFSQSIISTLT

SEQ ID NO: 10 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTYXLXYKFYXPKKATELKHLQCLEEYLKYLEEVLXLAQSKNFHLRPRDLISNINVIVLELKGSETTFXCEYADETATIVEFLNRWITFSQSIISTLT

SEQ ID NO: 11 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEYLKYLEEVLXLAQSKNFHLRPRDLISNINVIVLELKGSETTFXCEYADETATIVEFLNRWITFSQSIISTLT

SEQ ID NO: 12 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEYLKPLEEVLXLAQSKNFHLRPRDLISNINVIVLELKGSETTFXCEYADETATIVEFLNRWITFSQSIISTLT

SEQ ID NO: 13 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFYFYXPKKATELKHLQCLEEELKPLEEVLXLAQSKNFHLRPRDLISNINVIVLELKGSETTFXCEYADETATIVEFLNRWITFSQSIISTLT

SEQ ID NO: 14 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKYYXPKKATELKHLQCLEEELKPLEEVLXLAQSKNFHLRPRDLISNINVIVLELKGSETTFXCEYADETATIVEFLNRWITFSQSIISTLT

SEQ ID NO: 15 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPYLTRXLXFKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 16 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEYLLLDLQXILNGINNYKNPKLTRXLXFKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 17 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEYLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 18 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLYLQXILNGINNYKNPKLTRXLXFKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 19 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLYLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 20 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPYLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 21 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEYLKPLEEVLXLAQSKNFHLRPRDLISNINVIVLELKGSETTFXCEYADETATIVEFLNRWITFSQSIISTLT

SEQ ID NO: 22 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEYLKYLEEVLXLAQSKNFHLRPRDLISNINVIVLELKGSETTFXCEYADETATIVEFLNRWITFSQSIISTLT

SEQ ID NO: 23 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEELKPLEEVLXLAQSKNFHLRPRDLISNINVIVLELKGSETTFXCEYADETATIVEFLNRWITFCQSIISTLT

SEQ ID NO: 24 - protein, 20 aa, synthetic construct
  VARIANT 1..20: note = This sequence may encompass 1-10 GS repeating units
GSGSGSGSGSGSGSGSGSGS

SEQ ID NO: 25 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 26 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 27 - protein, 40 aa, synthetic construct
  VARIANT 1..40: note = This sequence may encompass 1-10 GGSG repeating units
GGSGGGSGGGSGGGSGGGSGGGSGGGSGGGSGGGSGGGSG

SEQ ID NO: 28 - protein, 50 aa, synthetic construct
  VARIANT 1..50: note = This sequence may encompass 1-10 GGSGG repeating units
GGSGGGGSGGGGSGGGGSGGGGSGGGGSGGGGSGGGGSGGGGSGGGGSGG

SEQ ID NO: 29 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 30 - protein, 15 aa, synthetic construct
GGGGSGGGGSGGGGS

SEQ ID NO: 31 - protein, 20 aa, synthetic construct
GGGGSGGGGSGGGGSGGGGS

SEQ ID NO: 32 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 33 - protein, 290 aa, Homo sapiens; identical to SEQ ID NO: 32

SEQ ID NO: 34 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 35 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 36 - protein, 157 aa, synthetic construct [sequence omitted]

SEQ ID NO: 37 - protein, 445 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 38 - protein, 214 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 447 aa, synthetic construct
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 214 aa, synthetic construct
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 452 aa, synthetic construct
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 219 aa, synthetic construct
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 443 aa, synthetic construct
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 214 aa, synthetic construct
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 214 aa, synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47-50 - skipped (4 entries)

SEQ ID NO: 51 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 52 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 119 aa, synthetic construct
QVQLVQSGVEVKKPGASVKVSCKASGYTFTNYYMYWVRQAPGQGLEWMGGFPSNGGTNFNEKFKNRVTLTTDSSTTTAYMELKSLQFDDTAVYYCARRDYRFDMGFDYWGQGTTVTVSS

SEQ ID NO: 54 - protein, 111 aa, synthetic construct [sequence omitted]

SEQ ID NO: 55 - protein, 113 aa, synthetic construct [sequence omitted]

SEQ ID NO: 56 - protein, 107 aa, synthetic construct [sequence omitted]

SEQ ID NO: 57 - protein, 459 aa, synthetic construct
QVQLVQSGGGLVQPGGSLRLSCAASGFTFSSYWMYWVRQVPGKGLEWVSAIDTGGGRTYYADSVKGRFAISRVNAKNTMYLQMNSLRAEDTAVYYCARDEGGGTGWGVLKDWPYGLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 214 aa, synthetic construct
QPVLTQPLSVSVALGQTARITCGGNNIGSKNVHWYQQKPGQAPVLVIYRDSNRPSGIPERFSGSNSGNTATLTISRAQAGDEADYYCQVWDSSTAVFGTGTKLTVLQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 439 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 60 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 443 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - protein, 214 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 442 aa, synthetic construct
OVQLVESGGGLVKPGGSLRLSCAASGFTFSNYGMSWIROAPGKGLEWVSTISGGGSNIYYADSVKGRFTISRDNAKNSLYLQMNSLRAEDTAVYYCVSYYYGIDFWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 64 - protein, 214 aa, synthetic construct
DIQMTQSPSSLSASVGDRVTITCKASQDVTTAVAWYQQKPGKAPKLLIYWASTRHTGVPSRFSGSGSGTDFTLTISSLQPEDFATYYCOOHYTIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 445 aa, synthetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 66 - protein, 218 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 444 aa, synthetic construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 68 - protein, 220 aa, synthetic construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 443 aa, synthetic construct
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 70 - protein, 220 aa, synthetic construct
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 450 aa, synthetic construct
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 72 - protein, 214 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 496 aa, synthetic construct
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 74 - protein, 271 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVQSGAEVKKPGASVKVSCKASGYTFTDYNMDWVRQAPGQGLEWMGDINPDNGVTIYNQKFEGRVTMTTDTSTSTAYMELRSLRSDDTAVYYCAREADYFYFDYWGQGTTLTVSSGGCGGGKVAACKEKVAALKEKVAALKEKVAALKE

SEQ ID NO: 75 - protein, 447 aa, synthetic construct
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 213 aa, synthetic construct
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 113 aa, synthetic construct
QVQLVESGGGVVQPGRSLRLTCKASGLTFSSSGMHWVRQAPGKGLEWVAVIWYDGSKRYYADSVKGRFTISRDNSKNTLFLQMNSLRAEDTAVYYCATNNDYWGQGTLVTVSS

SEQ ID NO: 78 - protein, 107 aa, synthetic construct
EIVLTQSPATLSLSPGERATLSCRASQSVSSYLAWYQQKPGQAPRLLIYTASNRATGIPARFSGSGSGTDFTLTISSLEPEDFAVYYCQQYSNWPRTFGQGTKVEIK

SEQ ID NO: 79 - protein, 120 aa, synthetic construct
EVQLQQSGPVLVKPGASVKMSCKASGYTFTSYYMYWVKQSHGKSLEWIGGVNPSNGGTNFNEKFKSKATLTVDKSSSTAYMELNSLTSEDSAVYYCARRDYRYDMGFDYWGQGTTLTVSS

SEQ ID NO: 80 - protein, 111 aa, synthetic construct
QIVLTQSPAIMSASPGEKVTMTCRASKGVSTSGYSYLHWYQQKPGSSPRLLIYLASYLESGVPVRFSGSGSGTSYSLTISRMEAEDAATYYCQHSRELPLTFGTGTRLEIK

SEQ ID NO: 81 - protein, 120 aa, synthetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSS

SEQ ID NO: 82 - protein, 111 aa, synthetic construct
EIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIK

SEQ ID NO: 83 - protein, 448 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 219 aa, synthetic construct
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 5 aa, synthetic construct
NYYMY

SEQ ID NO: 86 - protein, 17 aa, synthetic construct
GINPSNGGTNFNEKFKN

SEQ ID NO: 87 - protein, 11 aa, synthetic construct
RDYRFDMGFDY

SEQ ID NO: 88 - protein, 15 aa, synthetic construct
RASKGVSTSGYSYLH

SEQ ID NO: 89 - protein, 7 aa, synthetic construct
LASYLES

SEQ ID NO: 90 - protein, 9 aa, synthetic construct
QHSRDLPLT

SEQ ID NO: 91 - protein, 5 aa, synthetic construct
NSGMH

SEQ ID NO: 92 - protein, 17 aa, synthetic construct
VIWYDGSKRYYADSVKG

SEQ ID NO: 93 - protein, 4 aa, synthetic construct
NDDY

SEQ ID NO: 94 - protein, 11 aa, synthetic construct
RASQSVSSYLA

SEQ ID NO: 95 - protein, 7 aa, synthetic construct
DASNRAT

SEQ ID NO: 96 - protein, 9 aa, synthetic construct
QQSSNWPRT

SEQ ID NO: 97 - protein, 9 aa, synthetic construct
FTFSNYGMS

SEQ ID NO: 98 - protein, 10 aa, synthetic construct
TISGGGSNIY

SEQ ID NO: 99 - protein, 9 aa, synthetic construct
VSYYYGIDF

SEQ ID NO: 100 - protein, 11 aa, synthetic construct
KASQDVTTAVA

SEQ ID NO: 101 - protein, 7 aa, synthetic construct
WASTRHT

SEQ ID NO: 102 - protein, 9 aa, synthetic construct
QQHYTIPWT

SEQ ID NO: 103 - protein, 8 aa, synthetic construct
GLTFSSSG

SEQ ID NO: 104 - protein, 8 aa, synthetic construct
IWYDGSKR

SEQ ID NO: 105 - protein, 6 aa, synthetic construct
ATNNDY

SEQ ID NO: 106 - protein, 11 aa, synthetic construct
RASQSVSSYLA

SEQ ID NO: 107 - protein, 7 aa, synthetic construct
TASNRAT

SEQ ID NO: 108 - protein, 9 aa, synthetic construct
QQYSNWPRT

SEQ ID NO: 109 - protein, 831 aa, synthetic construct
MQIPQAPWPWWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQSKYGPPCPSCPAPEFLGGPSVFLFPPKPKDTLMISRTPEVTCWVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMDQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLMQIPQAPWPWWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 110 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 112 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 215 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 448 aa, synthetic construct
EVQLLESGGGLVQPGGSLRLSCAASGFTFSSYAMSWVRQAPGKGLEWVSGISGSGGFTYYADSVKGRFTISRDNSKNTLYLQMNSLRAEDTAVYYCAKPPRGYNYGPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 117 - protein, 214 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 118-119 - skipped (2 entries)

SEQ ID NO: 120 - protein, 118 aa, synthetic construct
QGQLQESGPSLVKPSQTLSLTCTVSGDSITRGYWNWIRKHPGKGLEYIGYISYTGSTYSNLSLKSRVTISRDTSKNQYYLKLSSVTAADTAVYYCATSTGWLDPVDYWGQGTLVTVSS

SEQ ID NO: 121 - protein, 107 aa, synthetic construct
DIVMTQSPDSLAVSLGERATINCKASQNVDTSVAWFQQKPGQPPKALIYSASFRYSGVPDRFSGSGSGTDFTLTISSLQAEDVAVYFCQQYYGYPFTFGQGTKLEIK

SEQ ID NO: 122 - protein, 123 aa, synthetic construct
EVQLVQSGGGLVKPGGSLRLSCAASGFTFSSYTMNWVRQAPGKGLEWVSSISSGSDYLYYADSVKGRFTISRDNAKNSLYLQMNSLRAEDTAVYYCARNELRWYPQAGAFDRWGQGTMVTVSS

SEQ ID NO: 123 - protein, 109 aa, synthetic construct
QSVVTQPPSMSAAPGQRVTISCSGSSSYIESSYVGWYQQLPGTAPRLLIYDDDMRPSGIPDRFSGSKSGTSATLAITGLQTGDEADYYCEIWRSGLGGVFGGGTKLTVL

SEQ ID NO: 124-125 - skipped (2 entries)

SEQ ID NO: 126 - protein, 453 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSPDYSPYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 216 aa, synthetic construct
QSVLTQPPSASGTPGQRVTISCSGSSSNIGSNTVNWYQQLPGTAPKLLIYGNSNRPSGVPDRFSGSKSGTSASLAISGLQSEDEADYYCQSYDSSLSGSVFGGGIKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 128 - protein, 5 aa, synthetic construct
SYTMN

SEQ ID NO: 129 - protein, 17 aa, synthetic construct
SISSGSDYLYYADSVKG

SEQ ID NO: 130 - protein, 14 aa, synthetic construct
NELRWYPQAGAFDR

SEQ ID NO: 131 - protein, 13 aa, synthetic construct
SGSSSYIESSYVG

SEQ ID NO: 132 - protein, 7 aa, synthetic construct
DDDMRPS

SEQ ID NO: 133 - protein, 10 aa, synthetic construct
EIWRSGLGGV

SEQ ID NO: 134 - protein, 360 aa, synthetic construct
QVQLVESGGGLVQPGGSLRLSCAASGKMSSRRCMAWFRQAPGKERERVAKLLTTSGSTYLADSVKGRFTISRDNSKNTVYLQMNSLRAEDTAVYYCAADSFEDPTCTLVTSSGAFQYWGQGTLVTVSSEPKSSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 759 aa, synthetic construct
DIQMTQSPASLSASVGDRVTITCQASQSIGTYLAWYQQKPGKPPKLLIYRAFILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSANYYPCWVRQAPGKGLEWIGCIYGGSSDITYDANWTKGRFTISRDNSKNTVYLQMNSLRAEDTAVYYCARSAWYSGWGGDLWGQGTLVTVSSGGGGSGGGGSGGGGSGGGGSIQMTQSPSSLSASVGDRVTITCQASQSISNRLAWYQQKPGKAPKLLIYSASTLASGVPSRFSGSGSGTDFTLTISSLQPEDFATYYCQSTYYGNDGNAFGTGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 136 - protein, 942 aa, synthetic construct
QMQLVQSGAEVKKPGSSVKVSCKASGGTFSSYAISWVRQAPGQGLEWMGRIIPILGIANYAQKFQGRVTITADKSTSTAYMELSSLRSEDTAVYYCAKPRDGYNLVAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPGGGGSEVQLQQSGPELVKPGPSMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSQMQLVQSGAEVKKPGSSVKVSCKASGGTFSSYAISWVRQAPGQGLEWMGRIIPILGIANYAQKFQGRVTITADKSTSTAYMELSSLRSEDTAVYYCAKPRDGYNLVAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRQLVLTQPPSVSGAPGQRVTISCTGSSSNIGAGYDVHWYQQLPGAAPKLLIYGDINRPSGVPDRFSGSKSGISASLAITGLQAEDEADYYCQSYDSSLSGGVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 222 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEP

SEQ ID NO: 140 - protein, 211 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 141 - protein, 448 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 142 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 453 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATNYCQQWSFNPPTFGQGTKVEIKRYVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 451 aa, synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 214 aa, synthetic construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 226 aa, synthetic construct
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 148 - protein, 214 aa, synthetic construct
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 126 aa, synthetic construct
QVTLRESGPALVKPTQTLTLTCTFSGFSLSTSGMCVSWIRQPPGKALEWLALIDWDDDKYYSTSLKTRLTISKDTSKNQVVLTMTNMDPVDTATYYCARILVDIVATITNDAFDVWGQGTMVTVSS

SEQ ID NO: 150 - protein, 110 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPPELTFGGGTKVEIKR

SEQ ID NO: 151 - protein, 230 aa, synthetic construct
  VARIANT 18: note = Any naturally occurring amino acid
  VARIANT 19: note = Any naturally occurring amino acid
  VARIANT 21: note = Any naturally occurring amino acid
  VARIANT 140: note = Any naturally occurring amino acid
  VARIANT 142: note = Any naturally occurring amino acid
  VARIANT 177: note = Any naturally occurring amino acid
PKSCDKTHTCPPCPAPEXXGXPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKXTPPVLDSDGSFFLYSKLTVDKSRWQQGNVFSCSVMHEALHNHYTQKSLSLSPG

SEQ ID NO: 152 - protein, 6 aa, synthetic construct
  VARIANT 6: note = Alanine or proline
LLQGPX

SEQ ID NO: 153 - protein, 8 aa, synthetic construct
GGLLQGPP

SEQ ID NO: 154 - protein, 48 aa, synthetic construct
QETNPTENLYFQQKNMQCQRRFYEALHDPNLNEEQRNARIRSIRDDDC

SEQ ID NO: 155 - protein, 61 aa, synthetic construct
QTADNQKNMQCQRRFYEALHDPNLNEEQRNARIRSIRDDCSQSANLLAEAQQLNDAQAPQA

SEQ ID NO: 156 - protein, 38 aa, synthetic construct
QETKNMQCQRRFYEALHDPNLNEEQRNARIRSIRDDDC

SEQ ID NO: 157 - protein, 38 aa, synthetic construct
QETFNKQCQRRFYEALHDPNLNEEQRNARIRSIRDDDC

SEQ ID NO: 158 - protein, 38 aa, synthetic construct
QETFNMQCQRRFYEALHDPNLNKEQRNARIRSIRDDDC

SEQ ID NO: 159 - protein, 37 aa, synthetic construct
QETFNMQCQRRFYEALHDPNLNEEQRNARIRSIKDDC

SEQ ID NO: 160 - protein, 35 aa, synthetic construct
QETMQCQRRFYEALHDPNLNEEQRNARIRSIKDDC

SEQ ID NO: 161 - protein, 34 aa, synthetic construct
QETQCQRRFYEALHDPNLNEEQRNARIRSIKDDC

SEQ ID NO: 162 - protein, 33 aa, synthetic construct
QETCQRRFYEALHDPNLNEEQRNARIRSIKDDC

SEQ ID NO: 163 - protein, 20 aa, synthetic construct
QETRGNCAYHKGQLVWCTYH

SEQ ID NO: 164 - protein, 20 aa, synthetic construct
QETRGNCAYHKGQIIWCTYH

SEQ ID NO: 165 - protein, 177 aa, unidentified
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 166 - protein, 154 aa, unidentified
MFHVSFRYIFGIPPLILVLLPVTSSDCHIKDKDGKAFGSVLMISINQLDKMTGTDSDCPNNEPNFFKKHLCDDTKEAAFLNRAARKLRQFLKMNISEEFNDHLLRVSDGTQTLVNCTSKEEKTIKEQKKNDPCFLKRLLREIKTCWNKILKGSI

SEQ ID NO: 167 - protein, 154 aa, unidentified
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 168 - protein, 176 aa, unidentified
MFHVSFRYIFGIPPLILVLLPVASSDCDISGKDGGAYQNVLMVNIDDLDNMINFDSNCLNNEPNFFKKHSCDDNKEASFLNRASRKLRQFLKMNISDDFKLHLSTVSQGTLTLLNCTSKGKGRKPPSLSEAQPTKNLEENKSSKEQKKQNDLCFLKILLQKIKTCWNKILRGIKEH

SEQ ID NO: 169 - protein, 176 aa, unidentified
MFHVSFRYIFGIPPLILVLLPVASSDCDFSGKDGGAYQNVLMVSIDDLDNMINFDSNCLNNEPNFFKKHSCDDNKEASFLNRAARKLKQFLKMNISDDFKLHLSTVSQGTLTLLNCTSKGKGRKPPSLGEAQPTKNLEENKSLKEQRKQNDLCFLKILLQKIKTCWNKILRGITEH

SEQ ID NO: 170 - protein, 193 aa, unidentified
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 171 - protein, 193 aa, unidentified
MAAEQVEDYCISFVEMKFINNTLYFVAENDEDLESDHFGKLEPKLSIIRNLNDQVLFINQGNQPVFEDMPDSDCSDNAPQTIFIIYMYKDSLTRGLAVTISVQCKKMSTLSCENKIVSFKEMNPPDNIDNEESDIIFFQRSVPGHDDKIQFESSLYKGYFLACKKENDLFKLILKKQDDNRDKSVMFTVQNQN

SEQ ID NO: 172 - protein, 193 aa, unidentified; identical to SEQ ID NO: 171

SEQ ID NO: 173 - protein, 194 aa, unidentified
MAAMSEEGSCVNFKEMMFIDNTLYLIPEDNGDLESDHFGRLHCTTAVIRSINDQVLFVDKRNPPVFEDMPDIDRTANESQTRLIIYMYKDSEVRGLAVTLSVKDGRMSTLSCKNKIISFEEMNPPENIDDIKSDLIFFQKRVPGHNKMEFESSLYEGHFLACQKEDDAFKLVLKRKDENGDKSVMFTLTNLHQS

SEQ ID NO: 174 - protein, 192 aa, unidentified
MAAMSEDSCVNFKEMMFIDNTLYFIPEENGDLESDNFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 175 - protein, 193 aa, unidentified
MAAGPVEDNCISLVEMKFIDNTLYFVAENDENLESDYFGRLEPKLSIIRNLNDQVLFINQGNQPVFEDMPDSDCTDNAPQTVFIIYMYKDSLTRGLAVTISVKCEKTSTLSCKNKIISFKEMSPPENINDEGNDIIFFQRSVPGHDDKIQFESSLYKGYFLACEKENDLFKLILKEKDENGDKSVMFTVQNQN

SEQ ID NO: 176 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNHKNPRLTRXLXFKFYXPKKATELKHLQCLEEELKPLEEVLXLAPSKNFHLRPRDLISDINVIVLELKGSETTFXCEYADETATIVEFLNRWITFSQSIISTLT

SEQ ID NO: 177 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNHKNPRLTRXLXFKFYXPKKATELKHLQCLEEELKPLEEVLXLAPSKNFHLRPRDLISNINVIVLELKGSETTFXCEYADETATIVEFLNRWITFSQSIISTLT

SEQ ID NO: 178 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNHKNPRLTRXLXFKFYXPKKATELKHLQCLEEELKPLEEALXLAPSKNFHLRPRDLISDINVIVLELKGSETTFXCEYADETATIVEFLNRWITFSQSIISTLT

SEQ ID NO: 179 - protein, 133 aa, synthetic construct
  SITE 23: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNHKNPRLTRALXFKFYXPKKATELKHLQCLEEELKPLEEVLXLAPSKNFHLRPRDLISNINVIVLELKGSETTFXCEYADETATIVEFLNRWITFSQSIISTLT

SEQ ID NO: 180 - protein, 133 aa, synthetic construct
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQAILNGINNHKNPRLTRXLXFKFYXPKKATELKHLQCLEEELKPLEEVLXLAPSKNFHLRPRDLISDINVIVLELKGSETTFXCEYADETATIVEFLNRWITFSQSIISTLT

SEQ ID NO: 181 - protein, 133 aa, synthetic construct
  SITE 23: note = Norleucine
  SITE 39: note = Norleucine
  SITE 41: note = Homoserine
  SITE 71: note = Homoserine
  SITE 104: note = Homoserine
APTSSSTKKTQLQLEHLLLDLQXILNGINNHKNPRLTRXLXFKFYAPKKATELKHLQCLEEELKPLEEVLXLAPSKNFHLRPRDLISNINVIVLELKGSETTFXCEYADETATIVEFLNRWITFSQSIISTLT

SEQ ID NO: 182 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPRLTRXLXFKFYXPKKATELKHLQCLEEELKPLEEALXLAPSKNFHLRPRDLISDINVIVLELKGSETTFXCEYADETATIVEFLNRWITFSQSIISTLT

SEQ ID NO: 183 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXBKFYXPKKATELKHLQCLEEELKPLEEVLXLAQSKNFHLRPRDLISDINVIVLELKGSETTFXCEYADETATIVEFLNRWITFSQSIISTLT

SEQ ID NO: 184 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEELKPLEEVLXLAQSKNFHLRPRDLISDINVIVLELKGSETTFXCEYADETATIVEFLNRWITFSQSIISTLT

SEQ ID NO: 185 - protein, 133 aa, synthetic construct; identical to SEQ ID NO: 184
  SITE 23: note = Norleucine
  SITE 39: note = Norleucine
  SITE 41: note = Homoserine
  SITE 46: note = Norleucine
  SITE 71: note = Homoserine
  SITE 88: note = O-(2-aminoethyl)-O'-(2-aminoethyl)octaethylene glycol aspartic acid
  SITE 104: note = Homoserine

SEQ ID NO: 186 - protein, 152 aa, unidentified
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 187 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine
DCDIEGKDGKQYESVLXVSIDQLLDSXKEIGSNCLXNEFNFFKRHICDANKEGXFLFRAARKLRQFLKXNSTGDFXLHLLKVSEGTTILLNCTGQVKGRKPAALGEAQPTKSLXENKSLKEQKKLNDLCFLKRLLQEIKTCWNKILXGTKEH

SEQ ID NO: 188 - protein, 17 aa, synthetic construct
  SITE 1: note = Acetyl arginine
  DISULFID 4..14: note = Intrachain disulfide bond
  SITE 8: note = Lysine-3-((3-((2,5-dioxopyrrolidin-1-yl)oxy)-3-oxop ropyl)disulfaneyl)propanoate
  SITE 17: note = Histidine amide
RGNCAYHKGQLVWCTYH

SEQ ID NO: 189 - protein, 5 aa, synthetic construct
GGGGS

(19) United States
(12) Patent Application Publication
(10) Pub. No.: US 2023/0201364 A1
(43) Pub. Date: Jun. 29, 2023

(54) ANTIBODY CONJUGATES AND MANUFACTURE THEREOF

(71) Applicant: Bright Peak Therapeutics AG, Basel (CH)

(72) Inventors: Vijaya Raghavan PATTABIRAMAN, Volketswil (CH); Bertolt KREFT, Kleinmachnow (DE); Jean-Philippe CARRALOT, Saint-Louis (FR); Rubén ALVAREZ SANCHEZ, Rosenau (FR); Magali MULLER, Pulversheim (FR); Matilde ARÉVALO-RUIZ, Basel (CH)

(21) Appl. No.: 17/861,176

(22) Filed: Jul. 9, 2022

Related U.S. Application Data
(60) Provisional application No. 63/219,989, filed on Jul. 9, 2021.
(60) Provisional application No. 63/219,981, filed on Jul. 9, 2021.
(60) Provisional application No. 63/219,985, filed on Jul. 9, 2021.
(60) Provisional application No. 63/219,992, filed on Jul. 9, 2021.
(60) Provisional application No. 63/219,995, filed on Jul. 9, 2021.

(51) Int. Cl. A61K 47/68; A61K 38/20; C07K 16/18; A61P 35/00

(52) U.S. Cl. CPC A61K 47/6813; A61K 38/2013; C07K 16/18; A61P 35/00

(57) ABSTRACT

The present disclosure relates to conjugate compositions comprising an antibody or an antigen binding fragment, a synthetic protein, and a linker. The disclosure further relates to methods of making the conjugate compositions and to methods of using the conjugate compositions for the treatment of diseases. In one aspect, the disclosure relates to the treatment of cancer using the conjugate compositions.

### CROSS REFERENCE

This application claims the benefit of U.S. Provisional Application No. 63/219,989 filed Jul. 9, 2021, U.S. Provisional Application No. 63/219,981 filed Jul. 9, 2021, U.S. Provisional Application No. 63/219,985 filed Jul. 9, 2021, U.S. Provisional Application No. 63/219,992 filed Jul. 9, 2021, and U.S. Provisional Application No. 63/219,995 filed Jul. 9, 2021, which applications are incorporated herein by reference in their entirety.

### SEQUENCE LISTING

The instant application contains a Sequence Listing which has been submitted electronically in XML file format and is hereby incorporated by reference in its entirety. Said XML copy, created on Aug. 17, 2022, is named 94917-0032_716201US_SL.xml and is 266,885 bytes in size.

### SUMMARY OF THE INVENTION

Provided herein are protein-antibody conjugates and methods of manufacture thereof. In some embodiments, the protein-antibody conjugates employ a linker (e.g., a chemical (non-peptiyl) linker to covalently attach a protein (e.g., a cytokine) and an antibody. In some embodiments, the constructs substantially retain the functionalities of both individual units and, in certain instances, can exhibit a synergy between the antibody mode of action and the protein mode of action. Such constructs offer a wide variety of therapeutic potential.

In some embodiments, the methods and conjugates provided herein are readily scalable and can be used to rapidly generate a wide variety of such constructs. In one aspect, the protein-antibody conjugates are prepared from “off the shelf” antibodies which require no genetic modification prior to use and can rapidly be derivatized with proteins (including cytokines) of interest. In some embodiments, the antibodies are conjugated to synthetic proteins which have a conjugation handle incorporated at a desired point of attachment during the synthesis, enabling rapid generation of a well-defined construct. In some embodiments, the structural platform herein enables attachment at non-terminal residues (e.g., not the N- or C-terminus) of one or both of the antibody or protein, thus providing greater flexibility than is available for traditional conjugates based around fusion protein technology.

Provided herein are conjugates comprising (a) antibodies and (b) a recombinant protein or a synthetic protein, as well as methods of making the same. In some embodiments, the conjugates herein are formed between a recombinant protein or a synthetic protein and an antibody that does not contain any mutations or other modifications in order to facilitate the conjugation. Thus, in some embodiments, the methods provided herein can be used to prepare an antibody-synthetic protein conjugate with any “off the shelf” antibody. In some embodiments, the methods provided herein utilize reagents to add linkers to the antibody, wherein the linkers contain a reactive group (“conjugation handle”) which can facilitate formation of a covalent bond with a suitable reactive group on the recombinant protein or the synthetic protein. In some embodiments, the synthetic proteins of the present disclosure are chemically synthesized to contain such suitable reactive groups at the time of synthesis, thus facilitating an easy conjugation between the protein and the antibody.

Disclosed herein are conjugates that exhibit one or more of the following: synergistic efficacy, improved tolerability, direct targeting of tumor infiltrating leukocytes (TILs), significant dose reduction, simplified CMC (chemical conjugation to existing antibody products, superior efficacy, ability to treat a broader patient population; conjugation of cytokines to antibodies with different isotypes, expand specific immune cell populations, highly chemoselective conjugation; site selective conjugation, and varieties of payloads can be attached to modified antibodies.

In one aspect, disclosed herein is a conjugate comprising: (a) an antibody or an antigen binding fragment; (b) a recombinant protein or a synthetic protein of from about 50 to about 500 amino acid residues in length; and (c) one or more linkers connecting the antibody or the antigen binding fragment to the recombinant protein or the synthetic protein.

In another aspect, disclosed herein is a population of conjugates of any one of claims 1-95, wherein a linker of each of at least about 80%, at least about 85%, at least about 90%, at least about 95%, at least about 96% at least about 97%, at least about 98%, or at least about 99% of the conjugates is attached to the same amino acid residue position of each recombinant protein or each synthetic protein.

In another aspect, disclosed herein is a method of preparing a conjugate, comprising: a) chemically synthesizing a synthetic protein or preparing a recombinant protein, wherein the protein comprises a protein conjugation handle; b) providing an antibody or an antigen binding fragment, wherein the antibody or an antigen binding fragment comprises an antibody conjugation handle, the antibody conjugation handle is complementary to the protein conjugation handle; and c) forming a covalent bond between the protein conjugation handle and the antibody conjugation handle.

In another aspect, disclosed herein is a method of preparing a conjugate, comprising: a) chemically synthesizing a synthetic protein or preparing a recombinant protein, wherein the protein comprises a protein conjugation handle; b) providing an antibody or an antigen binding fragment, wherein the antibody or an antigen binding fragment comprises an antibody conjugation handle; c) providing a bi-functional reagent having a first reagent conjugation handle and a second reagent conjugation handle, wherein the first reagent conjugation handle is complementary to the protein conjugation handle, and wherein the second reagent conjugation handle is complementary to the antibody conjugation handle; d) forming first covalent bond between the protein conjugation handle and the first reagent conjugation handle; and e) forming a second covalent bond between the antibody conjugation handle and the second reagent conjugation handle.

In another aspect, disclosed herein is use of a conjugate described herein in the manufacture of a medicament for treating a disease or disorder in a subject in need thereof.

In another aspect, disclosed herein is a method of treating a disease or disorder in a subject in need thereof, comprising administering to the subject a conjugate described herein or a population of conjugates described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates an anti-PD-1-IL-2 immunocytokine of the disclosure and the interaction of the anti-PD-1-IL-2 immunocytokine with an activated T cell through IL2Rβ/γ upregulation and PD-1 inhibition.

FIG. 1B shows the structure of modified conjugatable cytokine CMP-003. CMP-003 has an amino acid sequence of SEQ ID NO: 3 and comprises an azide bearing polymer attached to residue F42Y and an additional polymer attached to residue Y45.

FIG. 2A shows site-selective modification of anti-PD1 antibody by chemical modification technology to introduce one or two conjugation handles.

FIG. 2B shows Q-TOF mass spectra of unmodified pembrolizumab and pembrolizumab with DBCO conjugation handle.

FIG. 2C shows site-selective conjugation of modified IL-2 cytokine to generate a PD1-IL2 with DAR1, DAR 2 or mixed DAR between 1 and 2.

FIG. 2D shows TIC chromatogram (top) and intact RP-HPLC (bottom) profile of crude Pembrolizumab-IL2 (CMP-003) conjugation reaction.

FIG. 2E shows Q-TOF mass spec profile of crude Pembrolizumab-IL2 (CMP-003) conjugation reaction showing the formation of DAR1 and DAR 2 species.

FIG. 2F shows intact RP-HPLC (Top left) profile of purified Pembrolizumab-IL2 (CMP-003) immunocytokine.

FIG. 2G shows Q-TOF mass spec profile of purified Pembrolizumab-IL2 (CMP-003) with mixed DAR.

FIG. 2H shows SEC-HPLC of purified Pembrolizumab-IL2 (CMP-003) immunocytokine.

FIG. 3A shows plots measuring ability of the unmodified and of conjugated anti-PD1 antibodies to bind with PD-1 ligand, with the figure showing normalized ELISA signal on the y-axis and dosage of the unmodified and of conjugated anti-PD1 antibodies on the x-axis. The unconjugated reference antibody and the conjugated antibodies tested in this figure are composition CMP-004 (Pembrolizumab) and CMP-005, CMP-007, CMP-001, respectively.

FIG. 3B shows an analogous plot to that of FIG. 3A with CMP-033 (Durvalumab) and CMP-034 with binding to PD-L1 being assessed.

FIG. 3C shows an analogous plot to that of FIG. 3A and FIG. 3B with CMP-091 (parent antibody biosimilar adalimumab) and CMP-089 with binding to TNFα being assessed.

FIG. 4A shows plots measuring ability of the unmodified and of conjugated anti-PD1 antibodies to interfere with PD1/PDL1 pathway, with the figure showing mean luminescence intensity of effector cells NFAT-RE reporter on the y-axis and dosage of the unmodified and of conjugated anti-PD1 antibodies on the x-axis. The unconjugated reference antibody and the conjugated antibodies tested in this figure are composition Pembrolizumab (CMP-004) and CMP-005. The modified IL-2 polypeptides tested in this figure are Proleukin and CMP-002.

FIG. 4B shows an analogous plot to that of FIG. 4A with parent anti-PD-L1 antibody CMP-033 (Durvalumab) and conjugate CMP-034

FIG. 5A shows plots measuring ability of the unmodified and of conjugated anti-PD1 antibodies to bind to human neonatal Fc receptor (FcRn) at pH 6, with the figure showing mean AlphaLISA® FcRn-IgG signal on the y-axis and dosage of the unmodified and of conjugated anti-PD1 antibodies on the x-axis. The unconjugated reference antibody and the conjugated antibodies tested in this figure are composition CMP-004 (Pembrolizumab) and CMP-005, CMP-007, CMP-001, respectively.

FIG. 5B shows an analogous plot to that of FIG. 5A with parent anti-PD-L1 antibody CMP-033 (Durvalumab) and conjugate CMP-034.

FIG. 5C shows an analogous plot to that of FIG. 5A and FIG. 5B with parent anti-TNFa antibody CMP-091 (biosimilar Adalimumab) and conjugates CMP-089 (DAR1) and CMP-090 (DAR2).

FIG. 6A shows plots measuring ability of the unmodified and of conjugated anti-PD1 antibodies to bind to human Fc gamma receptor I (CD64), with the figure showing mean AlphaLISA® FcγRI-IgG signal on the y-axis and dosage of the unmodified and of conjugated anti-PD1 antibodies on the x-axis. The unconjugated reference antibodies is CMP-004 (Pembrolizumab); and the conjugated antibodies and CMP-005, CMP-007, and CMP-001.

FIG. 6B shows plots measuring ability of the unmodified and of conjugated anti-PD1 antibodies to bind to human Fc gamma receptor IIa (CD32a), with the figure showing mean AlphaLISA® FcγRIIa-IgG signal on the y-axis and dosage of the unmodified and of conjugated anti-PD1 antibodies on the x-axis. T The unconjugated reference antibodies is CMP-004 (Pembrolizumab); and the conjugated antibodies and CMP-005, CMP-007, and CMP-001.

FIG. 6C shows plots measuring ability of the unmodified and of conjugated anti-PD1 antibodies to bind to human Fc gamma receptor IIIa (CD16), with the figure showing mean AlphaLISA® FcγRIIIa-IgG signal on the y-axis and dosage of the unmodified and of conjugated anti-PD1 antibodies on the x-axis. The unconjugated reference antibodies is CMP-004 (Pembrolizumab); and the conjugated antibodies and CMP-005, CMP-007, and CMP-001.

FIG. 6D shows analogous plots to those of FIGS. 6A-6C for parent anti-PD-L1 antibody CMP-033 (Durvalumab) and conjugate CMP-034. The figure shows CD64 binding (left), CD32a binding (center), and CD16 binding (right).

FIG. 6E shows analogous plots to those of FIGS. 6A-6D for parent anti-TNFa antibody CMP-091 (biosimilar Adalimumab) and conjugates CMP-089 and CMP-090.

FIG. 7A shows plots measuring the effect of the modified IL-2 polypeptides unconjugated and conjugated to the anti-PD1 antibody on the inducement of T_{eff} and T_{reg} cells in an in vitro sample of human T-cells, with the figure showing mean fluorescence intensity for phosphorylated signal transducer and activator of transcription 5 (pSTAT5) on the y-axis and dosage of modified IL-2 polypeptide and immunocytokines on the x-axis. The modified IL-2 polypeptide tested at top left is CMP-002. The immunocytokines tested at top right is CMP-005, bottom left is CMP-007, and bottom right is CMP-001.

FIG. 7B shows plots with dose response curves for STAT5 phosphorylation in CD8 memory and CD8 naïve cells with CMP-095 (synthetic IL-7), CMP-039, and CMP-041.

FIG. 8A shows plots measuring the level of surface expression of PD-1/CD279 on resting memory (CD45RA−) and naïve (CD45RA+) CD8+ T_{eff} cells freshly isolate from peripheral blood of healthy donors.

FIG. 8B shows plots measuring the effect of the modified IL-2 polypeptides unconjugated and conjugated to the anti-PD1 antibody on the inducement of on resting memory (CD45RA−) and naïve (CD45RA+) CD8+ T_{eff} cells in an in vitro sample of human T-cells, with the figure showing mean fluorescence intensity for phosphorylated signal transducer and activator of transcription 5 (pSTAT5) on the y-axis and dosage of modified IL-2 polypeptide and immunocytokines on the x-axis. The modified IL-2 polypeptide tested in this figure is CMP-002 and the immunocytokines tested in this figure are CMP-005 and CMP-006 as a control.

FIG. 9A shows plots measuring the effect of the modified IL-2 polypeptides unconjugated and conjugated to the anti-PD1 antibody on the activation of resting naïve (CD45RA+) CD8+ T_{eff} cells in an in vitro sample of human T-cells in the presence or absence of excess amounts of unconjugated anti-PD1 antibody CMP-004 (Pembrolizumab), with the figure showing mean fluorescence intensity for phosphorylated signal transducer and activator of transcription 5 (pSTAT5) on the y-axis and dosage of modified IL-2 polypeptide and immunocytokines on the x-axis. The modified IL-2 polypeptide tested in this figure is CMP-002 and the immunocytokines tested in this figure are CMP-005 and CMP-006 as a control

FIG. 9B shows plots measuring the effect of the modified IL-2 polypeptides unconjugated and conjugated to the anti-PD1 antibody on the activation of resting memory (CD45RA−) CD8+ T_{eff} cells in an in vitro sample of human T-cells in the presence or absence of excess amounts of unconjugated anti-PD1 antibody CMP-004 (Pembrolizumab), with the figure showing mean fluorescence intensity for phosphorylated signal transducer and activator of transcription 5 (pSTAT5) on the y-axis and dosage of modified IL-2 polypeptide and immunocytokines on the x-axis. The modified IL-2 polypeptide tested in this figure is CMP-002 and the immunocytokines tested in this figure are CMP-005 and CMP-006 as a control.

FIG. 10A shows a plot describing the effect of PD-1 targeted and untargeted immunocytokines on the growth of CT26 syngeneic colon carcinoma tumors in hPD1 humanized BALB/c mice. The immunocytokine tested in this figure is Composition A tested as a single agent at 1, and 2.5 mg/kg after a single injection schedule. Control Her2-targeted immunocytokine Composition O (Trastuzumab antibody conjugated to IL-2 polypeptide) was also tested at 2.5 mg/kg. (mean±SEM).

FIG. 10B shows a bar chart describing the effect PD-1 targeted and untargeted immunocytokines on the growth of CT26 syngeneic colon carcinoma tumors in hPD1 humanized BALB/c mice 7 days after treatment. The immunocytokine tested in this figure is Composition A tested as a single agent at 1, and 2.5 mg/kg after a single injection schedule. Control Her2-targeted immunocytokine Composition O (Trastuzumab antibody conjugated to IL-2 polypeptide) was also tested at 2.5 mg/kg. (mean±SEM; ** one-way ANOVA P-value<0.001)

FIG. 11A shows a schematic of an experimental design testing the effect of CMP-092 on keyhole limpet hemocyanin (KLH)-induced delayed type hypersensitivity.

FIG. 11B shows paw thickness difference between the left paw challenged with KLH compared to baseline reported in mm as a measure of swelling at 24 hrs post-challenge.

FIG. 11C shows paw thickness difference between the left paw challenged with KLH compared to baseline reported in mm as a measure of swelling at 48 hrs post-challenge.

FIG. 11D shows paw thickness difference between the left paw challenged with KLH compared to baseline reported in mm as a measure of swelling at 72 hrs post-challenge.

FIG. 12 shows a plot describing the effect of unconjugated and conjugated anti-PD1 antibody on the growth of CT26 syngeneic colon carcinoma tumors in hPD1 BALB/c mice. The immunocytokine tested in this figure is CMP-041 tested as a single agent at 1, 3, and 10 mg/kg in a QW×2 injection schedule. Unconjugated anti-PD1 antibody CMP-105 (LZM-009) was also tested at 10 mg/kg (mean±SEM) in the same schedule.

FIG. 13 shows tumor growth inhibition in mice subcutaneously injected with EL4 cells overexpressing human CD20. Mice were treated with the unconjugated parent antibody CMP-032 (biosimilar Rituximab) at 10 mg/kg BIW and with the immunocytokine CMP-030 at 1.25 and 2.5 mg/kg QW.

FIG. 14 shows a cartoon image of an immunocytokine with a DAR of 2 with CMP-003 as the conjugated protein.

FIG. 15 provides a schematic representation of conditionally activatable immunocytokine.

FIG. 16 illustrates the preparation of an immunocytokine payload 1-mAB-payload 2 with two different payloads using chemically orthogonal capping groups.

FIG. 17 illustrates examples of orthogonal payloads that can be conjugated to mAB-IL2 immunoconjugates.

### DETAILED DESCRIPTION OF THE INVENTION

### Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Referred to herein are groups which are “attached” or “covalently attached” to residues of IL-2 polypeptides. As used herein, “attached” or “covalently attached” means that the group is tethered to the indicated reside, and such tethering can include a linking group (i.e., a linker). Thus, for a group “attached” or “covalently attached” to a residue, it is expressly contemplated that such linking groups are also encompassed.

Binding affinity refers to the strength of a binding interaction between a single molecule and its ligand/binding partner. A higher binding affinity refers to a higher strength bond than a lower binding affinity. In some instances, binding affinity is measured by the dissociation constant (KD) between the two relevant molecules. When comparing KD values, a binding interaction with a lower value will have a higher binding affinity than a binding interaction with a higher value. For a protein-ligand interaction, KD is calculated according to the following formula:

$${K}_{D} = \frac{[ L ] [ P ]}{[ LP ]}$$

where [L] is the concentration of the ligand, [P] is the concentration of the protein, and [LP] is the concentration of the ligand/protein complex.

Referred to herein are certain amino acid sequences (e.g., polypeptide sequences) which have a certain percent sequence identity to a reference sequence or refer to a residue at a position corresponding to a position of a reference sequence. Sequence identity is measured by protein-protein BLAST algorithm using parameters of Matrix BLOSUM62, Gap Costs Existence:11, Extension:1, and Compositional Adjustments Conditional Compositional Score Matrix Adjustment. This alignment algorithm is also used to assess if a residue is at a “corresponding” position through an analysis of the alignment of the two sequences being compared.

The term “pharmaceutically acceptable” refers to approved or approvable by a regulatory agency of the federal or a state government or listed in the U.S. Pharmacopeia (U.S.P.) or other generally recognized pharmacopeia for use in animals, including humans.

A “pharmaceutically acceptable excipient, carrier, or diluent” refers to an excipient, carrier, or diluent that can be administered to a subject, together with an agent, and which does not destroy the pharmacological activity thereof and is nontoxic when administered in doses sufficient to deliver a therapeutic amount of the agent.

A “pharmaceutically acceptable salt” suitable for the disclosure may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Certain formulas and other illustrations provided herein depict triazole reaction products resulting from azide-alkyne cycloaddition reactions. While such formulas generally depict only a single regioisomer of the resulting triazole formed in the reaction, it is intended that the formulas encompass both resulting regioisomers. Thus, while the formulas depict only a single regioisomer

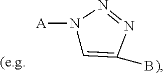

it is intended that the other regioisomer

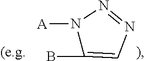

is is also encompassed.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not.

The term “moiety” refers to a specific segment or functional group of a molecule. Chemical moieties are often recognized chemical entities embedded in or appended to a molecule.

As used herein, the term “number average molecular weight” (Mn) means the statistical average molecular weight of all the individual units in a sample, and is defined by Formula (1):

$$Mn = \frac{\sum {N}_{i} {M}_{i}}{\sum {N}_{i}} \qquad Formula ( 1 )$$

where Mᵢ is the molecular weight of a unit and Nᵢ is the number of units of that molecular weight.

As used herein, the term “weight average molecular weight” (Mw) means the number defined by Formula (2):

$$Mw = \frac{\sum {N}_{i} {{M}_{i}}^{2}}{\sum {N}_{i} {M}_{i}} \qquad Formula ( 2 )$$

where Mᵢ is the molecular weight of a unit and Nᵢ is the number of units of that molecular weight.

As used herein, “peak molecular weight” (Mp) means the molecular weight of the highest peak in a given analytical method (e.g., mass spectrometry, size exclusion chromatography, dynamic light scattering, analytical centrifugation, etc.).

As used herein, “non-canonical” amino acids can refer to amino acid residues in D- or L-form that are not among the 20 canonical amino acids generally incorporated into naturally occurring proteins.

As used herein, “conjugation handle” refers to a reactive group capable of forming a bond upon contacting a complementary reactive group. In some instances, a conjugation handle preferably does not have a substantial reactivity with other molecules which do not comprise the intended complementary reactive group. Non-limiting examples of conjugation handles, their respective complementary conjugation handles, and corresponding reaction products can be found in the table below. While table headings place certain reactive groups under the title “conjugation handle” or “complementary conjugation handle,” it is intended that any reference to a conjugation handle can instead encompass the complementary conjugation handles listed in the table (e.g., a trans-cyclooctene can be a conjugation handle, in which case tetrazine would be the complementary conjugation handle). In some instances, amine conjugation handles and conjugation handles complementary to amines are less preferable for use in biological systems owing to the ubiquitous presence of amines in biological systems and the increased likelihood for off-target conjugation.

TABLE of
Conjugation Handles

| Conjugation | | Reaction |
|---|---|---|
| Handle | Complementary Conjugation Handle | Product |
| Sulfhydryl | alpha-halo-carbonyl (e.g., bromoacetamide), | thioether |
| | alpha-beta unsaturated carbonyl | |
| | (e.g., maleimide, acrylamide) | |
| Azide | alkyne (e.g., terminal alkyne, substituted | triazole |
| | cyclooctyne (e.g., dibenzocycloocytne | |
| | (DBCO), difluorocyclooctyne, | |
| | bicyclo[6.1.0]nonyne, etc.)) | |
| Phosphine | Azide/ester pair | amide |
| Tetrazine | trans-cyoclooctene | dihydro- |
| | | pyridazine |
| Amine | Activated ester (e.g., N-hydroxysuccinimide | amide |
| | ester, pentaflurophenyl ester) | |
| isocyanate | amine | urea |
| epoxide | amine | alkyl-amine |
| hydroxyl amine | aldehyde, ketone | oxime |
| hydrazide | aldehyde, ketone | hydrazone |
| potassium acyl | O-substituted hydroxylamine (e.g., O- | amide |
| trifluoroborate | carbamoylhydroxylamine) | |

Throughout the instant application, prefixes are used before the term “conjugation handle” to denote the functionality to which the conjugation handle is linked. For example, a “protein conjugation handle” is a conjugation handle attached to a protein (either directly or through a linker), an “antibody conjugation handle” is a conjugation handle attached to an antibody (either directly or through a linker), and a “linker conjugation handle” is a conjugation handle attached to a linker group (e.g., a bifunctional linker used to link a synthetic protein and an antibody).

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, Ci-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, -propyl, 1-methyl ethyl, -butyl, -pentyl, 1,1-dimethyl ethyl, 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—. Unless stated otherwise specifically in the specification, an alkylene group may be optionally substituted.

The term “alkenylene” or “alkenylene chain” refers to a straight or branched divalent hydrocarbon chain in which at least one carbon-carbon double bond is present linking the rest of the molecule to a radical group. In some embodiments, the alkenylene is —CH═CH—, —CH₂CH═CH—, or —CH═CHCH₂—. In some embodiments, the alkenylene is —CH═CH—. In some embodiments, the alkenylene is —CH₂CH═CH—. In some embodiments, the alkenylene is —CH═CHCH₂—.

The term “alkynyl” refers to a type of alkyl group in which at least one carbon-carbon triple bond is present. In one embodiment, an alkenyl group has the formula —C≡C—R_{X}, wherein R^{x} refers to the remaining portions of the alkynyl group. In some embodiments, R^{x} is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃, —C≡CCH₂CH, and —CH₂C^{o}CH.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —CH₂—O—CH₂—, —CH₂—N(alkyl)-CH₂—, —CH₂—N(aryl)-CH₂—, —OCH₂CH₂O—, —OCH₂CH₂OCH₂CH₂O—, or —OCH₂CH₂OCH₂CH₂OCH₂CH₂O—.

The term “heteocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heteroaryl” refers to an aryl group that includes one or more ring heteroatoms selected from nitrogen, oxygen, and sulfur. In some embodiments, heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C6-C9 heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7, 8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems.

The term “optionally substituted” or “substituted” means that the referenced group is optionally substituted with one or more additional group(s) individually and independently selected from D, halogen, —CN, —NH2, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄ alkyl. In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

As used herein, “AJICAP™ technology,” “AJICAP™ methods,” and similar terms refer to systems and methods (currently produced by Ajinomoto Bio-Pharma Services (“Ajinomoto”)) for the site-specific functionalization of antibodies and related molecules using affinity peptides to deliver the desired functionalization to the desired site. General protocols for the AJICAP™ methodology are found at least in PCT Publication No. WO2018199337A1, PCT Publication No. WO2019240288A1, PCT Publication No. WO2019240287A1, PCT Publication No. WO2020090979A1, Matsuda et al., Mol. Pharmaceutics 2021, 18, 4058-4066, and Yamada et al., AJICAP: Affinity Peptide Mediated Regiodivergent Functionalization of Native Antibodies. Angew. Chem., Int. Ed. 2019, 58, 5592-5597, and in particular Examples 2-4 of US Patent Publication No. US20200190165A1. In some embodiments, such methodologies site specifically incorporate the desired functionalization at lysine residues at a position selected from position 246, position 248, position 288, position 290, and position 317 of an antibody Fc region (e.g., an IgG1 Fc region) (EU numbering). In some embodiments, the desired functionalization is incorporated at residue position 248 of an antibody Fc region (EU numbering). In some embodiments, position 248 corresponds to the 18^{th} residue in a human IgG CH2 region (EU numbering).

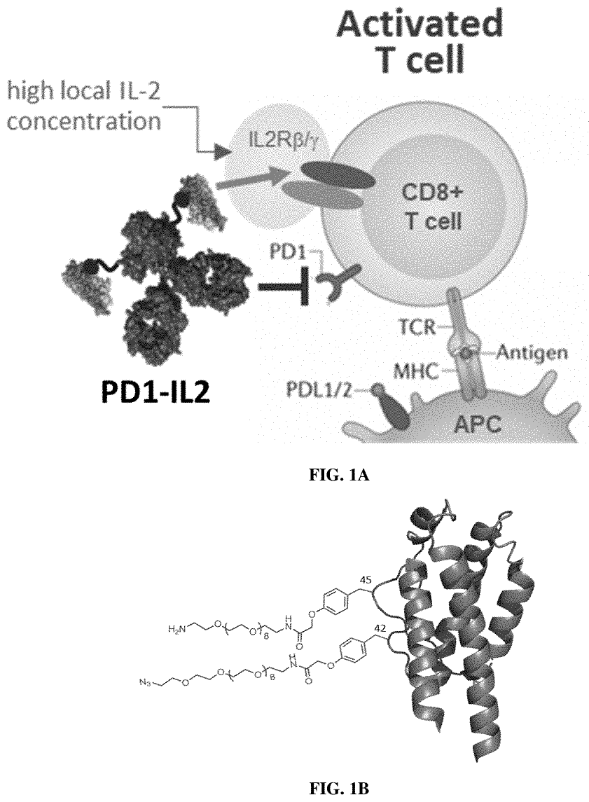

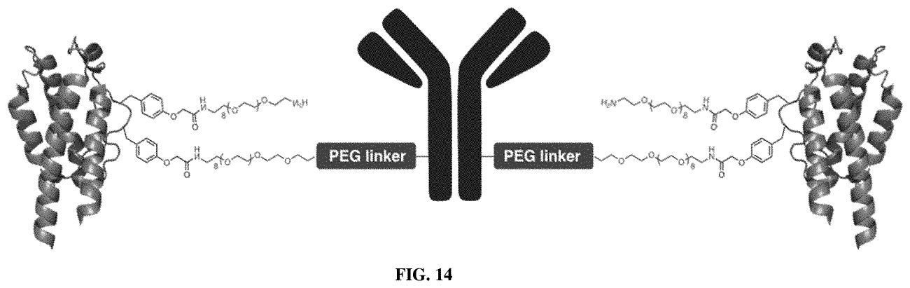

“CMP-003” refers to a modified IL-2 polypeptide having a sequence set forth in SEQ ID NO: 3 which contains a ˜0.5 kDa PEG group attached at residue Y45 and a 0.5 kDa PEG group capped with an azide functionality to facilitate conjugations at residue F42Y. A cartoon image of CMP-003 conjugated to an antibody with a DAR of 2 is shown in FIG. 14. An exemplary cartoon structure of CMP-003 is shown in FIG. 1B. CMP-003 and related modified IL-2 polypeptides are described in PCT Publication No. WO2021140416A2, which is hereby incorporated by reference as set forth in it entirety. The polymers attached to CMP-003 act to disrupt CMP-003's interaction with the IL-2 receptor alpha subunit and bias the molecule in favor of IL-2 receptor beta subunit signaling, thus enhancing the ability of the IL-2 polypeptide to expand and/or stimulate T_{eff} cells in vivo compared to WT IL-2.

“CMP-010” refers to a modified IL-2 polypeptide having a sequence set forth in SEQ ID NO: 3 which contains a ˜0.5 kDa PEG group attached at residue F42Y and a ˜0.5 kDa PEG group attached at residue Y45. CMP-010 comprises an azide conjugation handle attached to the N-terminus via an azide capped ˜0.5 kDa PEG group linked to the N-terminal amine through a glutaryl group. CMP-010 and related modified IL-2 polypeptides are described in PCT Publication No. WO2021140416A2, which is hereby incorporated by reference as set forth in it entirety. The polymers attached to CMP-010 act to disrupt CMP-010's interaction with the IL-2 receptor alpha subunit and bias the molecule in favor of IL-2 receptor beta subunit signaling, thus enhancing the ability of the IL-2 polypeptide to expand and/or stimulate T_{eff} cells in vivo compared to WT IL-2.

“CMP-002” refers to a modified IL-2 polypeptide having a sequence set forth in SEQ ID NO: 3 which contains a ˜0.5 kDa PEG group attached at residue F42Y and a second ˜0.5 kDa PEG group attached at residue Y45. CMP-002 and related modified IL-2 polypeptides are described in PCT Publication No. WO2021140416A2. The polymers attached to CMP-002 act to disrupt CMP-002's interaction with the IL-2 receptor alpha subunit and bias the molecule in favor of IL-2 receptor beta subunit signaling, thus enhancing the ability of the IL-2 polypeptide to expand and/or stimulate T_{eff} cells in vivo compared to WT IL-2.

“CMP-095” refers to a conjugable synthetic IL-7 polypeptide of SEQ ID NO: 187. CMP-095 comprises an azide moiety linked to the N-terminal amine through a glutaryl-PEG₉-linker.

“CMP-086” refers to a conjugatable synthetic IL-2 polypeptide of SEQ ID NO: 176. CMP-086 comprises an azide moiety linked to the N-terminal amine through a glutaryl-PEG₉-linker. CMP-086 contains amino acid substitutions relative to wild type IL-2 which biase the molecule in favor of interaction with the IL-2 receptor alpha subunit, thus enhancing the ability of the IL-2 polypeptide to expand and/or stimulated T_{reg} cells compared to T_{eff} cells.

### Conjugate Compositions

Provided herein are conjugates comprising (a) a polypeptide, such as an antibody or an antigen binding fragment, that binds to a target antigen, and (b) one or more proteins (e.g., therapeutic protein such as synthetic cytokines) or derivatives thereof. The conjugate compositions provided herein are effective for, in some embodiments, simultaneously delivering the protein (including synthetic proteins) and the antibody to a target cell. In some embodiments, this simultaneous delivery of both agents to the same cell has numerous benefits, including ensuring both agents are delivered to the same cell at the same time and reducing the concentration of the protein (recombinant or synthetic), the antibody, or both necessary to see a therapeutic or other benefit. In some instances, the antibody and the protein have different effects on the cell, such as blocking one signaling pathway (e.g., the antibody blocks receptor signaling of a first receptor) and activating another pathway (e.g., the protein binds to a second receptor to activate a certain activity or response from the cell).

The conjugate compositions provided herein utilize linkers to attach the antibodies to the synthetic proteins. In some embodiments, the linkers are attached to each moiety (the antibody and the synthetic protein) at specific residues or a specific subset of residues. In some embodiments, the linkers are attached to each moiety in a site-selective manner (e.g., at a pre-selected residue), such that a population of the conjugate is substantially uniform. This can be accomplished in a variety of ways as provided herein, including by site-selectively adding reagents for a conjugation reaction to a moiety to be conjugated, synthesizing or otherwise preparing a moiety to be conjugated with a desired reagent for a conjugation reaction, or a combination of these two approaches. Using these approaches, the sites of attachment (such as specific amino acid residues) of the linker to each moiety can be selected with precision. Additionally, these approaches allow a variety of linkers to be employed for the composition which are not limited to amino acid residues as required for fusion proteins. For example, linkers of the instant disclosure can be chemical polymers (e.g., polyethylene glycol, poly propylene glycol, polyesters, polyamides, and combinations thereof). This combination of linker choice and precision attachment to the moieties allows the linker to also perform the function of modulating the activity of one of the moieties, for example if the linker is attached to the synthetic protein at a position that interacts with a receptor of the synthetic protein.

FIG. 1A illustrates an exemplary immunocytokine comprising an anti-PD-1 polypeptide conjugated to an IL-2 cytokine. The anti-PD-1 antibody/IL-2 immunocytokines (referred to herein as PD1-IL2s) of the disclosure can have superior efficacy and potentially improved tolerability by a subject. In some embodiments, the anti-PD-1-IL-2 immunocytokines of the disclosure can directly target tumor-infiltrating lymphocytes (TILs). FIG. 1A thus shows a potential use of an immunocytokine and thus the utility of a general approach to creating protein-antibody conjugates (including conjugates with synthetic proteins, and in particular synthetic cytokines).

### Antibodies and Antigen Binding Fragments

In some embodiments, an antibody or an antigen binding fragment of the disclosure selectively binds to its target. An antibody selectively binds or preferentially binds to a target if it binds with greater affinity, avidity, more readily, and/or with greater duration than it binds to other substances. As such, “specific binding” or “preferential binding” does not necessarily require (although it can include) exclusive binding. Generally, but not necessarily, reference to specific binding means preferential binding where the affinity of the antibody an antigen binding fragment is at least at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the antibody for unrelated amino acid sequences. An antibody or an antigen binding fragment of the disclosure can block interaction of its target with a ligand, or block interaction of a ligand with its receptor.

As used herein, the term “antibody” refers to an immunoglobulin (Ig), polypeptide, or a protein having a binding domain which is, or is homologous to, an antigen-binding domain. The term further includes “antigen-binding fragments” and other interchangeable terms for similar binding fragments as described below. Native antibodies and native immunoglobulins (Igs) are generally heterotetrameric glycoproteins of about 150,000 Daltons, composed of two identical light chains and two identical heavy chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“V_{H}”) followed by a number of constant domains (“C_{H}”). Each light chain has a variable domain at one end (“V_{L}”) and a constant domain (“C_{L}”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

In some instances, an antibody or an antigen-binding fragment comprises an isolated antibody or antigen-binding fragment, a purified antibody or antigen-binding fragment, a recombinant antibody or antigen-binding fragment, a modified antibody or antigen-binding fragment, or a synthetic antibody or antigen-binding fragment.

Antibodies and antigen-binding fragments herein can be partly or wholly synthetically produced. An antibody or antigen-binding fragment can be a polypeptide or protein having a binding domain which can be, or can be homologous to, an antigen binding domain. In one instance, an antibody or an antigen-binding fragment can be produced in an appropriate in vivo animal model and then isolated and/or purified.

Depending on the amino acid sequence of the constant domain of its heavy chains, immunoglobulins (Igs) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. An Ig or portion thereof can, in some cases, be a human Ig. In some instances, a C_{H}3 domain can be from an immunoglobulin. In some cases, a chain or a part of an antibody or antigen binding fragment, a modified antibody or antigen-binding fragment, or a binding agent can be from an Ig. In such cases, an Ig can be IgG, an IgA, an IgD, an IgE, or an IgM, or is derived therefrom. In cases where the Ig is an IgG, it can be a subtype of IgG, wherein subtypes of IgG can include IgG1, an IgG2a, an IgG2b, an IgG3, and an IgG4. In some cases, a C_{H}3 domain can be from an immunoglobulin selected from the group consisting of an IgG, an IgA, an IgD, an IgE, and an IgM, or is derived therefrom. In some embodiments, a chain or a part of an antibody or antigen binding fragment described herein comprises an IgG or is derived therefrom. In some instances, a chain or a part of an antibody or antigen binding fragment comprises an IgG1 or is derived therefrom. In some instances, a chain or a part of an antibody or antigen binding fragment comprises an IgG4 or is derived therefrom. In some embodiments, a chain or a part of an antibody or antigen binding fragment described herein comprises an IgM, is derived therefrom, or is a monomeric form of IgM. In some embodiments, a chain or a part of an antibody or antigen binding fragment described herein comprises an IgE or is derived therefrom. In some embodiments, a chain or a part of an antibody or antigen binding fragment described herein comprises an IgD or is derived therefrom. In some embodiments, a chain or a part of an antibody or antigen binding fragment described herein comprises an IgA or is derived therefrom.

The “light chains” of antibodies (immunoglobulins) from any vertebrate species can be assigned to one of two clearly distinct types, called kappa (“κ” or “K”) or lambda (“λ”), based on the amino acid sequences of their constant domains.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al., Sequences of Proteins of Immunological Interest, (5th ed., 1991, National Institutes of Health, Bethesda Md.)); and (2) an approach based on crystallographic studies of antigen-antibody complexes (Al-Iazikani et al. (1997) J. Molec. Biol. 273:927-948)). As used herein, a CDR may refer to CDRs defined by either approach or by a combination of both approaches.

With respect to antibodies, the term “variable domain” refers to the variable domains of antibodies that are used in the binding and specificity of each particular antibody for its particular antigen. However, the variability is not evenly distributed throughout the variable domains of antibodies. Rather, it is concentrated in three segments called hypervariable regions (also known as CDRs) in both the light chain and the heavy chain variable domains. More highly conserved portions of variable domains are called the “framework regions” or “FRs.” The variable domains of unmodified heavy and light chains each contain four FRs (FR1, FR2, FR3, and FR4), largely adopting a β-sheet configuration interspersed with three CDRs which form loops connecting and, in some cases, part of the β-sheet structure. The CDRs in each chain are held together in close proximity by the FRs and, with the CDRs from the other chain, contribute to the formation of the antigen-binding site of antibodies (see, Kabat et al., Sequences of Proteins of Immunological Interest, 5th Ed. Public Health Service, National Institutes of Health, Bethesda, Md. (1991), pages 647-669).

The terms “hypervariable region” and “CDR” when used herein, refer to the amino acid residues of an antibody which are responsible for antigen-binding. The CDRs comprise amino acid residues from three sequence regions which bind in a complementary manner to an antigen and are known as CDR1, CDR2, and CDR3 for each of the V_{H} and V_{L} chains. In the light chain variable domain, the CDRs typically correspond to approximately residues 24-34 (CDRL1), 50-56 (CDRL2), and 89-97 (CDRL3), and in the heavy chain variable domain the CDRs typically correspond to approximately residues 31-35 (CDRH1), 50-65 (CDRH2), and 95-102 (CDRH3) according to Kabat et al., Id. It is understood that the CDRs of different antibodies may contain insertions, thus the amino acid numbering may differ. The Kabat numbering system accounts for such insertions with a numbering scheme that utilizes letters attached to specific residues (e.g., 27A, 27B, 27C, 27D, 27E, and 27F of CDRL1 in the light chain) to reflect any insertions in the numberings between different antibodies. Alternatively, in the light chain variable domain, the CDRs typically correspond to approximately residues 26-32 (CDRL1), 50-52 (CDRL2), and 91-96 (CDRL3), and in the heavy chain variable domain, the CDRs typically correspond to approximately residues 26-32 (CDRH1), 53-55 (CDRH2), and 96-101 (CDRH3) according to Chothia and Lesk (J. Mol. Biol., 196: 901-917 (1987)).

As used herein, “framework region,” “FW,” or “FR” refers to framework amino acid residues that form a part of the antigen binding pocket or groove. In some embodiments, the framework residues form a loop that is a part of the antigen binding pocket or groove and the amino acids residues in the loop may or may not contact the antigen. Framework regions generally comprise the regions between the CDRs. In the light chain variable domain, the FRs typically correspond to approximately residues 0-23 (FRL1), 35-49 (FRL2), 57-88 (FRL3), and 98-109 and in the heavy chain variable domain the FRs typically correspond to approximately residues 0-30 (FRH1), 36-49 (FRH2), 66-94 (FRH3), and 103-133 according to Kabat et al., Id. As discussed above with the Kabat numbering for the light chain, the heavy chain too accounts for insertions in a similar manner (e.g., 35A, 35B of CDRH1 in the heavy chain). Alternatively, in the light chain variable domain, the FRs typically correspond to approximately residues 0-25 (FRL1), 33-49 (FRL2) 53-90 (FRL3), and 97-109 (FRL4), and in the heavy chain variable domain, the FRs typically correspond to approximately residues 0-25 (FRH1), 33-52 (FRH2), 56-95 (FRH3), and 102-113 (FRH4) according to Chothia and Lesk, Id. The loop amino acids of a FR can be assessed and determined by inspection of the three-dimensional structure of an antibody heavy chain and/or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g., structural positions) are, generally, less diversified. The three-dimensional structure of the antibody variable domain can be derived from a crystal structure or protein modeling.

In the present disclosure, the following abbreviations (in the parentheses) are used in accordance with the customs, as necessary: heavy chain (H chain), light chain (L chain), heavy chain variable region (VH), light chain variable region (VL), complementarity determining region (CDR), first complementarity determining region (CDR1), second complementarity determining region (CDR2), third complementarity determining region (CDR3), heavy chain first complementarity determining region (VH CDR1), heavy chain second complementarity determining region (VH CDR2), heavy chain third complementarity determining region (VH CDR3), light chain first complementarity determining region (VL CDR1), light chain second complementarity determining region (VL CDR2), and light chain third complementarity determining region (VL CDR3).

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is generally defined to stretch from an amino acid residue at position Cys226, or from Pro230, to the carboxyl-terminus thereof. The numbering of the residues in the Fc region is that of the EU index as in Kabat et al. (Sequences of Proteins of Immunological Interest, 5th Ed. Public Health Service, National Institutes of Health, Bethesda, Md., 1991). The Fc region of an immunoglobulin generally comprises two constant domains, C_{H}2 and C_{H}3.

“Antibodies” useful in the present disclosure encompass, but are not limited to, monoclonal antibodies, polyclonal antibodies, chimeric antibodies, bispecific antibodies, multispecific antibodies, heteroconjugate antibodies, humanized antibodies, human antibodies, deimmunized antibodies, mutants thereof, fusions thereof, immunoconjugates thereof, antigen-binding fragments thereof, and/or any other modified configuration of the immunoglobulin molecule that comprises an antigen recognition site of the required specificity, including glycosylation variants of antibodies, amino acid sequence variants of antibodies, and covalently modified antibodies. In certain embodiments of the methods and conjugates provided herein, the antibody requires an Fc region to enable attachment of a linker between the antibody and the protein (e.g., attachment of the linker using an affinity peptide, such as in AJICAP™ technology).

In some instances, an antibody is a monoclonal antibody. As used herein, a “monoclonal antibody” refers to an antibody obtained from a population of substantially homogeneous antibodies, i.e., the individual antibodies comprising the population are identical except for possible naturally-occurring mutations that may be present in minor amounts. In contrast to polyclonal antibody preparations, which typically include different antibodies directed against different determinants (epitopes), each monoclonal antibody is directed against a single determinant on the antigen (epitope). The modifier “monoclonal” indicates the character of the antibody as being obtained from a substantially homogeneous population of antibodies and is not to be construed as requiring production of the antibody by any particular method.

In some instances, an antibody is a humanized antibody. As used herein, “humanized” antibodies refer to forms of non-human (e.g., murine) antibodies that are specific chimeric immunoglobulins, immunoglobulin chains, or fragments thereof that contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and biological activity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences but are included to further refine and optimize antibody performance. In general, a humanized antibody comprises substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in, for example, WO 99/58572. Other forms of humanized antibodies have one or more CDRs (one, two, three, four, five, or six) which are altered with respect to the original antibody, which are also termed one or more CDRs “derived from” one or more CDRs from the original antibody.

If needed, an antibody or an antigen binding fragment described herein can be assessed for immunogenicity and, as needed, be deimmunized (i.e., the antibody is made less immunoreactive by altering one or more T cell epitopes). As used herein, a “deimmunized antibody” means that one or more T cell epitopes in an antibody sequence have been modified such that a T cell response after administration of the antibody to a subject is reduced compared to an antibody that has not been deimmunized. Analysis of immunogenicity and T-cell epitopes present in the antibodies and antigen-binding fragments described herein can be carried out via the use of software and specific databases. Exemplary software and databases include iTope™ developed by Antitope of Cambridge, England. iTope™, is an in silico technology for analysis of peptide binding to human MHC class II alleles. The iTope™ software predicts peptide binding to human MHC class II alleles and thereby provides an initial screen for the location of such “potential T cell epitopes.” iTope™ software predicts favorable interactions between amino acid side chains of a peptide and specific binding pockets within the binding grooves of 34 human MHC class II alleles. The location of key binding residues is achieved by the in silico generation of 9mer peptides that overlap by one amino acid spanning the test antibody variable region sequence. Each 9mer peptide can be tested against each of the 34 MHC class II allotypes and scored based on their potential “fit” and interactions with the MHC class II binding groove. Peptides that produce a high mean binding score (>0.55 in the iTope™ scoring function) against >50% of the MHC class II alleles are considered as potential T cell epitopes. In such regions, the core 9 amino acid sequence for peptide binding within the MHC class II groove is analyzed to determine the MHC class II pocket residues (P1, P4, P6, P7, and P9) and the possible T cell receptor (TCR) contact residues (P-1, P2, P3, P5, P8). After identification of any T-cell epitopes, amino acid residue changes, substitutions, additions, and/or deletions can be introduced to remove the identified T-cell epitope. Such changes can be made so as to preserve antibody structure and function while still removing the identified epitope. Exemplary changes can include, but are not limited to, conservative amino acid changes.

An antibody can be a human antibody. As used herein, a “human antibody” means an antibody having an amino acid sequence corresponding to that of an antibody produced by a human and/or that has been made using any suitable technique for making human antibodies. This definition of a human antibody includes antibodies comprising at least one human heavy chain polypeptide or at least one human light chain polypeptide. One such example is an antibody comprising murine light chain and human heavy chain polypeptides. In one embodiment, the human antibody is selected from a phage library, where that phage library expresses human antibodies. Human antibodies can also be made by introducing human immunoglobulin loci into transgenic animals, e.g., mice in which the endogenous immunoglobulin genes have been partially or completely inactivated. Alternatively, the human antibody may be prepared by immortalizing human B lymphocytes that produce an antibody directed against a target antigen (such B lymphocytes may be recovered from an individual or may have been immunized in vitro).

Any of the antibodies herein can be bispecific. Bispecific antibodies are antibodies that have binding specificities for at least two different antigens and can be prepared using the antibodies disclosed herein. Traditionally, the recombinant production of bispecific antibodies was based on the coexpression of two immunoglobulin heavy chain-light chain pairs, with the two heavy chains having different specificities. Bispecific antibodies can be composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations.

According to one approach to making bispecific antibodies, antibody variable domains with the desired binding specificities (antibody-antigen combining sites) are fused to immunoglobulin constant domain sequences. The fusion can be with an immunoglobulin heavy chain constant domain, comprising at least part of the hinge, CH2 and CH3 regions. The first heavy chain constant region (CH1), containing the site necessary for light chain binding, can be present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In some instances, an antibody herein is a chimeric antibody. “Chimeric” forms of non-human (e.g., murine) antibodies include chimeric antibodies which contain minimal sequence derived from a non-human Ig. For the most part, chimeric antibodies are murine antibodies in which at least a portion of an immunoglobulin constant region (Fc), typically that of a human immunoglobulin, is inserted in place of the murine Fc. Chimeric or hybrid antibodies also may be prepared in vitro using suitable methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

Provided herein are antibodies and antigen-binding fragments thereof, modified antibodies and antigen-binding fragments thereof, and binding agents that specifically bind to one or more epitopes on one or more target antigens. In one instance, a binding agent selectively binds to an epitope on a single antigen. In another instance, a binding agent is bivalent and either selectively binds to two distinct epitopes on a single antigen or binds to two distinct epitopes on two distinct antigens. In another instance, a binding agent is multivalent (i.e., trivalent, quatravalent, etc.) and the binding agent binds to three or more distinct epitopes on a single antigen or binds to three or more distinct epitopes on two or more (multiple) antigens.

An antigen binding fragment of any of the antibodies herein are also contemplated. The terms “antigen-binding portion of an antibody,” “antigen-binding fragment,” “antigen-binding domain,” “antibody fragment,” or a “functional fragment of an antibody” are used interchangeably herein to refer to one or more fragments of an antibody that retain the ability to specifically bind to an antigen. Representative antigen-binding fragments include, but are not limited to, a Fab, a Fab′, a F(ab′)₂, a bispecific F(ab′)₂, a trispecific F(ab′)₂, a variable fragment (Fv), a single chain variable fragment (scFv), a dsFv, a bispecific scFv, a variable heavy domain, a variable light domain, a variable NAR domain, bispecific scFv, an AVIMER®, a minibody, a diabody, a bispecific diabody, triabody, a tetrabody, a minibody, a maxibody, a camelid, a VHH, a minibody, an intrabody, fusion proteins comprising an antibody portion (e.g., a domain antibody), a single chain binding polypeptide, a scFv-Fc, a Fab-Fc, a bispecific T cell engager (BiTE; two scFvs produced as a single polypeptide chain, where each scFv comprises an amino acid sequences a combination of CDRs or a combination of VL/VL described herein), a tetravalent tandem diabody (TandAb; an antibody fragment that is produced as a non-covalent homodimer folder in a head-to-tail arrangement, e.g., a TandAb comprising an scFv, where the scFv comprises an amino acid sequences a combination of CDRs or a combination of VL/VL described herein), a Dual-Affinity Re-targeting Antibody (DART; different scFvs joined by a stabilizing interchain disulphide bond), a bispecific antibody (bscAb; two single-chain Fv fragments joined via a glycine-serine linker), a single domain antibody (sdAb), a fusion protein, a bispecific disulfide-stabilized Fv antibody fragment (dsFv-dsFv′; two different disulfide-stabilized Fv antibody fragments connected by flexible linker peptides). In certain instances of the invention, a full length antibody (e.g., an antigen binding fragment and an Fc region) are preferred.

Heteroconjugate antibodies, comprising two covalently joined antibodies, are also within the scope of the disclosure. Suitable linkers may be used to multimerize binding agents. Non-limiting examples of linking peptides include, but are not limited to, (GS)ₙ (SEQ ID NO: 24), (GGS)ₙ (SEQ ID NO: 25), (GGGS)ₙ (SEQ ID NO: 26), (GGSG)ₙ (SEQ ID NO: 27), or (GGSGG)ₙ (SEQ ID NO: 28), (GGGGS)ₙ (SEQ ID NO: 29), wherein n is 1, 2, 3, 4, 5, 6, 7, 8, 9, or 10. For example, a linking peptide can be (GGGGS)₃ (SEQ ID NO: 30), which bridges approximately 3.5 nm between the carboxy terminus of one variable region and the amino terminus of the other variable region, or (GGGGS)₄ (SEQ ID NO: 31). Linkers of other sequences have been designed and used. Linkers can in turn be modified for additional functions, such as attachment of drugs or attachment to solid supports.

As used herein, the term “avidity” refers to the resistance of a complex of two or more agents to dissociation after dilution. Apparent affinities can be determined by methods such as an enzyme-linked immunosorbent assay (ELISA) or any other suitable technique. Avidities can be determined by methods such as a Scatchard analysis or any other suitable technique.

As used herein, the term “affinity” refers to the equilibrium constant for the reversible binding of two agents and is expressed as K_{D}. The binding affinity (K_{D}) of an antibody or antigen-binding fragment herein can be less than 500 nM, 475 nM, 450 nM, 425 nM, 400 nM, 375 nM, 350 nM, 325 nM, 300 nM, 275 nM, 250 nM, 225 nM, 200 nM, 175 nM, 150 nM, 125 nM, 100 nM, 90 nM, 80 nM, 70 nM, 50 nM, 50 nM, 49 nM, 48 nM, 47 nM, 46 nM, 45 nM, 44 nM, 43 nM, 42 nM, 41 nM, 40 nM, 39 nM, 38 nM, 37 nM, 36 nM, 35 nM, 34 nM, 33 nM, 32 nM, 31 nM, 30 nM, 29 nM, 28 nM, 27 nM, 26 nM, 25 nM, 24 nM, 23 nM, 22 nM, 21 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, 1 nM, 990 pM, 980 pM, 970 pM, 960 pM, 950 pM, 940 pM, 930 pM, 920 pM, 910 pM, 900 pM, 890 pM, 880 pM, 870 pM, 860 pM, 850 pM, 840 pM, 830 pM, 820 pM, 810 pM, 800 pM, 790 pM, 780 pM, 770 pM, 760 pM, 750 pM, 740 pM, 730 pM, 720 pM, 710 pM, 700 pM, 690 pM, 680 pM, 670 pM, 660 pM, 650 pM, 640 pM, 630 pM, 620 pM, 610 pM, 600 pM, 590 pM, 580 pM, 570 pM, 560 pM, 550 pM, 540 pM, 530 pM, 520 pM, 510 pM, 500 pM, 490 pM, 480 pM, 470 pM, 460 pM, 450 pM, 440 pM, 430 pM, 420 pM, 410 pM, 400 pM, 390 pM, 380 pM, 370 pM, 360 pM, 350 pM, 340 pM, 330 pM, 320 pM, 310 pM, 300 pM, 290 pM, 280 pM, 270 pM, 260 pM, 250 pM, 240 pM, 230 pM, 220 pM, 210 pM, 200 pM, 190 pM, 180 pM, 170 pM, or any integer therebetween. Binding affinity may be determined using surface plasmon resonance (SPR), KINEXA® Biosensor, scintillation proximity assays, enzyme linked immunosorbent assay (ELISA), ORIGEN immunoassay (IGEN), fluorescence quenching, fluorescence transfer, yeast display, or any combination thereof. Binding affinity may also be screened using a suitable bioassay.

As used herein, the term “avidity” refers to the resistance of a complex of two or more agents to dissociation after dilution. Apparent affinities can be determined by methods such as an enzyme linked immunosorbent assay (ELISA) or any other technique familiar to one of skill in the art. Avidities can be determined by methods such as a Scatchard analysis or any other technique familiar to one of skill in the art.

Also provided herein are affinity matured antibodies. The following methods may be used for adjusting the affinity of an antibody and for characterizing a CDR. One way of characterizing a CDR of an antibody and/or altering (such as improving) the binding affinity of a polypeptide, such as an antibody, is termed “library scanning mutagenesis.” Generally, library scanning mutagenesis works as follows. One or more amino acid position in the CDR is replaced with two or more (such as 3, 4, 5, 6, 7, 8, 9, 10, 11, 12, 13, 14, 15, 16, 17, 18, 19, or 20) amino acids. This generates small libraries of clones (in some embodiments, one for every amino acid position that is analyzed), each with a complexity of two or more members (if two or more amino acids are substituted at every position). Generally, the library also includes a clone comprising the native (unsubstituted) amino acid. A small number of clones, for example, about 20-80 clones (depending on the complexity of the library), from each library can be screened for binding specificity or affinity to the target polypeptide (or other binding target), and candidates with increased, the same, decreased, or no binding are identified. Binding affinity may be determined using Biacore surface plasmon resonance analysis, which detects differences in binding affinity of about 2-fold or greater.

In some instances, an antibody or antigen binding fragment is bi-specific or multi-specific and can specifically bind to more than one antigen. In some cases, such a bi-specific or multi-specific antibody or antigen binding fragment can specifically bind to 2 or more different antigens. In some cases, a bi-specific antibody or antigen-binding fragment can be a bivalent antibody or antigen-binding fragment. In some cases, a multi specific antibody or antigen-binding fragment can be a bivalent antibody or antigen-binding fragment, a trivalent antibody or antigen-binding fragment, or a quatravalent antibody or antigen-binding fragment.

An antibody or antigen binding fragment described herein can be isolated, purified, recombinant, or synthetic.

The antibodies described herein may be made by any suitable method. Antibodies can often be produced in large quantities, particularly when utilizing high level expression vectors.

Representative examples of antibodies or antigen-binding fragments include, but are not limited to, those which selectively bind to a cancer antigen, an immune cell target molecule, a self-antigen, or a combination thereof.

Cancer antigens include, but are not limited to, programmed cell death 1 (PD1) programmed cell death ligand 1 (PDL1), CD5, CD20, CD19, CD22, CD30, CD33, CD40, CD44, CD52, CD74, CD103, CD137, CD123, CD152, a carcinoembryonic antigen (CEA), an integrin, an epidermal growth factor (EGF) receptor family member, a vascular epidermal growth factor (VEGF), a proteoglycan, a disialoganglioside, B7-H3, cancer antigen 125 (CA-125), epithelial cell adhesion molecule (EpCAM), vascular endothelial growth factor receptor 1, vascular endothelial growth factor receptor 2, a tumor associated glycoprotein, mucin 1 (MUC1), a tumor necrosis factor receptor, an insulin-like growth factor receptor, folate receptor α, transmembrane glycoprotein NMB, a C—C chemokine receptor, prostate specific membrane antigen (PSMA), recepteur d'origine nantais (RON) receptor, cytotoxic T-lymphocyte antigen 4 (CTLA4), Colon cancer antigen 19.9, gastric cancer mucin antigen 4.2, colorectal carcinoma antigen A33, ADAM-9, AFP oncofetal antigen-alpha-fetoprotein, ALCAM, BAGE, beta-catenin, Carboxypeptidase M, B1, CD23, CD25, CD27, CD28, CD36, CD45, CD46, CD52, CD56, CD79a/CD79b, CD317, CDK4, CO-43 (blood group Le^{b}), CO-514 (blood group Le^{a}), CTLA-1, Cytokeratin 8, DR5, E1 series (blood group B), Ephrin receptor A2 (EphA2), Erb (ErbB1, ErbB3, ErbB4), lung adenocarcinoma antigen F3, antigen FC10.2, GAGE-1, GAGE-2, GD2/GD3/GD49/GM2/GM3, GICA 19-9, gp37, gp75, gp100, HER-2/neu, human milk fat globule antigen, human papillomavirus-E6/human papillomavirus-E7, high molecular weight melanoma antigen (HMW-MAA), differentiation antigen (I antigen), I(Ma) as found in gastric adenocarcinomas, Integrin Alpha-V-Beta-6, Integrinβ6 (ITGβ6), Interleukin-13 Receptor α2 (IL13Rα2), JAM-3, KID3, KID31, KS 1/4 pan-carcinoma antigen, KSA (17-1A), human lung carcinoma antigen L6, human lung carcinoma antigen L20, LEA, LUCA-2, M1:22:25:8, M18, M39, MAGE-1, MAGE-3, MART, Myl, MUM-1, N-acetylglucosaminyltransferase, neoglycoprotein, NS-10, OFA-1 and OFA-2, Oncostatin M (Oncostatin Receptor Beta), rho15, prostate specific antigen (PSA), PSMA, polymorphic epithelial mucin antigen (PEMA), PIPA, prostatic acid phosphate, R24, ROR1, SSEA-1, SSEA-3, SSEA-4, sTn, T cell receptor derived peptide, T5A7, Tissue Antigen 37, TAG-72, TL5 (blood group A), a TNF-α receptor (TNFαR), TNFβR, TNFγR, TRA-1-85 (blood group H), Transferrin Receptor, TSTA tumor-specific transplantation antigen, VEGF-R, Y hapten, Le^{y}, 5T4, or a combination thereof.

Immune cell target molecules include, but are not limited to, PD-1, PD-L1, PD-L2, CTLA-4, CD28, B7-1 (CD80), B7-2 (CD86), ICOS ligand, ICOS, B7-H3, B7-H4, VISTA, B7-H7 (HHLA2), TMIGD2, 4-1BBL, 4-1BB, HVEM, BTLA, CD160, LIGHT, MHC Class I, MHC Class II, LAG3, OX40L, OX40, CD70, CD27, CD40, CD40L, GITRL, GITR, CD155, DNAM-1, TIGIT, CD96, CD48, 2B4, Galectin-9, TIM-3, Adenosine, Adenosine A2a Receptor, CEACAM1, CD47, SIRP alpha, BTN2A1, DC-SIGN, CD200, CD200R, TL1A, DR3, or a combination thereof.

Self-antigens include, but are not limited to, tumor necrosis factor alpha (TNFα), myelin basic protein (MBP), myelin oligodendrocyte glycoprotein (MOG), proteolipid protein (PLP), type II collagen (CII), vimentin, α-enolase, a clusterin, a histone, peptidyl arginine deiminase-4, transglutaminase 2 (TG2, TGM2), CD318, Peptidoglycan Recognition Protein 1 (PGLYRP1), or a combination thereof.

### Representative Antibodies and Antigen Binding Fragments

An antibody or antigen binding fragment described herein can selectively bind to a therapeutically relevant antigen such as, for example, a cancer antigen, an immune cell target molecule, a self-antigen, or any combination thereof. In one non-limiting aspect, provided herein for use in a conjugate are antibodies or antigen binding fragments that selectively bind to, for example, PD1, PD-L1, CD20, or TNFα.

In one embodiment, an antibody or antigen binding fragment selectively binds to a human PD1, wherein human PD1 has an amino acid sequence of

(SEQ ID NO: 32)
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET.

In another embodiment, an antibody or antigen binding fragment selectively binds to a human PD-L1, wherein human PD-L1 has an amino acid sequence of

(SEQ ID NO: 33)
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET.

In another embodiment, an antibody or antigen binding fragment selectively binds to a human CD20, wherein human CD20 has an amino acid sequence of

(SEQ ID NO: 34)
MTTPRNSVNGTFPAEPMKGPIAMQSGPKPLFRRMSSLVGPTQSFFMRESK
TLGAVQIMNGLFHIALGGLLMIPAGIYAPICVTVWYPLWGGIMYIISGSL
LAATEKNSRKCLVKGKMIMNSLSLFAAISGMILSIMDILNIKISHFLKME
SLNFIRAHTPYINIYNCEPANPSEKNSPSTQYCYSIQSLFLGILSVMLIF
AFFQELVIAGIVENEWKRTCSRPKSNIVLLSAEEKKEQTIEIKEEVVGLT
ETSSQPKNEEDIEIIPIQEEEEEETETNFPEPPQDQESSPIENDSSP.

In another embodiment, an antibody or antigen binding fragment selectively binds to a human TNF-alpha. A human TNFα transmembrane protein can have an amino acid sequence of MSTESMIRDVELAEEALPKKTGGPQGSRRCLFLSLFSFLIVAGATTLFCLLHFGVIGPQRE EFPRDLSLISPLAQAVRSSSRTPSDKPVAHVVANPQAEGQLQWLNRRANALLANGVELR DNQLVVPSEGLYLIYSQVLFKGQGCPSTHVLLTHTISRIAVSYQTKVNLLSAIKSPCQRET PEGAEAKPWYEPIYLGGVFQLEKGDRLSAEINRPDYLDFAESGQVYFGIIAL (SEQ ID NO: 35). A recombinant, mature human TNFα can have an amino acid sequence of

(SEQ ID NO: 36)
VRSSSRTPSDKPVAHVVANPQAEGQLQWLNRRANALLANGVELRDNQLVV
PSEGLYLIYSQVLFKGQGCPSTHVLLTHTISRIAVSYQTKVNLLSAIKSP
CQRETPEGAEAKPWYEPIYLGGVFQLEKGDRLSAEINRPDYLDFAESGQV
YFGIIAL.

### Anti-PD-1 Antibodies

In one embodiment, an anti-PD1 antibody or an anti-PD1 antigen binding fragment of the disclosure comprises a combination of a heavy chain variable region (VH) and a light chain variable region (VL) described herein. In another embodiment, an anti-PD1 antibody or an anti-PD1 antigen binding fragment of the disclosure comprises a combination of complementarity determining regions (VH CDR1, VH CDR2, VH CDR3, VL CDR1, VL CDR2, and VL CDR3) described herein. In one embodiment, an anti-PD-1 antibody or an anti-PD-1 antigen binding fragment of the disclosure comprises a modified Tislelizumab, Baizean, 0KVO411B3N, BGB-A317, hu317-1/IgG4mt2, Sintilimab, Tyvyt, IBI-308, Toripalimab, TeRuiPuLi, Terepril, Tuoyi, JS-001, TAB-001, Camrelizumab, HR-301210, INCSHR-01210, SHR-1210, Cemiplimab, Cemiplimab-rwlc, LIBTAYO®, 6QVL057INT, H4H7798N, REGN-2810, SAR-439684, Lambrolizumab, Pembrolizumab, KEYTRUDA®, MK-3475, SCH-900475, h409A11, Nivolumab, Nivolumab BMS, OPDIVO®, BMS-936558, MDX-1106, ONO-4538, Prolgolimab, Forteca, BCD-100, Penpulimab, AK-105, Zimberelimab, AB-122, GLS-010, WBP-3055, Balstilimab, 1Q2QT5M7EO, AGEN-2034, AGEN-2034w, Genolimzumab, Geptanolimab, APL-501, CBT-501, GB-226, Dostarlimab, ANB-011, GSK-4057190A, P0GVQ9A4S5, TSR-042, WBP-285, Serplulimab, HLX-10, CS-1003, Retifanlimab, 2Y3T5IF01Z, INCMGA-00012, INCMGA-0012, MGA-012, Sasanlimab, LZZ0IC2EWP, PF-06801591, RN-888, Spartalizumab, NVP-LZV-184, PDR-001, QOG25L6Z8Z, Relatlimab/nivolumab, BMS-986213, Cetrelimab, JNJ-3283, JNJ-63723283, LYK98WP91F, Tebotelimab, MGD-013, BCD-217, BAT-1306, HX-008, MEDI-5752, JTX-4014, Cadonilimab, AK-104, BI-754091, Pidilizumab, CT-011, MDV-9300, YBL-006, AMG-256, RG-6279, RO-7284755, BH-2950, IBI-315, RG-6139, RO-7247669, ONO-4685, AK-112, 609-A, LY-3434172, T-3011, MAX-10181, AMG-404, IBI-318, MGD-019, INCB-086550, ONCR-177, LY-3462817, RG-7769, RO-7121661, F-520, XmAb-23104, Pd-1-pik, SG-001, S-95016, Sym-021, LZM-009, Budigalimab, 6VDO4TY3OO, ABBV-181, PR-1648817, CC-90006, XmAb-20717, 2661380, AMP-224, B7-DCIg, EMB-02, ANB-030, PRS-332, [89Zr]Deferoxamide-pembrolizumab, 89Zr-Df-Pembrolizumab, [89Zr]Df-Pembrolizumab, STI-1110, STI-A1110, CX-188, mPD-1 Pb-Tx, MCLA-134, 244C8, ENUM 224C8, ENUM C8, 388D4, ENUM 388D4, ENUM D4, MEDI0680, or AMP-514. In some embodiments, the anti-PD-1 polypeptide is modified pembrolizumab. In some embodiments, the anti-PD-1 polypeptide is modified with mAB3. In some embodiments, the anti-PD-1 polypeptide is modified with mAB4.

Table 1A below provides amino acid sequences of exemplary anti-PD-1 polypeptides and anti-PD-1 antigen binding fragments that can be modified to prepare anti-PD-1 immunoconjugates. TABLE 1A also shows provides combinations of CDRs that can be utilized in a modified anti-PD-1 immunoconjugate. Reference to an anti-PD-1 polypeptide herein may alternatively refer to an anti-PD-1 antigen binding fragment.

TABLE 1A

| Antibody or | | |
|---|---|---|
| Ag-binding | | SEQ ID |
| fragment | Sequence | NO |
| Tislelizumab, | QVQLQESGPGLVKPSETLSLTCTVSGFSLTSYGVHWIRQPPGKGL | 37 |
| Baizean, | EWIGVIYADGSTNYNPSLKSRVTISKDTSKNQVSLKLSSVTAADT | |
| 0KVO411B3N, | AVYYCARAYGNYWYIDVWGQGTTVTVSSASTKGPSVFPLAPCS | |
| BGB-A317, | RSTSESTAALGCLVKDYFPEPVTVSWNSGALTSGVHTFPAVLQSS | |
| hu317- | GLYSLSSVVTVPSSSLGTKTYTCNVDHKPSNTKVDKRVESKYGP | |
| 1/IgG4mt2 | PCPPCPAPPVAGGPSVFLFPPKPKDTLMISRTPEVTCVVVAVSQE | |
| VH | DPEVQFNWYVDGVEVHNAKTKPREEQFNSTYRVVSVLTVVHQD | |
| | WLNGKEYKCKVSNKGLPSSIEKTISKAKGQPREPQVYTLPPSQEE | |
| | MTKNQVSLTCLVKGFYPSDIAVEWESNGQPENNYKTTPPVLDSD | |
| | GSFFLYSKLTVDKSRWQEGNVFSCSVMHEALHNHYTQKSLSLSL | |
| | GK | |
| Tislelizumab, | DIVMTQSPDSLAVSLGERATINCKSSESVSNDVAWYQQKPGQPP | 38 |
| Baizean, | KLLINYAFHRFTGVPDRFSGSGYGTDFTLTISSLQAEDVAVYYCH | |
| 0KVO411B3N, | QAYSSPYTFGQGTKLEIKRTVAAPSVFIFPPSDEQLKSGTASVVCL | |
| BGB-A317, | LNNFYPREAKVQWKVDNALQSGNSQESVTEQDSKDSTYSLSSTL | |
| hu317- | TLSKADYEKHKVYACEVTHQGLSSPVTKSFNRGEC | |
| 1/IgG4mt2 | | |
| VL | | |
| Sintilimab, | QVQLVQSGAEVKKPGSSVKVSCKASGGTFSSYAISWVRQAPGQ | 39 |
| Tyvyt, IBI-308 | GLEWMGLIIPMFDTAGYAQKFQGRVAITVDESTSTAYMELSSLR | |
| VH | SEDTAVYYCARAEHSSTGTFDYWGQGTLVTVSSASTKGPSVFPL | |
| | APCSRSTSESTAALGCLVKDYFPEPVTVSWNSGALTSGVHTFPAV | |
| | LQSSGLYSLSSVVTVPSSSLGTKTYTCNVDHKPSNTKVDKRVESK | |
| | YGPPCPPCPAPEFLGGPSVFLFPPKPKDTLMISRTPEVTCVVVDVS | |
| | QEDPEVQFNWYVDGVEVHNAKTKPREEQFNSTYRVVSVLTVLH | |
| | QDWLNGKEYKCKVSNKGLPSSIEKTISKAKGQPREPQVYTLPPSQ | |
| | EEMTKNQVSLTCLVKGFYPSDIAVEWESNGQPENNYKTTPPVLD | |
| | SDGSFFLYSRLTVDKSRWQEGNVFSCSVMHEALHNHYTQKSLSL | |
| | SLGK | |
| Sintilimab, | DIQMTQSPSSVSASVGDRVTITCRASQGISSWLAWYQQKPGKAP | 40 |
| Tyvyt, IBI-308 | KLLISAASSLQSGVPSRFSGSGSGTDFTLTISSLQPEDFATYYCQQ | |
| VL | ANHLPFTFGGGTKVEIKRTVAAPSVFIFPPSDEQLKSGTASVVCLL | |
| | NNFYPREAKVQWKVDNALQSGNSQESVTEQDSKDSTYSLSSTLT | |
| | LSKADYEKHKVYACEVTHQGLSSPVTKSFNRGEC | |
| Toripalimab, | QGQLVQSGAEVKKPGASVKVSCKASGYTFTDYEMHWVRQAPIH | 41 |
| TeRuiPuLi, | GLEWIGVIESETGGTAYNQKFKGRVTITADKSTSTAYMELSSLRS | |
| Terepril, Tuoyi, | EDTAVYYCAREGITTVATTYYWYFDVWGQGTTVTVSSASTKGP | |
| JS-001, TAB- | SVFPLAPCSRSTSESTAALGCLVKDYFPEPVTVSWNSGALTSGVH | |
| 001 | TFPAVLQSSGLYSLSSVVTVPSSSLGTKTYTCNVDHKPSNTKVDK | |
| VH | RVESKYGPPCPPCPAPEFLGGPSVFLFPPKPKDTLMISRTPEVTCV | |
| | VVDVSQEDPEVQFNWYVDGVEVHNAKTKPREEQFNSTYRVVSV | |
| | LTVLHQDWLNGKEYKCKVSNKGLPSSIEKTISKAKGQPREPQVY | |
| | TLPPSQEEMTKNQVSLTCLVKGFYPSDIAVEWESNGQPENNYKT | |
| | TPPVLDSDGSFFLYSRLTVDKSRWQEGNVFSCSVMHEALHNHYT | |
| | QKSLSLSLGK | |
| Toripalimab, | DVVMTQSPLSLPVTLGQPASISCRSSQSIVHSNGNTYLEWYLQKP | 42 |
| TeRuiPuLi, | GQSPQLLIYKVSNRFSGVPDRFSGSGSGTDFTLKISRVEAEDVGV | |
| Terepril, Tuoyi, | YYCFQGSHVPLTFGQGTKLEIKRTVAAPSVFIFPPSDEQLKSGTAS | |
| JS-001, TAB- | VVCLLNNFYPREAKVQWKVDNALQSGNSQESVTEQDSKDSTYS | |
| 001 | LSSTLTLSKADYEKHKVYACEVTHQGLSSPVTKSFNRGEC | |
| VL | | |
| Camrelizumab, | EVQLVESGGGLVQPGGSLRLSCAASGFTFSSYMMSWVRQAPGK | 43 |
| HR-301210, | GLEWVATISGGGANTYYPDSVKGRFTISRDNAKNSLYLQMNSLR | |
| INCSHR-01210, | AEDTAVYYCARQLYYFDYWGQGTTVTVSSASTKGPSVFPLAPCS | |
| SHR-1210 | RSTSESTAALGCLVKDYFPEPVTVSWNSGALTSGVHTFPAVLQSS | |
| VH | GLYSLSSVVTVPSSSLGTKTYTCNVDHKPSNTKVDKRVESKYGP | |
| | PCPPCPAPEFLGGPSVFLFPPKPKDTLMISRTPEVTCVVVDVSQED | |
| | PEVQFNWYVDGVEVHNAKTKPREEQFNSTYRVVSVLTVLHQD | |
| | WLNGKEYKCKVSNKGLPSSIEKTISKAKGQPREPQVYTLPPSQEE | |
| | MTKNQVSLTCLVKGFYPSDIAVEWESNGQPENNYKTTPPVLDSD | |
| | GSFFLYSRLTVDKSRWQEGNVFSCSVMHEALHNHYTQKSLSLSL | |
| | GK | |
| Camrelizumab, | DIQMTQSPSSLSASVGDRVTITCLASQTIGTWLTWYQQKPGKAP | 44 |
| HR-301210, | KLLIYTATSLADGVPSRFSGSGSGTDFTLTISSLQPEDFATYYCQQ | |
| INCSHR-01210, | VYSIPWTFGGGTKVEIKRTVAAPSVFIFPPSDEQLKSGTASVVCLL | |
| SHR-1210 | NNFYPREAKVQWKVDNALQSGNSQESVTEQDSKDSTYSLSSTLT | |
| VL | LSKADYEKHKVYACEVTHQGLSSPVTKSFNRGEC | |
| Cemiplimab, | EVQLLESGGVLVQPGGSLRLSCAASGFTFSNFGMTWVRQAPGK | 45 |
| Cemiplimab- | GLEWVSGISGGGRDTYFADSVKGRFTISRDNSKNTLYLQMNSLK | |
| rwlc, | GEDTAVYYCVKWGNIYFDYWGQGTLVTVSSASTKGPSVFPLAP | |
| LIBTAYO ®, | CSRSTSESTAALGCLVKDYFPEPVTVSWNSGALTSGVHTFPAVL | |
| 6QVL057INT, | QSSGLYSLSSVVTVPSSSLGTKTYTCNVDHKPSNTKVDKRVESK | |
| H4H7798N, | YGPPCPPCPAPEFLGGPSVFLFPPKPKDTLMISRTPEVTCVVVDVS | |
| REGN-2810, | QEDPEVQFNWYVDGVEVHNAKTKPREEQFNSTYRVVSVLTVLH | |
| SAR-439684 | QDWLNGKEYKCKVSNKGLPSSIEKTISKAKGQPREPQVYTLPPSQ | |
| VH | EEMTKNQVSLTCLVKGFYPSDIAVEWESNGQPENNYKTTPPVLD | |
| | SDGSFFLYSRLTVDKSRWQEGNVFSCSVMHEALHNHYTQKSLSL | |
| | SLGK | |
| Cemiplimab, | DIQMTQSPSSLSASVGDSITITCRASLSINTFLNWYQQKPGKAPNL | 46 |
| Cemiplimab- | LIYAASSLHGGVPSRFSGSGSGTDFTLTIRTLQPEDFATYYCQQSS | |
| rwlc, | NTPFTFGPGTVVDFRRTVAAPSVFIFPPSDEQLKSGTASVVCLLN | |
| LIBTAYO ®, | NFYPREAKVQWKVDNALQSGNSQESVTEQDSKDSTYSLSSTLTL | |
| 6QVL057INT, | SKADYEKHKVYACEVTHQGLSSPVTKSFNRGEC | |
| H4H7798N, | (Disulfide bridge) | |
| REGN-2810, | H22-H96, H131-L214, H144-H200, H223-H′223, | |
| SAR-439684 | H226-H′226, H258-H318, H364-H422, H′22-H′96, H131- | |
| VL | L′214, H114-H′200, H′258-H′318, H364-H422, L23-L88, | |
| | L134-L194, L′23-L′88, L′134-L′194) | |
| Lambrolizumab, | QVQLVQSGVEVKKPGASVKVSCKASGYTFTNYYMYWVRQAPG | 51 |
| Pembrolizumab, | QGLEWMGGINPSNGGTNFNEKFKNRVTLTTDSSTTTAYMELKSL | |
| KEYTRUDA ®, | QFDDTAVYYCARRDYRFDMGFDYWGQGTTVTVSSASTKGPSVF | |
| MK-3475, SCH- | PLAPCSRSTSESTAALGCLVKDYFPEPVTVSWNSGALTSGVHTFP | |
| 900475, | AVLQSSGLYSLSSVVTVPSSSLGTKTYTCNVDHKPSNTKVDKRV | |
| h409All | ESKYGPPCPPCPAPEFLGGPSVFLFPPKPKDTLMISRTPEVTCVVV | |
| VH | DVSQEDPEVQFNWYVDGVEVHNAKTKPREEQFNSTYRVVSVLT | |
| | VLHQDWLNGKEYKCKVSNKGLPSSIEKTISKAKGQPREPQVYTL | |
| | PPSQEEMTKNQVSLTCLVKGFYPSDIAVEWESNGQPENNYKTTP | |
| | PVLDSDGSFFLYSRLTVDKSRWQEGNVFSCSVMHEALHNHYTQ | |
| | KSLSLSLGK | |
| Lambrolizumab, | EIVLTQSPATLSLSPGERATLSCRASKGVSTSGYSYLHWYQQKPG | 52 |
| Pembrolizumab, | QAPRLLIYLASYLESGVPARFSGSGSGTDFTLTISSLEPEDFAVYY | |
| KEYTRUDA ®, | CQHSRDLPLTFGGGTKVEIKRTVAAPSVFIFPPSDEQLKSGTASVV | |
| MK-3475, SCH- | CLLNNFYPREAKVQWKVDNALQSGNSQESVTEQDSKDSTYSLSS | |
| 900475, | TLTLSKADYEKHKVYACEVTHQGLSSPVTKSFNRGEC | |
| h409A11 | | |
| VL | | |
| Lambrolizumab, | QVQLVQSGVEVKKPGASVKVSCKASGYTFTNYYMYWVRQAPG | 53 |
| Pembrolizumab, | QGLEWMGGFPSNGGTNFNEKFKNRVTLTTDSSTTTAYMELKSL | |
| KEYTRUDA®, | QFDDTAVYYCARRDYRFDMGFDYWGQGTTVTVSS | |
| MK-3475, SCH- | (Disulfide bridge) | |
| 900475, | H22-H96, H134-L218, H147-H203, H226-H′226, H229- | |
| h409A11 | H′229, H261-H321, H367-H425, H′22-H′96, H134- | |
| VH | L′218, H147-H203, H261-H321, H′367-H′425, L23-L92, | |
| | L138-L198, L′23-L′92, L′138-L′198) | |
| Lambrolizumab, | EIVLTQSPATLSLSPGERATLSCRASKGVSTSGYSYLHWYQQKPG | 54 |
| Pembrolizumab, | QAPRLLIYLASYLESGVPARFSGSGSGTDFTLTISSLEPEDFAVYY | |
| KEYTRUDA ®, | CQHSRDLPLTFGGGTKVEIK | |
| MK-3475, SCH- | | |
| 900475, | | |
| h409A11 | | |
| VL | | |
| Nivolumab, | QVQLVESGGGVVQPGRSLRLDCKASGITFSNSGMHWVRQAPGK | 55 |
| Nivolumab | GLEWVAVIWYDGSKRYYADSVKGRFTISRDNSKNTLFLQMNSL | |
| BMS, | RAEDTAVYYC ATNDDYWGQGTLVTVS S | |
| OPDIVO ®, | | |
| BMS-936558, | | |
| MDX-1106, | | |
| ONO-4538 | | |
| VH | | |
| Nivolumab, | EIVLTQSPATLSLSPGERATLSCRASQSVSSYLAWYQQKPGQAPR | 56 |
| Nivolumab | LLIYDASNRATGIPARFSGSGSGTDFTLTISSLEPEDFAVYYCQQS | |
| BMS, | SNWPRTFGQGTKVEIK | |
| OPDIVO ®, | (Disulfide bridge) | |
| BMS-936558, | H22-H96, H127-L214, H140-H196, H219-H′219, H222- | |
| MDX-1106, | H′222, H254-H314, H360-H418, H′22-H′96, H′127- | |
| ONO-4538 | L′214, H′140-H′196, H′254-H′314, H′360-H′418, | |
| VL | L-23-L88, L134-L194, L′23-L′88, L134-L194) | |
| Prolgolimab, | QVQLVQSGGGLVQPGGSLRLSCAASGFTFSSYWMYWVRQVPGK | 57 |
| Forteca, BCD- | GLEWVSAIDTGGGRTYYADSVKGRFAISRVNAKNTMYLQMNSL | |
| 100 | RAEDTAVYYCARDEGGGTGWGVLKDWPYGLDAWGQGTLVTV | |
| VH | SSASTKGPSVFPLAPSSKSTSGGTAALGCLVKDYFPEPVTVSWNS | |
| | GALTSGVHTFPAVLQSSGLYSLSSVVTVPSSSLGTQTYICNVNHK | |
| | PSNTKVDKRVEPKSCDKTHTCPPCPAPEAAGGPSVFLFPPKPKDT | |
| | LMISRTPEVTCVVVDVSHEDPEVKFNWYVDGVEVHNAKTKPRE | |
| | EQYNSTYRVVSVLTVLHQDWLNGKEYKCKVSNKALPAPIEKTIS | |
| | KAKGQPREPQVYTLPPSREEMTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPGK | |
| Prolgolimab, | QPVLTQPLSVSVALGQTARITCGGNNIGSKNVHWYQQKPGQAPV | 58 |
| Forteca, BCD- | LVIYRDSNRPSGIPERFSGSNSGNTATLTISRAQAGDEADYYCQV | |
| 100 | WDSSTAVFGTGTKLTVLQRTVAAPSVFIFPPSDEQLKSGTASVVC | |
| VL | LLNNFYPREAKVQWKVDNALQSGNSQESVTEQDSKDSTYSLSST | |
| | LTLSKADYEKHKVYACEVTHQGLSSPVTKSFNRGEC | |
| Balstilimab, | QVQLVESGGGVVQPGRSLRLSCAASGFTFSSYGMHWVRQAPGK | 59 |
| 1Q2QT5M7EO, | GLEWVAVIWYDGSNKYYADSVKGRFTISRDNSKNTLYLQMNSL | |
| AGEN-2034, | RAEDTAVYYCASNGDHWGQGTLVTVSSASTKGPSVFPLAPCSRS | |
| AGEN-2034w | TSESTAALGCLVKDYFPEPVTVSWNSGALTSGVHTFPAVLQSSG | |
| VH | LYSLSSVVTVPSSSLGTKTYTCNVDHKPSNTKVDKRVESKYGPP | |
| | CPPCPAPEFLGGPSVFLFPPKPKDTLMISRTPEVTCVVVDVSQEDP | |
| | EVQFNWYVDGVEVHNAKTKPREEQFNSTYRVVSVLTVLHQDW | |
| | LNGKEYKCKVSNKGLPSSIEKTISKAKGQPREPQVYTLPPSQEEM | |
| | TKNQVSLTCLVKGFYPSDIAVEWESNGQPENNYKTTPPVLDSDG | |
| | SFFLYSRLTVDKSRWQEGNVFSCSVMHEALHNHYTQKSLSLSLG | |
| Balstilimab, | EIVMTQSPATLSVSPGERATLSCRASQSVSSNLAWYQQKPGQAP | 60 |
| 1Q2QT5M7EO, | RLLIYGASTRATGIPARFSGSGSGTEFTLTISSLQSEDFAVYYCQQ | |
| AGEN-2034, | YNNWPRTFGQGTKVEIKRTVAAPSVFIFPPSDEQLKSGTASVVCL | |
| AGEN-2034w | LNNFYPREAKVQWKVDNALQSGNSQESVTEQDSKDSTYSLSSTL | |
| VL | TLSKADYEKHKVYACEVTHQGLSSPVTKSFNRGEC | |
| Dostarlimab, | EVQLLESGGGLVQPGGSLRLSCAASGFTFSSYDMSWVRQAPGKG | 61 |
| ANB-011, | LEWVSTISGGGSYTYYQDSVKGRFTISRDNSKNTLYLQMNSLRA | |
| GSK- | EDTAVYYCASPYYAMDYWGQGTTVTVSSASTKGPSVFPLAPCS | |
| 4057190A, | RSTSESTAALGCLVKDYFPEPVTVSWNSGALTSGVHTFPAVLQSS | |
| P0GVQ9A4S5, | GLYSLSSVVTVPSSSLGTKTYTCNVDHKPSNTKVDKRVESKYGP | |
| TSR-042, WBP- | PCPPCPAPEFLGGPSVFLFPPKPKDTLMISRTPEVTCVVVDVSQED | |
| 285 | PEVQFNWYVDGVEVHNAKTKPREEQFNSTYRVVSVLTVLHQD | |
| VH | WLNGKEYKCKVSNKGLPSSIEKTISKAKGQPREPQVYTLPPSQEE | |
| | MTKNQVSLTCLVKGFYPSDIAVEWESNGQPENNYKTTPPVLDSD | |
| | GSFFLYSRLTVDKSRWQEGNVFSCSVMHEALHNHYTQKSLSLSL | |
| | GK | |
| Dostarlimab, | DIQLTQSPSFLSAYVGDRVTITCKASQDVGTAVAWYQQKPGKAP | 62 |
| ANB-011, | KLLIYWASTLHTGVPSRFSGSGSGTEFTLTISSLQPEDFATYYCQH | |
| GSK- | YSSYPWTFGQGTKLEIKRTVAAPSVFIFPPSDEQLKSGTASVVCLL | |
| 4057190A, | NNFYPREAKVQWKVDNALQSGNSQESVTEQDSKDSTYSLSSTLT | |
| P0GVQ9A4S5, | LSKADYEKHKVYACEVTHQGLSSPVTKSFNRGEC | |
| TSR-042, WBP- | | |
| 285 | | |
| VL | | |
| Serplulimab, | OVQLVESGGGLVKPGGSLRLSCAASGFTFSNYGMSWIROAPGKG | 63 |
| HLX-10 | LEWVSTISGGGSNIYYADSVKGRFTISRDNAKNSLYLQMNSLRA | |
| VH | EDTAVYYCVSYYYGIDFWGQGTSVTVSSASTKGPSVFPLAPCSR | |
| | STSESTAALGCLVKDYFPEPVTVSWNSGALTSGVHTFPAVLQSSG | |
| | LYSLSSVVTVPSSSLGTKTYTCNVDHKPSNTKVDKRVESKYGPP | |
| | CPPCPAPEFLGGPSVFLFPPKPKDTLMISRTPEVTCVVVDVSQEDP | |
| | EVQFNWYVDGVEVHNAKTKPREEQFNSTYRVVSVLTVLHQDW | |
| | LNGKEYKCKVSKGLPSSIEKTISKAKGQPREPQVYTLPPSQEEMT | |
| | KNQVSLTCLVKGFYPSDIAVEWESNGQPENNYKTTPPVLDSDGS | |
| | FFLYSRLTVDKSRWQEGNVFSCSVMHEALHNHYTQKSLSLSLGK | |
| Serplulimab, | DIQMTQSPSSLSASVGDRVTITCKASQDVTTAVAWYQQKPGKAP | 64 |
| HLX-10 | KLLIYWASTRHTGVPSRFSGSGSGTDFTLTISSLQPEDFATYYCOO | |
| VL | HYTIPWTFGGGTKLEIKRTVAAPSVFIFPPSDEQLKSGTASVVCLL | |
| | NNFYPREAKVQWKVDNALQSGNSQESVTEQDSKDSTYSLSSTLT | |
| | LSKADYEKHKVYACEVTHQGLSSPVTKSFNRGEC | |
| Retifanlimab, | QVQLVQSGAEVKKPGASVKVSCKASGYSFTSYWMNWVRQAPG | 65 |
| 2Y3T5IF01Z, | QGLEWIGVIHPSDSETWLDQKFKDRVTITVDKSTSTAYMELSSLR | |
| INCMGA- | SEDTAVYYCAREHYGTSPFAYWGQGTLVTVSSASTKGPSVFPLA | |
| 00012, | PCSRSTSESTAALGCLVKDYFPEPVTVSWNSGALTSGVHTFPAVL | |
| INCMGA-0012, | QSSGLYSLSSVVTVPSSSLGTKTYTCNVDHKPSNTKVDKRVESK | |
| MGA-012 | YGPPCPPCPAPEFLGGPSVFLFPPKPKDTLMISRTPEVTCVVVDVS | |
| VH | QEDPEVQFNWYVDGVEVHNAKTKPREEQFNSTYRVVSVLTVLH | |
| | QDWLNGKEYKCKVSNKGLPSSIEKTISKAKGQPREPQVYTLPPSQ | |
| | EEMTKNQVSLTCLVKGFYPSDIAVEWESNGQPENNYKTTPPVLD | |
| | SDGSFFLYSRLTVDKSRWQEGNVFSCSVMHEALHNHYTQKSLSL | |
| | SLG | |
| Retifanlimab, | EIVLTQSPATLSLSPGERATLSCRASESVDNYGMSFMNWFQQKP | 66 |
| 2Y3T5IF01Z, | GQPPKLLIHAASNQGSGVPSRFSGSGSGTDFTLTISSLEPEDFAVY | |
| INCMGA- | FCQQSKEVPYTFGGGTKVEIKRTVAAPSVFIFPPSDEQLKSGTASV | |
| 00012, | VCLLNNFYPREAKVQWKVDNALQSGNSQESVTEQDSKDSTYSL | |
| INCMGA-0012, | SSTLTLSKADYEKHKVYACEVTHQGLSSPVTKSFNRGEC | |
| MGA-012 | | |
| VL | | |
| Sasanlimab, | QVQLVQSGAEVKKPGASVKVSCKASGYTFTSYWINWVRQAPGQ | 67 |
| LZZ0IC2EWP, | GLEWMGNIYPGSSLTNYNEKFKNRVTMTRDTSTSTVYMELSSLR | |
| PF-06801591, | SEDTAVYYCARLSTGTFAYWGQGTLVTVSSASTKGPSVFPLAPC | |
| RN-888 | SRSTSESTAALGCLVKDYFPEPVTVSWNSGALTSGVHTFPAVLQS | |
| VH | SGLYSLSSVVTVPSSSLGTKTYTCNVDHKPSNTKVDKRVESKYG | |
| | PPCPPCPAPEFLGGPSVFLFPPKPKDTLMISRTPEVTCVVVDVSQE | |
| | DPEVQFNWYVDGVEVHNAKTKPREEQFNSTYRVVSVLTVLHQD | |
| | WLNGKEYKCKVSNKGLPSSIEKTISKAKGQPREPQVYTLPPSQEE | |
| | MTKNQVSLTCLVKGFYPSDIAVEWESNGQPENNYKTTPPVLDSD | |
| | GSFFLYSRLTVDKSRWQEGNVFSCSVMHEALHNHYTQKSLSLSL | |
| | GK | |
| Sasanlimab, | DIVMTQSPDSLAVSLGERATINCKSSQSLWDSGNQKNFLTWYQQ | 68 |
| LZZ0IC2EWP, | KPGQPPKLLIYWTSYRESGVPDRFSGSGSGTDFTLTISSLQAEDVA | |
| PF-06801591, | VYYCQNDYFYPHTFGGGTKVEIKRTVAAPSVFIFPPSDEQLKSGT | |
| RN-888 | ASVVCLLNNFYPREAKVQWKVDNALQSGNSQESVTEQDSKDST | |
| VL | YSLSSTLTLSKADYEKHKVYACEVTHQGLSSPVTKSFNRGEC | |
| Spartalizumab, | EVQLVQSGAEVKKPGESLRISCKGSGYTFTTYWMHWVRQATGQ | 69 |
| NVP-LZV-184, | GLEWMGNIYPGTGGSNFDEKFKNRVTITADKSTSTAYMELSSLR | |
| PDR-001, | SEDTAVYYCTRWTTGTGAYWGQGTTVTVSSASTKGPSVFPLAP | |
| QOG25L6Z8Z | CSRSTSESTAALGCLVKDYFPEPVTVSWNSGALTSGVHTFPAVL | |
| VH | QSSGLYSLSSVVTVPSSSLGTKTYTCNVDHKPSNTKVDKRVESK | |
| | YGPPCPPCPAPEFLGGPSVFLFPPKPKDTLMISRTPEVTCVVVDVS | |
| | QEDPEVQFNWYVDGVEVHNAKTKPREEQFNSTYRVVSVLTVLH | |
| | QDWLNGKEYKCKVSNKGLPSSIEKTISKAKGQPREPQVYTLPPSQ | |
| | EEMTKNQVSLTCLVKGFYPSDIAVEWESNGQPENNYKTTPPVLD | |
| | SDGSFFLYSRLTVDKSRWQEGNVFSCSVMHEALHNHYTQKSLSL | |
| | SLG | |
| Spartalizumab, | EIVLTQSPATLSLSPGERATLSCKSSQSLLDSGNQKNFLTWYQQK | 70 |
| NVP-LZV-184, | PGQAPRLLIYWASTRESGVPSRFSGSGSGTDFTFTISSLEAEDAAT | |
| PDR-001, | YYCQNDYSYPYTFGQGTKVEIKRTVAAPSVFIFPPSDEQLKSGTA | |
| QOG25L6Z8Z | SVVCLLNNFYPREAKVQWKVDNALQSGNSQESVTEQDSKDSTY | |
| VL | SLSSTLTLSKADYEKHKVYACEVTHQGLSSPVTKSFNRGEC | |
| Cetrelimab, | QVQLVQSGAEVKKPGSSVKVSCKASGGTFSSYAISWVRQAPGQ | 71 |
| JNJ-3283, JNJ- | GLEWMGGIIPIFDTANYAQKFQGRVTITADESTSTAYMELSSLRS | |
| 63723283, | EDTAVYYCARPGLAAAYDTGSLDYWGQGTLVTVSSASTKGPSV | |
| LYK98WP91F | FPLAPCSRSTSESTAALGCLVKDYFPEPVTVSWNSGALTSGVHTF | |
| VH | PAVLQSSGLYSLSSVVTVPSSSLGTKTYTCNVDHKPSNTKVDKR | |
| | VESKYGPPCPPCPAPEFLGGPSVFLFPPKPKDTLMISRTPEVTCVV | |
| | VDVSQEDPEVQFNWYVDGVEVHNAKTKPREEQFNSTYRVVSVL | |
| | TVLHQDWLNGKEYKCKVSNKGLPSSIEKTISKAKGQPREPQVYT | |
| | LPPSQEEMTKNQVSLTCLVKGFYPSDIAVEWESNGQPENNYKTT | |
| | PPVLDSDGSFFLYSRLTVDKSRWQEGNVFSCSVMHEALHNHYTQ | |
| | KSLSLSLGK | |
| Cetrelimab, | EIVLTQSPATLSLSPGERATLSCRASQSVRSYLAWYQQKPGQAPR | 72 |
| JNJ-3283, JNJ- | LLIYDASNRATGIPARFSGSGSGTDFTLTISSLEPEDFAVYYCQQR | |
| 63723283, | NYWPLTFGQGTKVEIKRTVAAPSVFIFPPSDEQLKSGTASVVCLL | |
| LYK98WP91F | NNFYPREAKVQWKVDNALQSGNSQESVTEQDSKDSTYSLSSTLT | |
| VL | LSKADYEKHKVYACEVTHQGLSSPVTKSFNRGEC | |
| Tebotelimab, | DIQMTQSPSSLSASVGDRVTITCRASQDVSSVVAWYQQKPGKAP | 73 |
| MGD-013 | KLLIYSASYRYTGVPSRFSGSGSGTDFTLTISSLQPEDFATYYCQQ | |
| VH | HYSTPWTFGGGTKLEIKGGGSGGGGQVQLVQSGAEVKKPGASV | |
| | KVSCKASGYSFTSYWMNWVRQAPGQGLEWIGVIHPSDSETWLD | |
| | QKFKDRVTITVDKSTSTAYMELSSLRSEDTAVYYCAREHYGTSP | |
| | FAYWGQGTLVTVSSGGCGGGEVAACEKEVAALEKEVAALEKE | |
| | VAALEKESKYGPPCPPCPAPEFLGGPSVFLFPPKPKDTLYITREPE | |
| | VTCVVVDVSQEDPEVQFNWYVDGVEVHNAKTKPREEQFNSTYR | |
| | VVSVLTVLHQDWLNGKEYKCKVSNKGLPSSIEKTISKAKGQPRE | |
| | PQVYTLPPSQEEMTKNQVSLTCLVKGFYPSDIAVEWESNGQPEN | |
| | NYKTTPPVLDSDGSFFLYSRLTVDKSRWQEGNVFSCSVMHEALH | |
| | NHYTQKSLSLSLG | |
| Tebotelimab, | EIVLTQSPATLSLSPGERATLSCRASESVDNYGMSFMNWFQQKP | 74 |
| MGD-013 | GQPPKLLIHAASNQGSGVPSRFSGSGSGTDFTLTISSLEPEDFAVY | |
| VL | FCQQSKEVPYTFGGGTKVEIKGGGSGGGGQVQLVQSGAEVKKP | |
| | GASVKVSCKASGYTFTDYNMDWVRQAPGQGLEWMGDINPDNG | |
| | VTIYNQKFEGRVTMTTDTSTSTAYMELRSLRSDDTAVYYCAREA | |
| | DYFYFDYWGQGTTLTVSSGGCGGGKVAACKEKVAALKEKVAA | |
| | LKEKVAALKE | |
| Pidilizumab, | QVQLVQSGSELKKPGASVKISCKASGYTFTNYGMNWVRQAPGQ | 75 |
| CT-011, MDV- | GLQWMGWINTDSGESTYAEEFKGRFVFSLDTSVNTAYLQITSLT | |
| 9300 | AEDTGMYFCVRVGYDALDYWGQGTLVTVSSASTKGPSVFPLAP | |
| VH | SSKSTSGGTAALGCLVKDYFPEPVTVSWNSGALTSGVHTFPAVL | |
| | QSSGLYSLSSVVTVPSSSLGTQTYICNVNHKPSNTKVDKRVEPKS | |
| | CDKTHTCPPCPAPELLGGPSVFLFPPKPKDTLMISRTPEVTCVVV | |
| | DVSHEDPEVKFNWYVDGVEVHNAKTKPREEQYNSTYRVVSVLT | |
| | VLHQDWLNGKEYKCKVSNKALPAPIEKTISKAKGQPREPQVYTL | |
| | PPSREEMTKNQVSLTCLVKGFYPSDIAVEWESNGQPENNYKTTP | |
| | PVLDSDGSFFLYSKLTVDKSRWQQGNVFSCSVMHEALHNHYTQ | |
| | KSLSLSPGK | |
| Pidilizumab, | EIVLTQSPSSLSASVGDRVTITCSARSSVSYMHWFQQKPGKAPKL | 76 |
| CT-011, MDV- | WIYRTSNLASGVPSRFSGSGSGTSYCLTINSLQPEDFATYYCQQR | |
| 9300 | SSFPLTFGGGTKLEIKRTVAAPSVFIFPPSDEQLKSGTASVVCLLN | |
| VL | NFYPREAKVQWKVDNALQSGNSQESVTEQDSKDSTYSLSSTLTL | |
| | SKADYEKHKVYACEVTHQGLSSPVTKSFNRGEC | |
| SG-001 VH | QVQLVESGGGVVQPGRSLRLTCKASGLTFSSSGMHWVRQAPGK | 77 |
| | GLEWVAVIWYDGSKRYYADSVKGRFTISRDNSKNTLFLQMNSL | |
| | RAEDTAVYYCATNNDYWGQGTLVTVSS | |
| SG-001 VL | EIVLTQSPATLSLSPGERATLSCRASQSVSSYLAWYQQKPGQAPR | 78 |
| | LLIYTASNRATGIPARFSGSGSGTDFTLTISSLEPEDFAVYYCQQY | |
| | SNWPRTFGQGTKVEIK | |
| LZM-009 VH | EVQLQQSGPVLVKPGASVKMSCKASGYTFTSYYMYWVKQSHG | 79 |
| | KSLEWIGGVNPSNGGTNFNEKFKSKATLTVDKSSSTAYMELNSL | |
| | TSEDSAVYYCARRDYRYDMGFDYWGQGTTLTVSS | |
| LZM-009 VL | QIVLTQSPAIMSASPGEKVTMTCRASKGVSTSGYSYLHWYQQKP | 80 |
| | GSSPRLLIYLASYLESGVPVRFSGSGSGTSYSLTISRMEAEDAATY | |
| | YCQHSRELPLTFGTGTRLEIK | |
| LZM-009 VH | QVQLVQSGAEVKKPGASVKVSCKASGYTFTSYYMYWVRQAPG | 81 |
| | QGLEWMGGVNPSNGGTNFNEKFKSRVTITADKSTSTAYMELSSL | |
| | RSEDTAVYYCARRDYRYDMGFDYWGQGTTVTVSS | |
| LZM-009 VL | EIVLTQSPATLSLSPGERATISCRASKGVSTSGYSYLHWYQQKPG | 82 |
| | QAPRLLIYLASYLESGVPARFSGSGSGTDFTLTISSLEPEDFATYY | |
| | CQHSRELPLTFGTGTKVEIK | |
| Budigalimab, | EIQLVQSGAEVKKPGSSVKVSCKASGYTFTHYGMNWVRQAPGQ | 83 |
| 6VDO4TY3OO, | GLEWVGWVNTYTGEPTYADDFKGRLTFTLDTSTSTAYMELSSL | |
| ABBV-181, PR- | RSEDTAVYYCTREGEGLGFGDWGQGTTVTVSSASTKGPSVFPLA | |
| 1648817 | PSSKSTSGGTAALGCLVKDYFPEPVTVSWNSGALTSGVHTFPAV | |
| VH | LQSSGLYSLSSVVTVPSSSLGTQTYICNVNHKPSNTKVDKKVEPK | |
| | SCDKTHTCPPCPAPEAAGGPSVFLFPPKPKDTLMISRTPEVTCVV | |
| | VDVSHEDPEVKFNWYVDGVEVHNAKTKPREEQYNSTYRVVSVL | |
| | TVLHQDWLNGKEYKCKVSNKALPAPIEKTISKAKGQPREPQVYT | |
| | LPPSREEMTKNQVSLTCLVKGFYPSDIAVEWESNGQPENNYKTT | |
| | PPVLDSDGSFFLYSKLTVDKSRWQQGNVFSCSVMHEALHNHYT | |
| | QKSLSLSPGK | |
| Budigalimab, | DVVMTQSPLSLPVTPGEPASISCRSSQSIVHSHGDTYLEWYLQKP | 84 |
| 6VDO4TY3OO, | GQSPQLLIYKVSNRFSGVPDRFSGSGSGTDFTLKISRVEAEDVGV | |
| ABBV-181, PR- | YYCFQGSHIPVTFGQGTKLEIKRTVAAPSVFIFPPSDEQLKSGTAS | |
| 1648817 | VVCLLNNFYPREAKVQWKVDNALQSGNSQESVTEQDSKDSTYS | |
| VL | LSSTLTLSKADYEKHKVYACEVTHQGLSSPVTKSFNRGEC | |
| Lambrolizumab, | NYYMY | 85 |
| Pembrolizumab, | | |
| KEYTRUDA ®, | | |
| MK-3475, SCH- | | |
| 900475, | | |
| h409A11 | | |
| VH CDR1 | | |
| Lambrolizumab, | GINPSNGGTNFNEKFKN | 86 |
| Pembrolizumab, | | |
| KEYTRUDA ®, | | |
| MK-3475, SCH- | | |
| 900475, | | |
| h409A11 | | |
| VH CDR2 | | |
| Lambrolizumab, | RDYRFDMGFDY | 87 |
| Pembrolizumab, | | |
| KEYTRUDA ®, | | |
| MK-3475, SCH- | | |
| 900475, | | |
| h409A11 | | |
| VH CDR3 | | |
| Lambrolizumab, | RASKGVSTSGYSYLH | 88 |
| Pembrolizumab, | | |
| KEYTRUD A®, | | |
| MK-3475, SCH- | | |
| 900475, | | |
| h409A11 | | |
| VL CDR1 | | |
| Lambrolizumab, | LASYLES | 89 |
| Pembrolizumab, | | |
| KEYTRUDA ®, | | |
| MK-3475, SCH- | | |
| 900475, | | |
| h409A11 | | |
| VL CDR2 | | |
| Lambrolizumab, | QHSRDLPLT | 90 |
| Pembrolizumab, | | |
| KEYTRUDA ®, | | |
| MK-3475, SCH- | | |
| 900475, | | |
| h409A11 | | |
| VL CDR3 | | |
| Nivolumab, | NSGMH | 91 |
| Nivolumab | | |
| BMS, | | |
| OPDIVO ®, | | |
| BMS-936558, | | |
| MDX-1106, | | |
| ONO-4538 | | |
| VH CDR1 | | |
| Nivolumab, | VIWYDGSKRYYADSVKG | 92 |
| Nivolumab | | |
| BMS, | | |
| OPDIVO ®, | | |
| BMS-936558, | | |
| MDX-1106, | | |
| ONO-4538 | | |
| VH CDR2 | | |
| Nivolumab, | NDDY | 93 |
| Nivolumab | | |
| BMS, | | |
| OPDIVO ®, | | |
| BMS-936558, | | |
| fragment | | |
| MDX-1106, | | |
| ONO-4538 | | |
| VH CDR3 | | |
| Nivolumab, | RASQSVSSYLA | 94 |
| Nivolumab | | |
| BMS, | | |
| OPDIVO ®, | | |
| BMS-936558, | | |
| MDX-1106, | | |
| ONO-4538 | | |
| VL CDR1 | | |
| Nivolumab, | DASNRAT | 95 |
| Nivolumab | | |
| BMS, | | |
| OPDIVO ®, | | |
| BMS-936558, | | |
| MDX-1106, | | |
| ONO-4538 | | |
| VL CDR2 | | |
| Nivolumab, | QQSSNWPRT | 96 |
| Nivolumab | | |
| BMS, | | |
| OPDIVO ®, | | |
| BMS-936558, | | |
| MDX-1106, | | |
| ONO-4538 | | |
| VL CDR3 | | |
| Serplulimab, | FTFSNYGMS | 97 |
| HLX-10 | | |
| VH CDR1 | | |
| Serplulimab, | TISGGGSNIY | 98 |
| HLX-10 | | |
| VH CDR2 | | |
| Serplulimab, | VSYYYGIDF | 99 |
| HLX-10 | | |
| VH CDR3 | | |
| Serplulimab, | KASQDVTTAVA | 100 |
| HLX-10 | | |
| VL CDR1 | | |
| Serplulimab, | WASTRHT | 101 |
| HLX-10 | | |
| VL CDR2 | | |
| Serplulimab, | QQHYTIPWT | 102 |
| HLX-10 | | |
| VL CDR3 | | |
| SG-001 | GLTFSSSG | 103 |
| VH CDR1 | | |
| SG-001 | IWYDGSKR | 104 |
| VH CDR2 | | |
| SG-001 | ATNNDY | 105 |
| VH CDR3 | | |
| SG-001 | RASQSVSSYLA | 106 |
| VL CDR1 | | |
| SG-001 | TASNRAT | 107 |
| VL CDR2 | | |
| SG-001 | QQYSNWPRT | 108 |
| VL CDR3 | | |
| PD-1-Fc- | MQIPQAPWPWWAVLQLGWRPGWFLDSPDRPWNPPTFSPALLVV | 109 |
| OX40L (Code), | TEGDNATFTCSFSNTSESFVLNWYRMSPSNQTDKLAAFPEDRSQP | |
| SL-279252 | GQDCRFRVTQLPNGRDFHMSVVRARRNDSGTYLCGAISLAPKA | |
| (Code), TAK- | QIKESLRAELRVTERRAEVPTAHPSPSPRPAGQFQSKYGPPCPSCP | |
| 252 (Code) | APEFLGGPSVFLFPPKPKDTLMISRTPEVTCWVDVSQEDPEVQFN | |
| | WYVDGVEVHNAKTKPREEQFNSTYRVVSVLTVLHQDWLSGKE | |
| | YKCKVSSKGLPSSIEKTISNATGQPREPQVYTLPPSQEEMTKNQV | |
| | SLTCLVKGFYPSDIAVEWESNGQPENNYKTTPPVLDSDGSFFLYS | |
| | RLTVDKSSWQEGNVFSCSVMHEALHNHYTQKSLSLSLGKIEGR | |
| | MDQVSHRYPRIQSIKVQFTEYKKEKGFILTSQKEDEIMKVQNNSV | |
| | IINCDGFYLISLKGYFSQEVNISLHYQKDEEPLFQLKKVRSVNSLM | |
| | VASLTYKDKVYLNVTTDNTSLDDFHVNGGELILIHQNPGEFCVL | |
| | MQIPQAPWPWWAVLQLGWRPGWFLDSPDRPWNPPTFSPALLVV | |
| | TEGDNATFTCSFSNTSESFVLNWYRMSPSNQTDKLAAFPEDRSQP | |
| | GQDCRFRVTQLPNGRDFHMSVVRARRNDSGTYLCGAISLAPKA | |
| | QIKESLRAELRVTERRAEVPTAHPSPSPRPAGQFQ | |
| | QVSHRYPRIQSIKVQFTEYKKEKGFILTSQKEDEIMKVQNNSVIIN | |
| | CDGFYLISLKGYFSQEVNISLHYQKDEEPLFQLKKVRSVNSLMVA | |
| | SLTYKDKVYLNVTTDNTSLDDFHVNGGELILIHQNPGEFCVL | |

An anti-PD-1 polypeptide or an anti-PD-1 antigen binding fragment can comprise a VH having an amino acid sequence of any one of SEQ ID NOS: 37, 39, 41, 43, 45, 51, 53, 55, 57, 59, 61, 63, 65, 67, 69, 71, 73, 75, 77, 79, 81, and 83. An anti-PD-1 polypeptide or an anti-PD-1 antigen binding fragment can comprise a VH having an amino acid sequence of any one of SEQ ID NOS: 38, 40, 42, 44, 46, 48, 50, 52, 54, 56, 58, 60, 62, 64, 66, 68, 70, 72, 74, 76, 78, 80, 82, and 84.

In one instance, an anti-PD-1 polypeptide or an anti-PD-1 antigen binding fragment comprises a VH having an amino acid sequence of SEQ ID NO: 37, and a VL having an amino acid sequence of SEQ ID NO: 38. In another instance, an anti-PD-1 polypeptide or an anti-PD-1 antigen binding fragment comprises a VH having an amino acid sequence of SEQ ID NO: 39, and a VL having an amino acid sequence of SEQ ID NO: 40. In another instance, an anti-PD-1 polypeptide or an anti-PD-1 antigen binding fragment comprises a VH having an amino acid sequence of SEQ ID NO: 41, and a VL having an amino acid sequence of SEQ ID NO: 42. In another instance, an anti-PD-1 polypeptide or an anti-PD-1 antigen binding fragment comprises a VH having an amino acid sequence of SEQ ID NO: 43, and a VL having an amino acid sequence of SEQ ID NO: 44. In another instance, an anti-PD-1 polypeptide or an anti-PD-1 antigen binding fragment comprises a VH having an amino acid sequence of SEQ ID NO: 45, and a VL having an amino acid sequence of SEQ ID NO: 46. In another instance, an anti-PD-1 polypeptide or an anti-PD-1 antigen binding fragment comprises a VH having an amino acid sequence of SEQ ID NO: 51, and a VL having an amino acid sequence of SEQ ID NO: 52. In another instance, an anti-PD-1 polypeptide or an anti-PD-1 antigen binding fragment comprises a VH having an amino acid sequence of SEQ ID NO: 53, and a VL having an amino acid sequence of SEQ ID NO: 54. In another instance, an anti-PD-1 polypeptide or an anti-PD-1 antigen binding fragment comprises a VH having an amino acid sequence of SEQ ID NO: 55, and a VL having an amino acid sequence of SEQ ID NO: 56. In another instance, an anti-PD-1 polypeptide or an anti-PD-1 antigen binding fragment comprises a VH having an amino acid sequence of SEQ ID NO: 57, and a VL having an amino acid sequence of SEQ ID NO: 58. In another instance, an anti-PD-1 polypeptide or an anti-PD-1 antigen binding fragment comprises a VH having an amino acid sequence of SEQ ID NO: 59, and a VL having an amino acid sequence of SEQ ID NO: 60. In another instance, an anti-PD-1 polypeptide or an anti-PD-1 antigen binding fragment comprises a VH having an amino acid sequence of SEQ ID NO: 61, and a VL having an amino acid sequence of SEQ ID NO: 62. In another instance, an anti-PD-1 polypeptide or an anti-PD-1 antigen binding fragment comprises a VH having an amino acid sequence of SEQ ID NO: 63, and a VL having an amino acid sequence of SEQ ID NO: 64. In another instance, an anti-PD-1 polypeptide or an anti-PD-1 antigen binding fragment comprises a VH having an amino acid sequence of SEQ ID NO: 65, and a VL having an amino acid sequence of SEQ ID NO: 66. In another instance, an anti-PD-1 polypeptide or an anti-PD-1 antigen binding fragment comprises a VH having an amino acid sequence of SEQ ID NO: 67, and a VL having an amino acid sequence of SEQ ID NO: 68. In another instance, an anti-PD-1 polypeptide or an anti-PD-1 antigen binding fragment comprises a VH having an amino acid sequence of SEQ ID NO: 69, and a VL having an amino acid sequence of SEQ ID NO: 70. In another instance, an anti-PD-1 polypeptide or an anti-PD-1 antigen binding fragment comprises a VH having an amino acid sequence of SEQ ID NO: 71, and a VL having an amino acid sequence of SEQ ID NO: 72. In another instance, an anti-PD-1 polypeptide or an anti-PD-1 antigen binding fragment comprises a VH having an amino acid sequence of SEQ ID NO: 73, and a VL having an amino acid sequence of SEQ ID NO: 74. In another instance, an anti-PD-1 polypeptide or an anti-PD-1 antigen binding fragment comprises a VH having an amino acid sequence of SEQ ID NO: 75, and a VL having an amino acid sequence of SEQ ID NO: 76. In another instance, an anti-PD-1 polypeptide or an anti-PD-1 antigen binding fragment comprises a VH having an amino acid sequence of SEQ ID NO: 77, and a VL having an amino acid sequence of SEQ ID NO: 78. In another instance, an anti-PD-1 polypeptide or an anti-PD-1 antigen binding fragment comprises a VH having an amino acid sequence of SEQ ID NO: 79, and a VL having an amino acid sequence of SEQ ID NO: 80. In another instance, an anti-PD-1 polypeptide or an anti-PD-1 antigen binding fragment comprises a VH having an amino acid sequence of SEQ ID NO: 81, and a VL having an amino acid sequence of SEQ ID NO: 82. In another instance, an anti-PD-1 polypeptide or an anti-PD-1 antigen binding fragment comprises a VH having an amino acid sequence of SEQ ID NO: 83, and a VL having an amino acid sequence of SEQ ID NO: 84.

In one instance, an anti-PD-1 polypeptide or an anti-PD-1 antigen binding fragment comprises a VH CHR1 having an amino acid sequence of SEQ ID NO: 85, a VH CHR2 having an amino acid sequence of SEQ ID NO: 86, a VH CHR3 having an amino acid sequence of SEQ ID NO: 87, VL CHR1 having an amino acid sequence of SEQ ID NO: 88, a VL CHR2 having an amino acid sequence of SEQ ID NO: 89, and a VL CHR3 having an amino acid sequence of SEQ ID NO: 90. In one instance, an anti-PD-1 polypeptide or an anti-PD-1 antigen binding fragment comprises a VH CHR1 having an amino acid sequence of SEQ ID NO: 91, a VH CHR2 having an amino acid sequence of SEQ ID NO: 92, a VH CHR3 having an amino acid sequence of SEQ ID NO: 93, VL CHR1 having an amino acid sequence of SEQ ID NO: 94, a VL CHR2 having an amino acid sequence of SEQ ID NO: 95, and a VL CHR3 having an amino acid sequence of SEQ ID NO: 96. In one instance, an anti-PD-1 polypeptide or an anti-PD-1 antigen binding fragment comprises a VH CHR1 having an amino acid sequence of SEQ ID NO: 97, a VH CHR2 having an amino acid sequence of SEQ ID NO: 98, a VH CHR3 having an amino acid sequence of SEQ ID NO: 99, VL CHR1 having an amino acid sequence of SEQ ID NO: 100, a VL CHR2 having an amino acid sequence of SEQ ID NO: 101, and a VL CHR3 having an amino acid sequence of SEQ ID NO: 102. In one instance, an anti-PD-1 polypeptide or an anti-PD-1 antigen binding fragment comprises a VH CHR1 having an amino acid sequence of SEQ ID NO: 103, a VH CHR2 having an amino acid sequence of SEQ ID NO: 104, a VH CHR3 having an amino acid sequence of SEQ ID NO: 105, VL CHR1 having an amino acid sequence of SEQ ID NO: 106, a VL CHR2 having an amino acid sequence of SEQ ID NO: 107, and a VL CHR3 having an amino acid sequence of SEQ ID NO: 108.

In one instance, an anti-PD-1 polypeptide comprises a fusion protein. Such fusion protein can be, for example, a two-sided Fc fusion protein comprising the extracellular domain (ECD) of programmed cell death 1 (PD-1) and the ECD of tumor necrosis factor (ligand) superfamily member 4 (TNFSF4 or OX40L) fused via hinge-CH2-CH3 Fc domain of human IgG4, expressed in CHO-K1 cells, where the fusion protein has an exemplary amino acid sequence of SEQ ID NO: 109.

### Anti-PD-L1 Antibodies

In one embodiment, an anti-PD-L1 antibody or an anti-PD-L1 antigen binding fragment of the disclosure comprises a combination of a heavy chain variable region (VH) and a light chain variable region (VL) described herein. In another embodiment, an anti-PD-L1 antibody or an anti-PD-L1 antigen binding fragment of the disclosure comprises a combination of complementarity determining regions (VH CDR1, VH CDR2, VH CDR3, VL CDR1, VL CDR2, and VL CDR3) described herein. In one embodiment, an anti-PD-L1 antibody or an anti-PD-L1 antigen binding fragment of the disclosure comprises a modified Modified Avelumab (Bavencio, 451238, KXG2PJ551I, MSB-0010682, MSB-0010718C, PF-06834635, CAS 1537032-82-8: EMD Serono, Merck & Co., Merck KGaA, Merck Serono, National Cancer Institute (NCI), Pfizer), Durvalumab (Imfinzi, 28X28X9OKV (UNII code), MEDI-4736, CAS 1428935-60-7: AstraZeneca, Celgene, Children's Hospital Los Angeles (CHLA), City of Hope National Medical Center, MedImmune, Memorial Sloan-Kettering Cancer Center, Mirati Therapeutics, National Cancer Institute (NCI), Samsung Medical Center (SMC), Washington University), Atezolizumab (Tecentriq, 52CMI0WC3Y, MPDL-3280A, RG-7446, RO-5541267, CAS 1380723-44-3: Academisch Medisch Centrum (AMC), Chugai Pharmaceutical, EORTC, Genentech, Immune Design (Merck & Co.), Memorial Sloan-Kettering Cancer Center, National Cancer Institute (NCI), Roche, Roche Center for Medical Genomics), Sugemalimab (CS-1001, WBP-3155: CStone Pharmaceuticals, EQRx, Pfizer), KN-046 (CAS 2256084-03-2: Jiangsu Alphamab Biopharmaceuticals, Sinovent), APL-502 (CBT-502, TQB-2450: Apollomics, Jiangsu Chia Tai Tianqing Pharmaceutical), Envafolimab (3D-025, ASC-22, KN-035, hu56V1-Fc-m1, CAS 2102192-68-5: 3D Medicines, Ascletis, Jiangsu Alphamab Biopharmaceuticals, Suzhou Alphamab, Tracon Pharmaceuticals, Inc.), Bintrafusp alfa (M-7824, MSB-0011359C, NW9K8C1JN3, CAS 1918149-01-5: EMD Serono, GlaxoSmithKline, Merck KGaA, National Cancer Institute (NCI)), STI-1014 (STI-A1014, ZKAB-001: Lee's Pharmaceutical, Sorrento Therapeutics), PD-L1 t-haNK (ImmunityBio, NantKwest), A-167 (HBM-9167, KL-A167: Harbour BioMed, Sichuan Kelun-Biotech Biopharmaceutical), IMC-001 (STI-3031, STI-A-1015, STI-A1015, ImmuneOncia Therapeutics, Sorrento Therapeutics), HTI-1088 (SHR-1316: Atridia, Jiangsu Hengrui), IO-103 (IO Biotech), CX-072 (CytomX Therapeutics), AUPM-170 (CA-170: Aurigene, Curis), GS-4224 (Gilead), ND-021 (NM21-1480, PRO-1480: CStone Pharmaceuticals, Numab Therapeutics), BNT-311 (DuoBody-PD-L1x4-1BB, GEN-1046: BioNTech, Genmab), BGB-A333 (BeiGene), IBI-322 (Innovent Biologics), NM-01 (Nanomab Technology, Shanghai First People's Hospital), LY-3434172 (Eli Lilly), LDP (Dragonboat Biopharmaceutical), CDX-527 (Celldex Therapeutics), IBI-318 (Innovent Biologics, Lilly), 89Zr-DFO-REGN3504 (Regeneron), ALPN-202 (CD80 vIgD-Fc: Alpine Immune Sciences), INCB-086550 (Incyte), LY-3415244 (Eli Lilly), SHR-1701 (Jiangsu Hengrui), JS-003 (JS003-30, JS003-SD: Shanghai Junshi Biosciences), HLX-20 (PL2 #3: Henlix Biotech, Shanghai Henlius Biotech), ES-101 (INBRX-105, INBRX-105-1: Elpiscience BioPharma, Inhibrx), MSB-2311 (MabSpace Biosciences), PD-1-Fc-OX40L (SL-279252, TAK-252: Heat Biologics, Shattuck Labs, Takeda), FS-118, FS118 mAb2, LAG-3/PD-L1 mAb2: F-star Therapeutics, Merck & Co., Merck KGaA), FAZ-053 (LAE-005: Laekna Therapeutics, Novartis), Lodapolimab (LY-3300054, NR4MAD6PPB, CAS 2118349-31-6: Eli Lilly), MCLA-145 (Incyte, Merus), BMS-189 (BMS-986189, PD-L1-Milla from Bristol-Myers Squibb), Cosibelimab (CK-301, TG-1501, CAS 2216751-26-5: Checkpoint Therapeutics, Dana-Farber Cancer Institute, Samsung Biologics, TG Therapeutics), IL-15Ralpha-SD/IL-15 (KD-033: Kadmon), WP-1066 (CAS 857064-38-1: M.D. Anderson Cancer Center, Moleculin Biotech), BMS-936559 (MDX-1105: Bristol-Myers Squibb, Medarex, National Institute Allergy Infect Dis.), BMS-986192 (Bristol-Myers Squibb), RC-98 (RemeGen), CD-200AR-L (CD200AR-L: OX2 Therapeutics, University of Minnesota), ATA-3271 (Atara Biotherapeutics), IBC-Ab002 (ImmunoBrain Checkpoint), BMX-101 (Biomunex Pharmaceuticals), AVA-04-VbP (Avacta), ACE-1708 (Acepodia Biotech), KY-1043 (Kymab, Provenance Biopharmaceuticals), ACE-05 (YBL-013: Y-Biologics), ONC-0055 (ONC0055, PRS-344 S-095012: Pieris Pharmaceuticals, Servier), TLJ-1-CK (I-Mab Biopharma), GR-1405 (Chinese Academy of Medical Sciences), PD1ACR-T (Taipei Medical University), N-809 (N-IL15/PDL1: ImmunityBio), CB-201 (Crescendo Biologics), MEDI-1109 (MedImmune), AVA-004 (AVA-04: Avacta), CA-327 (Aurigene, Curis), ALN-PDL (Alnylam Pharmaceuticals), KY-1003 (Kymab), CD22(aPD-L1)CAR-T cells (SL-22P: Hebei Senlang Biotechnology), ATA-2271 (M28z1XXPD1DNR CAR T cells: Atara Biotherapeutics), and Zeushield cytotoxic T lymphocytes (Second Xiangya Hosp Central South Univ.).

In some embodiments, the anti-PD-L1 polypeptide is modified with mAB3. In some embodiments, the anti-PD-L1 polypeptide is modified with mAB4.

TABLE 1B provides the sequences of exemplary anti-PD-L1 polypeptides and anti-PD-L1 antigen binding fragments that can be modified to prepare anti-PD-L1 immunoconjugates. TABLE 1B also provides exemplary combinations of CDRs that can be utilized in a modified anti-PD-L1 immunoconjugate. Reference to an anti-PD-L1 polypeptide herein may alternatively refer to an anti-PD-L1 antigen binding fragment.

TABLE 1B

| Drug Name (Generic, | | |
|---|---|---|
| Brand Name, Code | | SEQ ID |
| Name) | Sequence | NO: |
| Avelumab (Generic) | EVQLLESGGGLVQPGGSLRLSCAASGFTFSSYIMMWVR | 110 |
| Bavencio (Brand) | QAPGKGLEWVSSIYPSGGITFYADTVKGRFTISRDNSKN | |
| 451238 | TLYLQMNSLRAEDTAVYYCARIKLGTVTTVDYWGQGT | |
| KXG2PJ551I | LVTVSSASTKGPSVFPLAPSSKSTSGGTAALGCLVKDYF | |
| MSB-0010682 | PEPVTVSWNSGALTSGVHTFPAVLQSSGLYSLSSVVTV | |
| MSB-0010718C | PSSSLGTQTYICNVNHKPSNTKVDKKVEPKSCDKTHTC | |
| PF-06834635 | PPCPAPELLGGPSVFLFPPKPKDTLMISRTPEVTCVVVD | |
| VH | VSHEDPEVKFNWYVDGVEVHNAKTKPREEQYNSTYR | |
| | VVSVLTVLHQDWLNGKEYKCKVSNKALPAPIEKTISKA | |
| | KGQPREPQVYTLPPSRDELTKNQVSLTCLVKGFYPSDIA | |
| | VEWESNGQPENNYKTTPPVLDSDGSFFLYSKLTVDKSR | |
| | WQQGNVFSCSVMHEALHNHYTQKSLSLSPGK | |
| Avelumab (Generic) | QSALTQPASVSGSPGQSITISCTGTSSDVGGYNYVSWYQ | 111 |
| Bavencio (Brand) | QHPGKAPKLMIYDVSNRPSGVSNRFSGSKSGNTASLTIS | |
| 451238 | GLQAEDEADYYCSSYTSSSTRVFGTGTKVTVLGQPKAN | |
| KXG2PJ551I | PTVTLFPPSSEELQANKATLVCLISDFYPGAVTVAWKA | |
| MSB-0010682 | DGSPVKAGVETTKPSKQSNNKYAASSYLSLTPEQWKS | |
| MSB-0010718C | HRSYSCQVTHEGSTVEKTVAPTECS | |
| PF-06834635 | | |
| VL | | |
| Durvalumab (Generic) | EVQLVESGGGLVQPGGSLRLSCAASGFTFSRYWMSWV | 112 |
| Imfinzi (Brand) | RQAPGKGLEWVANIKQDGSEKYYVDSVKGRFTISRDN | |
| 28X28X9OKV (UNII | AKNSLYLQMNSLRAEDTAVYYCAREGGWFGELAFDY | |
| code) | WGQGTLVTVSSASTKGPSVFPLAPSSKSTSGGTAALGC | |
| MEDI-4736 | LVKDYFPEPVTVSWNSGALTSGVHTFPAVLQSSGLYSL | |
| VH | SSVVTVPSSSLGTQTYICNVNHKPSNTKVDKRVEPKSC | |
| | DKTHTCPPCPAPEFEGGPSVFLFPPKPKDTLMISRTPEVT | |
| | CVVVDVSHEDPEVKFNWYVDGVEVHNAKTKPREEQY | |
| | NSTYRVVSVLTVLHQDWLNGKEYKCKVSNKALPASIE | |
| | KTISKAKGQPREPQVYTLPPSREEMTKNQVSLTCLVKG | |
| | FYPSDIAVEWESNGQPENNYKTTPPVLDSDGSFFLYSKL | |
| | TVDKSRWQQGNVFSCSVMHEALHNHYTQKSLSLSPGK | |
| Durvalumab (Generic) | EIVLTQSPGTLSLSPGERATLSCRASQRVSSSYLAWYQQ | 113 |
| Imfinzi (Brand) | KPGQAPRLLIYDASSRATGIPDRFSGSGSGTDFTLTISRL | |
| 28X28X9OKV (UNII | EPEDFAVYYCQQYGSLPWTFGQGTKVEIKRTVAAPSVF | |
| code) | IFPPSDEQLKSGTASVVCLLNNFYPREAKVQWKVDNAL | |
| MEDI-4736 | QSGNSQESVTEQDSKDSTYSLSSTLTLSKADYEKHKVY | |
| VL | ACEVTHQGLSSPVTKSFNRGEC | |
| Atezolizumab (Generic) | EVQLVESGGGLVQPGGSLRLSCAASGFTFSDSWIHWVR | 114 |
| Tecentriq (Brand) | QAPGKGLEWVAWISPYGGSTYYADSVKGRFTISADTSK | |
| 52CMI0WC3Y | NTAYLQMNSLRAEDTAVYYCARRHWPGGFDYWGQG | |
| MPDL-3280A | TLVTVSSASTKGPSVFPLAPSSKSTSGGTAALGCLVKDY | |
| RG-7446 | FPEPVTVSWNSGALTSGVHTFPAVLQSSGLYSLSSVVT | |
| RO-5541267 | VPSSSLGTQTYICNVNHKPSNTKVDKKVEPKSCDKTHT | |
| VH | CPPCPAPELLGGPSVFLFPPKPKDTLMISRTPEVTCVVV | |
| | DVSHEDPEVKFNWYVDGVEVHNAKTKPREEQYASTY | |
| | RVVSVLTVLHQDWLNGKEYKCKVSNKALPAPIEKTISK | |
| | AKGQPREPQVYTLPPSREEMTKNQVSLTCLVKGFYPSD | |
| | IAVEWESNGQPENNYKTTPPVLDSDGSFFLYSKLTVDK | |
| | SRWQQGNVFSCSVMHEALHNHYTQKSLSLSPGK | |
| Atezolizumab (Generic) | DIQMTQSPSSLSASVGDRVTITCRASQDVSTAVAWYQQ | 115 |
| Tecentriq (Brand) | KPGKAPKLLIYSASFLYSGVPSRFSGSGSGTDFTLTISSL | |
| 52CMI0WC3Y | QPEDFATYYCQQYLYHPATFGQGTKVEIKRTVAAPSVF | |
| MPDL-3280A | IFPPSDEQLKSGTASVVCLLNNFYPREAKVQWKVDNAL | |
| RG-7446 | QSGNSQESVTEQDSKDSTYSLSSTLTLSKADYEKHKVY | |
| RO-5541267 | ACEVTHQGLSSPVTKSFNRGEC | |
| VL | | |
| Sugemalimab (Generic) | EVQLLESGGGLVQPGGSLRLSCAASGFTFSSYAMSWVR | 116 |
| CS-1001 | QAPGKGLEWVSGISGSGGFTYYADSVKGRFTISRDNSK | |
| WBP-3155 | NTLYLQMNSLRAEDTAVYYCAKPPRGYNYGPFDYWG | |
| VH | QGTLVTVSSASTKGPSVFPLAPCSRSTSESTAALGCLVK | |
| | DYFPEPVTVSWNSGALTSGVHTFPAVLQSSGLYSLSSV | |
| | VTVPSSSLGTKTYTCNVDHKPSNTKVDKRVESKYGPPC | |
| | PPCPAPEFLGGPSVFLFPPKPKDTLMISRTPEVTCVVVD | |
| | VSQEDPEVQFNWYVDGVEVHNAKTKPREEQFNSTYRV | |
| | VSVLTVLHQDWLNGKEYKCKVSNKGLPSSIEKTISKAK | |
| | GQPREPQVYTLPPSQEEMTKNQVSLTCLVKGFYPSDIA | |
| | VEWESNGQPENNYKTTPPVLDSDGSFFLYSRLTVDKSR | |
| | WQEGNVFSCSVMHEALHNHYTQKSLSLSLGK | |
| Sugemalimab (Generic) | SYVLTQPPSVSVAPGQTARITCGGNNIGSKSVHWYQQK | 117 |
| CS-1001 | PGQAPVLVVYDDSDRPSGIPERFSGSNSGNTATLTISRV | |
| WBP-3155 | EAGDEADYYCQVWDSSSDHVVFGGGTKLTVLGQPKA | |
| VL | APSVTLFPPSSEELQANKATLVCLISDFYPGAVTVAWK | |
| | ADSSPVKAGVETTTPSKQSNNKYAASSYLSLTPEQWKS | |
| | HRSYSCQVTHEGSTVEKTVAPTECS | |
| JS-003 | QGQLQESGPSLVKPSQTLSLTCTVSGDSITRGYWNWIR | 120 |
| JS003-30 | KHPGKGLEYIGYISYTGSTYSNLSLKSRVTISRDTSKNQ | |
| JS003-SD | YYLKLSSVTAADTAVYYCATSTGWLDPVDYWGQGTL | |
| VH | VTVSS | |
| JS-003 | DIVMTQSPDSLAVSLGERATINCKASQNVDTSVAWFQQ | 121 |
| JS003-30 | KPGQPPKALIYSASFRYSGVPDRFSGSGSGTDFTLTISSL | |
| JS003-SD | QAEDVAVYFCQQYYGYPFTFGQGTKLEIK | |
| VL | | |
| HLX-20 | EVQLVQSGGGLVKPGGSLRLSCAASGFTFSSYTMNWV | 122 |
| PL2#3 | RQAPGKGLEWVSSISSGSDYLYYADSVKGRFTISRDNA | |
| VH | KNSLYLQMNSLRAEDTAVYYCARNELRWYPQAGAFD | |
| | RWGQGTMVTVSS | |
| HLX-20 | QSVVTQPPSMSAAPGQRVTISCSGSSSYIESSYVGWYQQ | 123 |
| PL2#3 | LPGTAPRLLIYDDDMRPSGIPDRFSGSKSGTSATLAITGL | |
| VL | QTGDEADYYCEIWRSGLGGVFGGGTKLTVL | |
| Lodapolimab (Generic) | QVQLVQSGAEVKKPGSSVKVSCKASGGTFSSYAISWVR | 126 |
| LY-3300054 | QAPGQGLEWMGGIIPIFGTANYAQKFQGRVTITADKST | |
| NR4MAD6PPB | STAYMELSSLRSEDTAVYYCARSPDYSPYYYYGMDVW | |
| VH | GQGTTVTVSSASTKGPSVFPLAPSSKSTSGGTAALGCLV | |
| | KDYFPEPVTVSWNSGALTSGVHTFPAVLQSSGLYSLSS | |
| | VVTVPSSSLGTQTYICNVNHKPSNTKVDKRVEPKSCDK | |
| | THTCPPCPAPEAEGAPSVFLFPPKPKDTLMISRTPEVTCV | |
| | VVDVSHEDPEVKFNWYVDGVEVHNAKTKPREEQYNS | |
| | TYRVVSVLTVLHQDWLNGKEYKCKVSNKALPSSIEKTI | |
| | SKAKGQPREPQVYTLPPSREEMTKNQVSLTCLVKGFYP | |
| | SDIAVEWESNGQPENNYKTTPPVLDSDGSFFLYSKLTV | |
| | DKSRWQQGNVFSCSVMHEALHNHYTQKSLSLSPGK | |
| Lodapolimab (Generic) | QSVLTQPPSASGTPGQRVTISCSGSSSNIGSNTVNWYQQ | 127 |
| LY-3300054 | LPGTAPKLLIYGNSNRPSGVPDRFSGSKSGTSASLAISGL | |
| NR4MAD6PPB | QSEDEADYYCQSYDSSLSGSVFGGGIKLTVLGQPKAAP | |
| VL | SVTLFPPSSEELQANKATLVCLISDFYPGAVTVAWKAD | |
| | SSPVKAGVETTTPSKQSNNKYAASSYLSLTPEQWKSHR | |
| | SYSCQVTHEGSTVEKTVAPAECS | |
| HLX-20 | SYTMN | 128 |
| PL2#3 | | |
| VH CDR1 | | |
| HLX-20 | SISSGSDYLYYADSVKG | 129 |
| PL2#3 | | |
| VH CDR2 | | |
| HLX-20 | NELRWYPQAGAFDR | 130 |
| PL2#3 | | |
| VH CDR3 | | |
| HLX-20 | SGSSSYIESSYVG | 131 |
| PL2#3 | | |
| VL CDR1 | | |
| HLX-20 | DDDMRPS | 132 |
| PL2#3 | | |
| VL CDR2 | | |
| HLX-20 | EIWRSGLGGV | 133 |
| PL2#3 | | |
| VL CDR3 | | |
| Envafolimab (Generic) | QVQLVESGGGLVQPGGSLRLSCAASGKMSSRRCMAWF | 134 |
| 3D-025 | RQAPGKERERVAKLLTTSGSTYLADSVKGRFTISRDNS | |
| ASC-22 | KNTVYLQMNSLRAEDTAVYYCAADSFEDPTCTLVTSS | |
| KN-035 | GAFQYWGQGTLVTVSSEPKSSDKTHTCPPCPAPELLGG | |
| hu56V1-Fc-m1 | PSVFLFPPKPKDTLMISRTPEVTCVVVAVSHEDPEVKFN | |
| single-domain antibody | WYVDGVEVHNAKTKPREEQYNSTYRVVSVLTVLHQD | |
| | WLNGKEYKCKVSNKALPAGIEKTISKAKGQPREPQVYT | |
| | LPPSRDELTKNQVSLTCLVKGFYPSDIAVEWESNGQPE | |
| | NNYKTTPPVLDSDGSFFLYSKLTVDKSRWQQGNVFSCS | |
| | VMHEALHNHYTQKSLSLSPGK | |
| ND-021 | DIQMTQSPASLSASVGDRVTITCQASQSIGTYLAWYQQ | 135 |
| NM21-1480 | KPGKPPKLLIYRAFILASGVPSRFSGSGSGTDFTLTISSLQ | |
| PRO-1480 | PEDFATYYCQSNFYSDSTTIGPNAFGTGTKVTVLGGGG | |
| Tri-specific fusion | GSEVQLVESGGGLVQPGGSLRLSCAASGFSFSANYYPC | |
| single-chain antibody | WVRQAPGKGLEWIGCIYGGSSDITYDANWTKGRFTISR | |
| construct | DNSKNTVYLQMNSLRAEDTAVYYCARSAWYSGWGG | |
| | DLWGQGTLVTVSSGGGGSGGGGSGGGGSGGGGSIQMT | |
| | QSPSSLSASVGDRVTITCQASQSISNRLAWYQQKPGKAP | |
| | KLLIYSASTLASGVPSRFSGSGSGTDFTLTISSLQPEDFA | |
| | TYYCQSTYYGNDGNAFGTGTKVTVLGGGGGSEVQLVE | |
| | SGGGLVQPGGSLRLSCAASGFSFNSDYWIYWVRQAPG | |
| | KGLEWIASIYGGSSGNTQYASWAQGRFTISRDNSKNTV | |
| | YLQMNSLRAEDTAVYFCARGYVDYGGATDLWGQGTL | |
| | VTVSSGGGGSGGGGSIQMTQSPSSLSASVGDRVTITCQS | |
| | SESVYSNNQLSWYQQKPGQPPKLLIYDASDLASGVPSR | |
| | FSGSGSGTDFTLTISSLQPEDFATYYCAGGFSSSSDTAFG | |
| | GGTKLTVLGGGGGSGGGGSGGGGSGGGGSEVQLVESG | |
| | GGLVQPGGSLRLSCAASGFSLSSNAMGWVRQAPGKGL | |
| | EYIGIISVGGFTYYASWAKGRFTISRDNSKNTVYLQMNS | |
| | LRAEDTATYFCARDRHGGDSSGAFYLWGQGTLVTVSS | |
| ACE-05 | QMQLVQSGAEVKKPGSSVKVSCKASGGTFSSYAISWV | 136 |
| YBL-013 | RQAPGQGLEWMGRIIPILGIANYAQKFQGRVTITADKST | |
| Bispecific tetrameric | STAYMELSSLRSEDTAVYYCAKPRDGYNLVAFDIWGQ | |
| antibody-like cell | GTMVTVSSASTKGPSVFPLAPSSKSTSGGTAALGCLVK | |
| engager (ALiCE) | DYFPEPVTVSWNSGALTSGVHTFPAVLQSSGLYSLSSV | |
| comprising two identical | VTVPSSSLGTQTYICNVNHKPSNTKVDKKVEPKSCDKT | |
| light chains (LC) | HTCPPCPAPELLGGPGGGGSEVQLQQSGPELVKPGPSM | |
| consisting of antigen | KISCKASGYSFTGYTMNWVKQSHGKNLEWMGLINPYK | |
| binding domains | GVSTYNQKFKDKATLTVDKSSSTAYMELLSLTSEDSAV | |
| (ABDs) targeting | YYCARSGYYGDSDWYFDVWGQGTTLTVFS | |
| programmed cell death- | QMQLVQSGAEVKKPGSSVKVSCKASGGTFSSYAISWV | |
| ligand 1 (PD-L1), and | RQAPGQGLEWMGRIIPILGIANYAQKFQGRVTITADKST | |
| two different heavy | STAYMELSSLRSEDTAVYYCAKPRDGYNLVAFDIWGQ | |
| chain (HC)-like chains | GTMVTVSSASTKGPSVFPLAPSSKSTSGGTAALGCLVK | |
| (ACE-05-VH and ACE- | DYFPEPVTVSWNSGALTSGVHTFPAVLQSSGLYSLSSV | |
| 05-VL) each consisting | VTVPSSSLGTQTYICNVNHKPSNTKVDKKVEPKSCDKT | |
| of an anti-PD-L1 ABD | HTCPPCPAPELLGGPGGGGSDIQMTQTTSSLSASLGDRV | |
| and an anti-CD3 ABD; | TISCRASQDIRNYLNWYQQKPDGTVKLLIYYTSRLHSG | |
| wherein each HC | VPSKFSGSGSGTDYSLTISNLEQEDIATYFCQQGNTLPW | |
| comprises a G4S linker | TFAGGTKLEIKR | |
| (SEQ ID NO: 189) | QLVLTQPPSVSGAPGQRVTISCTGSSSNIGAGYDVHWY | |
| between the hinge | QQLPGAAPKLLIYGDINRPSGVPDRFSGSKSGISASLAIT | |
| region and the second | GLQAEDEADYYCQSYDSSLSGGVFGGGTKLTVLRTVA | |
| ABD | APSVFIFPPSDEQLKSGTASVVCLLNNFYPREAKVQWK | |
| | VDNALQSGNSQESVTEQDSKDSTYSLSSTLTLSKADYE | |
| | KHKVYACEVTHQGLSSPVTKSFNRGEC | |

An anti-PD-L1 polypeptide or an anti-PD-L1 antigen binding fragment comprises a VH having an amino acid sequence of any one of SEQ ID NOS: 110, 112, 114, 116, 120, 122, or 126. An anti-PD-L1 polypeptide or an anti-PD-L1 antigen binding fragment comprises a VH having an amino acid sequence of any one of SEQ ID NOS: 111, 113, 115, 117, 121, 123, or 127. In one instance, an anti-PD-L1 polypeptide or an anti-PD-L1 antigen binding fragment comprises a VH having an amino acid sequence of SEQ ID NO: 110, and a VL having an amino acid sequence of SEQ ID NO: 111. In another instance, an anti-PD-L1 polypeptide or an anti-PD-L1 antigen binding fragment comprises a VH having an amino acid sequence of SEQ ID NO: 112, and a VL having an amino acid sequence of SEQ ID NO: 113. In another instance, an anti-PD-L1 polypeptide or an anti-PD-L1 antigen binding fragment comprises a VH having an amino acid sequence of SEQ ID NO: 114, and a VL having an amino acid sequence of SEQ ID NO: 115. In another instance, an anti-PD-L1 polypeptide or an anti-PD-L1 antigen binding fragment comprises a VH having an amino acid sequence of SEQ ID NO: 116, and a VL having an amino acid sequence of SEQ ID NO: 117. In another instance, an anti-PD-L1 polypeptide or an anti-PD-L1 antigen binding fragment comprises a VH having an amino acid sequence of SEQ ID NO: 120, and a VL having an amino acid sequence of SEQ ID NO: 121. In another instance, an anti-PD-L1 polypeptide or an anti-PD-L1 antigen binding fragment comprises a VH having an amino acid sequence of SEQ ID NO: 122, and a VL having an amino acid sequence of SEQ ID NO: 123. In another instance, an anti-PD-L1 polypeptide or an anti-PD-L1 antigen binding fragment comprises a VH having an amino acid sequence of SEQ ID NO: 126, and a VL having an amino acid sequence of SEQ ID NO: 127.

In one instance, an anti-PD-L1 polypeptide or an anti-PD-L1 antigen binding fragment comprises a VH CHR1 having an amino acid sequence of SEQ ID NO: 128, a VH CHR2 having an amino acid sequence of SEQ ID NO: 129, a VH CHR3 having an amino acid sequence of SEQ ID NO: 130, VL CHR1 having an amino acid sequence of SEQ ID NO: 131, a VL CHR2 having an amino acid sequence of SEQ ID NO: 132, and a VL CHR3 having an amino acid sequence of SEQ ID NO: 133.

In one instance, an anti-PD-L1 polypeptide comprises a single domain binding antibody having an amino acid sequence of SEQ ID NO: 134, a tri-specific fusion single chain antibody construct having an amino acid sequence of SEQ ID NO: 135, or a bispecific tetrameric antibody like engager having an amino acid sequence of SEQ ID NO: 136.

### Anti-CD20 Antibodies

An anti-CD20 antibody or an anti-CD20 antigen binding fragment of the disclosure comprises a modified Rituximab (RITUXAN®), Ofatumumab (KESIMPTA®), Obinutuzumab (GAZYVA®), or Ocrelizumab (OCREVUS®). In one embodiment, an anti-CD20 antibody or an anti-CD20 antigen binding fragment of the disclosure comprises a combination of a heavy chain variable region (VH) and a light chain variable region (VL) of Rituximab (RITUXAN®), Ofatumumab (KESIMPTA®), Obinutuzumab (GAZYVA®), or Ocrelizumab (OCREVUS®). In another embodiment, an anti-CD20 antibody or an anti-CD20 antigen binding fragment of the disclosure comprises a combination of complementarity determining regions (VH CDR1, VH CDR2, VH CDR3, VL CDR1, VL CDR2, and VL CDR3) of Rituximab (RITUXAN®), Ofatumumab (KESIMPTA®), Obinutuzumab (GAZYVA®), or Ocrelizumab (OCREVUS®).

In one embodiment, an anti-CD20 antibody or an anti-CD20 antigen binding fragment of the disclosure comprises a fusion protein or a peptide immunotherapeutic agent. In one embodiment, an anti-CD20 agent of the disclosure comprises a cell such as, for example, a CART cell or a cytotoxic T lymphocyte.

TABLE 1C provides sequences of exemplary anti-CD20 polypeptides and anti-CD20 antigen binding fragments, a fusion protein, or a peptide immunotherapeutic agents, CART cell, and cytotoxic T lymphocytes that can be modified to prepare anti-CD20 immunoconjugates. TABLE 1C also provides exemplary combinations of CDRs that can be utilized in a modified anti-CD20 immunoconjugate. Reference to an anti-CD20 polypeptide herein may alternatively refer to an anti-CD20 antigen binding fragment.

TABLE 1C

| Drug Name | | |
|---|---|---|
| (Generic, Brand | | SEQ ID |
| Name, Code Name) | Sequence | NO |
| Rituximab | QVQLQQPGAELVKPGASVKMSCKASGYTFTSYNMHW | 137 |
| (RITUXAN ®) | VKQTPGRGLEWIGAIYPGNGDTSYNQKFKGKATLTAD | |
| heavy chain | KSSSTAYMQLSSLTSEDSAVYYCARSTYYGGDWYFNV | |
| | WGAGTTVTVSAASTKGPSVFPLAPSSKSTSGGTAALGC | |
| | LVKDYFPEPVTVSWNSGALTSGVHTFPAVLQSSGLYSL | |
| | SSVVTVPSSSLGTQTYICNVNHKPSNTKVDKKVEPKSCD | |
| | KTHTCPPCPAPELLGGPSVFLFPPKPKDTLMISRTPEVTC | |
| | VWDVSHEDPEVKFNWYVDGVEVHNAKTKPREEQYN | |
| | STYRVVSVLTVLHQDWLNGKEYKCKVSNKALPAPIEKT | |
| | ISKAKGQPREPQVYTLPPSRDELTKNQVSLTCLVKGFYP | |
| | SDIAVEWESNGQPENNYKTTPPVLDSDGSFFLYSKLTVD | |
| | KSRWQQGNVFSCSVMHEALHNHYTQKSLSLSPGK | |
| Rituximab | QIVLSQSPAILSASPGEKVTMTCRASSSVSYIHWFQQKP | 138 |
| (RITUXAN ®) | GSSPKPWIYATSNLASGVPVRFSGSGSGTSYSLTISRVEA | |
| light chain | EDAATYYCQQWTSNPPTFGGGTKLEIKRTVAAPSVFIFP | |
| | PSDEQLKSGTASVVCLLNNFYPREAKVQWKVDNALQS | |
| | GNSQESVTEQDSKDSTYSLSSTLTLSKADYEKHKVYAC | |
| | EVTHQGLSSPVTKSFNRGEC | |
| Ofatumumab | EVQLVESGGGLVQPGRSLRLSCAASGFTFNDYAMHWV | 139 |
| (KESIMPTA ®) | RQAPGKGLEWVSTISWNSGSIGYADSVKGRFTISRDNA | |
| heavy chain | KKSLYLQMNSLRAEDTALYYCAKDIQYGNYYYGMDV | |
| | WGQGTTVTVSSASTKGPSVFPLAPGSSKSTSGTAALGCL | |
| | VKDYFPEPVTVSWNSGALTSGVHTFPAVLQSSGLYSLSS | |
| | VVTVPSSSLGTQTYICNVNHKPSNTKVDKKVEP | |
| Ofatumumab | EIVLTQSPATLSLSPGERATLSCRASQSVSSYLAWYQQK | 140 |
| (KESIMPTA ®) | PGQAPRLLIYDASNRATGIPARFSGSGSGTDFTLTISSLEP | |
| light chain | EDFAVYYCQQRSNWPITFGQGTRLEIKRTVAAPSVFIFP | |
| | PSDEQLKSGTASVVCLLNNFYPREAKVQWKVDNALQS | |
| | GNSQESVTEQDSKDSTYSLSSTLTLSKADYEKHKVYAC | |
| | EVTHQGLSSPVTKSFNR | |
| Obinutuzumab | QVQLVQSGAEVKKPGSSVKVSCKASGYAFSYSWINWV | 141 |
| (GAZYVA ®) | RQAPGQGLEWMGRIFPGDGDTDYNGKFKGRVTITADK | |
| heavy chain | STSTAYMELSSLRSEDTAVYYCARNVFDGYWLVYWGQ | |
| | GTLVTVSSASTKGPSVFPLAPSSKSTSGGTAALGCLVKD | |
| | YFPEPVTVSWNSGALTSGVHTFPAVLQSSGLYSLSSVVT | |
| | VPSSSLGTQTYICNVNHKPSNTKVDKKVEPKSCDKTHT | |
| | CPPCPAPELLGGPSVFLFPPKPKDTLMISRTPEVTCVVVD | |
| | VSHEDPEVKFNWYVDGVEVHNAKTKPREEQYNSTYRV | |
| | VSVLTVLHQDWLNGKEYKCKVSNKALPAPIEKTISKAK | |
| | GQPREPQVYTLPPSRDELTKNQVSLTCLVKGFYPSDIAV | |
| | EWESNGQPENNYKTTPPVLDSDGSFFLYSKLTVDKSRW | |
| | QQGNVFSCSVMHEALHNHYTQKSLSLSPG | |
| Obinutuzumab | DIVMTQTPLSLPVTPGEPASISCRSSKSLLHSNGITYLYW | 142 |
| (GAZYVA ®) | YLQKPGQSPQLLIYQMSNLVSGVPDRFSGSGSGTDFTLK | |
| light chain | ISRVEAEDVGVYYCAQNLELPYTFGGGTKVEIKRTVAA | |
| | PSVFIFPPSDEQLKSGTASVVCLLNNFYPREAKVQWKV | |
| | DNALQSGNSQESVTEQDSKDSTYSLSSTLTLSKADYEK | |
| | HKVYACEVTHQGLSSPVTKSFNRGEC | |
| Ocrelizumab | EVQLVESGGGLVQPGGSLRLSCAASGYTFTSYNMHWV | 143 |
| (OCREVUS ®) | RQAPGKGLEWVGAIYPGNGDTSYNQKFKGRFTISVDKS | |
| heavy chain | KNTLYLQMNSLRAEDTAVYYCARVVVYYSNSYWYFD | |
| artificial | VWGQGTLVTVSSASTKGPSVFPLAPSSKSTSGGTAALG | |
| sequence | CLVKDYFPEPVTVSWNSGALTSGVHTFPAVLQSSGLYS | |
| | LSSVVTVPSSSLGTQTYICNVNHKPSNTKVDKKVEPKSC | |
| | DKTHTCPPCPAPELLGGPSVFLFPPKPKDTLMISRTPEVT | |
| | CVVVDVSHEDPEVKFNWYVDGVEVHNAKTKPREEQY | |
| | NSTYRVVSVLTVLHQDWLNGKEYKCKVSNKALPAPIE | |
| | KTISKAKGQPREPQVYTLPPSREEMTKNQVSLTCLVKGF | |
| | YPSDIAVEWESNGQPENNYKTTPPVLDSDGSFFLYSKLT | |
| | VDKSRWQQGNVFSCSVMHEALHNHYTQKSLSLSPGK | |
| Ocrelizumab | DIQMTQSPSSLSASVGDRVTITCRASSSVSYMHWYQQK | 144 |
| (OCREVUS ®) | PGKAPKPLIYAPSNLASGVPSRFSGSGSGTDFTLTISSLQP | |
| light chain | EDFATNYCQQWSFNPPTFGQGTKVEIKRYVAAPSVFIFP | |
| artificial | PSDEQLKSGTASVVCLLNNFYPREAKVQWKVDNALQS | |
| sequence | GNSQESVTEQDSKDSTYSLSSTLTLSKADYEKHKVYAC | |
| | EVTHQGLSSPVTKSFNRGEC | |

In some embodiments, the anti-CD20 polypeptide is modified with mAB3. In some embodiments, the anti-CD20 polypeptide is modified with mAB4.

An anti-CD20 polypeptide or an anti-CD20 antigen binding fragment can comprise a VH having an amino acid sequence of SEQ ID NO: 137, 139, 141, or 143. An anti-CD20 polypeptide or an anti-CD20 antigen binding fragment can comprise a VL having an amino acid sequence of SEQ ID NO: 138, 140, 142, or 144. In one instance, an anti-CD20 polypeptide or an anti-CD20 antigen binding fragment comprises a VH having an amino acid sequence of SEQ ID NO: 137 and a VL having an amino acid sequence of SEQ ID NO: 138. In another instance, an anti-CD20 polypeptide or an anti-CD20 antigen binding fragment comprises a VH having an amino acid sequence of SEQ ID NO: 139 and a VL having an amino acid sequence of SEQ ID NO: 140. In another instance, an anti-CD20 polypeptide or an anti-CD20 antigen binding fragment comprises a VH having an amino acid sequence of SEQ ID NO: 141 and a VL having an amino acid sequence of SEQ ID NO: 142. In another instance, an anti-CD20 polypeptide or an anti-CD20 antigen binding fragment comprises a VH having an amino acid sequence of SEQ ID NO: 143, and a VL having an amino acid sequence of SEQ ID NO: 144.

### Anti-TNFα Antibodies

In one embodiment, an anti-TNFα antibody or an anti-TNFα antigen binding fragment of the disclosure comprises a combination of a heavy chain variable region (VH) and a light chain variable region (VL) described herein. In another embodiment, an anti-CD20 antibody or an anti-TNFα antigen binding fragment of the disclosure comprises a combination of complementarity determining regions (VH CDR1, VH CDR2, VH CDR3, VL CDR1, VL CDR2, and VL CDR3) described herein. In one embodiment, an anti-TNFα antibody or an anti-TNFα antigen binding fragment of the disclosure comprises a modified Adalimumab (HUMIRA®). In one embodiment, an anti-TNFα antibody or an anti-TNFα antigen binding fragment of the disclosure comprises a modified INFLIXIMAB (AVSOLA®).

In one embodiment, an antd-TNFα antibody or an anti-TNFα antigen binding fragment of the disclosure comprises a fusion protein or a peptide immunotherapeutic agent.

In one embodiment, an anti-TNF agent of the disclosure comprises a cell such as, for example, a CART cell or a cytotoxic T lymphocyte.

TABLE 1D provides the sequences of exemplary anti-TNFα polypeptides and anti-TNFα antigen binding fragments, fusion protein or a peptide immunotherapeutic agents, CART cell, and cytotoxic T lymphocytes that can be modified to prepare anti-TNFα immunoconjugates. TABLE 1D also provides exemplary combinations of CDRs that can be utilized in a modified anti-TNFα immunoconjugate. Reference to an anti-TNFα polypeptide herein may alternatively refer to an anti-TNFα antigen binding fragment.

TABLE ID

| Drug Name (Generic, | | |
|---|---|---|
| Brand Name, Code | | SEQ ID |
| Name) | Sequence | NO: |
| Adalimumab | EVQLVESGGGLVQPGRSLRLSCAASGFTFDDYAM | 145 |
| (HUMIRA ®) Heavy | HWVRQAPGKGLEWVSAITWNSGHIDYADSVEGRF | |
| chain | TISRDNAKNSLYLQMNSLRAEDTAVYYCAKVSYL | |
| | STASSLDYWGQGTLVTVSSASTKGPSVFPLAPSSK | |
| | STSGGTAALGCLVKDYFPEPVTVSWNSGALTSGV | |
| | HTFPAVLQSSGLYSLSSVVTVPSSSLGTQTYICNVN | |
| | HKPSNTKVDKKVEPKSCDKTHTCPPCPAPELLGGP | |
| | SVFLFPPKPKDTLMISRTPEVTCVVVDVSHEDPEV | |
| | KFNWYVDGVEVHNAKTKPREEQYNSTYRVVSVL | |
| | TVLHQDWLNGKEYKCKVSNKALPAPIEKTISKAK | |
| | GQPREPQVYTLPPSRDELTKNQVSLTCLVKGFYPS | |
| | DIAVEWESNGQPENNYKTTPPVLDSDGSFFLYSKL | |
| | TVDKSRWQQGNVFSCSVMHEALHNHYTQKSLSLS | |
| | PGK | |
| Adalimumab | DIQMTQSPSSLSASVGDRVTITCRASQGIRNYLAW | 146 |
| (HUMIRA ®) Light | YQQKPGKAPKLLIYAASTLQSGVPSRFSGSGSGTD | |
| chain | FTLTISSLQPEDVATYYCQRYNRAPYTFGQGTKVEI | |
| | KRTVAAPSVFIFPPSDEQLKSGTASVVCLLNNFYPR | |
| | EAKVQWKVDNALQSGNSQESVTEQDSKDSTYSLS | |
| | STLTLSKADYEKHKVYACEVTHQGLSSPVTKSFNR | |
| | GEC | |
| INFLIXIMAB | EVKLEESGGGLVQPGGSMKLSCVASGFIFSNHWM | 147 |
| (AVSOLA ®)VH | NWVRQSPEKGLEWVAEIRSKSINSATHYAESVKG | |
| | RFTISRDDSKSAVYLQMTDLRTEDTGVYYCSRNY | |
| | YGSTYDYWGQGTTLTVSSASTKGPSVFPLAPSSKS | |
| | TSGGTAALGCLVKDYFPEPVTVSWNSGALTSGVH | |
| | TFPAVLQSSGLYSLSSVVTVPSSSLGTQTYICNVNH | |
| | KPSNTKVDKKVEPKSCDKT | |
| INFLIXIMAB | DILLTQSPAILSVSPGERVSFSCRASQFVGSSIHWYQ | 148 |
| (AVSOLA ®) VL | QRTNGSPRLLIKYASESMSGIPSRFSGSGSGTDFTLS | |
| | INTVESEDIADYYCQQSHSWPFTFGSGTNLEVKRT | |
| | VAAPSVFIFPPSDEQLKSGTASVVCLLNNFYPREAK | |
| | VQWKVDNALQSGNSQESVTEQDSKDSTYSLSSTL | |
| | TLSKADYEKHKVYACEVTHQGLSSPVTKSFNRGE | |
| | C | |
| 4H16 VH | QVTLRESGPALVKPTQTLTLTCTFSGFSLSTSGMCV | 149 |
| | SWIRQPPGKALEWLALIDWDDDKYYSTSLKTRLTI | |
| | SKDTSKNQVVLTMTNMDPVDTATYYCARILVDIV | |
| | ATITNDAFDVWGQGTMVTVSS | |
| 4H16 VL | DIQMTQSPSSLSASVGDRVTITCQASQDISNYLNW | 150 |
| | YQQKPGKAPKLLIYDASNLETGVPSRFSGSGSGTD | |
| | FTFTISSLQPEDIATYYCQQYDNLPPELTFGGGTKV | |
| | EIKR | |

In some embodiments, the anti-TNFα polypeptide is modified with mAB3. In some embodiments, the anti-TNFα polypeptide is modified with mAB4.

An anti-TNFα polypeptide or an anti-TNFα antigen binding fragment comprises a VH having an amino acid sequence of SEQ ID NO: 145 or 147. An anti-TNFα polypeptide or an anti-TNFα antigen binding fragment comprises a VL having an amino acid sequence of SEQ ID NO: 146 or 148.

In one instance, an anti-TNFα polypeptide or an anti-TNF antigen binding fragment comprises a VH having an amino acid sequence of SEQ ID NO: 145 and a VL having an amino acid sequence of SEQ ID NO: 146. In another instance, an anti-TNFα polypeptide or an anti-TNFα antigen binding fragment comprises a VH having an amino acid sequence of SEQ ID NO: 147 and a VL having an amino acid sequence of SEQ ID NO: 148. In another instance, an anti-TNFα polypeptide or an anti-TNFα antigen binding fragment comprises a VH having an amino acid sequence of SEQ ID NO: 149 and a VL having an amino acid sequence of SEQ ID NO: 150.

### Modification to Fc Regions

Disclosed herein are antibodies or antigen binding fragments, wherein the antibodies or antigen binding fragments comprise an Fc region, and wherein the Fc region comprises at least one covalently linked linker (e.g., a chemical linker). In some embodiments, the chemical linker is covalently attached to a lysine, or cysteine residue. In some embodiments, the chemical linker is covalently attached to a lysine residue. In some embodiments, the chemical linker is covalently attached to a constant region of the antibodies or antigen binding fragments.

The Fc region can be of any appropriate immunoglobulin isotype. In some embodiments, the Fc region is an IgG Fc region, an IgA Fc region, an IgD Fc region, an IgM Fc region, or an IgE Fc region. In some embodiments, the Fc region is an IgG Fc region, an IgA Fc region, or an IgD Fc region. In some embodiments, the Fc region is a human Fc region. In some embodiments, the Fc region is a humanized. Fc region. In some embodiments, the Fc region is an IgG Fc region. In some instances, an IgG Fc region is an IgG1 Fc region, an IgG2a Fc region, or an IgG4 Fc region.

One or more mutations may be introduced in an Fc region to reduce Fc-mediated effector functions of an antibody or antigen-binding fragment such as, for example, antibody-dependent cellular cytotoxicity (ADCC) and/or complement function. In some instances, a modified Fc comprises a humanized IgG4 kappa isotype that contains a S229P Fc mutation. In some instances, a modified Fc comprises a human IgG1 kappa where the heavy chain CH2 domain is engineered with a triple mutation such as, for example: (a) L238P, L239E, and P335S; or (2) K248; K288; and K317.

In some embodiments, the Fc region has an amino acid sequence at least 80%, at least 85%, at least 90%, at least 91%, at least 92%, at least 93%, at least 94%, at least 95%, at least 96%, at least 97%, at least 98%, or at least 99% identical to a sequence as set forth in SEQ ID NO: 151 (Pro Lys Ser Cys Asp Lys Thr His Thr Cys Pro Pro Cys Pro Ala Pro Glu Xaa Xaa Gly Xaa Pro Ser Val Phe Leu Phe Pro Pro Lys Pro Lys Asp Thr Leu Met Ile Ser Arg Thr Pro Glu Val Thr Cys Val Val Val Asp Val Ser His Glu Asp Pro Glu Val Lys Phe Asn Trp Tyr Val Asp Gly Val Glu Val His Asn Ala Lys Thr Lys Pro Arg Glu Glu Gln Tyr Asp Ser Thr Tyr Arg Val Val Ser Val Leu Thr Val Leu His Gln Asp Trp Leu Asn Gly Lys Glu Tyr Lys Cys Lys Val Ser Asn Lys Ala Leu Pro Ala Pro Ile Glu Lys Thr Ile Ser Lys Ala Lys Gly Gln Pro Arg Glu Pro Gln Val Tyr Thr Leu Pro Pro Ser Arg Xaa Glu Xaa Thr Lys Asn Gln Val Ser Leu Thr Cys Leu Val Lys Gly Phe Tyr Pro Ser Asp Ile Ala Val Glu Trp Glu Ser Asn Gly Gln Pro Glu Asn Asn Tyr Lys Xaa Thr Pro Pro Val Leu Asp Ser Asp Gly Ser Phe Phe Leu Tyr Ser Lys Leu Thr Val Asp Lys Ser Arg Trp Gln Gln Gly Asn Val Phe Ser Cys Ser Val Met His Glu Ala Leu His Asn His Tyr Thr Gln Lys Ser Leu Ser Leu Ser Pro Gly, where Xaa can be any naturally occurring amino acid). In some embodiments, the Fc region comprises one or more mutations which make the Fc region susceptible to modification or conjugation at a particular residue, such as by incorporation of a cysteine residue at a position which does not contain a cysteine in SEQ ID NO: 151. Alternatively, the Fc region could be modified to incorporate a modified natural amino acid or an unnatural amino acid which comprises a conjugation handle, such as one connected to the modified natural amino acid or unnatural amino acid through a linker. In some embodiments, the Fc region does not comprise any mutations which facilitate the attachment of a linker to a cytokine (e.g., an IL-2 polypeptide, an IL7 polypeptide, or an IL18 polypeptide, etc.). In some embodiments, the chemical linker is attached to a native residue as set forth in SEQ ID NO: 151. In some embodiments, the chemical linker is attached to a native lysine residue of SEQ ID NO: 151.

In some embodiments, the chemical linker is attached to the Fc region at an amino acid residue at one of positions 10-90 of SEQ ID NO: 151. In some embodiments, the chemical linker is attached to the Fc region at an amino acid residue at one of positions 10-20, 10-30, 10-40, 10-50, 10-60, 10-70, 1-80, 10-90, 10-100, 10-110, 10-120, 10-130, 10-140, 10-150, 10-160, 10-170, 10-180, 10-190, or 10-200 of SEQ ID NO: 151. In some embodiments, the chemical linker is attached to the Fc region at an amino acid residue at one of positions 10-30, 50-70, or 80-100 of SEQ ID NO: 151. In some embodiments, the chemical linker is attached to the Fc region at an amino acid residue at one of positions 15-26, 55-65, or 85-90 of SEQ ID NO: 151. In some embodiments, the chemical linker is attached to the Fc region at an amino acid residue at one of positions 16, 18, 58, 60, or 87 of SEQ ID NO: 151. In some embodiments, the chemical linker is attached to the Fc region at an amino acid residue at one of positions K16, K18, K58, K60 or K87 of SEQ ID NO: 151. In some embodiments, the chemical linker is attached to the Fc region at an amino acid residue at position 16 of SEQ ID NO: 151. In some embodiments, the chemical linker is attached to the Fc region at an amino acid residue at position 18 of SEQ ID NO: 151. In some embodiments, the chemical linker is attached to the Fc region at an amino acid residue at position 58 of SEQ ID NO: 151. In some embodiments, the chemical linker is attached to the Fc region at an amino acid residue at position 60 of SEQ ID NO: 151. In some embodiments, the chemical linker is attached to the Fc region at an amino acid residue at position 87 of SEQ ID NO: 151.

In some embodiments, the chemical linker is attached to the Fc region at an amino acid residue at any one of positions 10-90 of SEQ ID NO: 151. In some embodiments, the chemical linker is attached to the Fc region at an amino acid residue at any one of positions 10-20, 10-30, 10-40, 10-50, 10-60, 10-70, 1-80, 10-90, 10-100, 10-110, 10-120, 10-130, 10-140, 10-150, 10-160, 10-170, 10-180, 10-190, or 10-200 of SEQ ID NO: 151. In some embodiments, the chemical linker is attached to the Fc region at an amino acid residue at any one of positions 20-40, 65-85, or 90-110 of SEQ ID NO: 151. In some embodiments, the chemical linker is attached to the Fc region at an amino acid residue at one of positions 10-30, 50-70, or 80-100 of SEQ ID NO: 151. In some embodiments, the chemical linker is attached to the Fc region at an amino acid residue at any one of positions 25-35, 70-80, or 95-105 of SEQ ID NO: 151. In some embodiments, the chemical linker is attached to the Fc region at an amino acid residue at one of positions 15-26, 55-65, or 85-90 of SEQ ID NO: 151. In some embodiments, the chemical linker is attached to the Fc region at an amino acid residue at any one of positions 30, 32, 72, 74, 79 or 101 of SEQ ID NO: 151. In some embodiments, the chemical linker is attached to the Fc region at an amino acid residue at any one of positions K30, K32, K72, K74, Q79, or K101 of SEQ ID NO: 151. In some embodiments, the chemical linker is attached to the Fc region at amino acid residue 30 of SEQ ID NO: 151. In some embodiments, the chemical linker is attached to the Fc region at amino acid residue 32 of SEQ ID NO: 151. In some embodiments, the chemical linker is attached to the Fc region at amino acid residue 72 of SEQ ID NO: 151. In some embodiments, the chemical linker is attached to the Fc region at amino acid residue 74 of SEQ ID NO: 151. In some embodiments, the chemical linker is attached to the Fc region at amino acid residue 79 of SEQ ID NO: 151. In some embodiments, the chemical linker is attached to the Fc region at amino acid residue 101 of SEQ ID NO: 151.

The chemical linker can be covalently attached to one amino acid residue of an Fc region of the antibody or antigen binding fragment. In some embodiments, the chemical linker is covalently attached to a non-terminal residue of the Fc region. In some embodiments, the non-terminal residue is in the CH1, CH2, or CH3 region of the antibody or antigen binding fragment. In some embodiments, the non-terminal residue is in the CH2 region of the of the antibody or antigen binding fragment.

In some embodiments, the chemical linker is covalently attached at an amino acid residue of the antibody or antigen binding fragment such that the function of the polypeptide is maintained (e.g., without denaturing the polypeptide). For example, when the polypeptide is an antibody such as a human IgG (e.g., human IgG1), exposed lysine residues and exposed tyrosine residues are present at the following positions (refer to web site: www.imgt.org/IMGTScientificChart/Numbering/Hu_IGHGnber.html by EU numbering). Exemplary exposed Lysine Residues: CH2 domain (position 246, position 248, position 274, position 288, position 290, position 317, position 320, position 322, and position 338) CH3 domain (position 360, position 414, and position 439). Exemplary exposed Tyrosine Residues: CH2 domain (position 278, position 296, and position 300) CH3 domain (position 436).

The human IgG, such as human IgG1, may also be modified with a lysine or tyrosine residue at any one of the positions listed above in order provide a residue which is ideally surface exposed for subsequent modification.

In some embodiments, the chemical linker is covalently attached at an amino acid residue in the constant region of an antibody or antigen binding fragment. In some embodiments, the chemical linker is covalently attached at an amino acid residue in the CH1, CH2, or CH3 region. In some embodiments, the chemical inker is covalently attached at an amino acid residue in the CH2 region. In some embodiments, the chemical linker may be covalently attached to one residue selected from the following groups of residues following EU numbering in human IgG Fc: amino acid residues 1-478, amino acid residues 2-478, amino acid residues 1-477, amino acid residues 2-477, amino acid residues 10-467, amino acid residues 30-447, amino acid residues 50-427, amino acid residues 100-377, amino acid residues 150-327, amino acid residues 200-327, amino acid residues 240-327, and amino acid residues 240-320.

In some embodiments, the chemical linker is covalently attached to one lysine residue of a human IgG Fc region. In some embodiments, the chemical linker is covalently attached at Lys 246 of an Fc region of the antibody or antigen binding fragment, wherein amino acid residue position number is based on Eu numbering. In some embodiments, the chemical linker is covalently attached at Lys 248 of an Fc region of the antibody or antigen binding fragment, wherein amino acid residue position number is based on Eu numbering. In some embodiments, the chemical linker is covalently attached at Lys 288 of an Fc region of the antibody or antigen binding fragment, wherein amino acid residue position number is based on Eu numbering. In some embodiments, the chemical linker is covalently attached at Lys 290 of an Fc region of the antibody or antigen binding fragment, wherein amino acid residue position number is based on Eu numbering. In some embodiments, the chemical linker is covalently attached at Lys 317 of the antibody or antigen binding fragment, wherein amino acid residue position number is based on Eu numbering.

In some embodiments, the chemical linker can be covalently attached to an amino acid residue selected from a subset of amino acid residues. In some embodiments, the subset comprises two three, four, five, six, seven, eight, nine, or ten amino acid residues of an Fc region of the antibody or antigen binding fragment. In some embodiments, the chemical linker can be covalently attached to one of two lysine residues of an Fc region of the antibody or antigen binding fragment.

In some embodiments, the antibody or antigen binding fragment will comprise two linkers covalently attached to the Fc region of the antibody or antigen binding fragment. In some embodiments, each of the two linkers will be covalently attached to a different heavy chain of the antibody or antigen binding fragment. In some embodiments, each of the two linkers will be covalently attached to a different heavy chain of the antibody or antigen binding fragment at a residue position which is the same. In some embodiments, each of the two linkers will be covalently attached to a different heavy chain of the antibody or antigen binding fragment at a residue position which is different. When the two linkers are covalently attached to residue positions which differ, any combination of the residue positions provided herein may be used in combination.

In some embodiments, a first chemical linker is covalently attached at Lys 248 of a first Fc region of the antibody or antigen binding fragment, and a second chemical linker is covalently attached at Lys 288 of a second Fc region of the antibody or antigen binding fragment, wherein residue position number is based on Eu numbering. In some embodiments, a first chemical linker is covalently attached at Lys 246 of a first Fc region of the antibody or antigen binding fragment, and a second chemical linker is covalently attached at Lys 288 of a second Fc region of the antibody or antigen binding fragment, wherein residue position number is based on Eu numbering. In some embodiments, a first chemical linker is covalently attached at Lys 248 of a first Fc region of the antibody or antigen binding fragment, and a second chemical linker is covalently attached at Lys 317 of a second Fc region of the antibody or antigen binding fragment, wherein residue position number is based on Eu numbering. In some embodiments, a first chemical linker is covalently attached at Lys 246 of a first Fc region of the antibody or antigen binding fragment, and a second chemical linker is covalently attached at Lys 317 of a second Fc region of the antibody or antigen binding fragment, wherein residue position number is based on Eu numbering. In some embodiments, a first chemical linker is covalently attached at Lys 288 of a first Fc region of the antibody or antigen binding fragment, and a second chemical linker is covalently attached at Lys 317 of a second Fc region of the antibody or antigen binding fragment, wherein residue position number is based on Eu numbering.

### Method of Modifying an Fc Region

Also provided herein are method of preparing a modified Fc region of an antibody or antigen binding fragment, such as for the attachment of a linker, a conjugation handle, or the cytokine to the antibody or antigen binding fragment. A variety of methods for site-specific modification of Fc regions of antibody or antigen binding fragment are known in the art.

Modification with an Affinity Peptide Configured to Site-Specifically Attach Linker to the Antibody

In some embodiments, an Fc region is modified to incorporate a linker, a conjugation handle, or a combination thereof. In some embodiments, the modification is performed by contacting the Fc region with an affinity peptide bearing a payload configured to attach a linker or other group to the Fc region, such as at a specific residue of the Fc region. In some embodiments, the linker is attached using a reactive group (e.g., a N-hydroxysuccinimide ester) which forms a bond with a residue of the Fc region. In some embodiments, the affinity peptide comprises a cleavable linker. The cleavable linker is configured on the affinity peptide such that after the linker or other group is attached to the Fc region, the affinity peptide can be removed, leaving behind only the desired linker or other group attached to the Fc region. The linker or other group can then be used further to add attach additional groups, such as a cytokine or a linker attached to a cytokine, to the Fc region. In such embodiments, a compatible antibody must contain a compatible Fc region (e.g., an IgG).

Non-limiting examples of such affinity peptides can be found at least in PCT Publication No. WO2018199337A1, PCT Publication No. WO2019240288A1, PCT Publication No. WO2019240287A1, and PCT Publication No. WO2020090979A1, each of which is incorporated by reference as if set forth herein in its entirety. In some embodiments, the affinity peptide is a peptide which has been modified to deliver the linker/conjugation handle payload one or more specific residues of the Fc region of the antibody. In some embodiments, the affinity peptide has at least 80%, 85%, 90%, 91%, 92%, 93%, 94%, 95%, 96%, 97%, 98%, 99%, or 100% sequence identify to a peptide selected from among (1) QETNPTENLYFQQKNMQCQRRFYEALHDPNLNEEQRNARIRSIRDDDC (SEQ ID NO: 154); (2) QTADNQKNMQCQRRFYEALHDPNLNEEQRNARIRSIRDDCSQSANLLAEAQQLNDAQAPQA (SEQ ID NO: 155); (3) QETKNMQCQRRFYEALHDPNLNEEQRNARIRSIRDDDC (SEQ ID NO: 156); (4) QETFNKQCQRRFYEALHDPNLNEEQRNARIRSIRDDDC (SEQ ID NO: 157); (5) QETFNMQCQRRFYEALHDPNLNKEQRNARIRSIRDDDC (SEQ ID NO: 158); (6) QETFNMQCQRRFYEALHDPNLNEEQRNARIRSIKDDC (SEQ ID NO: 159); (7) QETMQCQRRFYEALHDPNLNEEQRNARIRSIKDDC (SEQ ID NO: 160); (8) QETQCQRRFYEALHDPNLNEEQRNARIRSIKDDC (SEQ ID NO: 161); (9) QETCQRRFYEALHDPNLNEEQRNARIRSIKDDC (SEQ ID NO: 162); (10) QETRGNCAYHKGQLVWCTYH (SEQ ID NO: 163); and (11) QETRGNCAYHKGQIIWCTYH (SEQ ID NO: 164), or a corresponding peptide which has been truncated at the N-terminus by one, two, three, four, or five residues. An exemplary affinity peptide with cleavable linker and conjugation handle payload capable of attaching the payload to residue K248 of an antibody as provided herein is shown below (as reported in Matsuda et al., “Chemical Site-Specific Conjugation Platform to Improve the Pharmacokinetics and Therapeutic Index of Antibody-Drug Conjugates,” Mol. Pharmaceutics 2021, 18, 11, 4058-4066.

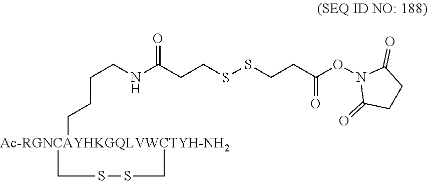

Alternative affinity peptides targeting alternative residues of the Fc region are described in the references cited above for AJICAP™ technology, and such affinity peptides can be used to attach the desired functionality to an alternative residue of the Fc region (e.g., K246, K288, etc.). For example, the disulfide group of the above affinity peptide could instead be replaced with a thioester to provide a sulfhydryl protecting group as a cleavable portion of the linking group (e.g., the relevant portion of the affinity peptide would have a structure of

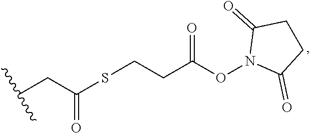 [SMILES: *CC(=O)SCCC(=O)ON1C(=O)CCC1=O]

or another of the cleavable linkers discussed below).

The affinity peptide of the disclosure can comprise a cleavable linker. In some embodiments, the cleavable linker of the affinity peptide connects the affinity peptide to the group which is to be attached to the Fc region and is configured such that the peptide can be cleaved after the group comprising the linker or conjugation handle has been attached. In some embodiments, the cleavable linker is a divalent group. In some embodiments, the cleavable linker can comprise a thioester group, an ester group, a sulfane group; a methanimine group; an oxyvinyl group; a thiopropanoate group; an ethane-1,2-diol group; an (imidazole-1-yl)methan-1-one group; a seleno ether group; a silylether group; a di-oxysilane group; an ether group; a di-oxymethane group; a tetraoxospiro[5.5]undecane group; an acetamidoethyl phosphoramidite group; a bis(methylthio)-pyrazolopyrazole-dione group; a 2-oxo-2-phenylethyl formate group; a 4-oxybenzylcarbamate group; a 2-(4-hydroxy-oxyphenyl)diazinyl)benzoic acid group; a 4-amino-2-(2-amino-2-oxoethyl)-4-oxobut-2-enoic acid group; a 2-(2-methylenehydrazineyl)pyridine group; an N′-methyleneformohydrazide group; or an isopropylcarbamate group, any of which is unsubstituted or substituted. Composition and points of attachment of the cleavable linker to the affinity peptide, as well as related methods of use, are described in, at least, PCT Publication No. WO2018199337A1, PCT Publication No. WO2019240288A1, PCT Publication No. WO2019240287A1, and PCT Publication No. WO2020090979A1.

In some embodiments, the cleavable linker is:

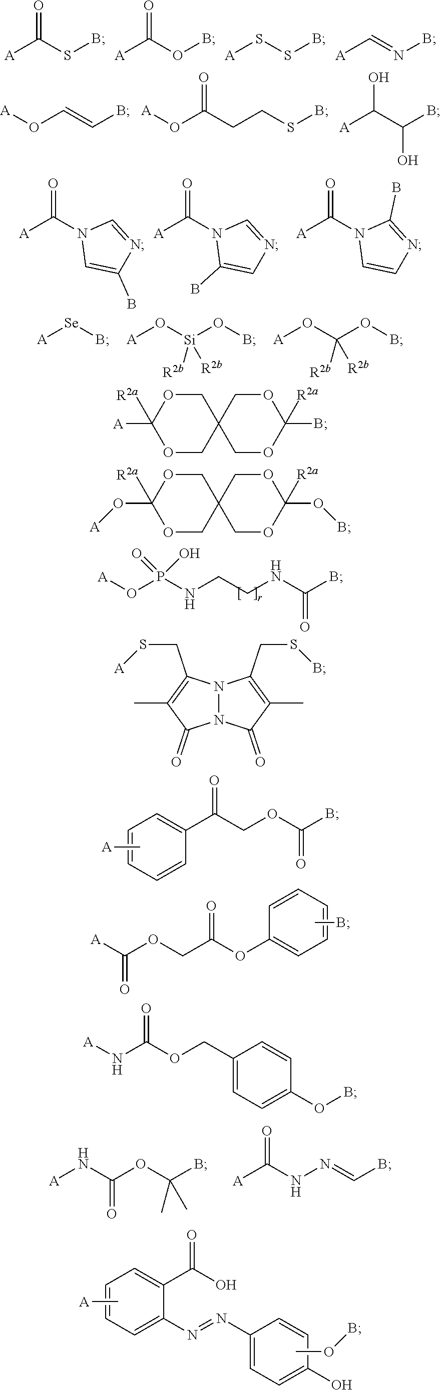

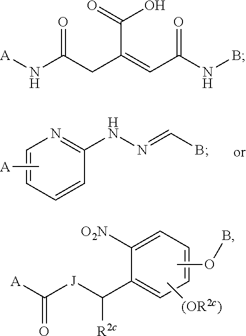

wherein:

one of A or B is a point of attachment the linker and the other of A or B is a point of attachment to the affinity peptide;

each R^{2a} is independently H or optionally substituted alkyl;

each R^{2b} is independently H or optionally substituted alkyl;

R^{2c} is a H or optionally substituted alkyl;

J is a methylene, a N, a S, a Si, or an O atom; and
r is 1, 2, 3, 4, 5, 6, 7, 8, 9, or 10.

The affinity peptide comprises a reactive group which is configured to enable the covalent attachment of the linker/conjugation handle to the Fc region. In some embodiments, the reactive group is selective for a functional group of a specific amino acid residue, such as a lysine residue, tyrosine residue, serine residue, cysteine residue, or an unnatural amino acid residue of the Fc region incorporated to facilitate the attachment of the linker. The reactive group may be any suitable functional group, such as an activated ester for reaction with a lysine (e.g., N-hydroxysuccinimide ester or a derivate thereof, a pentafluorophenyl ester, etc.) or a sulfhydryl reactive group for reaction with a cysteine (e.g., a Michael acceptor, such as an alpha-beta unsaturated carbonyl or a maleimide). In some embodiments, the reactive group is:

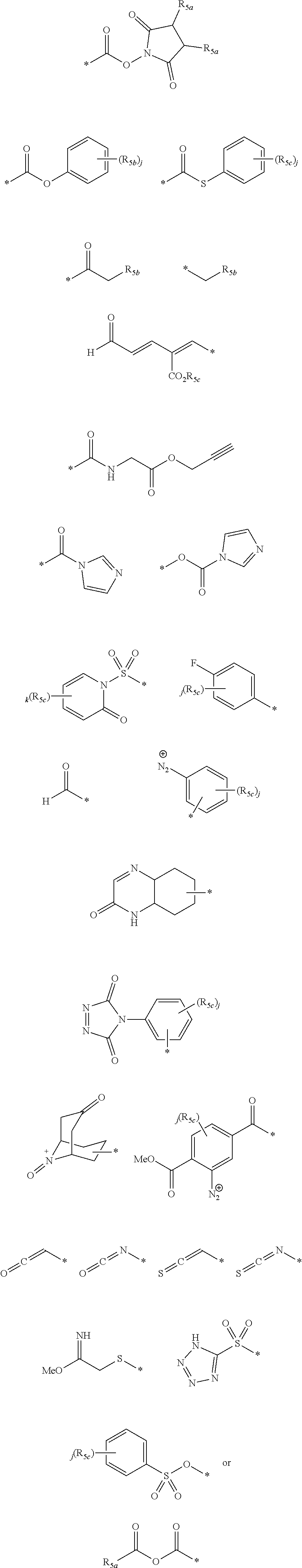

each R₅ₐ, R_{5b}, and R_{5c} is independently H, halogen, or optionally substituted alkyl;

each j is 1, 2, 3, 4, or 5; and
each k is 1, 2, 3, 4, or 5.

In some embodiments, the affinity peptide is used to deliver a reactive moiety to the desired amino acid residue such that the reactive moiety is exposed upon cleavage of the cleavable linker. By way of non-limiting example, the reactive group forms a covalent bond with a desired residue of the Fc region of the polypeptide which selectively binds to antibody or antigen binding fragment due to an interaction between the affinity peptide and the Fc region. Following this covalent bond formation, the cleavable linker is cleaved under appropriate conditions to reveal a reactive moiety (e.g., if the cleavable linker comprises a thioester, a free sulfhydryl group is attached to the Fc region following cleavage of the cleavable linker). This new reactive moiety can then be used to subsequently add an additional moiety, such as a conjugation handle, by way of reagent comprising the conjugation handle tethered to a sulfhydryl reactive group (e.g., alpha-halogenated carbonyl group, alpha-beta unsaturated carbonyl group, maleimide group, etc.).

In some embodiments, an affinity peptide is used to deliver a free sulfhydryl group to a lysine of the Fc region. In some embodiments, the free sulfhydryl group is then reacted with a bifunctional linking reagent to attach a new conjugation handle to the Fc region. In some embodiments, the new conjugation handle is then used to form the linker to the attached cytokine. In some embodiments, the new conjugation handle is an alkyne functional group. In some embodiments, the new conjugation handle is a DBCO functional group.

Exemplary bifunctional linking reagents useful for this purpose are of a formula A-B-C, wherein A is the sulfhydryl reactive conjugation handle (e.g., maleimide, α,β-unsaturated carbonyl, a-halogenated carbonyl), B is a lining group, and C is the new conjugation handle (e.g., an alkyne such as DBCO). Specific non-limiting examples of bifunctional linking reagents include

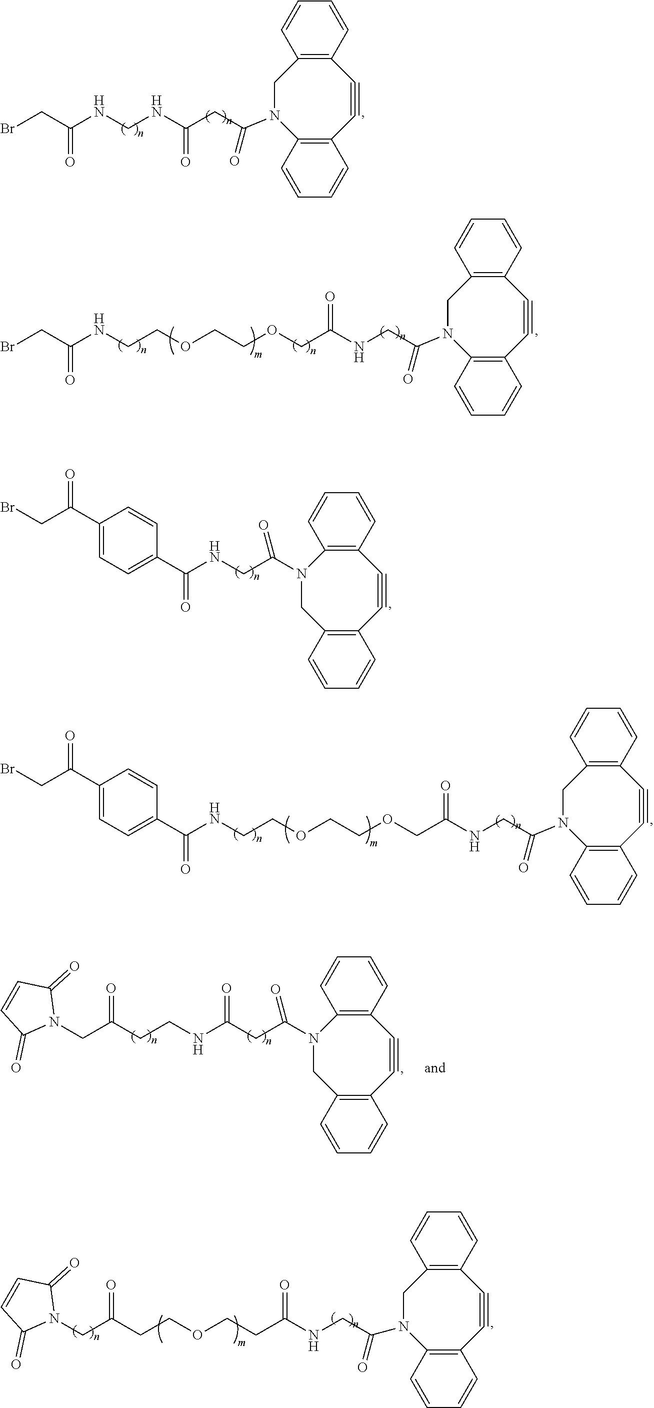

wherein each n is independently an integer from 1-6 and each m is independently an integer from 1-30, and related molecules (e.g., isomers).

Alternatively, the affinity peptide can be configured such that a conjugation handle is added to the Fc region (such as by a linker group) immediately after covalent bond formation between the reactive group and a residue of the Fc region. In such cases, the affinity peptide is cleaved and the conjugation handle is immediately ready for subsequent conjugation to the IL-2 polypeptide (or other cytokine).

### Alternative Methods of Attachment—Enzyme Mediated

While the affinity peptide mediated modification of an Fc region of an antibody provide supra possesses many advantages over other methods which can be used to site-specifically modify the Fc region (e.g., ease of use, ability to rapidly generate many different antibody conjugates, ability to use many “off-the-shelf” commercial antibodies without the need to do time consuming protein engineering, etc.), other methods of performing the modification are also contemplated as being within the scope of the present disclosure

In some embodiments, the present disclosure relates generally to transglutaminase-mediated site-specific antibody-drug conjugates (ADCs) comprising: 1) glutamine-containing tags, endogenous glutamines (e.g., native glutamines without engineering, such as glutamines in variable domains, CDRs, etc.), and/or endogenous glutamines made reactive by antibody engineering or an engineered transglutaminase; and 2) amine donor agents comprising amine donor units, linkers, and agent moieties. Non-limiting examples of such transglutaminase mediated site-specific modifications can be found at least in publications WO2020188061, US2022133904, US2019194641, US2021128743, U.S. Pat. Nos. 9,764,038, 10,675,359, 9,717,803, 10,434,180, 9,427,478, which are incorporated by reference as if set forth herein in their entirety.

In another aspect, the disclosure provides an engineered Fc-containing polypeptide conjugate comprising the formula: (Fc-containing polypeptide-T-A), wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the Fc-containing polypeptide, wherein the acyl donor glutamine-containing tag comprises an amino acid sequence XXQX, wherein X is any amino acid (e.g., X can be the same or different amino acid), and wherein the engineered Fc-containing polypeptide conjugate comprises an amino acid substitution from glutamine to asparagine at position 295 (Q295N; EU numbering scheme).

In some embodiments, the acyl donor glutamine-containing tag is not spatially adjacent to a reactive Lys (e.g., the ability to form a covalent bond as an amine donor in the presence of an acyl donor and a transglutaminase) in the polypeptide or the Fc-containing polypeptide. In some embodiments, the polypeptide or the Fc-containing polypeptide comprises an amino acid modification at the last amino acid position in the carboxyl terminus relative to a wild-type polypeptide at the same position. The amino acid modification can be an amino acid deletion, insertion, substitution, mutation, or any combination thereof.

In some embodiments, the polypeptide conjugate comprises a full length antibody heavy chain and an antibody light chain, wherein the acyl donor glutamine-containing tag is located at the carboxyl terminus of a heavy chain, a light chain, or both the heavy chain and the light chain.

In some embodiments, the polypeptide conjugate comprises an antibody, wherein the antibody is a monoclonal antibody, a polyclonal antibody, a human antibody, a humanized antibody, a chimeric antibody, a bispecific antibody, a minibody, a diabody, or an antibody fragment. In some embodiments, the antibody is an IgG.

In another aspect, described herein is a method for preparing an engineered Fc-containing polypeptide conjugate comprising the formula: (Fc-containing polypeptide-T-A), wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the Fc-containing polypeptide, wherein the acyl donor glutamine-containing tag comprises an amino acid sequence XXQX, wherein X is any amino acid (e.g., X can be the same or a different amino acid), and wherein the engineered Fc-containing polypeptide conjugate comprises an amino acid substitution from glutamine to asparagine at position 295 (Q295N; EU numbering scheme), comprising the steps of: a) providing an engineered (Fc-containing polypeptide)-T molecule comprising the Fc-containing polypeptide and the acyl donor glutamine-containing tag; b) contacting the amine donor agent with the engineered (Fc-containing polypeptide)-T molecule in the presence of a transglutaminase; and c) allowing the engineered (Fc-containing polypeptide)-T to covalently link to the amine donor agent to form the engineered Fc-containing polypeptide conjugate.

In another aspect, described herein is a method for preparing an engineered polypeptide conjugate comprising the formula: polypeptide-T-A, wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the polypeptide, and wherein the acyl donor glutamine-containing tag comprises an amino acid sequence LLQGPX (SEQ ID NO: 152), wherein X is A or P, or GGLLQGPP (SEQ ID NO: 153), comprising the steps of: a) providing an engineered polypeptide-T molecule comprising the polypeptide and the acyl donor glutamine-containing tag; b) contacting the amine donor agent with the engineered polypeptide-T molecule in the presence of a transglutaminase; and c) allowing the engineered polypeptide-T to covalently link to the amine donor agent to form the engineered Fc-containing polypeptide conjugate.

In some embodiments, the engineered polypeptide conjugate (e.g., the engineered Fc-containing polypeptide conjugate, the engineered Fab-containing polypeptide conjugate, or the engineered antibody conjugate) as described herein has conjugation efficiency of at least about 51%. In another aspect, the invention provides a pharmaceutical composition comprising the engineered polypeptide conjugate as described herein (e.g., the engineered Fc-containing polypeptide conjugate, the engineered Fab-containing polypeptide conjugate, or the engineered antibody conjugate) and a pharmaceutically acceptable excipient.

In some embodiments, described herein is a method for conjugating a moiety of interest (Z) to an antibody, comprising the steps of: (a) providing an antibody having (e.g., within the primary sequence of a constant region) at least one acceptor amino acid residue (e.g., a naturally occurring amino acid) that is reactive with a linking reagent (linker) in the presence of a coupling enzyme, e.g., a transamidase; and (b) reacting said antibody with a linking reagent (e.g., a linker comprising a primary amine) comprising a reactive group (R), optionally a protected reactive group or optionally an unprotected reactive group, in the presence of an enzyme capable of causing the formation of a covalent bond between the acceptor amino acid residue and the linking reagent (other than at the R moiety), under conditions sufficient to obtain an antibody comprising an acceptor amino acid residue linked (covalently) to a reactive group (R) via the linking reagent. Optionally, said acceptor residue of the antibody or antibody fragment is flanked at the +2 position by a non-aspartic acid residue. Optionally, the residue at the +2 position is a non-aspartic acid residue. In one embodiment, the residue at the +2 position is a non-aspartic acid, non-glutamine residue. In one embodiment, the residue at the +2 position is a non-aspartic acid, non-asparagine residue. In one embodiment, the residue at the +2 position is a non-negatively charged amino acid (an amino acid other than an aspartic acid or a glutamic acid). Optionally, the acceptor glutamine is in an Fc domain of an antibody heavy chain, optionally further-within the CH2 domain Optionally, the antibody is free of heavy chain N297-linked glycosylation. Optionally, the acceptor glutamine is at position 295 and the residue at the +2 position is the residue at position 297 (EU index numbering) of an antibody heavy chain.

In one aspect, described herein is a method for conjugating a moiety of interest (Z) to an antibody, comprising the steps of: (a) providing an antibody having at least one acceptor glutamine residue; and (b) reacting said antibody with a linker comprising a primary amine (a lysine-based linker) comprising a reactive group (R), preferably a protected reactive group, in the presence of a transglutaminase (TGase), under conditions sufficient to obtain an antibody comprising an acceptor glutamine linked (covalently) to a reactive group (R) via said linker. Optionally, said acceptor glutamine residue of the antibody or antibody fragment is flanked at the +2 position by a non-aspartic acid residue. Optionally, the residue at the +2 position is a non-aspartic acid residue. In one embodiment, the residue at the +2 position is a non-aspartic acid, non-glutamine residue. In one embodiment, the residue at the +2 position is a non-aspartic acid, non-asparagine residue. In one embodiment, the residue at the +2 position is a non-negatively charged amino acid (an amino acid other than an aspartic acid or a glutamic acid). Optionally, the acceptor glutamine is in an Fc domain of an antibody heavy chain, optionally further-within the CH2 domain Optionally, the antibody is free of heavy chain N297-linked glycosylation. Optionally, the acceptor glutamine is at position 295 and the residue at the +2 position is the residue at position 297 (EU index numbering) of an antibody heavy chain.

The antibody comprising an acceptor residue or acceptor glutamine residue linked to a reactive group (R) via a linker comprising a primary amine (a lysine-based linker) can thereafter be reacted with a reaction partner comprising a moiety of interest (Z) to generate an antibody comprising an acceptor residue or acceptor glutamine residue linked to a moiety of interest (Z) via the linker. Thus, in one embodiment, the method further comprises a step (c): reacting (i) an antibody of step b) comprising an acceptor glutamine linked to a reactive group (R) via a linker comprising a primary amine (a lysine-based linker), optionally immobilized on a solid support, with (ii) a compound comprising a moiety of interest (Z) and a reactive group (R′) capable of reacting with reactive group R, under conditions sufficient to obtain an antibody comprising an acceptor glutamine linked to a moiety of interest (Z) via a linker comprising a primary amine (a lysine-based linker). Preferably, said compound comprising a moiety of interest (Z) and a reactive group (R′) capable of reacting with reactive group R is provided at a less than 80 times, 40 times, 20 times, 10 times, 5 times or 4 molar equivalents to the antibody. In one embodiment, the antibody comprises two acceptor glutamines and the compound comprising a moiety of interest (Z) and a reactive group (R′) is provided at 10 or less molar equivalents to the antibody. In one embodiment, the antibody comprises two acceptor glutamines and the compound comprising a moiety of interest (Z) and a reactive group (R′) is provided at 5 or less molar equivalents to the antibody. In one embodiment, the antibody comprises four acceptor glutamines and the compound comprising a moiety of interest (Z) and a reactive group (R′) is provided at 20 or less molar equivalents to the antibody. In one embodiment, the antibody comprises four acceptor glutamines and the compound comprising a moiety of interest (Z) and a reactive group (R′) is provided at 10 or less molar equivalents to the antibody. In one embodiment, steps (b) and/or (c) are carried out in aqueous conditions. Optionally, step (c) comprises: immobilizing a sample of an antibody comprising a functionalized acceptor glutamine residue on a solid support to provide a sample comprising immobilized antibodies, reacting the sample comprising immobilized antibodies with a compound, optionally recovering any unreacted compound and re-introducing such recovered compound to the solid support for reaction with immobilized antibodies, and eluting the antibody conjugates to provide a composition comprising a Z moiety.

### Conjugation Handle Chemistry

In some embodiments, the appropriately modified Fc region of the antibody or antigen binding fragment will comprise a conjugation handle which is used to conjugate the antibody or antigen binding fragment to an cytokine or a derivative thereof.

Any suitable reactive group capable of reacting with a complementary reactive group attached to the synthetic cytokine or derivative thereof can be used as the conjugation handle. In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cycloctenes, or a metal-mediated process such as olefin metathesis and Suzuki-Miyaura or Sonogashira cross-coupling.

In some embodiments, the conjugation handle comprises a reagent for a “copper-free” alkyne azide triazole-forming reaction. Non-limiting examples of alkynes for said alkyne azide triazole forming reaction include cyclooctyne reagents (e.g., (1R,8S,9s)-Bicyclo[6.1.0]non-4-yn-9-ylmethanol containing reagents, dibenzocyclooctyne-amine reagents, difluorocyclooctynes, or derivatives thereof). In some embodiments, the alkyne functional group is attached to the Fc region. In some embodiments, the azide functional group is attached to the Fc region.

In some embodiments, the conjugation handle comprises a reactive group selected from azide, alkyne, tetrazine, halide, sulfhydryl, disulfide, maleimide, activated ester, alkene, aldehyde, ketone, imine, hydrazine, and hydrazide. In some embodiments, the synthetic cytokine or derivative thereof comprises a reactive group complementary to the conjugation handle of the Fc region. In some embodiments, the conjugation handle and the complementary conjugation handle comprise “CLICK” chemistry reagents. Exemplary groups of click chemistry residue are shown in Hein et al., “Click Chemistry, A Powerful Tool for Pharmaceutical Sciences,” Pharmaceutical Research, volume 25, pages 2216-2230 (2008); Thirumurugan et al., “Click Chemistry for Drug Development and Diverse Chemical-Biology Applications,” Chem. Rev. 2013, 113, 7, 4905-4979; US20160107999A1; U.S. Pat. No. 10,266,502B2; and US20190204330A1, each of which is incorporated by reference in its entirety.

### Linker Structure

In some embodiments, the linker used to attach the antibody or antigen binding fragment and the synthetic cytokine or derivative thereof comprises points of attachment at both moieties. The points of attachment can be any of the residues for facilitating the attachment as provided herein. The linker structure can be any suitable structure for creating the spatial attachment between the two moieties. In some embodiments, the linker provides covalent attachment of both moieties. In some embodiments, the linker is a chemical linker (e.g., not an expressed polypeptide as in a fusion protein).

In some embodiments, the linker comprises a polymer. In some embodiments, the linker comprises a water soluble polymer. In some embodiments, the linker comprises poly(alkylene oxide), polysaccharide, poly(vinyl pyrrolidone), poly(vinyl alcohol), polyoxazoline, poly(acryloylmorpholine), or a combination thereof. In some embodiments, the linker comprises poly(alkylene oxide). In some embodiments, the poly(alkylene oxide) is polyethylene glycol or polypropylene glycol, or a combination thereof. In some embodiments, the poly(alkylene oxide) is polyethylene glycol.

In some embodiments, the linker is a bifunctional linker. In some embodiments, the bifunctional linker comprises an amide group, an ester group, an ether group, a thioether group, or a carbonyl group. In some embodiments, the linker comprises a non-polymer linker. In some embodiments, the linker comprises a non-polymer, bifunctional linker. In some embodiments, the non-polymer, bifunctional linker comprises succinimidyl 4-(N-maleimidomethyl)cyclohexane-1-carboxylate; Maleimidocaproyl; Valine-citrulline; Allyl(4-methoxyphenyl)dimethylsilane; 6-(Allyloxycarbonylamino)-1-hexanol; 4-Aminobutyraldehyde diethyl acetal; or (E)-N-(2-Aminoethyl)-4-{2-[4-(3-azidopropoxy)phenyl]diazenyl}benzamide hydrochloride.

In some embodiments, a portion of the linker is made up of a bifunctional linking reagent which has been reacted with appropriate groups attached to the antibody or the other protein (e.g., the recombinant protein or the synthetic protein). In some embodiments, bifunctional linking reagents have a formula A-B-C, wherein A is a first conjugation handle, B is a linking group, and C is a second conjugation handle. In some embodiments, the first conjugation handle is first reacted with a suitable group attached to a first moiety of the eventual conjugate precursor (e.g., an antibody which is desired to be turned into a conjugate). In some embodiments, the second conjugation handle is then reacted with a second suitable group attached to the second moiety of the eventual conjugate (e.g., the protein, such as a synthetic cytokine). In some embodiments, bifunctional linking reagents of the disclosure comprise a sulfhydryl specific conjugation handle (e.g., maleimide, alpha-halo carbonyl, etc.) and the second conjugation handle is an alkyne (e.g., a DBCO reagent).

The linker can be branched or linear. In some embodiments, the linker is linear. In some embodiments, the linker is branched. In some embodiments, the linker comprises a linear portion (e.g., between the first point of attachment and the second point of attachment) of a chain of at least 10, 20, 50, 100, 500, 1000, 2000, 3000, or 5000 atoms. In some embodiments, the linker comprises a linear portion of a chain of at least 10, 20, 30, 40, or 50 atoms. In some embodiments, the linker comprises a linear portion of at least 10 atoms. In some embodiments, the linker comprises a linear portion of at most 20, 30, 40, 50, 60, 70, 80, 90, or 100 atoms. In some embodiments, the linker is branched and comprises a linear portion of a chain of at least 10, 20, 50, 100, 500, 1000, 2000, 3000, or 5000 atoms. In some embodiments, the linker is unbranched and comprises a chain of at most about 40, 50, 60, 70, 80, 90, or 100 atoms.

In some embodiments, the linker has a molecular weight of from about 200 Daltons to about 2000 Daltons. In some embodiments, the linker has a molecular weight of at least about 1,000 Daltons, at least about 5,000 Daltons, at least about 10,000 Daltons, at least about 15,000 Daltons, at least about 20,000 Daltons, at least about 25,000 Daltons, or at least about 30,000 Daltons. In some embodiments, the linker as a molecular weight of at most about 100,000 Daltons, at most about 50,000 Daltons, at most about 40,000 Daltons, at most about 30,000 Daltons, at most about 25,000 Daltons, at most about 20,000 Daltons at most about 15,000 Daltons, at most about 10,000 Daltons, or at most about 5,000 Daltons.

In some embodiments, the linker comprises a reaction product one or more pairs of conjugation handles and a complementary conjugation handle thereof. In some embodiments, the reaction product comprises a triazole, a hydrazone, pyridazine, a sulfide, a disulfide, an amide, an ester, an ether, an oxime, an alkene, or any combination thereof. In some embodiments, the reaction product comprises a triazole. The reaction product can be separated from the first point of attachment and the second point of attachment by any portion of the linker. In some embodiments, the reaction product is substantially in the center of the linker. In some embodiments, the reaction product is substantially closer to one point of attachment than the other.

In some embodiments, the linker comprises a structure of Formula (X)

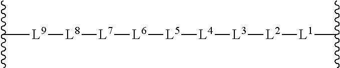

wherein each of L¹, L², L³, L⁴, L⁵, L⁶, L⁸, and L⁹ is independently —O—, —NR^{L}—, —N(R^{L})₂⁺—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O— CH(CH₃)—CH₂)_{qd}—, a reaction product of a conjugation handle and a complementary conjugation handle, or absent;
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
wherein each

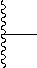

is a point of attachment to the polypeptide which selectively binds to the antibody or antigen binding fragment or to the synthetic cytokine or derivative thereof.

In some embodiments, the linker comprises a structure of Formula (X′):

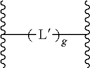

wherein each L′ is independently —O—, —NR^{L}—, —(C₁-C₆ alkylene)NR^{L}—, —NR^{L}(C₁-C₆ alkylene)-, —N(R^{L})₂⁺—, —(C₁-C₆ alkylene)N(R^{L})₂⁺—, —N(R^{L})₂⁺—(C₁-C₆ alkylene)-, —OP(═O)(OR^{L})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O) (C₁-C₆ alkylene)-, —C(═O)O—, —OC(═O)—, —OC(═O)O—, —C(═O)NR^{L}—, —C(═O)NR^{L}(C₁-C₆ alkylene)-, —(C₁-C₆ alkylene)C(═O)NR^{L}—, —NR^{L}C(═O)—, —(C₁-C₆ alkylene)NR^{L}C(═O)—, —NR^{L}C(═O)(C₁-C₆ alkylene)-, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O—CH(CH₃)—CH₂)_{qd}—, a reaction product of a conjugation handle and a complementary conjugation handle, or absent; (C₁-C₆ alkylene);
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl;
each of qa, qb, qc and qd is independently an integer from 1-100; and
g is an integer from 1-100;
wherein each

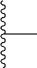

is a point of attachment to the modified IL-2 polypeptide or the antibody or antigen binding fragment.

In some embodiments, the linker of Formula (X) or of Formula (X^{a}) or of Formula (X′) comprises the structure:

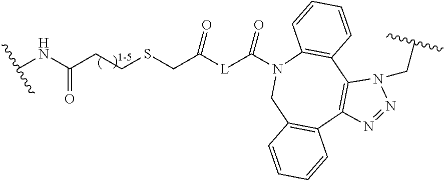

wherein

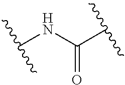 [SMILES: *NC(*)=O]

is the first point of attachment to a lysine residue of the polypeptide which selectively binds to CD20;
L is a linking group; and

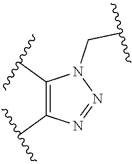 [SMILES: *Cn1nnc(*)c1*]

is a point of attachment to a linking group which connects to the first point of attachment of the protein (e.g., the recombinant protein, the synthetic protein, the cytokine, the synthetic cytokine, or other protein provided herein),
or a regioisomer thereof.

In some embodiments, L has a structure

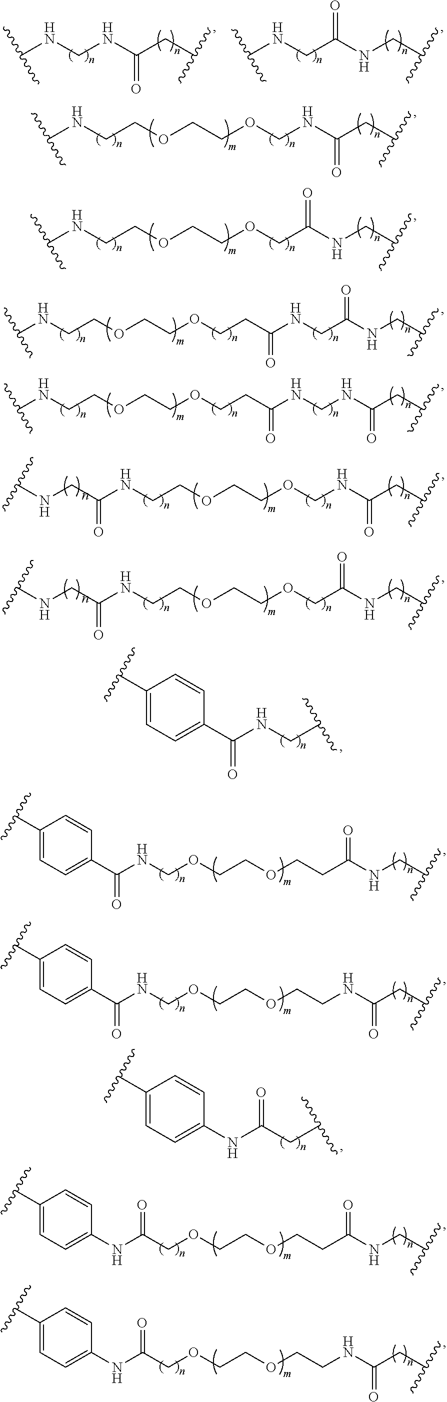

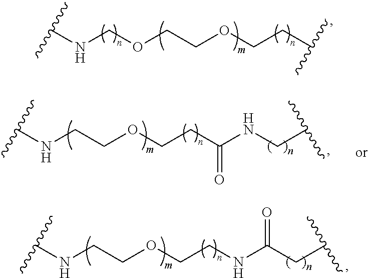

wherein each n is independently an integer from 1-6 and each m is an integer from 1-30. In some embodiments, each m is independently 2 or 3. In some embodiments, each m is an integer from 1-24, from 1-18, from 1-12, or from 1-6.

In some embodiments, the linker of Formula (X) or of Formula (X^{a}) or of Formula (X′) comprises the structure:

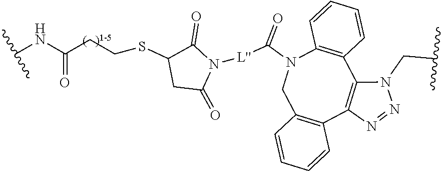

wherein

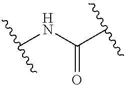 [SMILES: *NC(*)=O]

is the first point of attachment to a lysine residue of the antibody;
L″ is a linking group; and

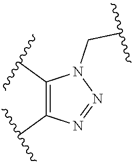 [SMILES: *Cn1nnc(*)c1*]

is a point of attachment to a linking group which connects to the first point of attachment of the protein (e.g., the protein attached to the antibody),
or a regioisomer thereof.

In some embodiments, L″ has a structure

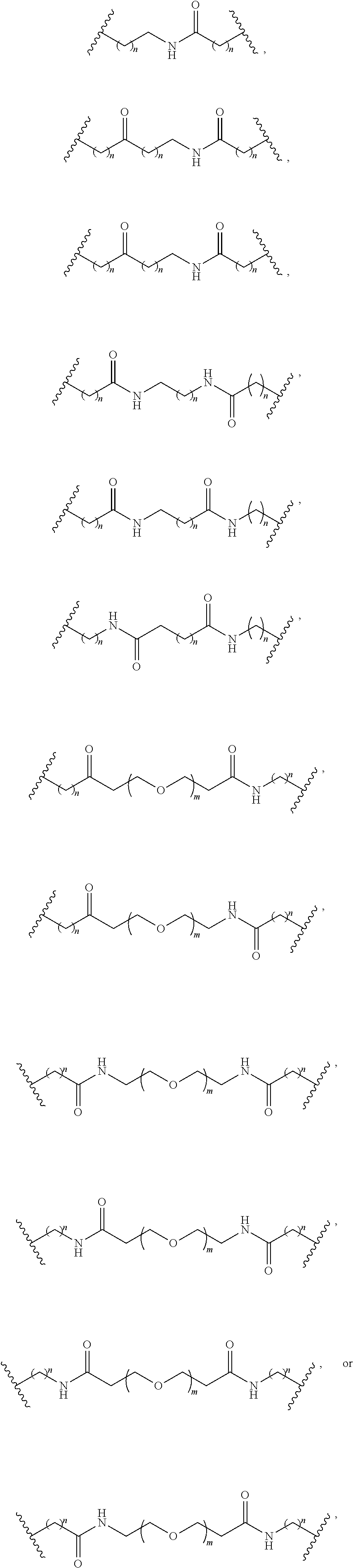

wherein each n is independently an integer from 1-6 and each m is independently an integer from 1-30. In some embodiments, each m is independently 2 or 3. In some embodiments, each m is an integer from 1-24, from 1-18, from 1-12, or from 1-6.

In some embodiments, L or L″ comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more subunits each independently selected from

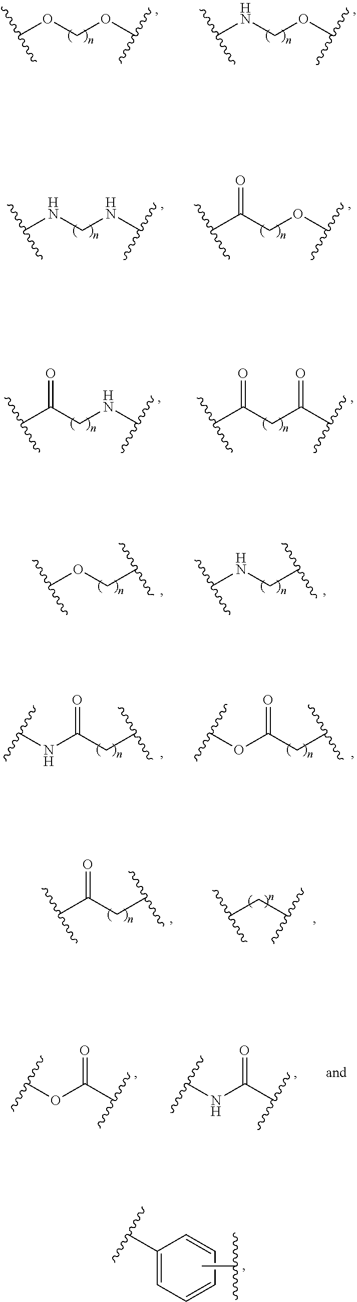

wherein each n is independently an integer from 1-30. In some embodiments, each n is independently an integer from 1-6. In some embodiments, L or L″ comprise 1, 2, 3, 4, 5, 6, 7, 8, 9, or 10 of the subunits

In some embodiments, L or L″ is a structure of Formula (X″)

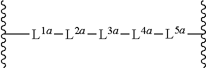

wherein each of L^{1a}, L^{2a}, L^{3a}, L^{4a}, L^{5a}, is independently —O—, —NR^{La}—, —(C₁-C₆ alkylene)NR^{La}—, —NR^{La}(C₁-C₆ alkylene)-, —N(R^{L})₂⁺—, —(C₁-C₆ alkylene)N(R^{La})₂⁺(C₁-C₆ alkylene)-, —N(R^{La})₂⁺—, —OP(═O)(OR^{La})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O)(C₁-C₆ alkylene)-, —C(═O)O—, —OC(═O)—, —OC(═O)O—, —C(═O)NR^{La}—, —C(═O)NR^{La}(C₁-C₆ alkylene)-, —(C₁-C₆ alkylene)C(═O)NR^{La}—, —NR^{La}C(═O)—, —(C₁-C₆ alkylene)NR^{La}C(═O)—, —NR^{La}C(═O)(C₁-C₆ alkylene)-, —OC(═O)NR^{La}—, —NR^{La}C(═O)O—, —NR^{La}C(═O)NR^{La}—, —NR^{La}C(═S)NR^{La}—, —CR^{La}═N—, —N═CR^{La}, —NR^{La}S(═O)₂—, —S(═O)₂NR^{La}—, —C(═O)NR^{La}S(═O)₂—, —S(═O)₂NR^{La}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qe}—, —(O—CH₂—CH₂)_{qf}—, —(CH₂—CH(CH₃)—O)_{qg}, —(O—CH(CH₃)—CH₂)_{qh}—, a reaction product of a conjugation handle and a complementary conjugation handle, or absent;
each R^{La} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qe, qf, qg and qh is independently an integer from 1-100.

In some embodiments, L or L″ comprises a linear chain of 2 to 10, 2 to 15, 2 to 20, 2 to 25, or 2 to 30 atoms. In some embodiments, the linear chain comprises one or more alkyl groups (e.g., lower alkyl (C₁-C₄)), one or more aromatic groups (e.g., phenyl), one or more amide groups, one or more ether groups, one or more ester groups, or any combination thereof.

In some embodiments, the linking group which connects to the first point of attachment (e.g., the point of attachment to the cytokine) comprises poly(ethylene glycol). In some embodiments, the linking group comprises about 2 to about 30 poly(ethylene glycol) units. In some embodiments, the linking group which connects to the first point of attachment (e.g., the point of attachment to the cytokine) is a functionality attached to a cytokine provided herein which comprises an azide (e.g., the triazole is the reaction product of the azide).

In some embodiments, L is —O—, —NR^{L}—, —N(R^{L})₂⁺—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O—CH(CH₃)—CH₂)_{qd}—, wherein R^{L} hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and each of qa, qb, qc and qd is independently an integer from 1-100.

In some embodiments, each reaction product of a conjugation handle and a complementary conjugation handle comprises a triazole, a hydrazone, pyridazine, a sulfide, a disulfide, an amide, an ester, an ether, an oxime, or an alkene. In some embodiments, each reaction product of a conjugation handle and a complementary conjugation handle comprises a triazole. In some embodiments, each reaction product of a conjugation handle and a complementary conjugation handle comprise a structure of:

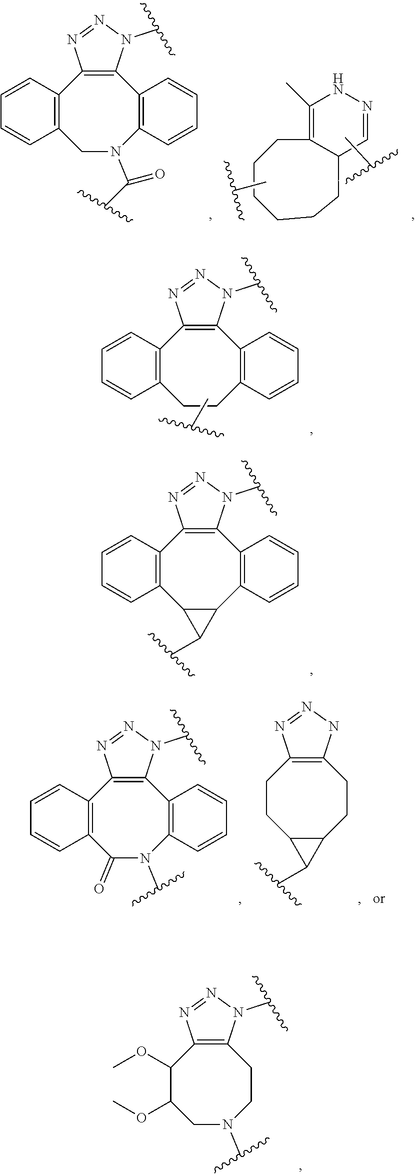

or a regioisomer or derivative thereof.

In some embodiments, the linker is a cleavable linker. In some embodiments, the cleavable linker is cleaved at, near, or in a tumor microenvironment. In some embodiments, the tumor is mechanically or physically cleaved at, near, or in the tumor microenvironment. In some embodiments, the tumor is chemically cleaved at, near, or in a tumor microenvironment. In some embodiments, the cleavable linker is a reduction sensitive linker. In some embodiments, the cleavable linker is an oxidation sensitive linker. In some embodiments, the cleavable linker is cleaved as a result of pH at, near, or in the tumor microenvironment. In some embodiments, the linker by a tumor metabolite at, near, or in the tumor microenvironment. In some embodiments, the cleavable linker is cleaved by a protease at, near, or in the tumor microenvironment.

### Proteins Attached to Antibodies

The conjugate compositions provided herein comprise an antibody attached to another protein (e.g, a recombinant protein, a synthetic protein, a cytokine, a synthetic cytokine, etc.) through a linker. In some embodiments, the protein can be any protein. In some embodiments, the protein comprises a defined secondary or tertiary structure associated with the protein (e.g., the protein is folded in a particular manner). In some embodiments, a fully folded protein is able to be conjugated to an antibody in order to retain the function of the protein when attached to the antibody. In some embodiments, the conjugate retains the function of the protein after attachment to the antibody. In some embodiments, there is a size range of protein which is most suitable for attachment to the antibody for retention of activity, stability of the conjugate, and other factors. In some embodiments, choice of linker position impacts the activity of the protein in a desired way (e.g., biases the protein towards a different activity, or reduces the activity of the attached protein).

In some embodiments, the protein is a synthetic protein (e.g., prepared from one or more synthetically prepared fragment peptides). In some embodiments, the synthetic protein comprises from about 50 to about 300 amino acid residues, from about 50 to about 250 amino acid residues, from about 50 to about 200 amino acid residues, from about 75 to about 300 amino acid residues, from about 75 to about 250 amino acid residues, from about 75 to about 200 amino acid residues, from about 100 to about 300 amino acid residues, from about 100 to about 250 amino acid residues, or from about 100 to about 200 amino acid residues. In some embodiments, the synthetic protein comprises from about 100 to about 300 amino acid residues. In some embodiments, the synthetic protein comprises from about 100 to about 250 amino acid residues. In some embodiments, the synthetic protein comprises from about 100 to about 200 amino acid residues. In some embodiments, the synthetic protein is a synthetic cytokine.

A synthetic cytokine as provided herein can be any cytokine. Non-limiting examples of cytokines which can potentially be synthesized include interleukins (e.g., IL-2, IL-18, IL-7, IL-17), TNF family cytokines (e.g., TNFa, CD70, TNFSF14), interferons (e.g., IFNγ, IFNα. IFNβ), TGF-β family cytokines (e.g., TGFB1, TGFB2, TGFB3), chemokines (e.g., CCL2, CCL3, CXCL9, CXCL10) and others. In some embodiments, cytokines which can be synthesized is an interleukin. In some embodiments, the interleukin is an IL-1 family cytokine (e.g., IL-18, IL-1β, IL-33), an IL-2 family cytokine (e.g., IL-2, IL-4, IL-7, IL-15, IL-21), an IL-6 family interleukin (e.g., IL-6, IL-11, IL-31), an IL-10 family cytokine (e.g., IL-10, IL-19, IL-20, IL-22), an IL-12 family cytokine (e.g., IL-12, IL-23, IL-27, IL-35) and an IL-17 family cytokine (e.g., IL-17, IL-17F, IL-25).

In some embodiments, the protein is a recombinant protein. In some embodiments, the recombinant protein comprises from about 50 to about 300 amino acid residues, from about 50 to about 250 amino acid residues, from about 50 to about 200 amino acid residues, from about 75 to about 300 amino acid residues, from about 75 to about 250 amino acid residues, from about 75 to about 200 amino acid residues, from about 100 to about 300 amino acid residues, from about 100 to about 250 amino acid residues, or from about 100 to about 200 amino acid residues. In some embodiments, the recombinant protein comprises from about 100 to about 300 amino acid residues. In some embodiments, the recombinant protein comprises from about 100 to about 250 amino acid residues. In some embodiments, the recombinant protein comprises from about 100 to about 200 amino acid residues. In some embodiments, the recombinant protein is a cytokine.

A recombinant cytokine as provided herein can be any cytokine. Non-limiting examples of cytokines which can be prepared recombinantly include interleukins (e.g., IL-2, IL-18, IL-7, IL-17), TNF family cytokines (e.g., TNFa, CD70, TNFSF14), interferons (e.g., IFNγ, IFNα. IFNβ), TGF-β family cytokines (e.g., TGFB1, TGFB2, TGFB3), chemokines (e.g., CCL2, CCL3, CXCL9, CXCL10) and others. In some embodiments, the recombinant cytokine is an interleukin. In some embodiments, the recombinant cytokine is selected from an IL-1 family cytokine (e.g., IL-18, IL-1β, IL-33), an IL-2 family cytokine (e.g., IL-2, IL-4, IL-7, IL-15, IL-21), an IL-6 family interleukin (e.g., IL-6, IL-11, IL-31), an IL-10 family cytokine (e.g., IL-10, IL-19, IL-20, IL-22), an IL-12 family cytokine (e.g., IL-12, IL-23, IL-27, IL-35) and an IL-17 family cytokine (e.g., IL-17, IL-17F, IL-25).

### Cytokines and Derivatives Thereof

Cytokines are proteins produced in the body that are important in cell signaling. Cytokines can modulate the immune system, and cytokine therapy utilizes the immunomodulatory properties of the molecules to enhance the immune system of a subject. Disclosed herein are cytokines (e.g., modified cytokines and/or synthetic cytokines) conjugated to an antibody or antigen binding fragment (e.g., an antibody or antigen binding fragment described above) which can exhibit enhanced biological activity.

Modifications to the polypeptides described herein encompass mutations, addition of various functionalities, deletion of amino acids, addition of amino acids, or any other alteration of the wild-type version of the protein or protein fragment. Functionalities which may be added to polypeptides include polymers, linkers, alkyl groups, detectable molecules such as chromophores or fluorophores, reactive functional groups, or any combination thereof. In some embodiments, functionalities are added to individual amino acids of the polypeptides. In some embodiments, functionalities are added site-specifically to the polypeptides.

In one aspect, provided herein is a modified cytokine comprising natural amino acid substitutions. In some embodiments, the modified cytokine comprises up to seven natural amino acid substitutions. In some embodiments, the modified cytokine comprises up to six amino acid substitutions. In some embodiments, the modified cytokine comprises up to five amino acid substitutions. In some embodiments, the modified cytokine comprises up to four amino acid substitutions. In some embodiments, the modified cytokine comprises up to three amino acid substitutions. In some embodiments, the modified cytokine comprises from three to seven, three to six, three to five, three to four, four to seven, four to six, four to five, five to seven, five to six, or six to seven natural amino acid substitutions. In some embodiments, the modified cytokine comprises at least one, at least two, at least three, at least four, at least five, or at least six amino acid substitutions.

In some embodiments, a modified cytokine described herein comprises at least 3, at least 4, at least 5, at least 6, at least 7, or at least 9 amino acid substitutions. In some embodiments, the modified cytokine comprises 3 to 9 amino acid substitutions. In some embodiments, the modified cytokine comprises 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the modified cytokine comprises 3 amino acid substitutions, 4 amino acid substitutions, 5 amino acid substitutions, 6 amino acid substitutions, 7 amino acid substitutions, or 9 amino acid substitutions. In some embodiments, the modified cytokine comprises at most 4 amino acid substitutions, 5 amino acid substitutions, 6 amino acid substitutions, 7 amino acid substitutions, or 9 amino acid substitutions.

In some embodiments, a modified cytokine (e.g., a modified IL-2 polypeptide, a modified IL-7 polypeptide, a modified IL-18 polypeptide, etc.) described herein comprises one or more modifications at one or more amino acid residues. In some embodiments, the residue position numbering of a modified IL-2 polypeptide is based on SEQ ID NO: 1 as a reference sequence. In some embodiments, the residue position numbering of the modified IL-2 polypeptide is based on a wild-type human IL-2 polypeptide as a reference sequence. In some instances, the modified IL-7 polypeptide is modified compared to a reference IL-7 amino acid sequence provided in Table 8B (e.g., any one of SEQ ID NOS: 165-169, 186, or 187 (preferably 186)). In some instances, the modified IL-18 polypeptide is modified compared to a reference IL-18 amino acid sequence provided in Table 8C (e.g., any one of SEQ ID NOS: 170-175).

Modifications to the proteins attached to antibodies described herein encompass mutations, addition of various functionalities, deletion of amino acids, addition of amino acids, or any other alteration of the wild-type version of the protein or protein fragment. Functionalities which may be added to polypeptides include polymers, linkers, alkyl groups, detectable molecules such as chromophores or fluorophores, reactive functional groups, or any combination thereof. In some embodiments, functionalities are added to individual amino acids of the polypeptides. In some embodiments, functionalities are added site-specifically to the polypeptides. In some embodiments, the functionality comprises at least a portion of the linker used to attach the cytokine to the antibody or antigen binding fragment.

A modified protein (e.g, a modified cytokine) as described herein can comprise one or more non-canonical amino acids. Non-canonical amino acids include, but are not limited to N-alpha-(9-Fluorenylmethyloxycarbonyl)-L-biphenylalanine (Fmoc-L-Bip-OH) and N-alpha-(9-Fluorenylmethyloxycarbonyl)-O-benzyl-L-tyrosine (Fmoc-L-Tyr(Bzl)-OH. Exemplary non-canonical amino acids include p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl)alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, p-Boronophenylalanine, O-propargyltyrosine, L-phosphoserine, phosphonoserine, phosphonotyrosine, p-bromophenylalanine, selenocysteine, p-amino-L-phenylalanine, isopropyl-L-phenylalanine, azido-lysine (AzK), an analogue of a tyrosine amino acid; an analogue of a glutamine amino acid; an analogue of a phenylalanine amino acid; an analogue of a serine amino acid; an analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, β-amino acid; a cyclic amino acid other than proline or histidine; an aromatic amino acid other than phenylalanine, tyrosine or tryptophan; or a combination thereof. In some embodiments, the non-canonical amino acids are selected from β-amino acids, homoamino acids, cyclic amino acids and amino acids with derivatized side chains. In some embodiments, the non-canonical amino acids comprise β-alanine, β-aminopropionic acid, piperidinic acid, aminocaproic acid, aminoheptanoic acid, aminopimelic acid, desmosine, diaminopimelic acid, N^{α}-ethylglycine, N^{α}-ethylasparagine, hydroxylysine, allo-hydroxylysine, isodemosine, allo-isoleucine, ω-methylarginine, N^{α}-methylglycine, N^{α}-methylisoleucine, N^{α}-methylvaline, γ-carboxyglutamate, ε-N,N,N-trimethyllysine, ε-N-acetyllysine, O-phosphoserine, N^{α}-acetylserine, N^{α}-formylmethionine, 3-methylhistidine, 5-hydroxylysine, and/or other similar amino acids.

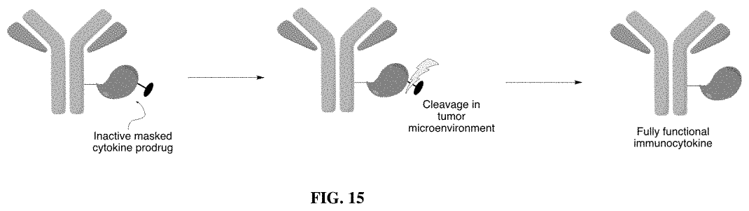

In some embodiments, a cytokine attached to an antibody as provided herein is an inactive masked version of the cytokine which acts as a prodrug. An exemplary cytokine conjugate is shown in FIG. 15. In some embodiments, such a cytokine is selectively activated by cleavage of the masking group in the tumor microenvironment to yield a fully functional immunocytokine.

### Points of Attachment of Linker to Cytokines

Provided herein are compositions comprising antibodies or antigen binding fragments (which selectively bind to a target antigen) that are connected to a modified or synthetic cytokine by a linker. As discussed supra, the linker can be attached at a first point of attachment to the antibody or antigen binding fragment. The second point of attachment of the linker is attached to a modified or synthetic cytokine as provided herein. In some instances, the modified or synthetic cytokine is a modified IL-2 polypeptide. In other instances, the modified or synthetic cytokine is a modified IL-7 polypeptide. The point of attachment to the IL-7 polypeptide may selected such that the interaction of the IL-7 polypeptide with at least one IL-7 receptor is decreased or blocked. In other instances, the modified or synthetic cytokine is a modified IL-18 polypeptide. The point of attachment to the IL-18 polypeptide may selected such that the interaction of the IL-18 polypeptide with at least one IL-18 receptor is decreased or blocked.

A linker can be attached to an amino acid residue which is a natural amino acid residue of a cytokine described herein. In some embodiments, the linker is attached to an amino acid residue which is a modified version of the natural amino acid residue of an IL-2 polypeptide as set forth in any one of SEQ ID NOS: 1-23 or 176-185, an IL-7 polypeptide as set forth in any one of SEQ ID NOS: 165-169, 186, or 187, or an IL-18 polypeptide as set forth in SEQ ID NOS: 170-175.

Non-limiting examples of such modification include incorporation or attachment of a conjugation handle to the natural amino acid residue (including through a linker), or attachment of the linker to the natural amino acid using any compatible method. In some embodiments, the linker is attached to an amino acid residue which is a substituted amino acid residue compared to the IL-2 polypeptide of any one of SEQ ID NOS: 1-23 or 176-185, an IL-7 polypeptide as set forth in any one of SEQ ID NOS: 165-169, 186, or 187, or an IL-18 polypeptide as set forth in SEQ ID NOS: 170-175. The substitution can be for a naturally occurring amino acid which is more amenable to attachment of additional functional groups (e.g., aspartic acid, cysteine, glutamic acid, lysine, serine, threonine, or tyrosine), a derivative of modified version of any naturally occurring amino acid, or any unnatural amino acid (e.g., an amino acid containing a desired reactive group, such as a CLICK chemistry reagent such as an azide, alkyne, etc.). Non-limiting examples of amino acids which can be substituted include, but are not limited to N-alpha-(9-Fluorenylmethyloxycarbonyl)-L-biphenylalanine (Fmoc-L-Bip-OH) and N-alpha-(9-Fluorenylmethyloxycarbonyl)-O-benzyl-L-tyrosine (Fmoc-L-Tyr(Bzl)-OH. Exemplary non-canonical amino acids include p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl)alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, p-Boronophenylalanine, O-propargyltyrosine, L-phosphoserine, phosphonoserine, phosphonotyrosine, p-bromophenylalanine, selenocysteine, p-amino-L-phenylalanine, isopropyl-L-phenylalanine, azido-lysine (AzK), an analogue of a tyrosine amino acid; an analogue of a glutamine amino acid; an analogue of a phenylalanine amino acid; an analogue of a serine amino acid; an analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, a R-amino acid; a cyclic amino acid other than proline or histidine; an aromatic amino acid other than phenylalanine, tyrosine or tryptophan; or a combination thereof. In some embodiments, the non-canonical amino acids are selected from R-amino acids, homoamino acids, cyclic amino acids and amino acids with derivatized side chains. In some embodiments, the non-canonical amino acids comprise β-alanine, β-aminopropionic acid, piperidinic acid, aminocaproic acid, aminoheptanoic acid, aminopimelic acid, desmosine, diaminopimelic acid, N^{α}-ethylglycine, N^{α}-ethylasparagine, hydroxylysine, allo-hydroxylysine, isodemosine, allo-isoleucine, ω-methylarginine, N^{α}-methylglycine, N^{α}-methylisoleucine, N^{α}-methylvaline, γ-carboxyglutamate, ε-N,N,N-trimethyllysine, ε-N-acetyllysine, O-phosphoserine, N^{α}-acetylserine, N^{α}-formylmethionine, 3-methylhistidine, 5-hydroxylysine, and/or other similar amino acids.

In some embodiments, the linker is attached at an unnatural amino acid residue. In some embodiments, the unnatural amino acid residue comprises a conjugation handle. In some embodiments, the conjugation handle facilitates the addition of the linker to the modified IL-2 polypeptide. The conjugation handle can be any of the conjugation handles provided herein. In some embodiments, the linker is covalently attached site-specifically to the unnatural amino acid. Non-limiting examples of amino acid residues comprising conjugation handles can be found, for example, in PCT Pub. Nos. WO2015054658A1, WO2014036492A1, and WO2021133839A1 WO2006069246A2, and WO2007079130A2, each of which is incorporated by reference as if set forth in its entirety.

In some embodiments, the linker is attached to an amino acid residue which has been substituted with a natural amino acid. In some embodiments, the linker is attached to an amino acid residue which has been substituted with a cysteine, lysine, or tyrosine residue. In some embodiments, the linker is attached to a residue which has been substituted with a cysteine residue. In some embodiments, the linker is attached to an amino acid residue which has been substituted with a lysine residue. In some embodiments, the linker is attached to an amino acid residue which has been substituted with a tyrosine residue.

In some embodiments, a protein (e.g., a chemically synthesized cytokine) comprises a conjugation handle attached to one or more residues to facilitate attachment of the linker to the antibody or antigen binding fragment. The conjugation handle may be any such conjugation handle provided herein and may be attached at any residue to which the linker may be attached. In some embodiments, the conjugation handle is attached to, for example, residue 1 (e.g., the N-terminal amine) of a protein. In some embodiments, the conjugation handle comprises an azide or an alkyne. Alternatively, in some embodiments, the conjugation handle is incorporated into an unnatural or modified natural amino acid of a recombinant protein. Recombinant protein with unnatural amino acids can be made using methods as described in, for example, Patent Cooperation Treaty Publication Nos. WO2016115168, WO2002085923, WO2005019415, and WO2005003294.

In some embodiments, the linker is attached to an amino acid residue which has been substituted with a natural amino acid. In some embodiments, the linker is attached to an amino acid residue which has been substituted with a cysteine, lysine, or tyrosine residue. In some embodiments, the linker is attached to a residue which has been substituted with a cysteine residue. In some embodiments, the linker is attached to an amino acid residue which has been substituted with a lysine residue. In some embodiments, the linker is attached to an amino acid residue which has been substituted with a tyrosine residue.

### IL-2 Polypeptides

Interleukin-2 (IL-2) is a cytokine signaling molecule important in regulating the immune system. IL-2 is implicated in helping the immune system differentiate between foreign and endogenous cell types, thereby preventing the immune system from attacking a subject's own cells. IL-2 accomplishes its activity through interactions with IL-2 receptors (IL-2R) expressed by lymphocytes. Through these binding interactions, IL-2 can modulate a subject's populations of T-effector (T_{eff}) cells, natural killer (NK) cells, and regulatory T-cells (T_{reg}).

IL-2 has been used to treat cancer, both alone and in combination with other therapies. However, use of IL-2 as a treatment has been limited by the toxicity of IL-2, undesirable side effects such as vascular leak syndrome, and the short half-life of IL-2. Conjugation of IL-2 to an antibody or antigen binding fragment of the disclosure can improve IL-2 polypeptide selectivity, enhance the therapeutic potential of IL-2, and minimize the risk of side effects from administering IL-2 therapies. The present disclosure describes antibodies or antigen binding fragments conjugated to a modified and/or synthetic interleukin-2 (IL-2) polypeptide and the use of the conjugates as therapeutic agents. Modified IL-2 polypeptides provided herein can be used as immunotherapies or as parts of other immunotherapy regimens. Such modified IL-2 polypeptides may display binding characteristics for the IL-2 receptor (IL-2R) that differ from wild-type IL-2. In one aspect, modified IL-2 polypeptides described herein have decreased affinity for the IL-2R αβγ complex (IL-2Rα). In some embodiments, the modified IL-2 polypeptides have an increased affinity for the IL-2R βγ complex (IL-2Rβ). In some embodiments, the binding affinity between the modified IL-2 polypeptides and IL-2Rβ is the same as or lower than the binding affinity between a wild-type IL-2 and IL-2Rβ. Non-limiting examples of IL-2 amino acid sequences to be utilized in embodiments described herein are provided below in Table 8A. An IL-2 polypeptide utilized in conjugate described herein can have, for example, an amino acid sequence that is at least 80%, 85%, 90%, 91%, 92%, 93%, 94%, 95%, 96%, 97%, 98%, 99%, or 100% identical to any one of the sequences in Table 8A (e.g., any one of SEQ ID NOs: 1-23, or any one of SEQ ID NOs: 176-185).

In some embodiments, the linker is attached to a modified or synthetic IL-2 polypeptide at an amino acid residue. In some embodiments, the linker is attached at an amino acid residue corresponding to any one of amino acid residues 1-133 of SEQ ID NO: 1. In some embodiments, the linker is attached at a non-terminal amino acid residue (e.g., any one of amino acid residues 2-132 of SEQ ID NO: 1, or any one of amino acid residues 1-133 of SEQ ID NO: 1, wherein either the N-terminus or C-terminus has been extended by one or more amino acid residues). In some embodiments, the linker is attached at a non-terminal amino acid residue of the IL-2 polypeptide, wherein the IL-2 polypeptide comprises either an N-terminal truncation or a C-terminal truncation relative to SEQ ID NO: 1.

In some embodiments, the linker is attached to the IL-2 polypeptide at an amino acid residue which interacts with an IL-2 receptor (IL-2R) protein or subunit. In some embodiments, the linker is attached at an amino acid residue which interacts with the IL-2R alpha subunit (IL-2Rα), the IL-2R beta subunit (IL-2Rβ), or the IL-2R gamma subunit (IL-2Rγ). In some embodiments, the linker is attached at an amino acid residue which interacts with the IL-2R alpha subunit (IL-2Rα). In some embodiments, the linker is attached at an amino acid residue which interacts with the IL-2R beta subunit (IL-2Rβ). In some embodiments, the linker is attached at an amino acid residue which interacts with the IL-2R gamma subunit (IL-2Rγ). In some embodiments, the point of attachment to the IL-2 polypeptide is selected such that the interaction of the IL-2 polypeptide with at least one IL-2 receptor subunit is decreased or blocked. In some embodiments, the point of attachment is selected such that interaction of the IL-2 polypeptide with the IL-2Rα is reduced or blocked. In some embodiments, the point of attachment is selected such that interaction of the IL-2 polypeptide with the IL-2Rβ is reduced or blocked.

In some embodiments, the modified IL-2 polypeptides display activity which differs from a wild type IL-2. These modified biological activities provided herein below apply, in some embodiments, to the IL-2 polypeptide alone (e.g., not conjugated or otherwise attached to the polypeptide which binds the antigen or antigen binding fragment) as well as when the IL-2 polypeptide is conjugated or otherwise to the polypeptide which binds the antigen or antigen binding fragment (e.g., the modified biological activity is retained upon conjugation or attachment). Thus, when a modified IL-2 polypeptide is described herein as having an indicated activity, it is also contemplated that immunocytokine compositions provided herein (e.g., the IL-2 polypeptide attached to the polypeptide which binds the antigen or antigen binding fragment) has the same activity.

In some embodiments, a modified IL-2 polypeptide described herein comprises one or more modifications at one or more amino acid residues. In some embodiments, the residue position numbering of the modified IL-2 polypeptide is based on SEQ ID NO:1 as a reference sequence. In some embodiments, the residue position numbering of the modified IL-2 polypeptide is based on a wild-type human IL-2 polypeptide as a reference sequence. In some instances, a modified IL-2 polypeptide described herein comprises an amino acid sequence of any one of SEQ ID NOS: 1-23.

In some embodiments, a modified IL-2 polypeptide provided herein comprises an N-terminal deletion. In some embodiments, the N-terminal deletion is of 1, 2, 3, 4, 5, 6, 7, 8, 9, 10, 11, 12, 13, 14, 15, or more amino acids. In some embodiments, the N-terminal deletion is of at least 1 amino acid. In some embodiments, the N-terminal deletion is of at least 1, 2, 3, 4, 5, 6, 7, 8, 9, or 10 amino acids. In some embodiments, the N-terminal deletion is from 1 to 15 amino acids. In some embodiments, the N-terminal deletion is a deletion of a single amino acid.

In some embodiments, a modified IL-2 polypeptide provided herein is synthetic. In some embodiments, the modified IL-2 polypeptide comprises a homoserine (Hse) residue located in any one of amino acid residues 35-45. In some embodiments, the modified IL-2 polypeptide comprises a Hse residue located in any one of amino acid residues 61-81. In some embodiments, the modified IL-2 polypeptide comprises a Hse residue located in any one of amino acid residues 94-114. In some embodiments, the modified IL-2 polypeptide comprises 1, 2, 3, or more Hse residues. In some embodiments, the modified IL-2 polypeptide comprises Hse41, Hse71, Hse104, or a combination thereof. In some embodiments, the modified IL-2 polypeptide comprises Hse41, Hse71, and Hse104. In some embodiments, the modified IL-2 polypeptide comprises at least two amino acid substitutions, wherein the at least two amino acid substitutions are selected from (a) a homoserine (Hse) residue located in any one of amino acid residues 35-45; (b) a homoserine residue located in any one of amino acid residues 61-81; and (c) a homoserine residue located in any one of amino acid residues 94-114. In some embodiments, the modified IL-2 polypeptide comprises Hse41 and Hse71. In some embodiments, the modified IL-2 polypeptide comprises Hse41 and Hse104. In some embodiments, the modified IL-2 polypeptide comprises Hse71 and Hse104. In some embodiments, the modified IL-2 polypeptide comprises Hse41. In some embodiments, the modified IL-2 polypeptide comprises Hse71. In some embodiments, the modified IL-2 polypeptide comprises Hse104. In some embodiments, the modified IL-2 polypeptide comprises 1, 2, 3, or more norleucine (Nle) residues. In some embodiments, the modified IL-2 polypeptide comprises a Nle residue located in any one of residues 18-28. In some embodiments, the modified IL-2 polypeptide comprises one or more Nle residues located in any one of amino acid residues 34-50. In some embodiments, the modified IL-2 polypeptide comprises a Nle residue located in any one of amino acid residues 20-60. In some embodiments, the modified IL-2 polypeptide comprises three Nle substitutions. In some embodiments, the modified IL-2 polypeptide comprises Nle23, Nle39, and Nle46. In some embodiments, the modified IL-2 polypeptide comprises each of the substitution in SEQ ID NO: 3 relative to WT IL-2 (SEQ ID NO: 1), and any of the other substitutions or modifications as provided herein.

### IL-2 Polypeptides Biased to IL-2 Receptor Beta Subunit

In some embodiments of the instant disclosure, it is preferable that the IL-2 polypeptide is biased in favor of signaling through the IL-2 receptor beta subunit compared to wild type IL-2. In some embodiments, this is accomplished through one or both of a) inhibiting or diminishing binding of the IL-2 polypeptide to the IL-2 receptor alpha subunit (e.g., with a mutation at a residue contacting the alpha subunit, with addition of a polymer to the residue contacting the alpha subunit, or through attachment of the linker to the antibody to the residue contacting the alpha subunit) and/or b) enhancing the binding of the IL-2 polypeptide to the beta subunit of the IL-2 receptor (e.g., with a mutation at a residue contacting the beta subunit which enhances binding). In some embodiments, the IL-2 polypeptide of the immunocytokine composition provided herein is biased towards the IL-2 receptor beta subunit compared to wild type IL-2. Non-limiting examples of IL-2 polypeptides with modifications which are biased towards IL-2 receptor beta signaling are described in, for example, PCT Publication Nos. WO2021140416A2, WO2012065086A1, WO2019028419A1, WO2012107417A1, WO2018119114A1, WO2012062228A2, WO2019104092A1, WO2012088446A1, and WO2015164815A1, each of which is hereby incorporated by reference as if set forth herein in its entirety.

In some embodiments, the linker is attached to the IL-2 polypeptide at a residue which disrupts binding of the IL-2 polypeptide with the IL-2 receptor alpha subunit (IL-2Rα). Examples of these residues include residues 3, 5, 34, 35, 36, 37, 38, 40, 41, 42, 43, 44, 45, 60, 61, 62, 63, 64, 65, 67, 68, 69, 71, 72, 103, 104, 105, and 107, as described in, for example, PCT Pub. Nos. WO2019028419A1, WO2020056066A1, WO2021140416A2, and WO2021216478A1 each of which is hereby incorporated by reference as if set forth in its entirety.

In some embodiments, the linker is attached to the IL-2 polypeptide at an amino acid residue at any one of positions 30-110, wherein residue position numbering of the modified IL-2 polypeptide is based on SEQ ID NO: 1 as a reference sequence. In some embodiments, the linker is attached to the IL-2 polypeptide at an amino acid residue at any one of positions 30-50, 30-70, 30-100, 40-50, 40-70, 40-100, or 40-110. In some embodiments, the linker is attached to the IL-2 polypeptide at an amino acid residue at any one of positions 35, 37, 38, 41, 42, 43, 44, 45, 60, 61, 62, 64, 65, 68, 69, 71, 72, 104, 105, and 107, wherein amino acid residue position numbering of the modified IL-2 polypeptide is based on SEQ ID NO: 1 as a reference sequence. In some embodiments, the linker is attached to the IL-2 polypeptide at an amino acid residue at any one of positions 35, 37, 38, 41, 43, 44, 45, 60, 61, 62, 64, 65, 68, 69, 71, 72, 104, 105, and 107, wherein amino acid residue position numbering of the modified IL-2 polypeptide is based on SEQ ID NO: 1 as a reference sequence. In some embodiments, the linker is attached to the IL-2 polypeptide at an amino acid residue at any one of positions 35, 37, 38, 41, 42, 43, 44, 60, 61, 62, 64, 65, 68, 69, 71, 72, 104, 105, and 107, wherein amino acid residue position numbering of the modified IL-2 polypeptide is based on SEQ ID NO: 1 as a reference sequence. In some embodiments, the linker is attached to the IL-2 polypeptide at an amino acid residue at any one of positions 35, 37, 38, 41, 43, 44, 60, 61, 62, 64, 65, 68, 69, 71, 72, 104, 105, and 107, wherein amino acid residue position numbering of the modified IL-2 polypeptide is based on SEQ ID NO: 1 as a reference sequence In some embodiments, the linker is attached to the IL-2 polypeptide at an amino acid residue at any one of positions 35, 37, 38, 39, 40, 41, 42, 43, 44, 45, or 46. In some embodiments, the linker is attached to the IL-2 polypeptide at an amino acid residue at any one of positions 41, 42, 43, 44, and 45, wherein amino acid residue position numbering of the modified IL-2 polypeptide is based on SEQ ID NO: 1 as a reference sequence. In some embodiments, the linker is attached at amino acid residue 42 or 45. In some embodiments, the linker is attached at amino acid residue 42. In some embodiments, the linker is attached at amino acid residue 45.

In some embodiments, the linker is attached to an amino acid residue which has been substituted with a natural amino acid. In some embodiments, the linker is attached to an amino acid residue which has been substituted with a cysteine, lysine, or tyrosine residue. In some embodiments, the linker is attached to a residue which has been substituted with a cysteine residue. In some embodiments, the linker is attached to an amino acid residue which has been substituted with a lysine residue. In some embodiments, the linker is attached to an amino acid residue which has been substituted with a tyrosine residue. In some embodiments, where the cytokine comprises a IL-2 polypeptide, the linker is attached to amino acid residue K35, F42Y, K43, F44Y, or Y45. In some embodiments, where the cytokine comprises a IL-2 polypeptide, the linker is attached to amino acid residue F42Y or Y45. In some embodiments, where the cytokine comprises a IL-2 polypeptide, the linker is attached to amino acid residue F42Y. In some embodiments, the linker is attached to amino acid residue Y45.

In some embodiments, the modified cytokines described herein described herein contain one or more modified amino acid residues. Such modifications can take the form of mutations of a wild type IL-2 polypeptide such as the amino acid sequence of SEQ ID NO: 1, addition and/or deletion of amino acids from the sequence of SEQ ID NO: 1, or the addition of moieties to amino acid residues. In some embodiments, the modified IL-2 polypeptide described herein contains a deletion of the first amino acid from the sequence of SEQ ID NO: 1. In some embodiments, the modified IL-2 polypeptide described herein comprises a C125S mutation, using the sequence of SEQ ID NO: 1 as a reference sequence. Moieties which can be added to amino acid residues include, but are not limited to, polymers, linkers, spacers, and combinations thereof. When added to certain amino acid residues, these moieties can modulate the activity or other properties of the modified IL-2 polypeptide compared to wild-type IL-2. In some embodiments, the modified IL-2 polypeptides comprise two modifications in the range of amino acid residues 35-46. In some embodiments, one modification is in the range of amino acid residues 40-43. In some embodiments, one modification is at amino acid residue 42. In some embodiments, one modification is in the range of amino acid residues 44-46. In some embodiments, one modification is at amino acid residue 45.

In some embodiments, a modified IL-2 polypeptide provided herein comprises an amino acid sequence of any one of SEQ ID NOs: 3-23 provided in Table 8A. In some embodiments, the modified IL-2 polypeptide comprises an amino acid sequence at least 80%, 85%, 90%, 95%, 99%, or 100% identical to the sequence of any one of SEQ ID NOs: 3-23. In some embodiments, the modified IL-2 polypeptide comprises an amino acid sequence of SEQ ID NO: 3. In some embodiments, the modified IL-2 polypeptide comprises an amino acid sequence at least 80%, 85%, 90%, 95%, 99%, or 100% identical to the sequence of SEQ ID NO: 3. In some embodiments, the modified IL-2 polypeptide comprises an amino acid sequence of SEQ ID NO: 4. In some embodiments, the modified IL-2 polypeptide comprises an amino acid sequence at least 80%, 85%, 90%, 95%, 99%, or 100% identical to the sequence of SEQ ID NO: 4. In some embodiments, the modified IL-2 polypeptide comprises an amino acid sequence of SEQ ID NO: 9. In some embodiments, the modified IL-2 polypeptide comprises an amino acid sequence at least 80%, 85%, 90%, 95%, 99%, or 100% identical to the sequence of SEQ ID NO: 9. In some embodiments, the modified IL-2 polypeptide comprises an amino acid sequence of SEQ ID NO: 10. In some embodiments, the modified IL-2 polypeptide comprises an amino acid sequence at least 80%, 85%, 90%, 95%, 99%, or 100% identical to the sequence of SEQ ID NO: 10. In some embodiments, the modified IL-2 polypeptide comprises an amino acid sequence of SEQ ID NO: 11. In some embodiments, the modified IL-2 polypeptide comprises an amino acid sequence at least 80%, 85%, 90%, 95%, 99%, or 100% identical to the sequence of SEQ ID NO: 11. In some embodiments, the modified IL-2 polypeptide comprises an amino acid sequence of SEQ ID NO: 12. In some embodiments, the modified IL-2 polypeptide comprises an amino acid sequence at least 80%, 85%, 90%, 95%, 99%, or 100% identical to the sequence of SEQ ID NO: 12. In some embodiments, the modified IL-2 polypeptide comprises an amino acid sequence of SEQ ID NO: 13. In some embodiments, the modified IL-2 polypeptide comprises an amino acid sequence at least 80%, 85%, 90%, 95%, 99%, or 100% identical to the sequence of SEQ ID NO: 13. In some embodiments, the modified IL-2 polypeptide comprises an amino acid sequence of SEQ ID NO: 14. In some embodiments, the modified IL-2 polypeptide comprises an amino acid sequence at least 80%, 85%, 90%, 95%, 99%, or 100% identical to the sequence of SEQ ID NO: 14. In some embodiments, the modified IL-2 polypeptide comprises an amino acid sequence of SEQ ID NO: 15. In some embodiments, the modified IL-2 polypeptide comprises an amino acid sequence at least 80%, 85%, 90%, 95%, 99%, or 100% identical to the sequence of SEQ ID NO: 15. In some embodiments, the modified IL-2 polypeptide comprises an amino acid sequence of SEQ ID NO: 17. In some embodiments, the modified IL-2 polypeptide comprises an amino acid sequence at least 80%, 85%, 90%, 95%, 99%, or 100% identical to the sequence of SEQ ID NO: 17. In some embodiments, the modified IL-2 polypeptide comprises an amino acid sequence of SEQ ID NO: 18. In some embodiments, the modified IL-2 polypeptide comprises an amino acid sequence at least 80%, 85%, 90%, 95%, 99%, or 100% identical to the sequence of SEQ ID NO: 18. In some embodiments, the modified IL-2 polypeptide comprises an amino acid sequence of SEQ ID NO: 19. In some embodiments, the modified IL-2 polypeptide comprises an amino acid sequence at least 80%, 85%, 90%, 95%, 99%, or 100% identical to the sequence of SEQ ID NO: 19. In some embodiments, the modified IL-2 polypeptide comprises an amino acid sequence of SEQ ID NO: 20. In some embodiments, the modified IL-2 polypeptide comprises an amino acid sequence at least 80%, 85%, 90%, 95%, 99%, or 100% identical to the sequence of SEQ ID NO: 20. In some embodiments, the modified IL-2 polypeptide comprises an amino acid sequence of SEQ ID NO: 21. In some embodiments, the modified IL-2 polypeptide comprises an amino acid sequence at least 80%, 85%, 90%, 95%, 99%, or 100% identical to the sequence of SEQ ID NO: 21. In some embodiments, the modified IL-2 polypeptide comprises an amino acid sequence of SEQ ID NO: 22. In some embodiments, the modified IL-2 polypeptide comprises an amino acid sequence at least 80%, 85%, 90%, 95%, 99%, or 100% identical to the sequence of SEQ ID NO: 22. In some embodiments, the modified IL-2 polypeptide comprises an amino acid sequence of SEQ ID NO: 23. In some embodiments, the modified IL-2 polypeptide comprises an amino acid sequence at least 80%, 85%, 90%, 95%, 99%, or 100% identical to the sequence of SEQ ID NO: 23.

In some embodiments, the modified IL-2 polypeptides described herein contain one or more polymers. For .example, the addition of polymers to certain amino acid residues can have the effect of disrupting the binding interaction of the modified IL-2 polypeptide with IL-2R, particularly the αβγ complex. In some embodiments, residues to which polymers are added to disrupt this interaction include F42 and Y45. In some embodiments, the polymer added to residue 42 or 45 also acts as the linker between the IL-2 polypeptide and the polypeptide which binds to the a cancer antigen, an immune cell target molecule, a self-antigen, or any combination thereof.

In some embodiments, the polymers are water-soluble polymers, such as polyethylene glycol (PEG) polymers. The F42 residue can be mutated to another residue to facilitate the addition of the PEG polymer (or the linker), for example to a tyrosine residue. Polymers may be added to either one or both of residues F42 and Y45, or mutants thereof. These polymers may be either in the form of a linker between the IL-2 polypeptide and the polypeptide which selectively binds to TNFα or may be an additional polymer in addition to the linker. Additionally, polymers may be added to modified IL-2 polypeptides in order to increase the half-life of the polypeptides. In some embodiments, the linker between the IL-2 polypeptide and the polypeptide which selectively binds to TNFα has the effect of increasing the half-life of the polypeptide conjugate. Alternatively, such half-life extending polymers can be added to the N-terminus of the modified IL-2 polypeptides, or another residue provide herein. The half-life extending polymers may be of any size, including up to about 6 kDa, up to about 25 kDa, or up to about 50 kDa. In some embodiments, the half-life extending polymers are PEG polymers. In some embodiments, the modified IL-2 polypeptide comprises one or more amino acid mutations selected from TABLE 2.

TABLE 2

| WT IL-2 | WT | |
|---|---|---|
| Residue | IL-2 | |
| Number* | Residue | Mutations |
| 35 | K | D, I, L, M, N, P, Q, T, Y |
| 36 | L | A, D, E, F, G, H, 1, K, M, N, P, R, S, W, Y |
| 38 | R | A, D, G, K, N, P, S, Y |
| 40 | L | D, G, N, S, Y |
| 41 | T | E, G, Y |
| 42 | F | A, D, E, G, I, K, L, N, Q, R, S, T, V, Y |
| 43 | K | H, Y |
| 44 | F | K, Y |
| 45 | Y | A, D, E, G, K, L, N, Q, R, S, T, V |
| 46 | M | I, Y |
| 61 | E | K, M, R, Y |
| 62 | E | D, L, T, Y |
| 64 | K | D, E, G, L, Q, R, Y |
| 65 | P | D, E, F, G, H, I, K, L, N, Q, R, S, T, V, W, Y |
| 66 | L | A, F, Y |
| 67 | E | A, Y |
| 68 | E | V, Y |
| 72 | L | A, D, E, G, K, N, Q, R, S, T, Y |
| 125 | C | S |

*Residue position numbering based on SEQ ID NO: 1 as a reference sequence.

In some embodiments, a modified IL-2 polypeptide provided herein comprises one or more amino acid mutations selected from TABLE 3.

TABLE 3

| WT IL-2 | | |
|---|---|---|
| Residue | WT IL-2 | |
| Number* | Residue | Mutations |
| 20 | D | T, Y |
| 35 | K | D, I, L, M, N, P, Q, T Y |
| 38 | R | A, D, G, K, N, P, S, Y |
| 42 | F | A, D, E, G, I, K, L, N, Q, R, S, T, V, Y |
| 43 | K | H, Y |
| 45 | Y | A, D, E, G, K, L, N, Q, R, S, T, V, Y |
| 62 | E | D, L, T, Y |
| 65 | P | D, E, F, G, H, I, K, L, N, Q, R, S, T, V, W, Y |
| 68 | E | V, Y |
| 72 | L | A, D, E, G, K, N, Q, R, S, T, Y |
| 125 | C | S |

*Residue position numbering based on SEQ ID NO: 1 as a reference sequence.

In some embodiments, a modified IL-2 polypeptide provided herein comprises one or more polymers selected from TABLE 4.

TABLE 4

| Polymer | |
|---|---|
| Identifier | |
| and | |
| Approx. | |
| Molecular | |
| Weight | Polymer Structure |
| Formula D 500 Da | 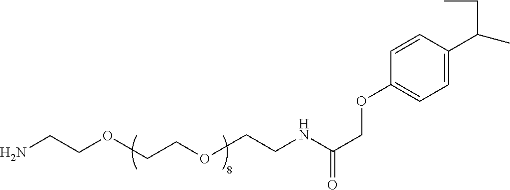 |

In some embodiments, a modified IL-2 polypeptide provided herein comprises mutation and polymers as provided in TABLE 5.

TABLE 5

| | Polymer | |
|---|---|---|
| | Residue | |
| Mutation* | Location | Polymer |
| F42Y | 42, 45 | Formula D (Residues 42, 45) |
| None | 45 | Formula D |
| F42A | 45 | Formula D |
| F42Y, L72G | 42, 45 | Formula D (Residues 42, 45) |
| F42Y, P65Y | 42, 65 | Formula D (Residues 42, 65) |
| F42Y, P65Y | 42, 45, 65 | Formula D (Residues 42, 45, 65) |
| R38Y, F42Y, | 38, 42, 45, | Formula D (Residues 38, 42, 45, 62, 68) |
| E62Y, E68Y | 62, 68 | |
| F42Y, L72Y | 42, 45, 72 | Formula D (Residues 42, 45, 75) |
| F42Y, Y45K | 42 | Formula D |
| F42A | 45 | Formula D |
| L72G | 45 | Formula D |
| F42Y | 42, 45 | Formula D (Residue 42), linker to the |
| | | antibody (Residue 45) |
| F42Y | 42, 45 | Formula D (Residues 45), linker to the |
| | | antibody (Residue 42) |

*Residue position numbering based on SEQ ID NO: 1 as a reference sequence.

In some instances, the modified cytokine described herein may be recombinant. The modified IL-2 cytokine described herein may also be synthesized chemically rather than expressed as recombinant polypeptides. For example, synthetic IL-2 polypeptides have been described, at least in US Patent Application Publication No US20190023760A1 and Asahina et al., Angew. Chem. Int. Ed. 2015, 54, 8226-8230, each of which is incorporated by reference as if set forth herein in its entirety. The modified cytokine can be made by synthesizing one or more fragments of the full-length modified cytokine, ligating the fragments together, and folding the ligated full-length polypeptide. In some embodiments, where the cytokine comprises a modified IL-2 polypeptide and the modified IL-2 polypeptide comprises an F42Y mutation in the amino acid sequence, a first PEG polymer of about 500 Da covalently attached to amino acid residue F42Y, a second PEG polymer of about 500 Da covalently attached to amino acid residue Y45, and an optional third PEG polymer of about 6 kDa covalently attached to the N-terminus of the modified IL-2 polypeptide. In some embodiments, the PEG polymer comprises a portion of the linker which attached the IL-2 polypeptide to the polypeptide which binds to the antigen or antigen binding fragment. In some instances, the cytokine comprises a modified IL-7 polypeptide. In some instances, the cytokine comprises a modified IL-18 polypeptide.

In some embodiments, a chemically synthesized cytokine comprises a conjugation handle attached to one or more residues to facilitate attachment of the linker to the antibody or antigen binding fragment. The conjugation handle may be any such conjugation handle provided herein and may be attached at any residue to which the linker may be attached. In some embodiments, where the cytokine is a modified IL-2 polypeptide, the conjugation handle is attached to, for example, residue 42 or 45 of an IL-2 polypeptide. In some embodiments, the conjugation handle comprises an azide or an alkyne. Alternatively, in some embodiments, the conjugation handle is incorporated into an unnatural or modified natural amino acid of a recombinant IL-2 polypeptide. Recombinant IL-2 polypeptides with unnatural amino acids can be made using methods as described in, for example, Patent Cooperation Treaty Publication Nos. WO2016115168, WO2002085923, WO2005019415, and WO2005003294.

In some embodiments, a modified IL-2 polypeptide described herein comprises a modification at an amino acid residue from the region of residues 35-46, wherein the residue numbering is based on SEQ ID NO: 1. In some embodiments, the modification is at K35, L46, T37, R38, M39, L40, T41, F42, K43, F44, Y45, or M46. In some embodiments, the modification is at F42. In some embodiments, the modification is at Y45. In some embodiments, the modified IL-2 polypeptide comprises a modification at the N-terminal residue. In some embodiments, the modified IL-2 polypeptide comprises a C125S mutation. In some embodiments, the modified IL-2 polypeptide comprises an A1 deletion. In some embodiments, the modification comprises attachment of the linker which attached the IL-2 polypeptide to the antibody or antigen binding fragment.

In some embodiments, a modified IL-2 polypeptide described herein comprises a first polymer covalently attached at an amino acid residue in any of residues 35-46, wherein amino acid residue position numbering of the modified IL-2 polypeptide is based on SEQ ID NO: 1 as a reference sequence. In some embodiments, the modified IL-2 polypeptide comprises a first polymer covalently attached at an amino acid residue in any of residues 39-43. In some embodiments, the modified IL-2 polypeptide comprises a first polymer covalently attached at amino acid residue F42. In some embodiments, the modified IL-2 polypeptide comprises a first polymer covalently attached at amino acid residue F42Y. In some embodiments, the modified IL-2 polypeptide comprises a first polymer covalently attached at an amino acid residue in any of residues 44-46. In some embodiments, the modified IL-2 polypeptide comprises a first polymer covalently attached at amino acid residue Y45. In some embodiments, the first polymer is part of the linker which attaches the IL-2 polypeptide to the antibody or antigen binding fragment. In some embodiments, the first polymer is a separate modification from the linker which attached the IL-2 polypeptide to the antibody or antigen binding fragment.

In some embodiments, a modified IL-2 polypeptide described herein comprises one or more PEGylated tyrosine located at an amino acid residue in the region from amino acid residue 35 to amino acid residue 45. In some embodiments, the one or more PEGylated tyrosine is located at amino acid residue 42, amino acid residue 45, or both. In some embodiments, the one or more PEGylated tyrosine is located at amino acid residue 42. In some embodiments, the one or more PEGylated tyrosine is located at amino acid residue 45. In some embodiments, the one or more PEGylated tyrosine is located at both amino acid residue 42 and amino acid residue 45. In some embodiments, the modified IL-2 polypeptide comprises two PEGylated tyrosines, each independently having a structure of Formula (I). A non-limiting set of modified IL-2 polypeptides provided herein with various linker points of attachment and polymers as provided herein is shown in Table 9 below.

TABLE 9

| | Linker | Polymer 1 | Polymer 2 |
|---|---|---|---|
| IL-2 | Point of | Point of | Point of |
| Construct | Attachment | Attachment | Attachment |
| A | N-terminus | Residue 42 | Residue 45 |
| B | N-terminus | Residue 42 | None |
| C | N-terminus | Residue 45 | None |
| D | Residue 42 | Residue 45 | None |
| E | Residue 42 | N-terminus | Residue 45 |
| F | Residue 42 | N-terminus | None |
| G | Residue 45 | Residue 42 | None |
| H | Residue 45 | N-terminus | Residue 42 |
| I | Residue 45 | N-terminus | None |
| J | N-terminus | Residue 65 | None |
| K | Residue 65 | N-terminus | None |

*Residue position numbering based on SEQ ID NO: 1 as a reference sequence

In one aspect, disclosed herein is a modified IL-2 polypeptide comprising one or more amino acid substitutions. In some embodiments, the modified IL-2 polypeptide comprises F42Y and Y45.

In one aspect, described herein is a modified polypeptide that comprises a modified interleukin-2 (IL-2) polypeptide, wherein the modified IL-2 polypeptide comprises a covalently attached first polymer. Described herein is a modified polypeptide comprising a modified interleukin-2 (IL-2) polypeptide, wherein the modified IL-2 polypeptide comprises a first polymer covalently attached at residue F42Y, and wherein residue position numbering of the modified IL-2 polypeptide is based on SEQ ID NO: 1 as a reference sequence. In some embodiments, the first polymer is the same as linker which attaches the IL-2 polypeptide the antibody or antigen binding fragment. In some embodiments, the first polymer is an additional polymer which is distinct from the linker.

In some embodiments, where the modified cytokine is a IL-2 polypeptide, in some cases, Tyr 45 and/or Phe 42 are substituted with non-canonical amino acids. In some embodiments, one or more amino acids located at positions provided in Table 2 and/or Table 3 are substituted with one or more non-canonical amino acids. In some embodiments, Tyr 45 and/or Phe 42 are substituted with modified tyrosine residues. In some embodiments, the modified tyrosine residues comprise an amino, azide, allyl, ester, and/or amide functional groups. In some embodiments, the modified tyrosine residue at position 42 or 45 is used as the point of attachment for the linker which attaches the IL-2 polypeptide to the antibody or antigen binding fragment. In some embodiments, the modified tyrosine residues at positions 42 and/or 45 have a structure built from precursors Structure 1, Structure 2, Structure 3, Structure 4, or Structure 5, wherein Structure 1 is:

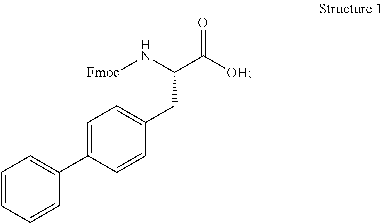

### Structure 2 is:

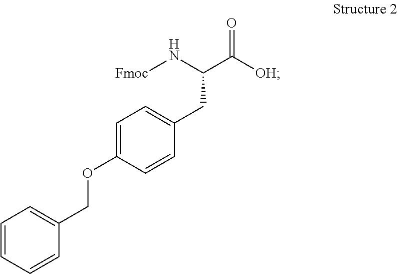

### Structure 3 is:

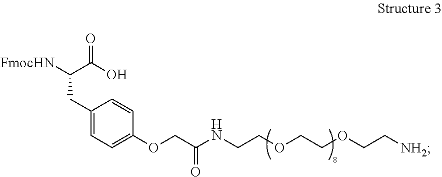

### Structure 4 is:

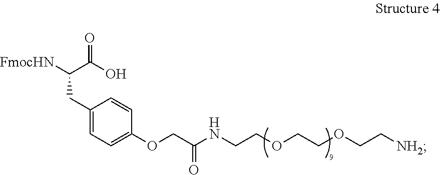

### and Structure 5 is:

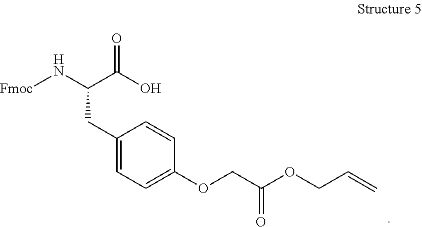

In some embodiments, the modified modified IL-2 polypeptide enhance a and/or activate T-effector (T_{eff}) or natural killer (NK) cell proliferation when administered to a subject. In some embodiments, the modified IL-2 polypeptide enhances and/or activates T_{eff} or NK cell proliferation while sparing regulatory T-cells (T_{reg}) when administered to a subject. In some embodiments, the modified IL-2 polypeptides increase CD8+ T and NK cells without increasing CD4+ regulator T cells when administered to a subject. In some embodiments, the modified IL-2 polypeptides produce a T_{eff}/T_{reg} ratio of close to 1 when administered to a subject. In some instances, the modified IL-2 polypeptide is a modified IL-2 polypeptide described herein, a modified IL-2 polypeptide provided in Table 8A (in particular SEQ ID NOs: 1-23) or Table 5, a modified IL-2 polypeptide having a mutation provided in Table 2 or Table 3, and/or a modified IL-2 polypeptide having a polymer provided in Table 4.

In some embodiments, a modified IL-2 polypeptide described herein expands a cell population of effector T cells (T_{eff} cells). In some embodiments, the modified cytokine expands a cell population of T_{eff} cells by at least 1%, at least 2%, at least 5%, at least 10%, at least 15%, at least 20%, at least 30%, at least 40%, at least 50%, at least 100%, or at least 200% when the modified cytokine is in contact with the population. In some embodiments, the modified cytokine expands a cell population of T_{eff} cells by at least 20% when the modified cytokine is in contact with the population. In some embodiments, the modified cytokine expands a cell population of T_{eff} cells by at least 30% when the modified cytokine is in contact with the population. In some embodiments, the modified cytokine expands a cell population of T_{eff} cells by at least 40% when the modified cytokine is in contact with the population. In some embodiments, the modified cytokine expands a cell population of T_{eff} cells by at least 50% when the modified cytokine is in contact with the population. In some embodiments, the modified cytokine expands a cell population of T_{eff} cells by at least 100% when the modified cytokine is in contact with the population. In some embodiments, the modified cytokine expands a cell population of T_{eff} cells by at least 200% when the modified cytokine is in contact with the population.

In some embodiments, a modified cytokine described herein expands a cell population of effector T cells (T_{eff} cells). In some embodiments, the modified cytokine expands a cell population of T_{eff} cells by at most 5%, at most 10%, at most 20%, at most 30%, at most 40%, at most 50%, at most 75%, at most 100%, or at most 500% when the modified cytokine is in contact with the population. In some embodiments, the modified cytokine expands a cell population of T_{eff} cells by at most 5%, when the modified cytokine is in contact with the population. In some embodiments, the modified cytokine expands a cell population of T_{eff} cells by at most 20%, when the modified cytokine is in contact with the population. In some embodiments, the modified cytokine expands a cell population of T_{eff} cells by at most 50%, when the modified cytokine is in contact with the population. In some embodiments, the modified cytokine expands a cell population of T_{eff} cells by at most 100%, when the modified cytokine is in contact with the population. In some embodiments, the modified cytokine expands a cell population of T_{eff} cells by at most 500%, when the modified cytokine is in contact with the population.

In some embodiments, a ratio of cell population expansion of T_{eff} cells over cell population expansion of T_{reg} cells expanded by a modified IL-2 polypeptide described herein is from about 0.1 to about 15, from about 0.5 to about 10, from about 0.75 to about 5, or from about 1 to about 2. In some embodiments, a ratio of cell population expansion of T_{eff} cells over cell population expansion of T_{reg} cells expanded by the modified cytokine is from 0.1 to 15. In some embodiments, a ratio of cell population expansion of T_{eff} cells over cell population expansion of T_{reg} cells expanded by the modified cytokine is from 0.1 to 0.5, from 0.1 to 0.75, from 0.1 to 1, from 0.1 to 2, from 0.1 to 5, from 0.1 to 10, from 0.1 to 15, from 0.5 to 0.75, from 0.5 to 1, from 0.5 to 2, from 0.5 to 5, from 0.5 to 10, from 0.5 to 15, from 0.75 to 1, 0.75 to 2, from 0.75 to 5, from 0.75 to 10, from 0.75 to 15, from 1 to 2, from 1 to 5, from 1 to 10, from 1 to 15, from 2 to 5, from 2 to 10, from 2 to 15, from 5 to 10, from 5 to 15, from 10 to 15, or any numbers or ranges therebetween. In some embodiments, a ratio of cell population expansion of T_{eff} cells over cell population expansion of T_{reg} cells expanded by the modified IL-2 polypeptide is about 0.1, 0.5, 0.75, 1, 2, 5, 10, or 15. In some embodiments, a ratio of cell population expansion of T_{eff} cells over cell population expansion of T_{reg} cells expanded by the modified IL-2 polypeptide is at least 0.1, 0.5, 0.75, 1, 2, 5, or 10. In some embodiments, a ratio of cell population expansion of T_{eff} cells over cell population expansion of T_{reg} cells expanded by the modified IL-2 polypeptide is at most 0.5, 0.75, 1, 2, 5, 10, or 15.

In some embodiments, a cell population expanded by a modified cytokine provided herein is an in vitro cell population, an in vivo cell population, or an ex vivo cell population. In some embodiments, the cell population is an in vitro cell population. In some embodiments, the cell population is an in vivo cell population. In some embodiments, the cell population is an ex vivo cell population. The cell population may be a population of CD4+ helper cells, CD8+ central memory cells, CD8+ effector memory cells, naïve CD8+ cells, Natural Killer (NK) cells, Natural killer T (NKT) cells, or a combination thereof. Alternatively, the cell population may be a population of T regulatory cells.

In some embodiments, the levels of cells are measured 1 hour after injection of the modified IL cytokine. In some embodiments, the levels of cells are measured 2 hours after injection of the modified cytokine. In some embodiments, the levels of cells are measured 4 hours after injection of the modified cytokine. In some embodiments, the levels of cells are measured 30 minutes after injection of the modified cytokine.

### Polymers Attached to IL-2 Polypeptides Biased to IL-2 Receptor Beta Subunit

In some embodiments, a modified cytokine described herein comprises a first polymer covalently attached to the N-terminus of the cytokine. In some embodiments, the modified cytokine comprises a second polymer covalently attached thereto. In some embodiments, the modified cytokine comprises a second and a third polymer covalently attached thereto. In some embodiments, where the cytokine comprises IL-2, the second polymer is covalently attached to amino acid residue 42 or 45, wherein residue position numbering of the modified IL-2 polypeptide is based on SEQ ID NO: 1 as a reference sequence. In some embodiments, the second polymer is covalently attached to amino acid residue F42Y or Y45, wherein the residue position numbering of the modified IL-2 polypeptide is based on SEQ ID NO: 1 as a reference sequence. In some embodiments, the second and third polymers are covalently attached to amino acid residue 42 and 45, wherein residue position numbering of the modified IL-2 polypeptide is based on SEQ ID NO: 1 as a reference sequence. In some embodiments, the second and third polymers are covalently attached to amino acid residue F42Y and Y45, wherein residue position numbering of the modified IL-2 polypeptide is based on SEQ ID NO: 1 as a reference sequence. In some embodiments, at least one of the first, second, or third polymers comprises at least a portion of the linker used to attach the IL-2 polypeptide to the antibody or antigen binding fragment.

In some embodiments, the attached polymer such as the first polymer has an average molecular weight of from about 120 Daltons to about 1,000 Daltons. In some embodiments, the polymer has an average molecular weight of from about 120 Daltons to about 250 Daltons, from about 120 Daltons to about 300 Daltons, from about 120 Daltons to about 400 Daltons, from about 120 Daltons to about 500 Daltons, from about 120 Daltons to about 1,000 Daltons, from about 250 Daltons to about 300 Daltons, from about 250 Daltons to about 400 Daltons, from about 250 Daltons to about 500 Daltons, from about 250 Daltons to about 1,000 Daltons, from about 300 Daltons to about 400 Daltons, from about 300 Daltons to about 500 Daltons, from about 300 Daltons to about 1,000 Daltons, from about 400 Daltons to about 500 Daltons, from about 400 Daltons to about 1,000 Daltons, or from about 500 Daltons to about 1,000 Daltons. In some embodiments, the polymer has an average molecular weight of about 120 Daltons, about 250 Daltons, about 300 Daltons, about 400 Daltons, about 500 Daltons, or about 1,000 Daltons. In some embodiments, the polymer has an average molecular weight of at least about 120 Daltons, about 250 Daltons, about 300 Daltons, about 400 Daltons, or about 500 Daltons. In some embodiments, the polymer has an average molecular weight of at most about 250 Daltons, about 300 Daltons, about 400 Daltons, about 500 Daltons, or about 1,000 Daltons.

In some embodiments, the attached polymer such as the first polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is poly(alkylene oxide) such as polyethylene glycol (e.g., polyethylene oxide). In some embodiments, the water-soluble polymer is polyethylene glycol. In some embodiments, the water-soluble polymer comprises modified poly(alkylene oxide). In some embodiments, the modified poly(alkylene oxide) comprises one or more linker groups. In some embodiments, the one or more linker groups comprise bifunctional linkers such as an amide group, an ester group, an ether group, a thioether group, a carbonyl group and alike. In some embodiments, the one or more linker groups comprise an amide linker group. In some embodiments, the modified poly(alkylene oxide) comprises one or more spacer groups. In some embodiments, the spacer groups comprise a substituted or unsubstituted C₁-C₆ alkylene group. In some embodiments, the spacer groups comprise —CH₂—, —CH₂CH₂—, or —CH₂CH₂CH₂—. In some embodiments, the linker group is the product of a biorthogonal reaction (e.g., biocompatible and selective reactions). In some embodiments, the bioorthogonal reaction is a Cu(I)-catalyzed or “copper-free” alkyne-azide triazole-forming reaction, the Staudinger ligation, inverse-electron-demand Diels-Alder (IEDDA) reaction, “photo-click” chemistry, or a metal-mediated process such as olefin metathesis and Suzuki-Miyaura or Sonogashira cross-coupling. In some embodiments, the first polymer is attached to the IL-2 polypeptide via click chemistry. In some embodiments, the first polymer comprises at least a portion of the linker which attaches the cytokine to the antibody or antigen binding fragment

In some embodiments, the water-soluble polymer comprises from 1 to 10 polyethylene glycol chains.

In some embodiments, a modified IL-2 polypeptide described herein further comprises a second polymer covalently attached to the modified IL-2 polypeptide. In some embodiments, the second polymer is covalently attached at an amino acid residue region from residue 40 to residue 50. In some embodiments, the second polymer is covalently attached at residue Y45. In some embodiments, the second polymer is covalently attached to the N-terminus of the modified IL-2 polypeptide. In some embodiments, second polymer comprises at least a portion of the linker which attaches the IL-2 polypeptide to the polypeptide which selectively binds to an antigen (e.g., the antibody or antigen binding fragment thereof).

In some embodiments, the second polymer has an average molecular weight of about 120 Daltons to about 1,000 Daltons. In some embodiments, the second polymer has an average molecular weight of about 120 Daltons to about 250 Daltons, about 120 Daltons to about 300 Daltons, about 120 Daltons to about 400 Daltons, about 120 Daltons to about 500 Daltons, about 120 Daltons to about 1,000 Daltons, about 250 Daltons to about 300 Daltons, about 250 Daltons to about 400 Daltons, about 250 Daltons to about 500 Daltons, about 250 Daltons to about 1,000 Daltons, about 300 Daltons to about 400 Daltons, about 300 Daltons to about 500 Daltons, about 300 Daltons to about 1,000 Daltons, about 400 Daltons to about 500 Daltons, about 400 Daltons to about 1,000 Daltons, or about 500 Daltons to about 1,000 Daltons. In some embodiments, the second polymer has an average molecular weight of about 120 Daltons, about 250 Daltons, about 300 Daltons, about 400 Daltons, about 500 Daltons, or about 1,000 Daltons. In some embodiments, the second polymer has an average molecular weight of at least about 120 Daltons, about 250 Daltons, about 300 Daltons, about 400 Daltons, or about 500 Daltons. In some embodiments, the second polymer has an average molecular weight of at most about 250 Daltons, about 300 Daltons, about 400 Daltons, about 500 Daltons, or about 1,000 Daltons.

In some embodiments, the second polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is poly(alkylene oxide). In some embodiments, the water-soluble polymer is poly(ethylene oxide). In some embodiments, the second polymer is attached to the IL-2 polypeptide via click chemistry. In some embodiments, the second polymer comprises at least a portion of the linker which attaches the IL-2 polypeptide to the antibody.

In some embodiments, the second water-soluble polymer comprises from 1 to 10 polyethylene glycol chains. In some embodiments, each of the first polymer and the second polymer independently comprises one polyethylene glycol chain with 3 to 25 ethylene glycol units. In some embodiments, each of the polyethylene glycol chains is independently linear or branched. In some embodiments, each of the polyethylene glycol chains is a linear polyethylene glycol. In some embodiments, each of the polyethylene glycol chains is a branched polyethylene glycol. For example, in some embodiments, each of the first and the second polymers comprises a linear polyethylene glycol chain.

In some embodiments, a modified IL-2 polypeptide described herein further comprises a third polymer covalently attached to the modified IL-2 polypeptide. In some embodiments, the third polymer is covalently attached at an amino acid residue region from amino acid residue 40 to amino acid residue 50. In some embodiments, the third polymer is covalently attached at amino acid residue Y45. In some embodiments, the third polymer is covalently attached to the N-terminus of the modified IL-2 polypeptide.

In some embodiments, the third polymer has an average molecular weight of from about 120 Daltons to about 1,000 Daltons. In some embodiments, the third polymer has an average molecular weight of from about 120 Daltons to about 250 Daltons, from about 120 Daltons to about 300 Daltons, from about 120 Daltons to about 400 Daltons, from about 120 Daltons to about 500 Daltons, from about 120 Daltons to about 1,000 Daltons, from about 250 Daltons to about 300 Daltons, from about 250 Daltons to about 400 Daltons, from about 250 Daltons to about 500 Daltons, from about 250 Daltons to about 1,000 Daltons, from about 300 Daltons to about 400 Daltons, from about 300 Daltons to about 500 Daltons, from about 300 Daltons to about 1,000 Daltons, from about 400 Daltons to about 500 Daltons, from about 400 Daltons to about 1,000 Daltons, or from about 500 Daltons to about 1,000 Daltons. In some embodiments, the third polymer has an average molecular weight of about 120 Daltons, about 250 Daltons, about 300 Daltons, about 400 Daltons, about 500 Daltons, or about 1,000 Daltons. In some embodiments, the third polymer has an average molecular weight of at least about 120 Daltons, about 250 Daltons, about 300 Daltons, about 400 Daltons, or about 500 Daltons. In some embodiments, the third polymer has an average molecular weight of at most about 250 Daltons, about 300 Daltons, about 400 Daltons, about 500 Daltons, or about 1,000 Daltons. In some embodiments, the modified IL-2 polypeptide comprises a third polymer having an average molecular weight of from about 1000 Daltons to about 10,000 Daltons covalently attached thereto. In some embodiments, the third polymer comprises at least a portion of the linker which attaches the cytokine to the antibody or antigen binding fragment.

In some embodiments, the third polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is poly(alkylene oxide). In some embodiments, the water-soluble polymer is polyethylene glycol. In some embodiments, the third polymer is attached to the cytokine via click chemistry. In some embodiments, the third polymer comprises at least a portion of the linker which attaches the cytokine to the antibody or antigen binding fragment.

In some embodiments, each of the polyethylene glycol chains is independently terminally capped with a hydroxy, an alkyl, an alkoxy, an amido, or an amino group. In some embodiments, each of the polyethylene glycol chains is independently terminally capped with an amino group. In some embodiments, each of the polyethylene glycol chains is independently terminally capped with an amido group. In some embodiments, each of the polyethylene glycol chains is independently terminally capped with an alkoxy group. In some embodiments, each of the polyethylene glycol chains is independently terminally capped with an alkyl group. In some embodiments, each of the polyethylene glycol chains is independently terminally capped with a hydroxy group.

In some embodiments, each of the polyethylene glycol chains is independently terminally capped with a hydroxy, an alkyl, an alkoxy, an amido, or an amino group. In some embodiments, each of the polyethylene glycol chains is independently terminally capped with an amino group. In some embodiments, each of the polyethylene glycol chains is independently terminally capped with an amido group. In some embodiments, each of the polyethylene glycol chains is independently terminally capped with an alkoxy group. In some embodiments, each of the polyethylene glycol chains is independently terminally capped with an alkyl group. In some embodiments, each of the polyethylene glycol chains is independently terminally capped with a hydroxy group.

In some embodiments, a water-soluble polymer that can be attached to a modified IL-2 polypeptide comprises a structure of Formula (D):

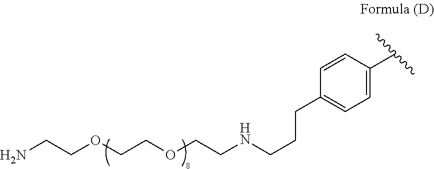

In some embodiments, the polymers are synthesized from suitable precursor materials. In some embodiments, the polymers are synthesized from the precursor materials of, Structure 6, Structure 7, Structure 8, or Structure 9, wherein Structure 6 is:

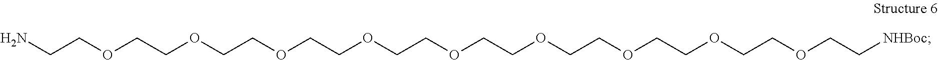 [SMILES: CC(C)(C)OC(=O)NCCOCCOCCOCCOCCOCCOCCOCCOCCOCCN]

### Structure 7 is:

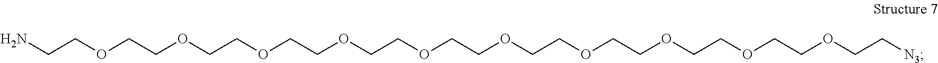 [SMILES: [N-]=[N+]=NCCOCCOCCOCCOCCOCCOCCOCCOCCOCCOCCN]

### Structure 8 is:

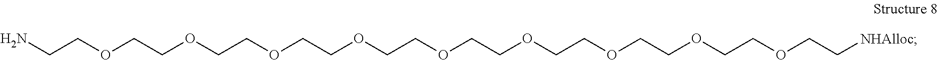

### and Structure 9 is:

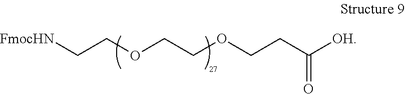

### IL-2 Polypeptides Biased to IL-2 Receptor Alpha Subunit

In one aspect, provided herein is a modified IL-2 polypeptide comprising one or more amino acid substitutions. In some embodiments, the amino acid substitutions affect the binding properties of the modified IL-2 polypeptide to IL-2 receptor subunits (e.g. alpha, beta, or gamma subunits) or to IL-2 receptor complexes (e.g. IL-2 receptor αβγ complex or βγ complex). In some embodiments, the amino acid substitutions are at positions on the interface of binding interactions between the modified IL-2 polypeptide and an IL-2 receptor subunit or an IL-2 receptor complex. In some embodiments, the amino acid substitutions cause an increase in affinity for the IL-2 receptor αβγ complex or alpha subunit. In some embodiments, the amino acid substitutions cause a decrease in affinity for the IL-2 receptor βγ complex or beta subunit.

In some embodiments, modified IL-2 polypeptides described herein have increased affinity for the IL-2R αβγ complex. In some embodiments, the modified IL-2 polypeptides have a reduced affinity for the IL-2R βγ complex. In some embodiments, the modified IL-2 polypeptides provided herein may comprise amino acid substitutions that enhance the binding affinity for the IL-2R alpha receptor subunit. In some embodiments, the modified IL-2 polypeptides provided herein comprise amino acid substitutions that lower the modified IL-2 polypeptides affinity for the IL-2R beta receptor subunit. In some embodiments, the modified IL-2 polypeptides have a biological activity of inducing or activating more T-regulatory (T_{reg}) cells when administered in vivo compared to a wild type IL-2 or aldesleukin. In some embodiments, the modified IL-2 polypeptides have a biological activity of inducing or activating fewer T-effector (T_{eff}) cells when administered in vivo compared to a wild type IL-2 or aldesleukin. In some embodiments, the modified IL-2 polypeptides provided herein have a substantially reduced ability (e.g., at least 100-fold lower ability) to induce or activate effector T cells when administered in vivo compared to a wild type IL-2 or aldesleukin.

In some embodiments, a substituted residue in the IL-2 polypeptide is selected such that the interaction of the IL-2 polypeptide with at least one IL-2 receptor subunit is decreased or blocked. In some embodiments, the substituted residue is selected such that interaction of the IL-2 polypeptide with an IL-2 receptor containing the IL-2R alpha subunit is unaffected or only slightly reduced. In some embodiments, the substituted residue is selected such that the interaction of the IL-2 polypeptide with the IL-2R beta subunit is substantially reduced or blocked.

Examples of amino acid substitutions and other modifications which bias an IL-2 polypeptide in favor of the IL-2 receptor alpha subunit are described in, for example, Rao et al., Protein Eng. 2003 December; 16(12):1081-7; Cassell et al., Curr Pharm Des. 2002; 8(24):2171-83; Rao et al., Biochemistry. 2005 Aug. 9; 44(31)10696-701; Mitra et al., Immunity. 2015 May 19; 42(5)826-38; U.S. Pat. No. 9,732,134 and US Patent Publication No.: US2020/0231644A1, each of which is incorporated by reference as if set forth herein in its entirety. In some embodiments, the modified IL-2 polypeptide comprises one of the amino acid substitutions provided therein.

In some embodiments, the modified IL-2 polypeptide comprises one or more amino acid substitutions selected form Table 6.

TABLE 6

| WT IL-2 | WT | |
|---|---|---|
| Residue | IL-2 | |
| Number* | Residue | Substitutions or modification |
| 1 | A | Deletion |
| 4 | S | P |
| 10 | T | A |
| 11 | Q | R |
| 12 | L | G, K, Q, S |
| 13 | Q | G |
| 15 | E | A, G, H, Q, S, T |
| 16 | H | A, D, G, K, M, N, Q, R, S, T, V, Y |
| 19 | L | A, D, E, G, H, N, R, S, T, V |
| 20 | D | A, E, F, G, H, L, M, S, T, W, |
| 21 | L | S, R, N |
| 22 | Q | N, H, K, Y, I |
| 23 | M | L, R, S, T, V |
| 29 | N | S |
| 30 | N | S |
| 31 | Y | H |
| 35 | K | R, E, D, Q |
| 37 | T | A, R |
| 38 | R | K |
| 42 | F | K |
| 48 | K | E, C |
| 69 | V | A |
| 71 | N | R |
| 74 | Q | P |
| 81 | R | A, G, S,T |
| 84 | D | A, E, G, 1, M, Q, R, S, T |
| 87 | S | R |
| 88 | N | A, D, E, F, G, H, 1, M, Q, R, S, T, V, W |
| 89 | I | V |
| 91 | V | D, E, G, K, S |
| 92 | I | K, R |
| 95 | E | G |
| 109 | D | C |
| 125 | C | S, E, K, H, W, I, V, A |
| 126 | Q | A, C, D, E, F, G, H, I, K, L, M, N, R, S, T, |
| | | Y |

*Residue position numbering based on SEQ ID NO: 1 as a reference sequence

In some embodiments, a modified IL-2 polypeptide provided herein comprises one or more amino acid substitutions selected from Table 7.

TABLE 7

| WT IL-2 Residue | WT IL-2 | |
|---|---|---|
| Number* | Residue | Mutations |
| 18 | L | R |
| 22 | Q | E |
| 23 | M | A |
| 29 | N | S |
| 31 | Y | H |
| 35 | K | R |
| 37 | T | A |
| 39 | M | A |
| 42 | F | (4-NH₂)-Phe |
| 46 | M | A |
| 48 | K | E |
| 69 | V | A |
| 71 | N | R |
| 74 | Q | P |
| 80 | L | F |
| 81 | R | D |
| 85 | L | V |
| 86 | I | V |
| 88 | N | D, Dgp (gp = O-(2-aminoethyl)-O′-(2- |
| | | aminoethyl)octaethylene glycol) |
| 89 | I | V |
| 92 | I | F |
| 126 | Q | T |

*Residue position numbering based on SEQ ID NO: 1 as a reference sequence

In some embodiments, the modified IL-2 polypeptide comprises one or more substitutions at one or more residues selected from 18, 22, 23, 29, 31, 35, 37, 39, 42, 46, 48, 69, 71, 74, 80, 81 85, 86, 88, 89, 92, and 126. In some embodiments, the modified IL-2 polypeptide comprises 1, 2, 3, 4, 5, 6, 7, or 8 substitutions at residues selected from 18, 22, 23, 29, 31, 35, 37, 39, 42, 46, 48, 69, 71, 74, 80, 81 85, 86, 88, 89, 92, and 126. In some embodiments, the modified IL-2 polypeptide comprises one or more substitutions selected from L18R, Q22E, M23A, N29S, Y31H, K35R, T37A, M39A, F42(4-NH₂)-Phe, M46A, K48E, V69A, N71R, Q74P, L80F, R81D, L85V, I86V, N88D, I89V, I92F, and Q126T. In some embodiments, the modified IL-2 polypeptide comprises 1, 2, 3, 4, 5, 6, 7, or 8 substitutions selected from L18R, Q22E, M23A, N29S, Y31H, K35R, T37A, M39A, F42(4-NH₂)-Phe, M46A, K48E, V69A, N71R, Q74P, L80F, R81D, L85V, I86V, N88D, I89V, I92F, and Q126T. In some embodiments, the modified IL-2 polypeptide comprises L18R. In some embodiments, the modified IL-2 polypeptide comprises Q22E. In some embodiments, the modified IL-2 polypeptide comprises M23A. In some embodiments, the modified IL-2 polypeptide comprises N29S. In some embodiments, the modified IL-2 polypeptide comprises Y31H. In some embodiments, the modified IL-2 polypeptide comprises K35R. In some embodiments, the modified IL-2 polypeptide comprises T37A. In some embodiments, the modified IL-2 polypeptide comprises M39A. In some embodiments, the modified IL-2 polypeptide comprises F42(4-NH₂)-Phe. In some embodiments, the modified IL-2 polypeptide comprises M46A. In some embodiments, the modified IL-2 polypeptide comprises K48E. In some embodiments, the modified IL-2 polypeptide comprises V69A. In some embodiments, the modified IL-2 polypeptide comprises N71R. In some embodiments, the modified IL-2 polypeptide comprises Q74P. In some embodiments, the modified IL-2 polypeptide comprises L80F. In some embodiments, the modified IL-2 polypeptide comprises R81D. In some embodiments, the modified IL-2 polypeptide comprises L85V. In some embodiments, the modified IL-2 polypeptide comprises I86V. In some embodiments, the modified IL-2 polypeptide comprises N88D. In some embodiments, the modified IL-2 polypeptide comprises I89V. In some embodiments, the modified IL-2 polypeptide comprises I92F. In some embodiments, the modified IL-2 polypeptide comprises Q126T.

In some embodiments, a modified IL-2 polypeptide provided herein comprises amino acid substitutions at at least one of Y31, K35, Q74, and N88D, wherein residue position numbering of the modified IL-2 polypeptide is based on SEQ ID NO: 1 as a reference sequence. In some embodiments, the modified IL-2 polypeptide comprises amino acid substitutions at at least two of Y31, K35, Q74, and N88. In some embodiments, the modified IL-2 polypeptide comprises amino acid substitutions at at least three of Y31, K35, Q74, and N88. In some embodiments, the modified IL-2 polypeptide. In some embodiments, the modified IL-2 polypeptide comprises amino acid substitutions at each of Y31, K35, Q74, and N88. In some embodiments, the modified IL-2 polypeptide comprises the amino acid substitutions Y31H, K35R, Q74P, and N88D. In some embodiments, the modified IL-2 polypeptide further comprises an optional C125 substitution (e.g., C125S or C125A). In some embodiments, the modified IL-2 polypeptide further comprises an optional A1 deletion or substitution of residue A1. In some embodiments, the modified IL-2 polypeptide further comprises an optional A1 deletion.

In some embodiments, a modified IL-2 polypeptide provided herein comprises natural amino acid substitutions at least one of Y31, K35, or Q74, wherein residue position numbering of the modified IL-2 polypeptide is based on SEQ ID NO:1 as a reference sequence. In some embodiments, the modified IL-2 polypeptide comprises natural amino acid substitutions at least two of Y31, K35, or Q74. In some embodiments, the modified IL-2 polypeptide. In some embodiments, the modified IL-2 polypeptide comprises natural amino acid substitutions at each of Y31, K35, and Q74. In some embodiments, the modified IL-2 polypeptide comprises the amino acid substitutions Y31H, K35R, and Q74P. In some embodiments, the modified IL-2 polypeptide further comprises an optional C125 mutation.

In some embodiments, a modified IL-2 polypeptide provided herein comprises a Y31 mutation wherein residue position numbering of the modified IL-2 polypeptide is based on SEQ ID NO:1 as a reference sequence. In some embodiments, the Y31 mutation is for an aromatic amino acid. In some embodiments, the Y31 mutation is for a basic amino acid. In some embodiments, the basic amino acid is weakly basic. In some embodiments, the Y31 mutation is selected from Y31F, Y31H, Y31W, Y31R, and Y31K. In some embodiments, the Y31 mutation is Y31H.

In some embodiments, a modified IL-2 polypeptide provided herein comprises a K35 mutation, wherein residue position numbering of the modified IL-2 polypeptide is based on SEQ ID NO:1 as a reference sequence. In some embodiments, the K35 mutation is for a basic amino acid. In some embodiments, the K35 mutation is for a positively charged amino acid. In some embodiments, the K35 mutation is K35R, K35E, K35D, or K35Q. In some embodiments, the K35 mutation is K35R.

In some embodiments, a modified IL-2 polypeptide provided herein comprises a Q74 mutation, wherein residue position numbering of the modified IL-2 polypeptide is based on SEQ ID NO:1 as a reference sequence. In some embodiments, the Q74 mutation is a cyclic amino acid. In some embodiments, the cyclic amino acid comprises a cyclic group covalently attached to the alpha carbon and the nitrogen attached to the alpha carbon. In some embodiments, the Q74 mutation is Q74P.

In some embodiments, a modified IL-2 polypeptide provided herein comprises a N88 substitution, wherein residue position numbering of the modified IL-2 polypeptide is based on SEQ ID NO:1 as a reference sequence. In some embodiments, the N88 substitution is a charged amino acid residue. In some embodiments, the N88 substitution is a negatively charged amino acid residue. In some embodiments, the N88 substitution is N88D or N88E. In some embodiments, the N88 substitution is N88D or N88E. In some embodiments, the N88 substitution is N88D.

In some embodiments, a modified IL-2 polypeptide comprises a C125 mutation, wherein residue position numbering of the modified IL-2 polypeptide is based on SEQ ID NO:1 as a reference sequence. In some embodiments, the C125 mutation stabilizes the modified IL-2 polypeptide. In some embodiments, the C125 mutation does not substantially alter the activity of the modified IL-2 polypeptide. In some embodiments, the modified IL-2 polypeptide comprises a C125S mutation. In some embodiments, the modified IL-2 polypeptide comprises a C125A substitution.

In some embodiment, a modified IL-2 polypeptide comprises a modification at residue A1, wherein residue position numbering of the modified IL-2 polypeptide is based on SEQ ID NO: 1 as a reference sequence. In some embodiments, the modification is an A1 deletion. In some embodiments, a modified IL-2 polypeptide provided herein comprises an N-terminal deletion. In some embodiments, the N-terminal deletion is of 1, 2, 3, 4, 5, 6, 7, 8, 9, 10, 11, 12, 13, 14, 15, or more amino acids. In some embodiments, the N-terminal deletion is of at least 1 amino acid. In some embodiments, the N-terminal deletion is of at least 1, 2, 3, 4, 5, 6, 7, 8, 9, or 10 amino acids. In some embodiments, the N-terminal deletion is from 1 to 15 amino acids. In some embodiments, the N-terminal deletion is a deletion of a single amino acid (e.g., an A1 deletion of SEQ ID NO: 1).

In some embodiments, the modified IL-2 polypeptide comprises additional amino acid substitutions. In some embodiments, the modified IL-2 polypeptide comprises an additional amino acid substitution that has an effect on binding to the IL-2 receptor alpha subunit or αβγ complex. In some embodiments, the modified IL-2 polypeptide comprises an additional amino acid substitution that has an effect on binding to the IL-2 receptor beta subunit or βγ complex. In some embodiments, the modified IL-2 polypeptide comprises at least one additional amino acid substitution selected from Table 6. In some embodiments, the modified IL-2 polypeptide comprises at least one amino acid substitution at residue E15, N29, N30, T37, K48, V69, N71, N88, I89, or I92. In some embodiments, the modified IL-2 polypeptide comprises 1, 2, 3, or 4 natural amino acid substitutions at residues selected from E15, N29, N30, T37, K48, V69, N71, N88, I89, or I92. In some embodiments, the modified IL-2 polypeptide comprises 1 natural amino acid substitutions at residues selected from E15, N29, N30, T37, K48, V69, N71, N88, I89, or I92. In some embodiments, the modified IL-2 polypeptide comprises 2 In some embodiments, the modified IL-2 polypeptide comprises up to 2 natural amino acid substitutions at residues selected from E15, N29, N30, T37, K48, V69, N71, N88, I89, or I92. In some embodiments, the modified IL-2 polypeptide comprises up to 3 natural amino acid substitutions at residues selected from E15, N29, N30, T37, K48, V69, N71, N88, I89, or I92. In some embodiments, the additional amino acid substitution comprises E15A, E15G, or E15S. In some embodiments, the additional amino acid substitution comprises N29S. In some embodiments, the additional amino acid substitution comprises N30S. In some embodiments, the additional amino acid substitution comprises T37A or T37R. In some embodiments, the additional amino acid substitution comprises K48E. In some embodiments, the additional amino acid substitution comprises V69A. In some embodiments, the additional amino acid substitution comprises N71R. In some embodiments, the additional amino acid substitution comprises N88A, N88D, N88E, N88F, N88G, N88H, N88I, N88M, N88Q, N88R, N88S, N88T, N88V, or N88W. In some embodiments, the additional amino acid substitution comprises N88D. In some embodiments, the additional amino acid substitution comprises I89V. In some embodiments, the additional amino acid substitution comprises I92K or I92R.

In some embodiments, a modified IL-2 polypeptide provided herein comprises mutations at Y31, K35, Q74, and optionally C125S. In some embodiments, the modified IL-2 polypeptide does not comprise any additional mutations which substantially affect binding to the IL-2 receptor alpha subunit or αβγ complex. In some embodiments, the modified IL-2 polypeptide does not comprise an additional amino acid substitution that has an effect on binding to the IL-2 receptor beta subunit or βγ complex. In some embodiments, the modified IL-2 polypeptide does not comprise any additional natural amino acid substitutions selected from positions identified in Table 6. In some embodiments, the modified IL-2 polypeptide does not comprise any additional natural amino acid substitutions selected from positions identified in Table 7. In some embodiments, the modified IL-2 polypeptide does not comprise any additional amino acid substitutions selected from Table 6. In some embodiments, the modified IL-2 polypeptide does not comprise any additional amino acid substitutions selected from Table 7. In some embodiments, the modified IL-2 polypeptide does not comprise any additional natural amino acid substitutions at residues E15, N29, N30, T37, K48, V69, N71, N88, I89, or I92. In some embodiments, the modified IL-2 polypeptide does not comprise any additional amino acid substitutions at residues E15, N29, N30, T37, K48, V69, N71, N88, I89, or I92. In some embodiments, the modified IL-2 polypeptide does not have a V69 mutation. In some embodiments, the modified IL-2 polypeptide does not have a V69A mutation. In some embodiments, the modified IL-2 polypeptide does not have a K48 mutation. In some embodiments, the modified IL-2 polypeptide does not have a K48E mutation. In some embodiments, the modified IL-2 polypeptide does not comprise a mutation at V69 or K48. In some embodiments, the modified IL-2 polypeptide does not comprise a mutation at either of V69 or K48. In some embodiments, the modified IL-2 polypeptide does not comprise a V69A or K48E mutation. In some embodiments, the modified IL-2 polypeptide does not comprise either a V69A or K48E mutation.

In some embodiments, a modified IL-2 polypeptide provided herein comprises substitutions at Y31, K35, Q74, N88, and optionally C125S. In some embodiments, the modified IL-2 polypeptide does not comprise any additional substitutions which substantially affect binding to the IL-2 receptor alpha subunit or αβγ complex. In some embodiments, the modified IL-2 polypeptide does not comprise an additional amino acid substitution that has an effect on binding to the IL-2 receptor beta subunit or βγ complex. In some embodiments, the modified IL-2 polypeptide does not comprise any additional natural amino acid substitutions selected from positions identified in Table 6. In some embodiments, the modified IL-2 polypeptide does not comprise any additional amino acid substitutions selected from Table 7. In some embodiments, the modified IL-2 polypeptide does not comprise any additional natural amino acid substitutions at residues E15, N29, N30, T37, K48, V69, N71, I89, or I92. In some embodiments, the modified IL-2 polypeptide does not comprise any additional amino acid substitutions at residues E15, N29, N30, T37, K48, V69, N71, I89, or I92. In some embodiments, the modified IL-2 polypeptide does not have a V69 substitution. In some embodiments, the modified IL-2 polypeptide does not have a V69A substitution. In some embodiments, the modified IL-2 polypeptide does not have a K48 substitution. In some embodiments, the modified IL-2 polypeptide does not have a K48E substitution. In some embodiments, the modified IL-2 polypeptide does not comprise a substitution at V69 or K48. In some embodiments, the modified IL-2 polypeptide does not comprise a substitution at either of V69 or K48. In some embodiments, the modified IL-2 polypeptide does not comprise a V69A or K48E substitution. In some embodiments, the modified IL-2 polypeptide does not comprise either a V69A or K48E substitution.

In one aspect, disclosed herein is a modified IL-2 polypeptide comprising one or more unnatural amino acid substitutions (e.g., for a synthetic IL-2 polypeptide). In some embodiments, the modified IL-2 polypeptide comprises at least two unnatural amino acid substitutions. In some embodiments, the modified IL-2 polypeptide comprises at least one amino acid substitution at a residue selected from Y31, K35, and Q74, wherein residue position numbering of the modified IL-2 polypeptide is based on SEQ ID NO:1 as a reference sequence.

In some embodiments, the IL-2 polypeptide can comprise a polymer (e.g., other than the linker) which disrupts binding between the modified IL-2 polypeptide and the IL-2 receptor beta subunit, or which otherwise biases the modified IL-2 polypeptide in favor of signaling through the alpha subunit. In some embodiments, the point of attachment of the polymer is selected such that interaction of the IL-2 polypeptide with an IL-2 receptor containing the IL-2R alpha receptor subunit is unaffected or only slightly reduced. In some embodiments, the point of attachment of the polymer is selected such that interaction of the IL-2 polypeptide with the IL-2 beta receptor subunit is substantially reduced. Examples of such residues are provided in US Publication No. US2020/0231644A1, which is hereby incorporated by reference as if set forth in its entirety. In some embodiments, the polymer is attached to a residue at position 8, 9, 11, 12, 15, 16, 18, 19, 20, 22, 23, 26, 81, 84, 87, 88, 91, 92, 94, 95, 116, 119, 120, 123, 125, 126, 127, 130, 131, 132, or 133 of the IL-2 polypeptide, wherein residue position numbering is based on SEQ ID NO: 1 as a reference sequence. In some embodiments, the polymer is attached to a residue at position 8, 9, 12, 15, 16, 19, 20, 22, 23, 26, 84, 88, 95, or 126 of the IL-2 polypeptide. In some embodiments, the polymer is attached to a residue at position 8, 9, or 16. In some embodiments, the polymer is attached to a residue at position 22, 26, 88, or 126 of the IL-2 polypeptide. In some embodiments, the polymer is attached to a residue at position 15, 20, 84, or 95 of the IL-2 polypeptide. In some embodiments, the polymer is attached to a residue at position 12, 19, or 23 of the IL-2 polypeptide. In some embodiments, the polymer is attached to a residue at position 22 or 26. In some embodiments, the polymer is attached to a residue at position 35 of the IL-2 polypeptide. In some embodiments, the polymer is attached at residue 88. In some embodiments, the polymer is attached at residue N88D. In some embodiments, the polymer attached at residue 88 is attached to an amino acid a modified aspartate residue, wherein the polymer attached to the residue has the structure

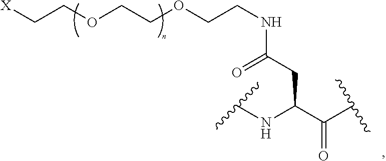

wherein n is an integer from 1-30, and wherein X is NH₂, —OCH₃, OH, —NH(C═O)CH₃, or a conjugation handle (e.g., azide). In some embodiments, n is an integer from 8-10. In some embodiments, X is —NH₂. When X is NH₂ and n is 9, the corresponding amino acid is optionally referred to herein as Dgp (D with a O-(2-aminoethyl)-O′-(2-aminoethyl)octaethylene glycol). For sequence identity purposes, it is intended herein that such an amino acid (which may also be referred to in a more general sense as a “modified D,” “D modified with a polymer,” or similar language) would qualify as the base amino acid from which the final structure is derived (e.g., such a residue would qualify as a D for sequence identify purposes).

In some embodiments, the linker is attached to the IL-2 polypeptide at an amino acid residue. In some embodiments, the linker is attached at an amino acid residue corresponding to any one of amino acid residues 1-133 of SEQ ID NO: 1. In some embodiments, the linker is attached to the N-terminal amino acid residue of the IL-2 polypeptide. In some embodiments, the linker is attached to the N-terminal amino group of the IL-2 polypeptide. In some embodiments, the linker is attached to the N-terminal amino group of the modified IL-2 polypeptide through by a reaction with an adduct attached to the N-terminal amino group having a structure

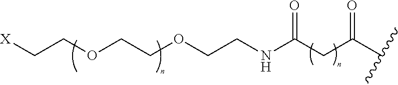

wherein each n is independently an integer from 1-30 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30), and wherein X is a conjugation handle (e.g., an azide or other conjugation handle provided herein, such as a DBCO group). In some embodiments, the adduct has the structure

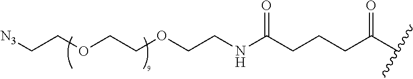

In some embodiments, the linker is attached at a non-terminal amino acid residue (e.g., any one of amino acid residues 2-132 of SEQ ID NO: 1, or any one of amino acid residues 1-133 of SEQ ID NO: 1, wherein either the N-terminus or C-terminus has been extended by one or more amino acid residues). In some embodiments, the chemical linker is attached at a non-terminal amino acid residue of the IL-2 polypeptide, wherein the IL-2 polypeptide comprises either an N-terminal truncation or a C-terminal truncation relative to SEQ ID NO: 1. In some embodiments, the chemical linker is attached to a side chain of a non-terminal amino acid residue.

In some embodiments, the linker is attached to the IL-2 polypeptide at an amino acid residue which interacts with an IL-2 receptor (IL-2R) protein or subunit. In some embodiments, the linker is attached at an amino acid residue which interacts with the IL-2R alpha subunit (IL-2Rα), the IL-2R beta subunit (IL-2Rβ), or the IL-2R gamma subunit (IL-2Rγ). In some embodiments, the chemical linker is attached at an amino acid residue which interacts with the IL-2R alpha subunit. In some embodiments, the chemical linker is attached at an amino acid residue which interacts with the IL-2R beta subunit. In some embodiments, the chemical linker is attached at an amino acid residue which interacts with the IL-2R gamma subunit.

In some embodiments, the point of attachment to the IL-2 polypeptide is selected such that the interaction of the IL-2 polypeptide with at least one IL-2 receptor subunit is decreased or blocked. In some embodiments, the point of attachment is selected such that interaction of the IL-2 polypeptide with an IL-2 receptor containing the IL-2R alpha receptor subunit is unaffected or only slightly reduced. In some embodiments, the point of attachment is selected such that interaction of the IL-2 polypeptide with the IL-2 beta receptor subunit is substantially reduced. Examples of such residues are provided in US Publication No. US2020/0231644A1, which is hereby incorporated by reference as if set forth in its entirety. In some embodiments, the linker is attached to a residue at position 8, 9, 11, 12, 15, 16, 18, 19, 20, 22, 23, 26, 81, 84, 87, 88, 91, 92, 94, 95, 116, 119, 120, 123, 125, 126, 127, 130, 131, 132, or 133 of the IL-2 polypeptide, wherein residue position numbering is based on SEQ ID NO: 1 as a reference sequence. In some embodiments, the linker is attached to a residue at position 8, 9, 12, 15, 16, 19, 20, 22, 23, 26, 84, 88, 95, or 126 of the IL-2 polypeptide. In some embodiments, the linker is attached to a residue at position 8, 9, or 16. In some embodiments, the linker is attached to a residue at position 22, 26, 88, or 126 of the IL-2 polypeptide. In some embodiments, the linker is attached to a residue at position 15, 20, 84, or 95 of the IL-2 polypeptide. In some embodiments, the linker is attached to a residue at position 12, 19, or 23 of the IL-2 polypeptide. In some embodiments, the linker is attached to a residue at position 22 or 26. In some embodiments, the linker is attached to a residue at position 35 of the IL-2 polypeptide.

In some embodiments, a modified IL-2 polypeptide described herein is capable of expanding regulatory T-cell (T_{reg}), CD4+ helper cell, CD8+ central memory cell, CD8+ effector memory cell, naïve CD8+ cell, Natural Killer (NK) cell, Natural killer T (NKT) cell populations, or a combination thereof. In some embodiments, a modified IL-2 polypeptide described herein is capable of expanding a regulatory T-cell (T_{reg}) cell population. In some embodiments, a modified IL-2 polypeptide described herein spares expansion of effector T-cells (T_{eff}).

In one aspect, described herein is a modified IL-2 polypeptide that exhibits a greater affinity for IL-2 receptor α subunit than an IL-2 polypeptide of SEQ ID NO: 1 and/or SEQ ID NO: 2. In some embodiments, the affinity to IL-2 receptor α subunit is measured by dissociation constant (K_{d}). As used herein, the phrase “the K_{d} of the modified IL-2 polypeptide/IL-2 receptor α subunit” means the dissociation constant of the binding interaction of the modified IL-2 polypeptide and CD25.

In some embodiments, the K_{d} of the modified IL-2 polypeptide/IL-2 receptor α subunit is less than 10 nM. In some embodiments the K_{d} of the modified IL-2 polypeptide/IL-2 receptor α subunit is less than 10 nM, less than 7.5 nM, less than 5 nM, less than 4 nM, or less than 3 nM. In some embodiments, the K_{d} of the modified IL-2 polypeptide/IL-2 receptor α subunit between about 1 nM and 0.1 nM. In some embodiments, the K_{d} of the modified IL-2 polypeptide/IL-2 receptor α subunit between about 10 nM and about 0.1 nM. In some embodiments, the K_{d} of the modified IL-2 polypeptide/IL-2 receptor α subunit between about 10 nM and about 1 nM. In some embodiments, the K_{d} of the modified IL-2 polypeptide/IL-2 receptor α subunit between about 7.5 nM and about 0.1 nM. In some embodiments, the K_{d} of the modified IL-2 polypeptide/IL-2 receptor α subunit between about 7.5 nM and about 1 nM. In some embodiments, the K_{d} of the modified IL-2 polypeptide/IL-2 receptor α subunit between about 5 nM and about 0.1 nM. In some embodiments, the K_{d} of the modified IL-2 polypeptide/IL-2 receptor α subunit between about 1 nM and about 1 nM. In some embodiments, the K_{d} is measured by surface plasmon resonance.

In some embodiments, the modified IL-2 polypeptide that exhibits at least about a 10%, 50%, 100%, 250%, or 500% greater affinity for IL-2 receptor α subunit than an IL-2 polypeptide of SEQ ID NO: 1 and/or SEQ ID NO: 2. In some embodiments, the modified IL-2 polypeptide exhibits at most about a 500%, 750%, or 1000% greater affinity for IL-2 receptor α subunit than an IL-2 polypeptide of SEQ ID NO: 1 and/or SEQ ID NO: 2.

In some embodiments, the modified IL-2 polypeptide exhibits about 1.5-fold to about 10-fold greater affinity for IL-2 receptor α subunit than an IL-2 polypeptide of SEQ ID NO: 1 and/or SEQ ID NO: 2.

In some embodiments, the modified IL-2 polypeptide exhibits substantially the same binding affinity for the IL-2Rα as compared to an IL-2 polypeptide of SEQ ID NO: 1 and/or SEQ ID NO: 2. In some embodiments, the modified IL-2 polypeptide exhibits a K_{d} with IL-2Rα that is within about 2-fold, about 4-fold, about 6-fold, about 8-fold, or about 10-fold of the K_{d} between an IL-2 polypeptide of SEQ ID NO: 1 and/or SEQ ID NO: 2 and IL-2Rα.

In some embodiments, the modified IL-2 polypeptide exhibits reduced affinity for the IL-2 receptor β subunit (IL-2Rβ) as compared to an IL-2 polypeptide of SEQ ID NO: 1 and/or SEQ ID NO: 2. In some embodiments, the modified IL-2 polypeptide exhibits at least about 10-fold, at least about 25-fold, at least about 50-fold, at least about 100-fold, or at least about 500-fold fold lower affinity for the IL-2Rβ. In some embodiments, the modified IL-2 polypeptide exhibits at least about 100-fold lower affinity for IL-2Rβ. In some embodiments, the modified IL-2 polypeptide exhibits substantially no affinity for IL-2Rβ. In some embodiments, the affinity is measured as the dissociation constant K_{d} (e.g., a lower affinity correlating with a higher dissociation constant).

In some embodiments, the modified IL-2 polypeptide exhibits a binding affinity for IL-2Rβ which is at least 500 nM, at least 1000 nM, at least 5000 nM, at least 10000 nM, at least 50000 nM, or at least 100000 nM.

In some embodiments, the modified IL-2 polypeptide exhibits an affinity for IL-2Ra which is greater than for IL-2Rb (e.g., a k_{d} for the modified IL-2 with IL-2Rα is lower than a k_{d} for the modified IL-2 with IL-2Rβ). In some embodiments, the modified IL-2 polypeptide exhibits an affinity for IL-2Rα which is at least about 30-fold greater, at least about 50-fold greater, at least about 75-fold greater, at least about 100-fold greater, at least about 500-fold greater, or at least about 1000-fold greater than for IL-2Rβ. In some embodiments, the modified IL-2 polypeptide exhibits an affinity for IL-2Rα which is at least about 100-fold greater than for IL-2Rβ. In some embodiments, the modified IL-2 polypeptide exhibits an affinity for IL-2Rα which is at least about 1000-fold greater than for IL-2Rβ.

In some embodiments, a modified IL-2 polypeptide has a half maximal effective concentration (EC₅₀) for activation of T_{reg} cells that is comparable (e.g., approximately the same) to an IL-2 polypeptide of SEQ ID NO: 1 and/or SEQ ID NO: 2. In some embodiments, activation of T_{reg} cells is measured by assessing change in STAT5 phosphorylation in a population of T cells when in contact with the modified IL-2 polypeptide. In some embodiments, a T_{reg} cell is identified by being CD4⁺ and FoxP3⁺. In some embodiments, a T_{reg} cell is identified by also showing elevated expression of CD25 (CD25^{Hi}). In some embodiments, the modified IL-2 polypeptide has an EC₅₀ for activation of T_{reg} cells of at most about 100 nM, at most about 75 nM, at most about 50 nM, at most about 40 nM, at most about 35 nM, at most about 30 nM, or at most about 25 nM. In some embodiments, the modified IL-2 polypeptide has an EC₅₀ for activation of T_{reg} cells of at most about 50 nM, at most about 40 nM, at most about 35 nM, at most about 30 nM, or at most about 25 nM, at most about 20 nM, at most about 15 nM, at most about 10 nM, or at most about 5 nM. In some embodiments, the modified IL-2 polypeptide has an EC₅₀ for activation of T_{reg} cells of at most about 100 nM. In some embodiments, the modified IL-2 polypeptide has an EC₅₀ for activation of T_{reg} cells of at most about 50 nM. In some embodiments, the modified IL-2 polypeptide has an EC₅₀ for activation of T_{reg} cells of at most about 25 nM. In some embodiments, the modified IL-2 polypeptide has an EC₅₀ for activation of T_{reg} cells of from about 0.1 nM to about 100 nM, from about 1 nM to about 100 nM, from about 0.1 nM to about 50 nM, from about 1 nM to about 50 nM, from about 0.1 nM to about 25 nM, from about 1 nM to about 25 nM, from about 0.1 nM to about 10 nM, or from about 1 nM to about 10 nM.

In some embodiments, the modified IL-2 polypeptide has an EC₅₀ for activation of T_{reg} cells that is at most 2-fold, at most 5-fold, at most 10-fold, at most 20-fold, at most 50-fold, at most 100-fold, at most 200-fold, at most 500-fold, or at most 1000-fold greater compared to an IL-2 polypeptide of SEQ ID NO: 1 and/or SEQ ID NO: 2. In some embodiments, the modified IL-2 polypeptide has an EC₅₀ for activation of T_{reg} cells that is at most 2-fold greater. In some embodiments, the modified IL-2 polypeptide has an EC₅₀ for activation of T_{reg} cells that is at most 5-fold greater. In some embodiments, the modified IL-2 polypeptide has an EC₅₀ for activation of T_{reg} cells that is at most 10-fold greater. In some embodiments, the modified IL-2 polypeptide has an EC₅₀ for activation of T_{reg} cells that is at most 50-fold greater. In some embodiments, the modified IL-2 polypeptide has an EC₅₀ for activation of T_{reg} cells that is at most 100-fold greater. In some embodiments, the modified IL-2 polypeptide has an EC₅₀ for activation of T_{reg} cells that is at most 200-fold greater. In some embodiments, the modified IL-2 polypeptide has an EC₅₀ for activation of T_{reg} cells that is at most 500-fold greater. In some embodiments, the modified IL-2 polypeptide has an EC₅₀ for activation of T_{reg} cells that is at most 1000-fold greater.

In some embodiments, a modified IL-2 polypeptide has a half maximal effective concentration (EC₅₀) for activation of T_{eff} cells that is substantially greater compared to an IL-2 polypeptide of SEQ ID NO: 1 and/or SEQ ID NO: 2. In some embodiments, the T_{eff} cell is 1, 2, or 3 of a CD8 T_{eff} cell (e.g., CD8⁺), a Naïve CD8 cell (e.g., CD8⁺, CD45RA⁺), or a CD4 Conv cell (e.g., CD4⁺, FoxP3⁻), or any combination thereof. In some embodiments, activation of cells is measured by assessing change in STAT5 phosphorylation in a population of T cells when in contact with the modified IL-2 polypeptide. In some embodiments, the modified IL-2 polypeptide has an EC₅₀ for activation of T_{eff} cells of at least about 10 nM, at least about 50 nM, at least about 100 nM, at least about 500 nM, at least about 1000 nM, at least about 2000 nM, at least about 3000 nM, at least about 4000 nM, or at least about 5000 nM. In some embodiments, the modified IL-2 polypeptide has an EC₅₀ for activation of T_{eff} cells of at least about 100 nM. In some embodiments, the modified IL-2 polypeptide has an EC₅₀ for activation of T_{eff} cells of at least about 500 nM. In some embodiments, the modified IL-2 polypeptide has an EC₅₀ for activation of T_{eff} cells of at least about 1000 nM. In some embodiments, the modified IL-2 polypeptide has an EC₅₀ for activation of T_{eff} cells of at least about 5000 nM. In some embodiments, the modified IL-2 polypeptide has an EC₅₀ for activation of T_{eff} cells of at least 10-fold, at least 20-fold, at least 50-fold, at least 100-fold, at least 500-fold, or at least 1000-fold greater compared to an IL-2 polypeptide of SEQ ID NO: 1 and/or SEQ ID NO: 2. In some embodiments, the modified IL-2 polypeptide has an EC₅₀ for activation of T_{eff} cells of at least 10-fold greater. In some embodiments, the modified IL-2 polypeptide has an EC₅₀ for activation of T_{eff} cells of at least 50-fold greater. In some embodiments, the modified IL-2 polypeptide has an EC₅₀ for activation of T_{eff} cells of at least 100-fold greater. In some embodiments, the modified IL-2 polypeptide has an EC₅₀ for activation of T_{eff} cells of at least 500-fold greater. In some embodiments, the modified IL-2 polypeptide has an EC₅₀ for activation of T_{eff} cells of at least 1000-fold greater.

In some embodiments, the modified IL-2 polypeptide exhibits a substantially greater ability to activate T_{reg} cells compared to T_{eff} cells. In some embodiments, a ratio of EC50 for activation of a T_{eff} cell type over EC50 for activation of a T_{reg} cell type is at least 10, at least 20, at least 50, at least 100, at least 150, at least 200, at least 250, or at least 300. In some embodiments, a ratio of EC50 for activation of a T_{eff} cell type over EC50 for activation of a T_{reg} cell type is at least 100. In some embodiments, a ratio of EC50 for activation of a T_{eff} cell type over EC50 for activation of a T_{reg} cell type is at least 200. In some embodiments, a ratio of EC50 for activation of a T_{eff} cell type over EC50 for activation of a T_{reg} cell type is at least 300. In some embodiments, a ratio of EC50 for activation of a T_{eff} cell type over EC50 for activation of a T_{reg} cell type is at least 500. In some embodiments, a ratio of EC50 for activation of a T_{eff} cell type over EC50 for activation of a T_{reg} cell type is at least 1000.

In some embodiments, a modified IL-2 polypeptide described herein expands a cell population of regulatory T cells (T_{reg} cells). In some embodiments, the modified IL-2 polypeptide expands a cell population of T_{reg} cells by at least 1%, at least 2%, at least 5%, at least 10%, at least 15%, at least 20%, at least 30%, at least 40%, at least 50%, at least 100%, or at least 200% when the modified IL-2 polypeptide is in contact with the population. In some embodiments, the modified IL-2 polypeptide expands a cell population of T_{reg} cells by at least 20% when the modified IL-2 polypeptide is in contact with the population. In some embodiments, the modified IL-2 polypeptide expands a cell population of T_{reg} cells by at least 30% when the modified IL-2 polypeptide is in contact with the population. In some embodiments, the modified IL-2 polypeptide expands a cell population of T_{reg} cells by at least 40% when the modified IL-2 polypeptide is in contact with the population. In some embodiments, the modified IL-2 polypeptide expands a cell population of T_{reg} cells by at least 50% when the modified IL-2 polypeptide is in contact with the population. In some embodiments, the modified IL-2 polypeptide expands a cell population of T_{reg} cells by at least 100% when the modified IL-2 polypeptide is in contact with the population. In some embodiments, the modified IL-2 polypeptide expands a cell population of T_{reg} cells by at least 200% when the modified IL-2 polypeptide is in contact with the population. In some embodiments, the expansion of T_{reg} cells is measured compared to a sample or subject not treated by an 11-2 polypeptide. In some embodiments, the expansion of T_{reg} cells is measured compared to a sample or subject treated with an IL-2 polypeptide of SEQ ID NO: 1 and/or SEQ ID NO: 2. In some embodiments, the expansion of T_{reg} cells is measured compared to a sample or subject treated with an IL-2 polypeptide of SEQ ID NO: 1 and/or SEQ ID NO: 2 at the same dose of IL-2 polypeptide as the modified IL-2 polypeptide.

In some embodiments, a modified IL-2 polypeptide has a half maximal effective concentration (EC₅₀) for activation of T_{reg} cells that is comparable to an IL-2 polypeptide of SEQ ID NO: 1 and/or SEQ ID NO: 2. In some embodiments, the modified IL-2 polypeptide has an EC₅₀ for activation of T_{reg} cells of at most about 0.01 nM, at most about 0.05 nM, at most about 0.1 nM, at most about 0.5 nM, at most about 1 nM, at most about 5 nM, at most about 10 nM, at most about 50 nM, or at most about 100 nM. In some embodiments, the modified IL-2 polypeptide has an EC₅₀ for activation of T_{reg} cells of at most about 0.01 nM. In some embodiments, the modified IL-2 polypeptide has an EC₅₀ for activation of T_{reg} cells of at most about 0.05 nM. In some embodiments, the modified IL-2 polypeptide has an EC₅₀ for activation of T_{reg} cells of at most about 0.1 nM. In some embodiments, the modified IL-2 polypeptide has an EC₅₀ for activation of T_{reg} cells of at most about 0.5 nM. In some embodiments, the modified IL-2 polypeptide has an EC₅₀ for activation of T_{reg} cells of at most about 1 nM. In some embodiments, the modified IL-2 polypeptide has an EC₅₀ for activation of T_{reg} cells from about 0.01 nM to about 100 nM, about 0.01 nM to about 50 nM, 0.01 nM to about 10 nM, 0.01 nM to about 5 nM, 0.01 nM to about 1 nM, about 0.01 nM to about 0.5 nM, or about 0.01 nM to about 0.1 nM. In some embodiments, the modified IL-2 polypeptide has an EC₅₀ for activation of T_{reg} cells from about 0.05 nM to about 100 nM, about 0.05 nM to about 50 nM, 0.05 nM to about 10 nM, 0.05 nM to about 5 nM, 0.05 nM to about 1 nM, about 0.05 nM to about 0.5 nM, or about 0.05 nM to about 0.1 nM.

In some embodiments, the modified IL-2 polypeptide has an EC₅₀ for activation of T_{reg} cells that is at most 2-fold, at most 5-fold, at most 10-fold, at most 20-fold, at most 50-fold, at most 100-fold, at most 200-fold, at most 500-fold, or at most 1000-fold greater compared to an IL-2 polypeptide of SEQ ID NO: 1 and/or SEQ ID NO: 2. In some embodiments, the modified IL-2 polypeptide has an EC₅₀ for activation of T_{reg} cells that is at most 2-fold greater. In some embodiments, the modified IL-2 polypeptide has an EC₅₀ for activation of T_{reg} cells that is at most 5-fold greater. In some embodiments, the modified IL-2 polypeptide has an EC₅₀ for activation of T_{reg} cells that is at most 10-fold greater. In some embodiments, the modified IL-2 polypeptide has an EC₅₀ for activation of T_{reg} cells that is at most 50-fold greater. In some embodiments, the modified IL-2 polypeptide has an EC₅₀ for activation of T_{reg} cells that is at most 100-fold greater. In some embodiments, the modified IL-2 polypeptide has an EC₅₀ for activation of T_{reg} cells that is at most 200-fold greater. In some embodiments, the modified IL-2 polypeptide has an EC₅₀ for activation of T_{reg} cells that is at most 500-fold greater. In some embodiments, the modified IL-2 polypeptide has an EC₅₀ for activation of T_{reg} cells that is at most 1000-fold greater.

In some embodiments, a modified IL-2 polypeptide provided herein spares expansion of a population of effector T-cells (T_{eff} cells). In some embodiments, the modified IL-2 polypeptide expands a cell population of T_{eff} cells by at most 1%, at most 2%, at most 5%, at most 10%, at most 15%, at most 20%, at most 30%, at most 40%, at most 50%, at most 100%, or at most 200% when the modified IL-2 polypeptide is in contact with the population. In some embodiments, the modified IL-2 polypeptide expands a cell population of T_{eff} cells by at most 1%. In some embodiments, the modified IL-2 polypeptide expands a cell population of T_{eff} cells by at most 2%. In some embodiments, the modified IL-2 polypeptide expands a cell population of T_{eff} cells by at most 5%. In some embodiments, the modified IL-2 polypeptide expands a cell population of T_{eff} cells by at most 10%. In some embodiments, the modified IL-2 polypeptide expands a cell population of T_{eff} cells by at most 15%. In some embodiments, the modified IL-2 polypeptide expands a cell population of T_{eff} cells by at most 20%. In some embodiments, the expansion of T_{eff} cells is measured compared to a sample or subject not treated by an Il-2 polypeptide. In some embodiments, the expansion of T_{eff} cells is measured compared to a sample or subject treated with an IL-2 polypeptide of SEQ ID NO: 1 and/or SEQ ID NO: 2. In some embodiments, the expansion of T_{eff} cells is measured compared to a sample or subject treated with an IL-2 polypeptide of SEQ ID NO: 1 and/or SEQ ID NO: 2 at the same dose of IL-2 polypeptide as the modified IL-2 polypeptide.

In some embodiments, a modified IL-2 polypeptide has a half maximal effective concentration (EC₅₀) for activation of T_{eff} cells that is substantially greater compared to an IL-2 polypeptide of SEQ ID NO: 1 and/or SEQ ID NO: 2. In some embodiments, the modified IL-2 polypeptide has an EC₅₀ for activation of T_{eff} cells of at least about 10 nM, at least about 50 nM, at least about 100 nM, at least about 500 nM, at least about 1000 nM, at least about 5000 nM, at least about 10000 nM, at least about 50000 nM, or at least about 100000 nM. In some embodiments, the modified IL-2 polypeptide has an EC₅₀ for activation of T_{eff} cells of at least about 100000 nM. In some embodiments, the modified IL-2 polypeptide has an EC₅₀ for activation of T_{eff} cells of at least about 50000 nM. In some embodiments, the modified IL-2 polypeptide has an EC₅₀ for activation of eff cells of at least about 10000 nM. In some embodiments, the modified IL-2 polypeptide has an EC₅₀ for activation of T_{eff} cells of at least about 5000 nM. In some embodiments, the modified IL-2 polypeptide has an EC₅₀ for activation of T_{eff} cells of at least about 1000 nM. In some embodiments, the modified IL-2 polypeptide has an EC₅₀ for activation of T_{eff} cells of at least 10-fold, at least 20-fold, at least 50-fold, at least 100-fold, at least 500-fold, or at least 1000-fold greater compared to an IL-2 polypeptide of SEQ ID NO: 1 and/or SEQ ID NO: 2. In some embodiments, the modified IL-2 polypeptide has an EC₅₀ for activation of T_{eff} cells of at least 10-fold greater. In some embodiments, the modified IL-2 polypeptide has an EC₅₀ for activation of T_{eff} cells of at least 50-fold greater. In some embodiments, the modified IL-2 polypeptide has an EC₅₀ for activation of T_{eff} cells of at least 100-fold greater. In some embodiments, the modified IL-2 polypeptide has an EC₅₀ for activation of T_{eff} cells of at least 500-fold greater. In some embodiments, the modified IL-2 polypeptide has an EC₅₀ for activation of T_{eff} cells of at least 1000-fold greater. In some embodiments, the modified IL-2 polypeptide has an EC₅₀ for activation of T_{eff} cells of at least 10000-fold greater. In some embodiments, the modified IL-2 polypeptide has an EC₅₀ for activation of T_{eff} cells of at least 100000-fold greater.

In some embodiments, a cell population expanded by a modified IL-2 polypeptide provided herein is an in vitro cell population, an in vivo cell population, or an ex vivo cell population. In some embodiments, the cell population is an in vitro cell population. In some embodiments, the cell population is an in vivo cell population. In some embodiments, the cell population is an ex vivo cell population. The cell population may be a population of CD4+ helper cells, CD8+ central memory cells, CD8+ effector memory cells, naïve CD8+ cells, Natural Killer (NK) cells, Natural killer T (NKT) cells, or a combination thereof.

In some embodiments, the levels of cells are measured 1 hour after injection of the modified IL-2 polypeptide. In some embodiments, the levels of cells are measured 2 hours after injection of the modified IL-2 polypeptide. In some embodiments, the levels of cells are measured 4 hours after injection of the modified IL-2 polypeptide. In some embodiments, the levels of cells are measured 30 minutes after injection of the modified IL-2 polypeptide.

In some embodiments, a modified IL-2 described herein polypeptide expands a cell population of regulatory T cells (T_{reg} cells). In some embodiments, the modified IL-2 polypeptide expands a cell population of T_{reg} cells by at least 1%, at least 2%, at least 5%, at least 10%, at least 15%, at least 20%, at least 30%, at least 40%, at least 50%, at least 100%, or at least 200% when the modified IL-2 polypeptide is in contact with the population. In some embodiments, the modified IL-2 polypeptide expands a cell population of T_{reg} cells by at least 20% when the modified IL-2 polypeptide is in contact with the population. In some embodiments, the modified IL-2 polypeptide expands a cell population of T_{reg} cells by at least 30% when the modified IL-2 polypeptide is in contact with the population. In some embodiments, the modified IL-2 polypeptide expands a cell population of T_{reg} cells by at least 40% when the modified IL-2 polypeptide is in contact with the population. In some embodiments, the modified IL-2 polypeptide expands a cell population of T_{reg} cells by at least 50% when the modified IL-2 polypeptide is in contact with the population. In some embodiments, the modified IL-2 polypeptide expands a cell population of T_{reg} cells by at least 100% when the modified IL-2 polypeptide is in contact with the population. In some embodiments, the modified IL-2 polypeptide expands a cell population of T_{reg} cells by at least 200% when the modified IL-2 polypeptide is in contact with the population.

In some embodiments, a modified IL-2 polypeptide described herein expands a cell population of effector T cells (T_{eff} cells). In some embodiments, the modified IL-2 polypeptide expands a cell population of T_{eff} cells by at most 5%, at most 10%, at most 20%, at most 30%, at most 40%, at most 50%, at most 75%, at most 100%, or at most 500% when the modified IL-2 polypeptide is in contact with the population. In some embodiments, the modified IL-2 polypeptide expands a cell population of T_{eff} cells by at most 5%, when the modified IL-2 polypeptide is in contact with the population. In some embodiments, the modified IL-2 polypeptide expands a cell population of T_{eff} cells by at most 20%, when the modified IL-2 polypeptide is in contact with the population. In some embodiments, the modified IL-2 polypeptide expands a cell population of T_{eff} cells by at most 50%, when the modified IL-2 polypeptide is in contact with the population. In some embodiments, the modified IL-2 polypeptide expands a cell population of T_{eff} cells by at most 100%, when the modified IL-2 polypeptide is in contact with the population. In some embodiments, the modified IL-2 polypeptide expands a cell population of T_{eff} cells by at most 500%, when the modified IL-2 polypeptide is in contact with the population.

### IL-7 Polypeptides

Interleukin-7 or a polypeptide having a similar activity thereto (hereinafter, “IL-7” or “IL7) refers to an immunostimulatory cytokine which can promote immune responses mediated by B cells and T cells. In particular, IL-7 plays an important role in an adaptive immune system. IL-7 is mostly secreted by stromal cells in the bone marrow and thymus, but it is also produced by keratinocytes, dendritic cells, hepatocytes, neurons, and epithelial cells. IL-7 activates immune functions through the survival, development, and differentiation of T cells and B cells, survival of lymphoid cells, stimulation of activity of natural killer (NK) cell, etc. IL-7 can regulate development of lymph nodes through lymphoid tissue inducer (LTi) cells, promotes the survival and division of naive T cells or memory T cells, maintains naive T cells or memory T cells, and enhances immune response in humans by inducing secretion of IL-2 and interferon-γ. When a recombinant IL-7 is produced for the purpose of medicinal utilization, there are problems in that impurities increase compared to the general recombinant proteins, the amount of IL-7 degradation, and large-scale production cannot be easily achieved. However, since production of synthetic IL-7 requires a complicated denaturation process, the manufacturing process is not easy. Non-limiting examples of IL-7 amino acid sequences to be utilized in embodiments described herein are provided below in Table 8B. An IL-7 polypeptide utilized in conjugate described herein can have, for example, an amino acid sequence that is at least 80%, 85%, 90%, 91%, 92%, 93%, 94%, 95%, 96%, 97%, 98%, 99%, or 100% identical to any one of the sequences in Table 8B (e.g., any one of SEQ ID NOs: 165-169, or SEQ ID NO: 186, or SEQ ID NO: 187). SEQ ID NO: 186 is the sequence of the mature human IL-7. Unless otherwise specified, any reference to modification of a residue position of IL-7 herein (e.g., a substitution or addition of a polymer or conjugation handle) refer to SEQ ID NO: 186 as a reference sequence.

An IL-7 polypeptide attached to the antibody or antigen binding fragment can be any of the IL-7 polypeptides described herein (including any of the synthetic IL-7 polypeptides described herein). In some embodiments, an IL-7 polypeptide provided herein linked to an antibody or antigen binding fragment comprises an amino acid sequence having at least about 80% sequence identity to the sequence set forth in SEQ ID NO: 186. In some embodiments, the IL-7 polypeptide comprises an amino acid sequence having at least about 85% sequence identity to the sequence set forth in SEQ ID NO: 186. In some embodiments, the IL-7 polypeptide comprises an amino acid sequence having at least about 90% sequence identity to the sequence set forth in SEQ ID NO: 186. In some embodiments, the IL-7 polypeptide comprises an amino acid sequence having at least about 95% sequence identity to the sequence set forth in SEQ ID NO: 186. In some embodiments, the IL-7 polypeptide provided herein comprises an amino acid sequence having at least about 96% sequence identity to the sequence set forth in SEQ ID NO: 186. In some embodiments, the IL-7 polypeptide provided herein comprises an amino acid sequence having at least about 97% sequence identity to the sequence set forth in SEQ ID NO: 186. In some embodiments, the IL-7 polypeptide provided herein comprises an amino acid sequence having at least about 98% sequence identity to the sequence set forth in SEQ ID NO: 186. In some embodiments, the IL-7 polypeptide provided herein comprises an amino acid sequence having at least about 99% sequence identity to the sequence set forth in SEQ ID NO: 186. In some embodiments, the IL-7 polypeptide provided herein comprises an amino acid sequence identical to the sequence set forth in SEQ ID NO: 186.

In some embodiments, the IL-7 polypeptide is a synthetic IL-7 polypeptide. In some embodiments, the synthetic IL-7 polypeptide comprises a homoserine (Hse) residue located in any one of amino acid residues 31-41, based on SEQ ID NO: 186 as a reference sequence. In some embodiments, the synthetic IL-7 polypeptide comprises a Hse residue located in any one of amino acid residues 71-81. In some embodiments, the synthetic IL-7 polypeptide comprises a Hse residue located in any one of amino acid residues 109-119. In some embodiments, the synthetic IL-7 polypeptide comprises 1, 2, 3, or more Hse residues. In some embodiments, the synthetic IL-7 polypeptide comprises Hse36, Hse76, Hse114, or a combination thereof. In some embodiments, the synthetic IL-7 polypeptide comprises Hse36, Hse76, and Hse114. In some embodiments, the synthetic IL-7 polypeptide comprises at least two amino acid substitutions, wherein the at least two amino acid substitutions are selected from (a) a homoserine (Hse) residue located in any one of amino acid residues 31-41; (b) a homoserine residue located in any one of amino acid residues 71-81; and (c) a homoserine residue located in any one of amino acid residues 109-119. In some embodiments, the synthetic IL-7 polypeptide comprises Hse36 and Hse76. In some embodiments, the synthetic IL-7 polypeptide comprises Hse36 and Hse114. In some embodiments, the synthetic IL-7 polypeptide comprises Hse76 and Hse114. In some embodiments, the synthetic IL-7 polypeptide comprises Hse36. In some embodiments, the synthetic IL-7 polypeptide comprises Hse76. In some embodiments, the synthetic IL-7 polypeptide comprises Hse114. In some embodiments, the synthetic IL-7 polypeptide comprises 1, 2, 3, 4, 5, or more norleucine (Nle) residues. In some embodiments, the synthetic IL-7 polypeptide comprises a Nle residue located in any one of residues 12-22. In some embodiments, the synthetic IL-7 polypeptide comprises one or more Nle residues located in any one of amino acid residues 22-32. In some embodiments, the synthetic IL-7 polypeptide comprises a Nle residue located in any one of amino acid residues 49-59. In some embodiments, the synthetic IL-7 polypeptide comprises a Nle residue located in any one of amino acid residues 64-74. In some embodiments, the synthetic IL-7 polypeptide comprises a Nle residue located in any one of amino acid residues 142-152. In some embodiments, the synthetic IL-7 polypeptide comprises five Nle substitutions. In some embodiments, the synthetic IL-7 polypeptide comprises Nle17, Nle27, Nle54, Nle69, and Nle147. In some embodiments, the synthetic IL-7 polypeptide comprises SEQ ID NO: 187.

In some embodiments, the synthetic IL-7 polypeptide comprises an amino acid sequence at least 80%, 85%, 90%, 95%, 99%, or 100% identical to SEQ ID NO: 187. In some embodiments, the synthetic IL-7 polypeptide comprises an amino acid sequence of SEQ ID NO: 187. In some embodiments, the synthetic IL-7 polypeptide consists of an amino acid sequence at least 80%, 85%, 90%, 95%, 99%, or 100% identical to the sequence of SEQ ID NO: 187.

In some embodiments, the synthetic IL-7 polypeptide comprises an amino acid sequence at least about 75% identical to that of SEQ ID NO: 187. In some embodiments, the synthetic IL-7 polypeptide comprises an amino acid sequence at least about 80% identical to that of SEQ ID NO: 187. In some embodiments, the synthetic IL-7 polypeptide comprises an amino acid sequence at least about 85% identical to that of SEQ ID NO: 187. In some embodiments, the synthetic IL-7 polypeptide comprises an amino acid sequence at least about 90% identical to that of SEQ ID NO: 187. In some embodiments, the synthetic IL-7 polypeptide comprises an amino acid sequence at least about 95% identical to that of SEQ ID NO: 187. In some embodiments, the synthetic IL-7 polypeptide comprises an amino acid sequence at least about 96% identical to that of SEQ ID NO: 187. In some embodiments, the synthetic IL-7 polypeptide comprises an amino acid sequence at least about 97% identical to that of SEQ ID NO: 187. In some embodiments, the synthetic IL-7 polypeptide comprises an amino acid sequence at least about 98% identical to that of SEQ ID NO: 187. In some embodiments, the synthetic IL-7 polypeptide comprises an amino acid sequence at least about 99% identical to that of SEQ ID NO: 187. In some embodiments, the synthetic IL-7 polypeptide comprises an amino acid sequence identical to that of SEQ ID NO: 187.

In some embodiments, the synthetic IL-7 polypeptide comprises an amino acid sequence at least 80%, 85%, 90%, or 95% identical to SEQ ID NO: 186. In some embodiments, the synthetic IL-7 polypeptide consists of an amino acid sequence at least 80%, 85%, 90%, or 95% identical to the sequence of SEQ ID NO: 186.

In some embodiments, the linker is attached to the IL-7 polypeptide at an amino acid residue. In some embodiments, the chemical linker is attached at an amino acid residue corresponding to any one of amino acid residues 1-152 of SEQ ID NO: 1 (e.g., any one of amino acid residues 1-152 of SEQ ID NO: 1).

In some embodiments, the linker is attached to a terminal amino acid residue of the IL-7 polypeptide. In some embodiments, the linker is attached to the N-terminal residue or the C-terminal residue of the IL-7 polypeptide. In some embodiments, the linker is attached to the N-terminal amino group of the IL-7 polypeptide or the C-terminal carboxyl group of the IL-7 polypeptide. In some embodiments, the N-terminal residue is a residue corresponding to position 1 of SEQ ID NO: 1. In some embodiments, the IL-7 polypeptide comprises a truncation of one or more amino acid residues from the N-terminus of SEQ ID NO: 1 (e.g., a deletion of 1, 2, 3, 4, 5, 6, 7, 8, 9, 10, or more amino acid residues) and the linker is attached to the residue which now comprises the N-terminus (e.g., for a truncation of one amino acid, the linker is attached to a residue at a position corresponding to residue 2 of SEQ ID NO: 1). In some embodiments, the IL-7 polypeptide comprises a truncation of one or more amino acid residues from the C-terminus of SEQ ID NO: 1 (e.g., a deletion of 1, 2, 3, 4, 5, 6, 7, 8, 9, 10, or more amino acid residues) and the linker is attached to the residue which now comprises the C-terminus (e.g., for a truncation of one amino acid, the linker is attached to a residue at a position corresponding to residue 151 of SEQ ID NO: 1).

In some embodiments, the linker is attached to the N-terminal amino acid residue of the IL-7 polypeptide. In some embodiments, the linker is attached to the N-terminal amino group of the IL-7 polypeptide. In some embodiments, the linker is attached to the N-terminal amino group of the IL-7 polypeptide through by a reaction with an adduct attached to the N-terminal amino group having a structure

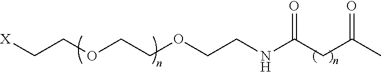

wherein each n is independently an integer from 1-30 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30), and wherein X is a conjugation handle (e.g., an azide or other conjugation handle provided herein, such as a DBCO group). In some embodiments, the adduct has the structure

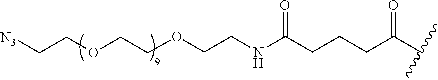

In some embodiments, the IL-7 polypeptide comprises a conjugation handle attached to one or more residues to facilitate attachment of the linker to the polypeptide which selectively binds to PD-1. The conjugation handle may be any such conjugation handle provided herein and may be attached at any residue to which the linker may be attached. In some embodiments, the conjugation handle is attached to the N-terminal residue of the polypeptide. In some embodiments, the conjugation handle comprises an azide or an alkyne.

In some embodiments, an IL-7 polypeptide described herein is capable of expanding CD4+ helper cell, CD8+ central memory cell, CD8+ effector memory cell, naïve CD8+ cell, Natural Killer (NK) cell, Natural killer T (NKT) cell populations, or a combination thereof. In some embodiments, a synthetic IL-7 polypeptide as described herein is capable of expanding CD4+ helper cell, CD8+ central memory cell, CD8+ effector memory cell, naïve CD8+ cell, Natural Killer (NK) cell, Natural killer T (NKT) cell populations, or a combination thereof. In some embodiments, an IL-7 polypeptide described herein is capable of expanding or inducing STAT5 phosphorylation in a CD8 naïve cell, a CD4 naïve cell, a CD8 memory cell, a CD4 memory cell, or a CD4 Treg cell, or any combination thereof. In some embodiments, a synthetic IL-7 polypeptide as provided herein is capable of expanding or activating one or more T-cell subtypes in a manner similar or substantially identical to a recombinant or wild type IL-7 polypeptide (e.g., exhibits an EC50 of no more than 100-fold greater than, or an EC50 of no more than 10-fold greater than a corresponding recombinant IL-7 polypeptide).

In some embodiments, the synthetic IL-7 polypeptide exhibits a half maximal effective concentration (EC50) for inducing STAT5 phosphorylation in at least one T-cell subtype which is comparable to a corresponding wild type or recombinant IL-7. In some embodiments, the EC50 of the synthetic IL-7 for inducing STAT5 phosphorylation in the at least one T-cell subtype is no more than 2-fold greater than, 3-fold greater than, 4-fold greater than, 5-fold greater than, 6-fold greater than, 7-fold greater than, 8-fold greater than, 9-fold greater than, 10-fold greater, 20-fold greater than, 50-fold greater than, or 100-fold greater than that of a corresponding recombinant IL-7. In some embodiments, the T-cell subtype is a CD8 naïve cell, a CD4 naïve cell, a CD8 memory cell, a CD4 memory cell, or a CD4 Treg cell. In some embodiments, the T-cell subtype is each of a CD8 naïve cell, a CD4 naïve cell, a CD8 memory cell, a CD4 memory cell, and a CD4 Treg cell.

In some embodiments, the IL-7 polypeptide conjugated to the polypeptide which binds specifically to PD-1 exhibits a half maximal effective concentration (EC50) for inducing STAT5 phosphorylation in at least one T-cell subtype which is comparable to wild type IL-7 when attached to the polypeptide which binds specifically to PD-1. In some embodiments, the EC50 of the IL-7 for inducing STAT5 phosphorylation in the at least one T-cell subtype is no more than 2-fold greater than, 3-fold greater than, 4-fold greater than, 5-fold greater than, 6-fold greater than, 7-fold greater than, 8-fold greater than, 9-fold greater than, 10-fold greater, 20-fold greater than, 50-fold greater than, or 100-fold greater than that of wild type IL-7. In some embodiments, the T-cell subtype is a CD8 naïve cell, a CD4 naïve cell, a CD8 memory cell, a CD4 memory cell, or a CD4 Treg cell. In some embodiments, the T-cell subtype is each of a CD8 naïve cell, a CD4 naïve cell, a CD8 memory cell, a CD4 memory cell, and a CD4 Treg cell.

In some embodiments, the IL-7 polypeptide conjugated to the polypeptide which binds specifically to PD-1 exhibits a half maximal effective concentration (EC50) for inducing STAT5 phosphorylation in at least one T-cell subtype which is the unconjugated IL-7 polypeptide (e.g., attaching the IL-7 polypeptide to the polypeptide which binds specifically to PD-1 does not substantially diminish the activity of the IL-7 polypeptide). In some embodiments, the EC50 of the IL-7 for inducing STAT5 phosphorylation in the at least one T-cell subtype is no more than 2-fold greater than, 3-fold greater than, 4-fold greater than, 5-fold greater than, 6-fold greater than, 7-fold greater than, 8-fold greater than, 9-fold greater than, 10-fold greater, 20-fold greater than, 50-fold greater than, or 100-fold greater than that the unconjugated IL-7. In some embodiments, the T-cell subtype is a CD8 naïve cell, a CD4 naïve cell, a CD8 memory cell, a CD4 memory cell, or a CD4 Treg cell. In some embodiments, the T-cell subtype is each of a CD8 naïve cell, a CD4 naïve cell, a CD8 memory cell, a CD4 memory cell, and a CD4 Treg cell.

### IL-18 Polypeptides

After stimulation with antigen plus IL-12, naïve T cells develop into IL-18 receptor (IL-18R)-expressing Th1 cells, which increase IFN-γ production in response to IL-18 stimulation. IL-18 is a proinflammatory cytokine that facilitates type 1 responses. IL-18 without IL-12, but with IL-2, stimulates NK cells, CD4⁺ NKT cells, and established Th1 cells, to produce IL-3, IL-9, and IL-13. Concomitant with IL-3, IL-18 stimulates mast cells and basophils to produce IL-4, IL-13, and chemical mediators (e.g., histamine). IL-18 is a member of the IL-1 family of cytokines. Murine and human IL-18 proteins consist of 192 and 193 amino acids, respectively. IL-18 is produced as a biologically inactive precursor, pro-IL-18, which lacks a signal peptide and requires proteolytic processing to become active. The cleavage of pro-IL-18 or pro-IL-10 depends mainly on the action of the intracellular cysteine protease caspase-1 in the NLRP3 inflammasome. The IL-18 receptor (IL-18R) consists of the inducible component IL-18Rα (IL-1 receptor-related protein [IL-1Rrp]) and the constitutively expressed component IL-18Rβ (IL-1R accessory protein-like [IL-1RAcPL]). Cytoplasmic domains of IL-18Rα and IL-18Rβ contain a common domain termed the Toll-like receptor (TLR)/IL-1R (TIR) domain. Upon stimulation with IL-18, IL-18Rα forms a high-affinity heterodimeric complex with IL-18Rβ, which mediates intracellular signal transduction. Cytoplasmic TIR domains of the receptor complex interact with myeloid differentiation primary response 88 (MyD88), a signal adaptor containing a TIR domain, via TIR-TIR interactions. Then, MyD88-induced events result in the activation of nuclear factor (NF)-κB and mitogen-activated protein kinase (MAPK) via association with the signal adaptors IL-1R-associated kinase (IRAK) 1-4 and tumor necrosis factor (TNF) receptor-activated factor (TRAF) 6, respectively, which eventually leads to the appropriate gene expressions, such as Ifng, Tnfa, Cd40l, and FasL. Non-limiting examples of IL-18 amino acid sequences to be utilized in embodiments described herein are provided below in Table 8C. An IL-18 polypeptide utilized in conjugate described herein can have, for example, an amino acid sequence that is at least 80%, 85%, 90%, 91%, 92%, 93%, 94%, 95%, 96%, 97%, 98%, 99%, or 100% identical to any one of the sequences in Table 8C.

### Retention of Antibody Activity

In some embodiments, an immunoconjugate composition provided herein (e.g., a polypeptide which binds to the antigen or antigen binding fragment (e.g., an anti-CD20 antibody such as rituximab) attached to an IL-2 polypeptide through a linker) maintains binding affinity associated with at least one of the components after formation of the linkage between the two groups. For example, in an immunoconjugate composition comprising an antibody or antigen binding fragment linked to an IL-2 polypeptide, in some embodiments the antibody or antigen binding fragment thereof retains binding to one or more Fc receptors. In some embodiments, the composition displays binding to one or more Fc receptors which is reduced by no more than about 5-fold, no more than about 10-fold, no more than about 15-fold, or no more than about 20-fold compared to the unconjugated antibody. In some embodiments, the one or more Fc receptors is the FcRn receptor, CD16a, the FcγRI receptor (CD64), the FcγRIIa receptor (CD32α), the FcγRIIβ receptor (CD32β), or any combination thereof. In some embodiments, binding of the composition to each of the FcRn receptor, CD16a, the FcγRI receptor (CD64), the FcγRIIa receptor (CD32α), and the FcγRIIβ receptor (CD32β) is reduced by no more than about 10-fold compared to the unconjugated antibody.

In some embodiments, binding of the polypeptide which binds to the antigen or antigen binding fragment (e.g., the antibody) is substantially unaffected by the conjugation with the protein (e.g., the cytokine). In some embodiments, the binding of the antibody or antigen binding fragment to the antigen is reduced by no more than about 5% compared to the unconjugated antibody. In some embodiments, the binding affinity of the antibody or antigen binding fragment to the antigen or is reduced by no more than about 1.1-fold, 1.2-fold, 1.5-fold, 2-fold, 3-fold, 4-fold, 5-fold, or 10-fold compared to the unconjugated antibody or antigen binding fragment.

### Orthogonal Payloads

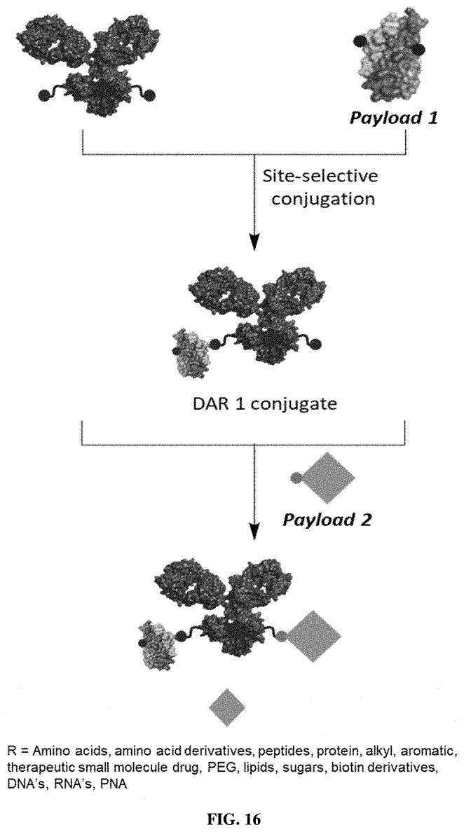

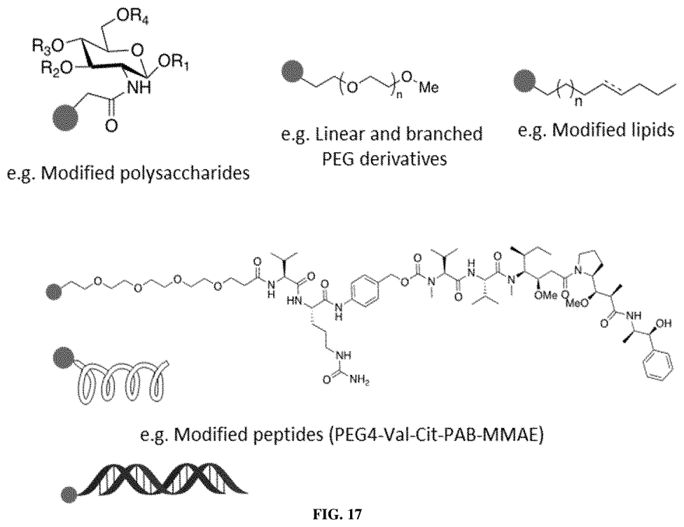

The antigen/antigen binding fragment-protein (e.g., cytokine) immunoconjugates of the disclosure can comprise dual orthogonal payloads. An exemplary process for making an immunoconjugate with dual orthogonal payloads is shown in FIG. 16. A plurality of exemplary dual orthogonal payloads compatable with the immunoconjugates provided herein are shown in FIG. 17. In one non-limiting instance, the antigen/antigen binding fragment—cytokine immunoconjugates can comprise one antigen or antigen binding fragment, one modified cytokine, and one payload that is linked to the antigen or antigen binding fragment by a orthogonal linking group. The orthogonal payload can be an amino acid, amino acid derivative, peptide, protein, cytokine, alkyl group, aryl or heteroaryl group, therapeutic small molecule drug, polyethylene glycol (PEG) moiety, lipid, sugar, biotin, biotin derivative, deoxyribonucleic acid (DNA), ribonucleic acid (RNA), or peptide nucleic acid (PNA), any of which is substituted, unsubstituted, modified, or unmodified. In some embodiments, the orthogonal payload is a therapeutic small molecule. In some embodiments, the orthogonal payload is a PEG moiety. In some embodiments, the orthogonal payload is an additional cytokine such as, for example, IL-7, or IL-18.

### Compositions

In one aspect, described herein is a pharmaceutical composition comprising: an antibody or antigen binding fragment linked to a modified cytokine described herein; and a pharmaceutically acceptable carrier or excipient. In some embodiments, the pharmaceutical composition further comprises one or more excipients, wherein the one or more excipients include, but are not limited to, selected from a carbohydrate, an inorganic salt, an antioxidant, a surfactant, a buffer, or any combination thereof. In some embodiments the pharmaceutical composition further comprises one, two, three, four, five, six, seven, eight, nine, ten, or more excipients, wherein the one or more excipients include, but are not limited to, a carbohydrate, an inorganic salt, an antioxidant, a surfactant, a buffer, or any combination thereof.

In some embodiments, the pharmaceutical composition further comprises a carbohydrate. In certain embodiments, the carbohydrate is selected from the group consisting of fructose, maltose, galactose, glucose, D-mannose, sorbose, lactose, sucrose, trehalose, cellobiose raffinose, melezitose, maltodextrins, dextrans, starches, mannitol, xylitol, maltitol, lactitol, xylitol, sorbitol (glucitol), pyranosyl sorbitol, myoinositol, cyclodextrins, and combinations thereof.

Alternately, or in addition, the pharmaceutical composition further comprises an inorganic salt. In certain embodiments, the inorganic salt is selected from the group consisting of sodium chloride, potassium chloride, magnesium chloride, calcium chloride, sodium phosphate, potassium phosphate, sodium sulfate, or combinations thereof.

Alternately, or in addition, the pharmaceutical composition further comprises an antioxidant. In certain embodiments, the antioxidant is selected from the group consisting of ascorbyl palmitate, butylated hydroxyanisole, butylated hydroxytoluene, potassium metabisulfite, propyl gallate, sodium metabisulfite, sodium thiosulfate, vitamin E, 3,4-dihydroxybenzoic acid, and combinations thereof.

Alternately, or in addition, the pharmaceutical composition further comprises a surfactant. In certain embodiments, the surfactant is selected from the group consisting of polysorbates, sorbitan esters, lipids, phospholipids, phosphatidylethanolamines, fatty acids, fatty acid esters, steroids, EDTA, zinc, and combinations thereof.

Alternately, or in addition, the pharmaceutical composition further comprises a buffer. In certain embodiments, the buffer is selected from the group consisting of citric acid, sodium phosphate, potassium phosphate, acetic acid, ethanolamine, histidine, amino acids, tartaric acid, succinic acid, fumaric acid, lactic acid, tris, HEPES, or combinations thereof.

In some embodiments, the pharmaceutical composition is formulated for parenteral or enteral administration. In some embodiments, the pharmaceutical composition is formulated for intravenous (IV) or subcutaneous administration. In some embodiments, the pharmaceutical composition is in a lyophilized form.

In one aspect, described herein is a liquid or lyophilized composition that comprises a described an antigen or antigen binding fragment linked to a modified cytokine. In some embodiments, the antigen or antigen binding fragment linked to the modified cytokine is a lyophilized powder. In some embodiments, the lyophilized powder is resuspended in a buffer solution. In some embodiments, the buffer solution comprises a buffer, a sugar, a salt, a surfactant, or any combination thereof. In some embodiments, the buffer solution comprises a phosphate salt. In some embodiments, the phosphate salt is sodium Na₂HPO₄. In some embodiments, the salt is sodium chloride. In some embodiments, the buffer solution comprises phosphate buffered saline. In some embodiments, the buffer solution comprises mannitol. In some embodiments, the lyophilized powder is suspended in a solution comprising about 10 mM Na₂HPO₄ buffer, about 0.022% SDS, and about 50 mg/mL mannitol, and having a pH of about 7.5.

### Dosage Forms

The antigen or antigen binding fragment linked to the modified cytokine described herein can be in a variety of dosage forms. In some embodiments, antigen or antigen binding fragment linked to the cytokine is dosed as a reconstituted lyophilized powder. In some embodiments, the antigen or antigen binding fragment linked to the modified cytokine is dosed as a suspension. In some embodiments, the antigen or antigen binding fragment linked to the modified cytokine is dosed as a solution. In some embodiments, the antigen or antigen binding fragment linked to the modified cytokine is dosed as an injectable solution. In some embodiments, the antigen or antigen binding fragment linked to the modified cytokine is dosed as an IV solution.

### Methods of Treatment

Methods of treating a cancer or a metastasis thereof, or an inflammatory disorder in a subject with a conjugate are contemplated herein. The conjugate can be a conjugate described herein. In such conjugates, an antibody or antigen binding fragment selectively binds to, for example, a cancer antigen, an immune cell target molecule, a self-antigen, or a combination thereof.

The cancer antigen may be, for example, programmed cell death 1 (PD1) programmed cell death ligand 1 (PDL1), CD5, CD20, CD19, CD22, CD30, CD33, CD40, CD44, CD52, CD74, CD103, CD137, CD123, CD152, a carcinoembryonic antigen (CEA), an integrin, an epidermal growth factor (EGF) receptor family member, a vascular epidermal growth factor (VEGF), a proteoglycan, a disialoganglioside, B7-H3, cancer antigen 125 (CA-125), epithelial cell adhesion molecule (EpCAM), vascular endothelial growth factor receptor 1, vascular endothelial growth factor receptor 2, a tumor associated glycoprotein, mucin 1 (MUC1), a tumor necrosis factor receptor, an insulin-like growth factor receptor, folate receptor α, transmembrane glycoprotein NMB, a C—C chemokine receptor, prostate specific membrane antigen (PSMA), recepteur d'origine nantais (RON) receptor, cytotoxic T-lymphocyte antigen 4 (CTLA4), Colon cancer antigen 19.9, gastric cancer mucin antigen 4.2, colorectal carcinoma antigen A33, ADAM-9, AFP oncofetal antigen-alpha-fetoprotein, ALCAM, BAGE, beta-catenin, Carboxypeptidase M, B1, CD23, CD25, CD27, CD28, CD36, CD45, CD46, CD52, CD56, CD79a/CD79b, CD317, CDK4, CO-43 (blood group Le^{b}), CO-514 (blood group Lea), CTLA-1, Cytokeratin 8, DR5, E1 series (blood group B), Ephrin receptor A2 (EphA2), Erb (ErbB1, ErbB3, ErbB4), lung adenocarcinoma antigen F3, antigen FC10.2, GAGE-1, GAGE-2, GD2/GD3/GD49/GM2/GM3, GICA 19-9, gp37, gp75, gp100, HER-2/neu, human milk fat globule antigen, human papillomavirus-E6/human papillomavirus-E7, high molecular weight melanoma antigen (HMW-MAA), differentiation antigen (I antigen), I(Ma) as found in gastric adenocarcinomas, Integrin Alpha-V-Beta-6, Integrinβ6 (ITGβ6), Interleukin-13 Receptor α2 (IL13Rα2), JAM-3, KID3, KID31, KS 1/4 pan-carcinoma antigen, KSA (17-1A), human lung carcinoma antigen L6, human lung carcinoma antigen L20, LEA, LUCA-2, M1:22:25:8, M18, M39, MAGE-1, MAGE-3, MART, Myl, MUM-1, N-acetylglucosaminyltransferase, neoglycoprotein, NS-10, OFA-1 and OFA-2, Oncostatin M (Oncostatin Receptor Beta), rho15, prostate specific antigen (PSA), PSMA, polymorphic epithelial mucin antigen (PEMA), PIPA, prostatic acid phosphate, R24, ROR1, SSEA-1, SSEA-3, SSEA-4, sTn, T cell receptor derived peptide, T5A7, Tissue Antigen 37, TAG-72, TL5 (blood group A), a TNF-α receptor (TNFαR), TNFβR, TNFγR, TRA-1-85 (blood group H), Transferrin Receptor, TSTA tumor-specific transplantation antigen, VEGF-R, Y hapten, Le^{y}, 5T4, or a combination thereof.

An immune cell target molecule may be, for example, PD-1, PD-L1, PD-L2, CTLA-4, CD28, B7-1 (CD80), B7-2 (CD86), ICOS ligand, ICOS, B7-H3, B7-H4, VISTA, B7-H7 (HHLA2), TMIGD2, 4-1BBL, 4-1BB, HVEM, BTLA, CD160, LIGHT, MHC Class I, MHC Class II, LAG3, OX40L, OX40, CD70, CD27, CD40, CD40L, GITRL, GITR, CD155, DNAM-1, TIGIT, CD96, CD48, 2B4, Galectin-9, TIM-3, Adenosine, Adenosine A2a Receptor, IDO, TDO, CEACAM1, CD47, SIRP alpha, BTN2A1, DC-SIGN, CD200, CD200R, TL1A, DR3, or a combination thereof.

A self-antigen may be, for example, tumor necrosis factor alpha (TNFα), myelin basic protein (MBP), myelin oligodendrocyte glycoprotein (MOG), proteolipid protein (PLP), type II collagen (CII), vimentin, α-enolase, a clusterin, a histone, peptidyl arginine deiminase-4, transglutaminase 2 (TG2, TGM2), CD318, Peptidoglycan Recognition Protein 1 (PGLYRP1), or a combination thereof.

In one aspect, described herein, is a method of treating a cancer or a metastasis thereof in a subject in need thereof, comprising: administering to the subject an effective amount of an antigen or antigen binding fragment (e.g., a polypeptide which selectively binds to PD-1, PD-L1, CD20, etc.) linked to a modified cytokine or a pharmaceutical composition as described herein.

In one aspect, described herein, is a method of treating cancer in a subject in need thereof, comprising: administering to the subject an effective amount of an antibody/antigen binding fragment-modified cytokine conjugate or a pharmaceutical composition as described herein. In some embodiments, the cancer is a solid cancer. A cancer or tumor can be, for example, a primary cancer or tumor or a metastatic cancer or tumor. Cancers and tumors to be treated include, but are not limited to, a melanoma, a lung cancer (e.g., a non-small cell lung cancer (NSCLC), a small cell lung cancer (SCLC), etc.), a carcinoma (e.g., a cutaneous squamous cell carcinoma (CSCC), a urothelial carcinoma (UC), a renal cell carcinoma (RCC), a hepatocellular carcinoma (HCC), a head and neck squamous cell carcinoma (HNSCC), an esophageal squamous cell carcinoma (ESCC), a gastroesophageal junction (GEJ) carcinoma, an endometrial carcinoma (EC), a Merkel cell carcinoma (MCC), etc.), a bladder cancer (BC), a microsatellite instability high (MSI-H)/mismatch repair-deficient (dMMR) solid tumor (e.g., a colorectal cancer (CRC)), a tumor mutation burden high (TMB-H) solid tumor, a triple-negative breast cancer (TNBC), a gastric cancer (GC), a cervical cancer (CC), a pleural mesothelioma (PM), classical Hodgkin's lymphoma (cHL), or a primary mediastinal large B cell lymphoma (PMBCL).

In another aspect, described herein, is a method of treating cancer in a subject in need thereof, comprising: administering to the subject an effective amount of an antibody/antigen binding fragment-modified cytokine conjugate or a pharmaceutical composition as described herein. In some embodiments, the cancer is a solid cancer. A cancer or tumor can be, for example, a primary cancer or tumor or a metastatic cancer or tumor. Cancers and tumors to be treated include, but are not limited to, a melanoma, a lung cancer (e.g., a non-small cell lung cancer (NSCLC), a small cell lung cancer (SCLC), etc.), a carcinoma (e.g., a cutaneous squamous cell carcinoma (CSCC), a urothelial carcinoma (UC), a renal cell carcinoma (RCC), a hepatocellular carcinoma (HCC), a head and neck squamous cell carcinoma (HNSCC), an esophageal squamous cell carcinoma (ESCC), a gastroesophageal junction (GEJ) carcinoma, an endometrial carcinoma (EC), a Merkel cell carcinoma (MCC), etc.), a bladder cancer (BC), a microsatellite instability high (MSI-H)/mismatch repair-deficient (dMMR) solid tumor (e.g., a colorectal cancer (CRC)), a tumor mutation burden high (TMB-H) solid tumor, a triple-negative breast cancer (TNBC), a gastric cancer (GC), a cervical cancer (CC), a pleural mesothelioma (PM), classical Hodgkin's lymphoma (cHL), or a primary mediastinal large B cell lymphoma (PMBCL).

Alternatively, or in addition, described herein is a method of treating cancer in a subject in need thereof, comprising administering to the subject an effective amount of an antibody/antigen binding fragment-modified cytokine conjugate or a pharmaceutical composition as described herein. A cancer can be a primary cancer or a metastatic cancer. In some embodiments, the cancer is a solid cancer or a blood cancer. In some instances, the cancer to be treated comprises a CD20-positive lymphoma. Lymphomas to be treated include, but are not limited to, a Hodgkin's Lymphoma (HL), a Non-Hodgkin's Lymphoma (NHL), a Follicular Lymphoma (FL), a diffuse large B-cell lymphoma, a mantle cell lymphoma, or a combination thereof. Alternatively, or in addition, the cancer to be treated comprises a CD20-positive leukemia. Leukemias to be treated include, but are not limited to, a Chronic Lymphocytic Leukemia (CLL), a hairy cell leukemia (HCL), or a combination thereof. Alternatively, or in addition, the cancer to be treated comprises a CD20-positive myeloma. Myelomas include, but are not limited to, a multiple myeloma. Alternatively, or in addition, the cancer to be treated comprises a CD20-positive thymoma. Alternatively, or in addition, the cancer to be treated comprises a CD20-positive melanoma.

Alternatively, or in addition, described herein is a method of treating an autoimmune disease in a subject in need thereof, comprising administering to the subject an effective amount of an antibody/antigen binding fragment-modified cytokine conjugate or a pharmaceutical composition as described herein. In some embodiments, the autoimmune disease comprises multiple sclerosis (MS), Rheumatoid Arthritis (RA), Granulomatosis with Polyangiitis (GPA), (Wegener's Granulomatosis), Microscopic Polyangiitis (MPA), Pemphigus Vulgaris (PV), or a combination thereof.

Alternatively, or in addition, described herein is a method of treating an inflammatory disorder in a subject in need thereof, comprising: administering to the subject an effective amount of an antibody/antigen binding fragment-modified cytokine conjugate or a pharmaceutical composition as described herein. In some embodiments, the inflammatory disorder comprises inflammation (e.g., cartilage inflammation), an autoimmune disease, an atopic disease, a paraneoplastic autoimmune disease, arthritis, rheumatoid arthritis (e.g., active), juvenile arthritis, juvenile idiopathic arthritis, juvenile rheumatoid arthritis, pauciarticular rheumatoid arthritis, pauciarticular juvenile rheumatoid arthritis, polyarticular juvenile rheumatoid arthritis, systemic onset juvenile rheumatoid arthritis, juvenile psoriatic arthritis, psoriatic arthritis, polyarticular rheumatoid arthritis, systemic onset rheumatoid arthritis, ankylosing spondylitis, juvenile ankylosing spondylitis, juvenile enteropathic arthritis, reactive arthritis, juvenile reactive arthritis, Reiter's syndrome, juvenile Reiter's syndrome, juvenile dermatomyositis, juvenile scleroderma, juvenile vasculitis, enteropathic arthritis, SEA syndrome (Seronegativity, Enthesopathy, Arthropathy syndrome), dermatomyositis, psoriatic arthritis, scleroderma, vasculitis, myolitis, polymyolitis, dermatomyolitis, polyarteritis nodossa, Wegener's granulomatosis, arteritis, ploymyalgia rheumatica, sarcoidosis, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, dermatitis herpetiformis, Behcet's disease, alopecia, alopecia areata, alopecia totalis, atherosclerosis, lupus, Still's disease, myasthenia gravis, inflammatory bowel disease (IBD), Crohn's disease, ulcerative colitis, celiac disease, asthma, COPD, rhinosinusitis, rhinosinusitis with polyps, eosinophilic esophagitis, eosinophilic bronchitis, Guillain-Barre disease, thyroiditis (e.g., Graves' disease), Addison's disease, Raynaud's phenomenon, autoimmune hepatitis, graft versus host disease, steroid refractory chronic graft versus host disease, transplantation rejection (e.g. kidney, lung, heart, skin, and the like), kidney damage, hepatitis C-induced vasculitis, spontaneous loss of pregnancy, vitiligo, focal segmental glomerulosclerosis (FSGS), minimal change disease, membranous nephropathy, ANCA-associated Glomerulonephropathy, Membranoproliferative Glomerulonephritis, IgA nephropathy, lupus nephritis, or a combination thereof.

A conjugate described herein can be administered to a subject in one or more doses. In some embodiments, the conjugate is administered in a single dose of the effective amount of the modified protein (e.g., modified cytokine), including further embodiments in which (i) the conjugate is administered once a day; or (ii) the conjugate is administered to the subject multiple times over the span of one day. In some embodiments, the conjugate is administered daily, every other day, 3 times a week, once a week, every 2 weeks, every 3 weeks, every 4 weeks, every 5 weeks, every 3 days, every 4 days, every 5 days, every 6 days, bi-weekly, 3 times a week, 4 times a week, 5 times a week, 6 times a week, once a month, twice a month, 3 times a month, once every 2 months, once every 3 months, once every 4 months, once every 5 months, or once every 6 months. Administration includes, but is not limited to, injection by any suitable route (e.g., parenteral, enteral, intravenous, subcutaneous, etc.).

An effective response is achieved when the subject experiences partial or total alleviation or reduction of signs or symptoms of illness, and specifically includes, without limitation, prolongation of survival. The expected progression-free survival times may be measured in months to years, depending on prognostic factors including the number of relapses, stage of disease, and other factors. Prolonging survival includes without limitation times of at least 1 month (mo), about at least 2 mos., about at least 3 mos., about at least 4 mos., about at least 6 mos., about at least 1 year, about at least 2 years, about at least 3 years, about at least 4 years, about at least 5 years, etc. Overall or progression-free survival can be also measured in months to years. Alternatively, an effective response may be that a subject's symptoms remain static and do not worsen. Further indications of treatment of indications are described in more detail below. In some instances, a cancer or tumor is reduced by at least 2%, 5%, 10%, 15%, 20%, 25%, 30%, 35%, 40%, 45%, 50%, 55%, 60%, 65%, 70%, 75%, 80%, 85%, 90%, 95%, or 100%.

Combination therapies with one or more additional active agents are contemplated herein. For example, a conjugate can be administered in combination with one or more of the following: a Disease-Modifying Antirheumatic Drug (DMARD), a Nonsteroidal Anti-Inflammatory Drug (NSAID), an aminosalicylate (a compound that contain 5-aminosalicylic acid (5-ASA)), a corticosteroid, an anti-IL12 antibody, or a Janus Kinase (JAK) inhibitor. In some instances, the DMARD is methotrexate, sulfasalazine, hydroxychloroquine, leflunomide, Azathioprine, etc. In some instances, the 5-ASA drug is sulfasalazine (Azulfidine®), a mesalamine (e.g., ASACOL® HD, PENTASA®, LIALDA™, APRISO®, DELZICOL™, etc.), olsalazine (DIPENTUM®), balsalazide (COLAZAL®), CANASA®, ROWASA®, etc. In some instances, the JAK inhibitor is a Janus kinase 1 (JAK1) inhibitor, a Janus kinase 2 (JAK2) inhibitor, a Janus kinase 3 (JAK3) inhibitor, or a combination thereof. In some instances, the anti-IL12 antibody comprises ustekinumab (STELARA®; anti-IL12/IL23). In some instances, the corticosteroid comprises a glucocorticoid such as, for example, hydrocortisone (CORTEF®), cortisone, ethamethasoneb (Celestone Soluspan (betamethasone sodium phosphate and betamethasone acetate), prednisone (Prednisone Intensol), prednisolone (ORAPRED®, Prelone), triamcinolone (Aristospan Intra-Articular, Aristospan Intralesional, Kenalog), ethylprednisolone (Medrol, Depo-Medrol, Solu-Medrol), dexamethasone, etc. In some instances, the NSAID is Aspirin, celecoxib (CELEBREX®, etc.), diclofenac (CAMBIA®, CATAFLAM®, VOLTAREN®-XR, ZIPSOR®, ZORVOLEX®, etc.), ibuprofen (MOTRIN®, ADVIL®, etc.), indomethacin (INDOCIN®, etc.), naproxen (ALEVE® ANAPROX®, NAPRELAN®, NAPROSYN®, etc.), oxaprozin (DAYPRO®, etc.), piroxicam (FELDENE®, etc.), or a combination thereof. Other appropriate combinations such as surgery, chemotherapy, radiation, physical therapy, psychological therapy, etc. are included herein.

### Methods of Manufacturing

In one aspect, described herein, is a method of making a composition, comprising providing antibody or antigen binding fragment, wherein the antibody or antigen binding fragment comprises a reactive group (e.g., a conjugation handle), contacting the reactive group with a complementary reactive group attached to a cytokine, and forming the composition. The resulting composition is any of the compositions provided herein.

In some embodiments, providing the antibody or antigen binding fragment comprising the reactive group comprises attaching the reactive group to the antibody or antigen binding fragment. In some embodiments, the reactive group is added site-specifically. In some embodiments, attaching the reactive group to the antibody or antigen binding fragment comprises contacting the antibody or antigen binding fragment with an affinity group comprising a reactive functionality which forms a bond with a specific residue of the antibody or antigen binding fragment. In some embodiments, attaching the reactive group to the antibody or antigen binding fragment comprises contacting the antibody or antigen binding fragment with an enzyme. In some embodiments, the enzyme is configured to site-specifically attach the reactive group to a specific residue of the antibody or antigen binding fragment. In some embodiments, the enzyme is glycosylation enzyme or a transglutaminase enzyme.

In some embodiments, the method further comprises attaching the complementary reactive group to the cytokine. In some embodiments, attaching the complementary reactive group to the cytokine comprises chemically synthesizing the cytokine.

In some embodiments, the method comprises making a modified cytokine. In some embodiments, the method of making a modified cytokine comprises synthesizing two or more fragments of the modified cytokine and ligating the fragments. In some embodiments, the method of making the modified cytokine comprises a. synthesizing two or more fragments of the modified cytokine, b. ligating the fragments; and c. folding the ligated fragments.

In some embodiments, the two or more fragments of the modified cytokine are synthesized chemically. In some embodiments, the two or more fragments of the modified IL cytokine are synthesized by solid phase peptide synthesis. In some embodiments, the two or more fragments of the modified IL cytokine are synthesized on an automated peptide synthesizer.

In some embodiments, the modified cytokine is ligated from 2, 3, 4, 5, 6, 7, 8, 9, 10, or more peptide fragments. In some embodiments, the modified cytokine is ligated from 2 peptide fragments. In some embodiments, the modified cytokine is ligated from 3 peptide fragments. In some embodiments, the modified IL cytokine is ligated from 4 peptide fragments. In some embodiments, the modified IL cytokine is ligated from 2 to 10 peptide fragments.

In some embodiments, the two or more fragments of the modified cytokine are ligated together. In some embodiments, three or more fragments of the modified cytokine are ligated in a sequential fashion. In some embodiments, three or more fragments of the modified IL cytokine are ligated in a one-pot reaction.

In some embodiments, ligated fragments are folded. In some embodiments, folding comprises forming one or more disulfide bonds within the modified cytokine. In some embodiments, the ligated fragments are subjected to a folding process. In some embodiments, the ligated fragments are folding using methods well known in the art. In some embodiments, the ligated polypeptide or the folded polypeptide are further modified by attaching one or more polymers thereto. In some embodiments, the ligated polypeptide or the folded polypeptide are further modified by PEGylation. In some embodiments, the modified IL cytokine is synthetic. In some embodiments, the modified IL cytokine is recombinant.

### Sequences (SEQ ID NOS) of Exemplary Cytokines

TABLE 8A
(IL-2 Polypeptides)

| | SEQ ID | |
|---|---|---|
| Substitutions | NO | Sequence |
| None (WT) | 1 | APTSSSTKKTQLQLEHLLLDLQMILNGINNYKNPKLTRML |
| | | TFKFYMPKKATELKHLQCLEEELKPLEEVLNLAQSKNFH |
| | | LRPRDLISNINVIVLELKGSETTFMCEYADETATIVEFLNR |
| | | WITFCQSIISTLT |
| ΔA1, C125S | 2 | PTSSSTKKTQLQLEHLLLDLQMILNGINNYKNPKLTRMLT |
| (Aldesleukin) | | FKFYMPKKATELKHLQCLEEELKPLEEVLNLAQSKNFHL |
| | | RPRDLISNINVIVLELKGSETTFMCEYADETATIVEFLNRW |
| | | ITFSQSIISTLT |
| M23Nle, | 3 | APTSSSTKKTQLQLEHLLLDLQ(Nle)ILNGINNYKNPKLTR |
| F42Y, | | (Nle)L(Hse)YKFY(Nle)PKKATELKHLQCLEEELKPLEEVL |
| M39Nle, | | (Hse)LAQSKNFHLRPRDLISNINVIVLELKGSETTF(Hse)CEYA |
| M46Nle, | | DETATIVEFLNRWITFSQSIISTLT |
| N71Hse, | | |
| M104Hse, | | |
| C125S | | |
| M23Nle, | 4 | APTSSSTKKTQLQLEHLLLDLQ(Nle)ILNGINNYKNPKLTR |
| M39Nle, | | (Nle)L(Hse)FKFY(Nle)PKKATELKHLQCLEEELKPLEEVL |
| T41Hse, | | (Hse)LAQSKNFHLRPRDLISNINVIVLELKGSETTF(Hse)CEYA |
| M46Hse, | | DETATIVEFLNRWITFSQSIISTLT |
| N71Hse, | | |
| M104Hse, | | |
| C125S | | |
| M23Nle, | 5 | APTSSSTKKTQLQLEHLLLDLQ(Nle)ILNGINNYKNPKLTR |
| M39Nle, | | (Nle)L(Hse)YKFY(Nle)PKKATELKHLQCLEEELKPLEEVL |
| T41Hse, | | (Hse)YAQSKNFHLRPRDLISNINVIVLELKGSETTF(Hse)CEYA |
| F42Y, | | DETATIVEFLNRWITFSQSIISTLT |
| M46Nle, | | |
| N71Hse, | | |
| L72Y, | | |
| M104Hse, | | |
| C125S | | |
| M23Nle, | 6 | APTSSSTKKTQLQLEHLLLDLQ(Nle)ILNGINNYKNPKLTR |
| M39Nle, | | (Nle)L(Hse)FKFY(Nle)PKKATELKHLQCLEEELKPLEEVL |
| T41Hse, | | (Hse)YAQSKNFHLRPRDLISNINVIVLELKGSETTF(Hse)CEYA |
| M46Nle, | | DETATIVEFLNRWITFSQSIISTLT |
| N71Hse, | | |
| L72Y, | | |
| M104Hse, | | |
| C125S | | |
| M23Nle, | 7 | APTSSSTKKTQLQLEHLLLDLQ(Nle)ILNGINNYKNPKLTR |
| M39Nle, | | (Nle)L(Hse)YKFY(Nle)PKKATELKHLQCLEEELKPLEEVL |
| T41Hse, | | (Hse)GAQSKNFHLRPRDLISNINVIVLELKGSETTF(Hse)CEYA |
| F42Y, | | DETATIVEFLNRWITFSQSIISTLT |
| M46Nle, | | |
| N71Hse, | | |
| L72G, | | |
| M104Hse, | | |
| C125S | | |
| M23Nle, | 8 | APTSSSTKKTQLQLEHLLLDLQ(Nle)ILNGINNYKNPKLTR |
| M39Nle, | | (Nle)L(Hse)YKFY(Nle)PKKATELKHLQCLEEELKYLEEVL |
| T41Hse, | | (Hse)LAQSKNFHLRPRDLISNINVIVLELKGSETTF(Hse)CEY |
| F42Y, | | ADETATIVEFLNRWITFSQSIISTLT |
| M46Nle, | | |
| P65Y, | | |
| N71Hse, | | |
| M104Hse, | | |
| C125S | | |
| M23Nle, | 9 | APTSSSTKKTQLQLEHLLLDLQ(Nle)ILNGINNYKNPKLTR |
| M39Nle, | | (Nle)L(Hse)FKFY(Nle)PKKATELKHLQCLEEELKYLEEVL |
| T41Hse, | | (Hse)LAQSKNFHLRPRDLISNINVIVLELKGSETTF(Hse)CEYA |
| M46Nle, | | DETATIVEFLNRWITFSQSIISTLT |
| P65Y, | | |
| N71Hse, | | |
| M104Hse, | | |
| C125S | | |
| M23Nle, | 10 | APTSSSTKKTQLQLEHLLLDLQ(Nle)ILNGINNYKNPKLTY |
| R38Y, | | (Nle)L(Hse)YKFY(Nle)PKKATELKHLQCLEEYLKYLEEVL |
| M39Nle, | | (Hse)LAQSKNFHLRPRDLISNINVIVLELKGSETTF(Hse)CEY |
| T41Hse, | | ADETATIVEFLNRWITFSQSIISTLT |
| F42Y, | | |
| M46Nle, | | |
| E62Y, P65Y, | | |
| N71Hse, | | |
| M104Hse, | | |
| C125S | | |
| M23Nle, | 11 | APTSSSTKKTQLQLEHLLLDLQ(Nle)ILNGINNYKNPKLTR |
| M39Nle, | | (Nle)L(Hse)FKFY(Nle)PKKATELKHLQCLEEYLKYLEEVL |
| T41Hse, | | (Hse)LAQSKNFHLRPRDLISNINVIVLELKGSETTF(Hse)CEY |
| M46Nle, | | ADETATIVEFLNRWITFSQSIISTLT |
| H64Nle, | | |
| E62Y, P65Y, | | |
| N71Hse, | | |
| M104Hse, | | |
| C125S | | |
| M23Nle, | 12 | APTSSSTKKTQLQLEHLLLDLQ(Nle)ILNGINNYKNPKLTR |
| M39Nle, | | (Nle)L(Hse)FKFY(Nle)PKKATELKHLQCLEEYLKPLEEVL |
| T41Hse, | | (Hse)LAQSKNFHLRPRDLISNINVIVLELKGSETTF(Hse)CEYA |
| M46Nle, | | DETATIVEFLNRWITFSQSIISTLT |
| E62Y, | | |
| N71Hse, | | |
| M104Hse, | | |
| C125S | | |
| M23Nle, | 13 | APTSSSTKKTQLQLEHLLLDLQ(Nle)ILNGINNYKNPKLTR |
| M39Nle, | | (Nle)L(Hse)FYFY(Nle)PKKATELKHLQCLEEELKPLEEVL |
| T41Hse, | | (Hse)LAQSKNFHLRPRDLISNINVIVLELKGSETTF(Hse)CEYA |
| K43Y, | | DETATIVEFLNRWITFSQSIISTLT |
| M46Nle, | | |
| N71Hse, | | |
| M104Hse, | | |
| C125S | | |
| M23Nle, | 14 | APTSSSTKKTQLQLEHLLLDLQ(Nle)ILNGINNYKNPKLTR |
| M39Nle, | | (Nle)L(Hse)FKYY(Nle)PKKATELKHLQCLEEELKPLEEVL |
| T41Hse, | | (Hse)LAQSKNFHLRPRDLISNINVIVLELKGSETTF(Hse)CEYA |
| F44Y, | | DETATIVEFLNRWITFSQSIISTLT |
| M46Nle, | | |
| N71Hse, | | |
| M104Hse, | | |
| C125S | | |
| M23Nle, | 15 | APTSSSTKKTQLQLEHLLLDLQ(Nle)ILNGINNYKNPYLTR |
| K35Y, | | (Nle)L(Hse)FKFY(Nle)PKKATELKHLQCLEEELKPLEEVL |
| M39Nle, | | (Hse)LAQSKNFHLRPRDLISNINVIVLELKGSETTF(Hse)CEYA |
| T41Hse, | | DETATIVEFLNRWITFSQSIISTLT |
| M46Nle, | | |
| N71Hse, | | |
| M104Hse, | | |
| C125S | | |
| H16Y, | 16 | APTSSSTKKTQLQLEYLLLDLQ(Nle)ILNGINNYKNPKLTR |
| M23Nle, | | (Nle)L(Hse)FKFY(Nle)PKKATELKHLQCLEEELKPLEEVL |
| M39Nle, | | (Hse)LAQSKNFHLRPRDLISNINVIVLELKGSETTF(Hse)CEYA |
| T41Hse, | | DETATIVEFLNRWITFSQSIISTLT |
| M46Hse, | | |
| N71Hse, | | |
| M104Hse, | | |
| C125S | | |
| H16Y, | 17 | APTSSSTKKTQLQLEYLLLDLQ(Nle)ILNGINNYKNPKLTR |
| M23Nle, | | (Nle)L(Hse)YKFY(Nle)PKKATELKHLQCLEEELKPLEEVL |
| M39Nle, | | (Hse)LAQSKNFHLRPRDLISNINVIVLELKGSETTF(Hse)CEYA |
| T41Hse, | | DETATIVEFLNRWITFSQSIISTLT |
| F41Y, | | |
| M45Nle, | | |
| N71Hse, | | |
| M104Hse, | | |
| C125S | | |
| D20Y, | 18 | APTSSSTKKTQLQLEHLLLYLQ(Nle)ILNGINNYKNPKLTR |
| M23Nle, | | (Nle)L(Hse)FKFY(Nle)PKKATELKHLQCLEEELKPLEEVL |
| M39Nle, | | (Hse)LAQSKNFHLRPRDLISNINVIVLELKGSETTF(Hse)CEYA |
| T41Hse, | | DETATIVEFLNRWITFSQSIISTLT |
| M46Nle, | | |
| N71Hse, | | |
| M104Hse, | | |
| C125S | | |
| D20Y, | 19 | APTSSSTKKTQLQLEHLLLYLQ(Nle)ILNGINNYKNPKLTR |
| M23Nle, | | (Nle)L(Hse)YKFY(Nle)PKKATELKHLQCLEEELKPLEEVL |
| M39Nle, | | (Hse)LAQSKNFHLRPRDLISNINVIVLELKGSETTF(Hse)CEYA |
| T41Hse, | | DETATIVEFLNRWITFSQSIISTLT |
| F42Y, | | |
| M46Nle, | | |
| N71Hse, | | |
| M104Hse, | | |
| C125S | | |
| M23Nle, | 20 | APTSSSTKKTQLQLEHLLLDLQ(Nle)ILNGINNYKNPYLTR |
| K35Y, | | (Nle)L(Hse)YKFY(Nle)PKKATELKHLQCLEEELKPLEEVL |
| M39Nle, | | (Hse)LAQSKNFHLRPRDLISNINVIVLELKGSETTF(Hse)CEYA |
| T41Hse, | | DETATIVEFLNRWITFSQSIISTLT |
| F42Y, | | |
| M46Nle, | | |
| N71Hse, | | |
| M104Hse, | | |
| C125S | | |
| M23Nle, | 21 | APTSSSTKKTQLQLEHLLLDLQ(Nle)ILNGINNYKNPKLTR |
| M39Nle, | | (Nle)L(Hse)YKFY(Nle)PKKATELKHLQCLEEYLKPLEEVL |
| T41Hse, | | (Hse)LAQSKNFHLRPRDLISNINVIVLELKGSETTF(Hse)CEY |
| F42Y, | | ADETATIVEFLNRWITFSQSIISTLT |
| M46Nle, | | |
| E62Y, | | |
| N71Hse, | | |
| M104Hse, | | |
| C125S | | |
| M23Nle, | 22 | APTSSSTKKTQLQLEHLLLDLQ(Nle)ILNGINNYKNPKLTR |
| M39Nle, | | (Nle)L(Hse)YKFY(Nle)PKKATELKHLQCLEEYLKYLEEVL |
| T41Hse, | | (Hse)LAQSKNFHLRPRDLISNINVIVLELKGSETTF(Hse)CEY |
| F42Y, | | ADETATIVEFLNRWITFSQSIISTLT |
| M46Nle, | | |
| E62Y, P65Y, | | |
| N71Hse, | | |
| M104Hse, | | |
| C125S | | |
| M23Nle, | 23 | APTSSSTKKTQLQLEHLLLDLQ(Nle)ILNGINNYKNPKLTR |
| M39Nle, | | (Nle)L(Hse)FKFY(Nle)PKKATELKHLQCLEEELKPLEEVL |
| T41Hse, | | (Hse)LAQSKNFHLRPRDLISNINVIVLELKGSETTF(Hse)CEYA |
| M46Nle, | | DETATIVEFLNRWITFCQSIISTLT |
| N71Hse, | | |
| M104Hse | | |
| M23Nle, | 176 | APTSSSTKKT QLQLEHLLLD LQXILNGINN |
| Y31H, K35R, | [[CMP- | HKNPRLTRXL ZFKFYXPKKA TELKHLQCLE |
| M39Nle, | 086]] | EELKPLEEVL ZLAPSKNFHL RPRDLISDIN VIVLELKGSE |
| T41Hse, | | TTFZCEYADE TATIVEFLNR WITFSQSIIS TLT |
| M46Nle | | |
| N71Hse, | | |
| Q74P, N88D, | | |
| M104Hse, | | |
| C125S | | |
| X = Nle, | | |
| Z = Hse | | |
| N-terminus | | |
| with glutaric | | |
| acid and | | |
| 0.5 kDa azido | | |
| PEG | | |
| M23Nle, | 177 | APTSSSTKKT QLQLEHLLLD LQXILNGINN |
| Y31H, K35R, | (CMP- | HKNPRLTRXL ZFKFYXPKKA TELKHLQCLE |
| M39Nle, | 080) | EELKPLEEVL ZLAPSKNFHL RPRDLISNIN VIVLELKGSE |
| T41Hse, | | TTFZCEYADE TATIVEFLNR WITFSQSIIS TLT |
| M46Nle, | | |
| N71Hse, | | |
| Q74P | | |
| M104Hse, | | |
| C125S | | |
| X = Nle, | | |
| Z = Hse | | |
| N-terminus | | |
| with glutaric | | |
| acid and | | |
| 0.5 kDa azido | | |
| PEG | | |
| M23Nle, | 178 | APTSSSTKKT QLQLEHLLLD LQXILNGINN |
| Y31H, K35R, | (CMP- | HKNPRLTRXL ZFKFYXPKKA TELKHLQCLE |
| M39Nle, | 084) | EELKPLEEAL ZLAPSKNFHL RPRDLISDIN VIVLELKGSE |
| T41Hse, | | TTFZCEYADE TATIVEFLNR WITFSQSIIS TLT |
| M46Nle, | | |
| V69A, | | |
| N71Hse, | | |
| Q74P, N88D, | | |
| M104Hse, | | |
| C125S | | |
| X = Nle, | | |
| Z = Hse | | |
| N-terminus | | |
| with glutaric | | |
| acid and | | |
| 0.5 kDa azido | | |
| PEG | | |
| M23Nle, | 179 | APTSSSTKKT QLQLEHLLLD LQXILNGINN |
| Y31H, K35R, | (CMP- | HKNPRLTRAL ZFKFYXPKKA TELKHLQCLE |
| M39A, | 081) | EELKPLEEVL ZLAPSKNFHL RPRDLISNIN VIVLELKGSE |
| T41Hse, | | TTFZCEYADE TATIVEFLNR WITFSQSIIS TLT |
| M46Nle | | |
| N71Hse, | | |
| Q74R | | |
| M104Hse, | | |
| C125S | | |
| X = Nle, | | |
| Z = Hse | | |
| N-terminus | | |
| with glutaric | | |
| acid and | | |
| 0.5 kDa azido | | |
| PEG | | |
| M23A, Y31H, | 180 | APTSSSTKKT QLQLEHLLLD LQAILNGINN |
| K35R, | (CMP- | HKNPRLTRXL ZFKFYXPKKA TELKHLQCLE |
| M39Nle, | 082) | EELKPLEEVL ZLAPSKNFHL RPRDLISDIN VIVLELKGSE |
| T41Hse, | | TTFZCEYADE TATIVEFLNR WITFSQSIIS TLT |
| M46Nle, | | |
| N71Hse, | | |
| Q74P | | |
| M104Hse, | | |
| C125S | | |
| X = Nle, | | |
| Z = Hse | | |
| N-terminus | | |
| with glutaric | | |
| acid and | | |
| 0.5 kDa azido | | |
| PEG | | |
| M23Nle, | 181 | APTSSSTKKT QLQLEHLLLD LQXILNGINN |
| Y31H, K35R, | (CMP- | HKNPRLTRXL ZFKFYAPKKA TELKHLQCLE |
| M39Nle, | 088) | EELKPLEEVL ZLAPSKNFHL RPRDLISNIN VIVLELKGSE |
| T41Hse, | | TTFZCEYADE TATIVEFLNR WITFSQSIIS TLT |
| M46A, | | |
| N71Hse, | | |
| Q74R | | |
| M104Hse, | | |
| C125S | | |
| X = Nle, | | |
| Z = Hse | | |
| N-terminus | | |
| with glutaric | | |
| acid and | | |
| 0.5 kDa azido | | |
| PEG | | |
| M23Nle, | 182 | APTSSSTKKT QLQLEHLLLD LQXILNGINN |
| K35R, | (CMP- | YKNPRLTRXL ZFKFYXPKKA TELKHLQCLE |
| M39Nle, | 078) | EELKPLEEAL ZLAPSKNFHL RPRDLISDIN VIVLELKGSE |
| T41Hse, | | TTFZCEYADE TATIVEFLNR WITFSQSIIS TLT |
| M46Nle, | | |
| V69A, | | |
| N71Hse, | | |
| Q74P, N88D, | | |
| M104Hse, | | |
| C125S | | |
| X = Nle, | | |
| Z = Hse | | |
| M23Nle, | 183 | APTSSSTKKT QLQLEHLLLD LQXILNGINN |
| M39Nle, | (CMP- | YKNPKLTRXL ZBKFYXPKKA TELKHLQCLE |
| T41Hse, | 083) | EELKPLEEVL ZLAQSKNFHL RPRDLISDIN VIVLELKGSE |
| F42(4-NH₂)- | | TTFZCEYADE TATIVEFLNR WITFSQSIIS TLT |
| Phe, M46Nle, | | |
| N71Hse, | | |
| N88D, | | |
| M104Hse, | | |
| C125S | | |
| B = (4-NH₂)- | | |
| Phe | | |
| X = Nle, | | |
| Z = Hse | | |
| M23Nle, | 184 | APTSSSTKKT QLQLEHLLLD LQXILNGINN |
| M39Nle, | (CMP- | YKNPKLTRXL ZFKFYXPKKA TELKHLQCLE |
| T41Hse, | 042) | EELKPLEEVL ZLAQSKNFHL RPRDLISDIN VIVLELKGSE |
| M46Nle, | | TTFZCEYADE TATIVEFLNR WITFSQSIIS TLT |
| N71Hse, | | |
| N88D, | | |
| M104Hse, | | |
| C125S | | |
| X = Nle, | | |
| Z = Hse | | |
| M23Nle, | 185 | APTSSSTKKT QLQLEHLLLD LQXILNGINN |
| M39Nle, | (CMP- | YKNPKLTRXL ZFKFYXPKKA TELKHLQCLE |
| T41Hse, | 056) | EELKPLEEVL ZLAQSKNFHL RPRDLIS(Dgp)IN |
| M46Nle, | | VIVLELKGSE TTFZCEYADE TATIVEFLNR WITFSQSIIS |
| N71Hse, | | TLT |
| N88Dgp, | | |
| M104Hse, | | |
| C125S | | |
| X = Nle, | | |
| Z = Hse | | |
| Dgp = D with a | | |
| O-(2- | | |
| aminoethyl)- | | |
| O’-(2- | | |
| aminoethyl) | | |
| octaethylene | | |
| glycol | | |

In Table 8A above, Nle is a norleucine residue and Hse is a homoserine residue. Additionally, Table 8A above refers to several IL-2 variants as containing “N-terminus with glutaric acid and 0.5 kDa azido PEG.” For sake of clarity, it is intended that this modification is included when the corresponding molecule is referred to by the CMP number associated with the SEQ ID NO, but this modification is not contemplated to be part of the amino acid sequence.

TABLE 8B
(IL-7 Polypeptides)

| SEQ ID | |
|---|---|
| NO | IL-7 Sequence |
| 165 | MFHVSFRYIFGLPPLILVLLPVASSDCDIEGKDGKQYESVLMVSIDQLLDS |
| | MKEIGSNCLNNEFNFFKRHICDANKEGMFLFRAARKLRQFLKMNSTGDFD |
| | LHLLKVSEGTTILLNCTGQVKGRKPAALGEAQPTKSLEENKSLKEQKKLN |
| | DLCFLKRLLQEIKTCWNKILMGTKEH |
| 166 | MFHVSFRYTFGIPPLILVLLPWSSDCHIKDKDGKAFGSVLMISINQLDKMT |
| | GTDSDCPNNEPNFFKKHLCDDTKEAAFLNRAARKLRQFLKMNISEEFNDH |
| | LLRVSDGTQTLVNCTSKEEKTIKEQKKNDPCFLKRLLREIKTCWNKILKGS |
| | I |
| 167 | MFHVSFRYIFGIPPLILVLLPVTSSECHIKDKEGKAYESVLMISIDELDKMTG |
| | TDSNCPNNEPNFFRKHVCDDTKEAAFLNRAARKLKQFLKMNISEEFNVHL |
| | LTVSQGTQTLVNCTSKEEKNVKEQKKNDACFLKRLLREIKTCWNKILKGS |
| | I |
| 168 | MFHVSFRYIFGIPPLILVLLPVASSDCDISGKDGGAYQNVLMVNIDDLDNM |
| | INFDSNCLNNEPNFFKKHSCDDNKEASFLNRASRKLRQFLKMNISDDFKLH |
| | LSTVSQGTLTLLNCTSKGKGRKPPSLSEAQPTKNLEENKSSKEQKKQNDL |
| | CFLKILLQKIKTCWNKILRGIKEH |
| 169 | MFHVSFRYIFGIPPLILVLLPVASSDCDFSGKDGGAYQNVLMVSIDDLDNM |
| | INFDSNCLNNEPNFFKKHSCDDNKEASFLNRAARKLKQFLKMNISDDFKL |
| | HLSTVSQGTLTLLNCTSKGKGRKPPSLGEAQPTKNLEENKSLKEQRKQND |
| | LCFLKILLQKIKTCWNKILRGITEH |
| 186 | DCDIEGKDGK QYESVLMVSI DQLLDSMKEI GSNCLNNEFN |
| Native | FFKRHICDAN KEGMFLFRAA RKLRQFLKMN STGDFDLHLL |
| IL-7, | KVSEGTTILL NCTGQVKGRK PAALGEAQPT KSLEENKSLK |
| mature | EQKKLNDLCF LKRLLQEIKT CWNKILMGTK EH |
| 187 | DCDIEGKDGK QYESVLXVSI DQLLDSXKEI GSNCLZNEFN |
| | FFKRHICDAN KEGXFLFRAA RKLRQFLKXN STGDFZLHLL |
| | KVSEGTTILL NCTGQVKGRK PAALGEAQPT KSLZENKSLK EQKKLNDLCF |
| | LKRLLQEIKT CWNKILXGTK EH |
| | X = Nle, Z = Hse |

TABLE 8C
(IL-18 Polypeptides)

| SEQ ID | |
|---|---|
| NO | IL-18 Sequence |
| 170 | MAAEPVEDNCINFVAMKFIDNTLYFIAEDDENLESDYFGKLESKLSVIRNL |
| | NDQVLFIDQGNRPLFEDMTDSDCRDNAPRTIFIISMYKDSQPRGMAVTISV |
| | KCEKISTLSCENKIISFKEMNPPDNIKDTKSDIIFFQRSVPGHDNKMQFESSS |
| | YEGYFLACEKERDLFKLILKKEDELGDRSIMFTVQNED |
| 171 | MAAEQVEDYCISFVEMKFINNTLYFVAENDEDLESDHFGKLEPKLSIIRNL |
| | NDQVLFINQGNQPVFEDMPDSDCSDNAPQTIFIIYMYKDSLTRGLAVTISV |
| | QCKKMSTLSCENKIVSFKEMNPPDNIDNEESDIIFFQRSVPGHDDKIQFESS |
| | LYKGYFLACKKENDLFKLILKKQDDNRDKSVMFTVQNQN |
| 172 | MAAEQVEDYCISFVEMKFINNTLYFVAENDEDLESDHFGKLEPKLSIIRNL |
| | NDQVLFINQGNQPVFEDMPDSDCSDNAPQTIFIIYMYKDSLTRGLAVTISV |
| | QCKKMSTLSCENKIVSFKEMNPPDNIDNEESDIIFFQRSVPGHDDKIQFESS |
| | LYKGYFLACKKENDLFKIILKKQDDNRDKSVMFTVQNQN |
| 173 | MAAMSEEGSCYNFKEMMFIDNTLYLIPEDNGDLESDHFGRLHCTTAVIRSI |
| | NDQVLFVDKRNPPVFEDMPDIDRTANESQTRLIIYMYKDSEVRGLAVTLS |
| | VKDGRMSTLSCKNKIISFEEMNPPENIDDIKSDLIFFQKRVPGHNKMEFESS |
| | LYEGHFLACQKEDDAFKLVLKRKDENGDKSVMFTLTNIHQS |
| 174 | MAAMSEDSCVNFKEMMFIDNTLYFIPEENGDLESDNFGRLHCTTAVIRNLN |
| | DQVLFVDKRQPVFEDMTDIDQSASEPQTRLIIYMYKDSEVRGLAVTLSVK |
| | DSKMSTLSCKNKIISFEEMDPPENIDDIQSDLIFFQKRVPGHNKMEFESSLY |
| | EGHFLACQKEDDAFKLILKKKDENGDKSVMFTLTNLHQS |
| 175 | MAAGPVEDNCISLVEMKFIDNTLYFVAENDENLESDYFGRLEPKLSIIRNL |
| | NDQVLFINQGNQPVFEDMPDSDCTDNAPQTVFIIYMYKDSLTRGLAVTISV |
| | KCEKTSTLSCKNKIISFKEMSPPENINDEGNDIIFFQRSVPGHDDKIQFESSL |
| | YKGYFLACEKENDLFKLILKEKDENGDKSVMFTVQNQN |

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### EXAMPLES

### Example 1: Preparation of Antibody/Protein-Conjugates

Antibodies conjugated to proteins (including chemically synthesized proteins such as the cytokines provided herein) are prepared utilizing a modified method derived from AJICAP™ technology (Ajinomoto Bio-Pharma Services). AJICAP™ technology is described at least in PCT Publication No. WO2018199337A1, PCT Publication No. WO2019240288A1, PCT Publication No. WO2019240287A1, PCT Publication No. WO2020090979A1, Matsuda et al., Mol. Pharmaceutics 2021, 18, 4058-4066, and Yamada et al., AJICAP: Affinity Peptide Mediated Regiodivergent Functionalization of Native Antibodies. Angew. Chem., Int. Ed. 2019, 58, 5592-5597, and in particular Examples 2-4 of US Patent Publication No. US20200190165A1). A brief summary of the methods disclosed therein for preparing the modified antibody used to conjugate to a protein or synthetic protein is as follows:

A modified antibody (e.g., a monoclonal antibody such as pembrolizumab, LZM-009, durvalumab, rituximab, etc.) comprising a DBCO conjugation handle is prepared utilizing methods described in and derived from, for example, Examples 2-4 of US Patent Application No. US20200190165A1.

Briefly, the antibody with a free sulfhydryl group attached to a lysine residue side chain in the Fc region is prepared by contacting the antibody with an affinity peptide configured to deliver a protected version of the sulfhydryl group (e.g., a thioester or reducible disulfide) to the lysine residue. An exemplary peptide capable of performing this reaction is shown below, as reported in Matsuda et al., Mol. Pharmaceutics 2021, 18, 4058-4066, which selectively attached the sulfhydryl group via the NHS ester at residue K248 of the Fc region of the antibody:

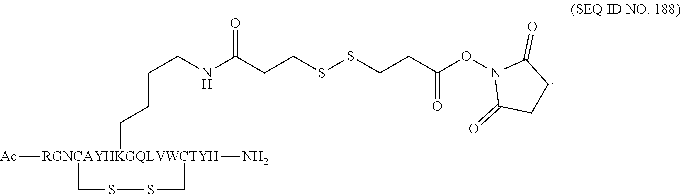

Alternative affinity peptides targeting alternative residues of the Fc region are described in the references cited above for AJICAP™ technology, and such affinity peptides can be used to attach the desired functionality to an alternative residue of the Fc region (e.g., K246, K288, etc.). For example, the disulfide group of the above affinity peptide could instead be replaced with a thioester to provide an sulfhydryl protecting group (e.g., the relevant portion of the affinity peptide would have a structure of

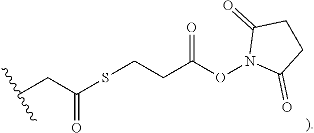 [SMILES: *CC(=O)SCCC(=O)ON1C(=O)CCC1=O]

The protecting group is then removed to reveal the free sulfhydryl (e.g., by hydrolysis of a thioester or reduction of a disulfide with TCEP). The free sulfhydryl is then reacted with a bifunctional reagent comprising a bromoacetamide group connected to the DBCO conjugation handle through a linking group (e.g., bromoacetamido-dPEG®₄-amido-DBCO). The method can be used to produce an antibody with one DBCO group present (DAR1) and/or two DBCO groups attached to the antibody (DAR2, one DBCO group linked to each Fc of the antibody).

In another embodiment, antibody comprising a single DBCO conjugation handle is prepared by first reacting excess anti-PD-L1 antibody with appropriately loaded affinity peptide to introduce a single sulfhydryl after appropriate removal of protecting group (e.g., disulfide reduction or thioester cleavage). A bifunctional linking group with a sulfhydryl reactive conjugation handle and DBCO conjugation handle (e.g., bromoacetamido-dPEG®₄-amido-DBCO) is then reacted with the single sulfhydryl to produce the single DBCO containing antibody. The single DBCO containing antibody is then conjugated with a suitable azide containing IL-2 (e.g., CMP-003) to achieve an anti-PD-L1-IL-2 immunoconjugate with a DAR of 1.

The desired azide containing modified protein or synthetic protein (e.g., azide modified IL-2 (CMP-003, CMP-086), azide modified IL-7 (CMP-095), or other desired protein) is then reacted with the DBCO modified antibody to produce the immunocytokine. The crude antibody/protein are then separated using hydrophobic interaction chromatography, ion exchange chromatography, size exclusion chromatography, trapping by DBCO-PEG, protein A chromatography, or DBCO resin column purification.

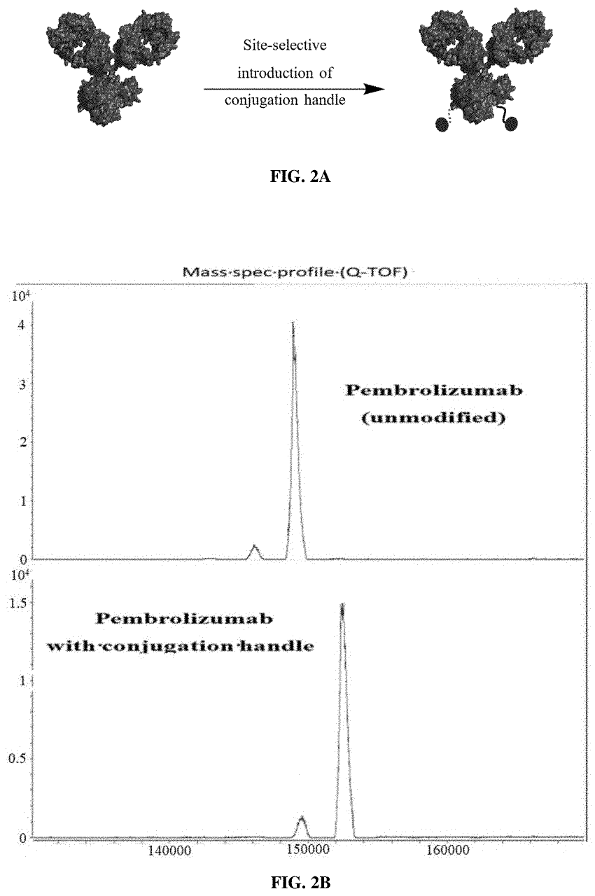

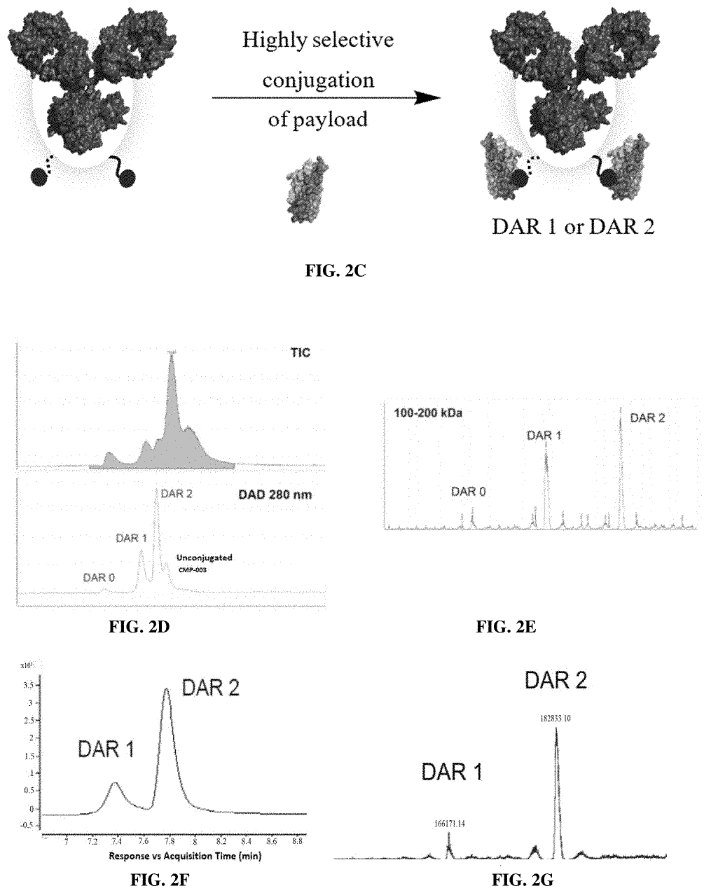

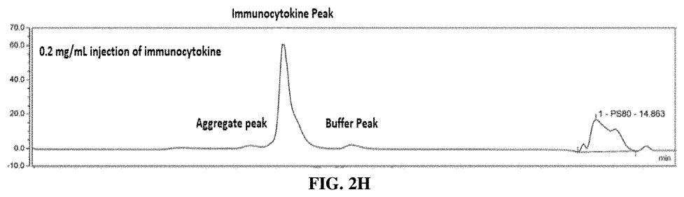

FIG. 2A shows site-selective modification of antibody by chemical modification technology to introduce one or two conjugation handles as provided herein. FIG. 2B shows Q-TOF mass spectra of unmodified pembrolizumab and pembrolizumab with conjugation with DBCO conjugation handle as an example. FIG. 2C shows site-selective conjugation of IL2 cytokine to generate a PD1-IL2 with DAR1, DAR 2 or mixed DAR between 1 and 2. FIG. 2D shows TIC chromatogram (top) and intact RP-HPLC (bottom) profile of crude pembrolizumab-IL2 (CMP-003) conjugation reaction. FIG. 2E shows Q-TOF mass spec profile of crude Pembrolizumab-IL2 (CMP-003) conjugation reaction showing the formation of DAR1 and DAR 2 species. FIG. 2F shows a SEC chromatogram of a purified Pembrolizumab-IL2 conjugate, wherein the IL-2 is CMP-003. FIG. 2G shows a mass spectrogram of a purified DAR˜2 Pembrolizumab-IL2 conjugate. FIG. 2H shows an analytical HPLC trace of a purified Pembrolizumab-IL-2 conjugate.

The Table 10 below summarizes immunoconjugates prepared according to the described methods. The CMP numbers referred to herein are defined below for each immunoconjugate.

TABLE 10
Exemplary Immunoconjugates

| | | | | | | | Aggre- |
|---|---|---|---|---|---|---|---|
| Com- | | Antigen | | | | | gates |
| posit- | | bound by | Attached | | Conjuga- | Endotoxin | (SEC- |
| ion | Antibody | antibody | polypeptide | DAR | tion site | (EU/ml) | HPLC) |
| CMP- | Pembrolizumab | PD-1 | CMP-003 | 1 | K248 | <5 | 1.5% |
| 001 | | | | | | | |
| CMP- | Pembrolizumab | PD-1 | CMP-003 | 1.5 | K248 | 2.5 | 0% |
| 005 | | | | | | | |
| CMP- | Pembrolizumab | PD-1 | CMP-003 | 2 | K248 | 8.9 | 0.9% |
| 007 | | | | | | | |
| CMP- | Pembrolizumab | PD-1 | CMP-003 | 1 | K288 | <5 | 1.78% |
| 103 | | | | | | | |
| CMP- | Pembrolizumab | PD-1 | CMP-003 | 2 | K288 | <5.5 | 1.02% |
| 025 | | | | | | | |
| CMP- | Pembrolizumab | PD-1 | CMP-010 | 1 | K248 | 69.7 | 2.53% |
| 012 | | | | | | | |
| CMP- | Pembrolizumab | PD-1 | CMP-010 | 2 | K248 | 75.6 | 2.65% |
| 018 | | | | | | | |
| CMP- | J43 | mPD-1 | CMP-003 | 1 | K248 | 46.8 | 1.43% |
| 014 | | | | | | | |
| CMP- | J43 | mPD-1 | CMP-003 | 2 | K248 | 13.7 | 2.27% |
| 019 | | | | | | | |
| CMP- | J43 | mPD-1 | CMP-010 | 1 | K248 | 40.9 | 1.43% |
| 017 | | | | | | | |
| CMP- | J43 | mPD-1 | CMP-010 | 2 | K248 | 33.2 | 1.47% |
| 101 | | | | | | | |
| CMP- | Humanized J43 | mPD-1 | CMP-003 | 1 | K248 | 6.1 | 0.38% |
| 024 | | | | | | | |
| CMP- | LZM-009 | PD-1 | CMP-003 | 1 | K248 | <5.0 | 2.1% |
| 016 | | | | | | | |
| CMP- | LZM-009 | PD-1 | CMP-003 | 2 | K248 | 17.5 | 1.7% |
| 027 | | | | | | | |
| CMP- | LZM-009 | PD-1 | CMP-003 | 1 | K288 | 7.81 | 1.48% |
| 102 | | | | | | | |
| CMP- | LZM-009 | PD-1 | CMP-003 | 1.9 | K288 | <0.006 | <5% |
| 028 | | | | | | | |
| CMP- | Biosimilar | CD20 | CMP-003 | 1 | K248 | | |
| 030 | Rituximab | | | | | | |
| CMP- | Durvalumab | PD-L1 | CMP-003 | 1.6 | K248 | 3.57 | 0% |
| 034 | | | | | | | |
| CMP- | pembrolizumab | PD-1 | CMP-095 | 1 | K248 | | |
| 039 | | | (IL-7) | | | | |
| CMP- | pembrolizumab | PD-1 | CMP-095 | 2 | K248 | | |
| 040 | | | (IL-7) | | | | |
| CMP- | LMZ-009 | PD-1 | CMP-095 | 1 | K248 | | |
| 041 | | | (IL-7) | | | | |
| CMP- | LMZ-009 | PD-1 | CMP-095 | 2 | K248 | | |
| 096 | | | (IL-7) | | | | |
| CMP- | Biosimilar | TNFα | CMP-086 | 1 | K248 | | |
| 089 | adalimumab | | | | | | |
| CMP- | Biosimilar | TNFα | CMP-086 | 2 | K248 | | |
| 090 | adalimumab | | | | | | |
| CMP- | Biosimilar | TNFα | CMP-086 | 1 | K248 | | |
| 092 | infliximab | | | | | | |
| CMP- | Biosimilar | TNFα | CMP-086 | 2 | K248 | | |
| 093 | infliximab | | | | | | |

### Example 2: Characterization of Antibody Antigen Binding in Immunoconjugates

The interaction of the unmodified and of conjugated antibodies their antigens were measured by ELISA assay.

For these PD-1 ELISAs, Corning high-binding half-area plates (Fisher Scientific, Reinach, Switzerland) were coated overnight at 4° C. with 25 μl of unmodified and of conjugated anti-PD1 antibodies at 5 μg/ml in PBS. Plates were then washed four times with 100 μl of PBS-0.02% Tween20. Plates surfaces were blocked with 25 μl of PBS-0.02% Tween20-1% BSA at 37° C. during 1 h. Plates were then washed four times with 100 μl of PBS-0.02% Tween20. Twenty-five microliters of recombinant biotinylated antigen (PD1/CD279 protein from AcroBiosystems (PD1-H82E4, Fisher Scientific, Reinach, Switzerland) were added in five-fold serial dilutions starting at 134 nM down to 0.002 nM into PBS-0.02% Tween20-0.1% BSA and incubated at 37° C. during 2 h. Plates were then washed four times with 100 μl of PBS-0.02% Tween20. Twenty-five microliters of Streptavidin-Horseradish peroxidase (#RABHRP3, Merck, Buchs, Switzerland) diluted at 1:500 into PBS-0.02% Tween20-01% BSA were added to each well and incubated at Room Temperature during 30 min. Plates were then washed four times with 100 μl of PBS-0.02% Tween20. Fifty microliters of TMB substrate reagent (#CL07, Merck, Buchs, Switzerland) were added to each well and incubated at 37° C. during 5 min. After 5 min at 37° C., Horseradish peroxidase reaction was stopped by adding 50 μl/well of 0.5M H₂SO₄ stop solution. ELISA signal was then measured at 450 nm on an EnSpire plate reader from Perkin Elmer (Schwerzenbach, Switzerland). ELISAs for other antibody/antigen pairs were performed using analogous methods. The Table 11 below shows results for the indicated constructs.

TABLE 11
Antigen Binding

| | | | | | Conjugation | |
|---|---|---|---|---|---|---|
| | | Antigen | | | site (Eu | |
| | | bound | | | numbering | |
| | | by | IL-2 | | of Fc | KD |
| Composition | Antibody | antibody | polypeptide | DAR | region) | (pM) |
| CMP-004 | Pembrolizumab | PD-1 | — | 0 | — | 37 |
| CMP-001 | Pembrolizumab | PD-1 | CMP-003 | 1 | K248 | 60 |
| CMP-005 | Pembrolizumab | PD-1 | CMP-003 | 1.5 | K248 | NT |
| CMP-007 | Pembrolizumab | PD-1 | CMP-003 | 2 | K248 | 56 |
| CMP-103 | Pembrolizumab | PD-1 | CMP-003 | 1 | K288 | 31 |
| CMP-025 | Pembrolizumab | PD-1 | CMP-003 | 2 | K288 | 29 |
| CMP-012 | Pembrolizumab | PD-1 | CMP-010 | 1 | K248 | 36 |
| CMP-018 | Pembrolizumab | PD-1 | CMP-010 | 2 | K248 | 41 |
| CMP-013 | J43 | PD-1 | — | 0 | — | >25000 |
| CMP-014 | J43 | PD-1 | CMP-003 | 1 | K248 | >25000 |
| CMP-019 | J43 | PD-1 | CMP-003 | 2 | K248 | >25000 |
| CMP-017 | J43 | PD-1 | CMP-010 | 1 | K248 | >25000 |
| CMP-101 | J43 | PD-1 | CMP-010 | 2 | K248 | >25000 |
| CMP-024 | Humanized J43 | PD-1 | CMP-003 | 1 | K248 | >25000 |
| CMP-105 | LZM-009 | PD-1 | — | 0 | — | 35 |
| CMP-016 | LZM-009 | PD-1 | CMP-003 | 1 | K248 | 37 |
| CMP-027 | LZM-009 | PD-1 | CMP-003 | 2 | K248 | NT |
| CMP-102 | LZM-009 | PD-1 | CMP-003 | 1 | K288 | 27 |
| CMP-028 | LZM-009 | PD-1 | CMP-003 | 1.9 | K288 | 41 |
| CMP-008 | Nivolumab/Opdivo | PD-1 | — | 0 | — | 66 |
| CMP-032 | generic rituximab | CD20 | | 0 | | 76 |
| CMP-030 | generic rituximab | CD20 | CMP-003 | 1 | K248 | 119 |
| CMP-033 | durvalumab | PD-L1 | | | | See |
| | | | | | | FIG. |
| | | | | | | 3B |
| CMP-034 | durvalumab | PD-L1 | CMP-003 | 1.6 | K248 | See |
| (CMP-034) | | | | | | FIG. |
| | | | | | | 3B |
| CMP-039 | pembrolizumab | PD-1 | CMP-095 | 1 | K248 | 38 |
| CMP-040 | pembrolizumab | PD-1 | CMP-095 | 2 | K248 | 28 |
| CMP-041 | LMZ-009 | PD-1 | CMP-095 | 1 | K248 | 35 |
| CMP-096 | LMZ-009 | PD-1 | CMP-095 | 2 | K248 | NT |
| CMP-091 | Biosimilar | TNFa | | | | See |
| | adalimumab | | | | | FIG. |
| | | | | | | 3C |
| CMP-089 | Biosimilar | TNFα | CMP-086 | 1 | K248 | See |
| | adalimumab | | | | | FIG. |
| | | | | | | 3C |
| CMP-090 | Biosimilar | TNFα | CMP-086 | 2 | K248 | NT |
| | adalimumab | | | | | |
| CMP-092 | infliximab | TNFα | CMP-086 | 1 | K248 | NT |
| CMP-093 | infliximab | TNFα | CMP-086 | 2 | K248 | NT |

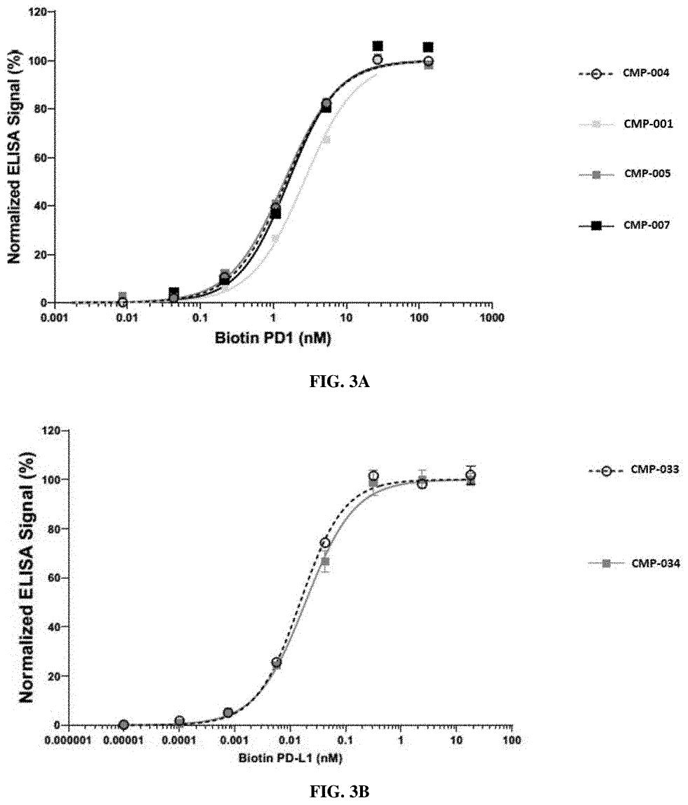

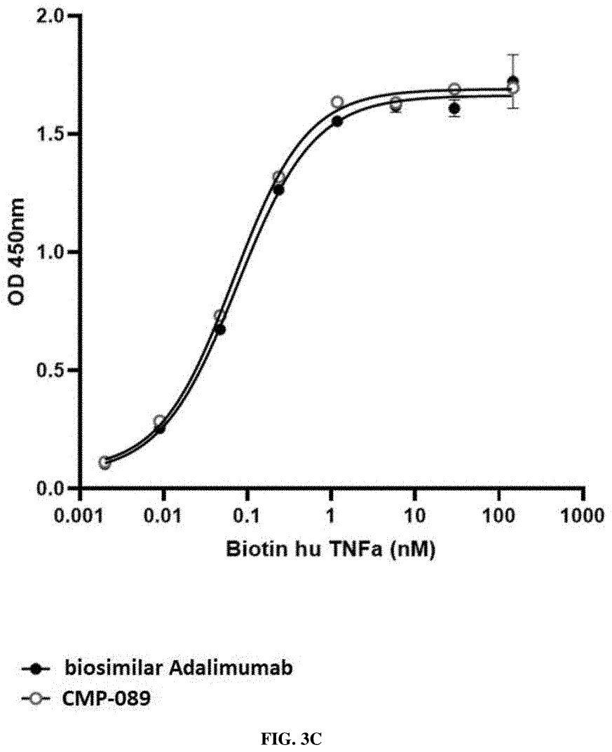

FIG. 3A shows plots measuring ability of the unmodified and of conjugated anti-PD1 antibodies to bind with PD1/CD279 ligand, with the figure showing normalized ELISA signal on the y-axis and dosage of the unmodified and of conjugated anti-PD1 antibodies on the x-axis. The compositions tested in this figure are composition Pembrolizumab (CMP-004), CMP-005, CMP-007, and CMP-001 respectively.

FIG. 3B shows an analogous plot for unmodified (CMP-033, Durvalumab) and conjugated (CMP-034) anti-PD-L1 antibodies and their ability to bind PD-L1.

FIG. 3C shows an analogous plot for unmodified (CMP-091, biosimilar Adalimumab) and conjugated (CMP-089) anti-TNFα antibodies and their ability to bind TNFα.

### Example 3—Antigen Blockade Assays for Select Immunoconjugates

For some immunoconjugates provided herein, the ability of the unmodified antibody and the conjugated antibody to block binding of the antigen with an antigen binding partners was tested (e.g., an anti-PD-1 antibody or corresponding immunoconjugate was tested for the ability to disrupt binding between PD-1 and PD-L1).

PD-1/PD-L1 blockade bioassay: A PD-1/PD-L1 blockade bioassay was used to determine the ability of the pembrolizumab-(IL-2 syntenin) immunoconjugates or anti-PD-L1-(IL-2 syntenin) immunoconjugates to block PD-1/PD-L1 interactions.

The ability of the unmodified and of conjugated anti-PD1 antibodies to interfere with PD1/PDL1 pathway was measured using the PD-1/PD-L1 Blockade Bioassay from Promega (Cat. #J1250, Madison, Wis., USA). PD-1/PD-L1 Blockade Bioassay is a bioluminescent cell-based assay based on the co-culture of effector cells with target cells mimicking an immunological synapse. Jurkat T cells expressing human PD-1 and a luciferase reporter driven by a NFAT response element (NFAT-RE) are activated by CHO-K1 cells expressing human PD-L1 and an engineered cell surface protein designed to activate Jurkat's cognate TCRs. Concurrent interaction PD-1/PD-L1 inhibits TCR signaling and represses NFAT-RE-mediated luminescence. Addition of either an anti-PD-1 or anti-PD-L1 antibody that blocks the PD-1/PD-L1 interaction releases the inhibitory signal, restoring TCR activation and resulting in a gain of signal of NFAT-RE luminescent reporter.

Briefly, PD-L1 aAPC/CHO-K1 Target cells were plated in white tissue culture −96 wells plates and cultured overnight at 37° C./5% CO2. Test molecules were measured in four-fold serial dilutions starting at 1 uM down to 0.002 nM and pre-incubated on target cells for 10 min before the addition of freshly thawed PD-1 Jurkat effector cells. After 6 h at 37° C./5% CO₂, activity NFAT-RE luminescent reporter was evaluated by the addition of Bio-Glo reagent and measured on an EnSpire plate reader (1 sec/well) from Perkin Elmer (Schwerzenbach, Switzerland).

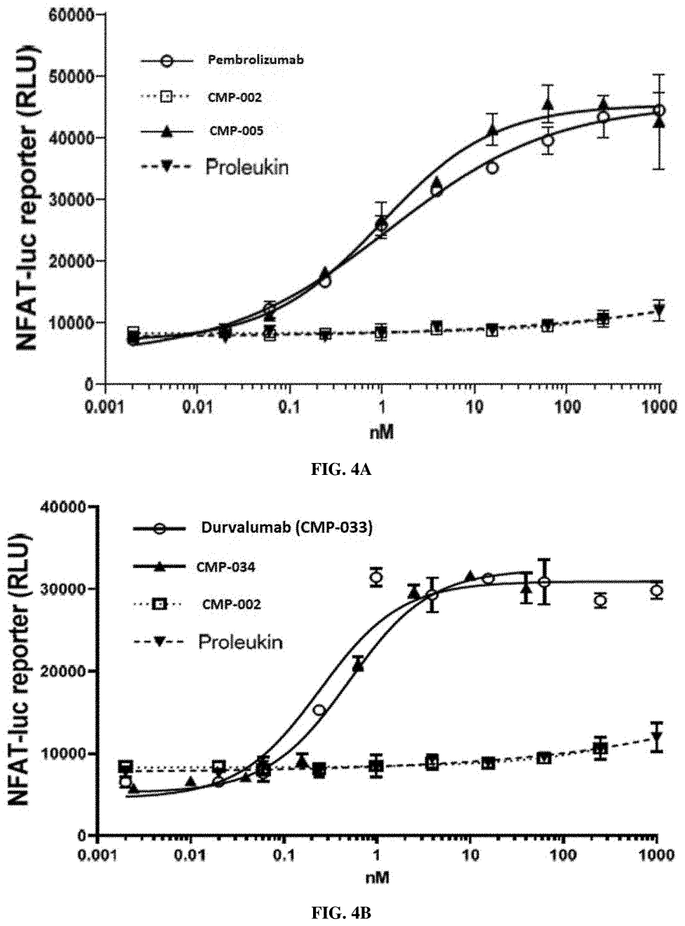

FIG. 4A shows plots measuring ability of the unmodified and of conjugated anti-PD1 antibodies to interfere with PD1/PDL1 pathway, with the figure showing mean luminescence intensity of effector cells NFAT-RE reporter on the y-axis and dosage of the unmodified and of conjugated anti-PD1 antibodies on the x-axis. The compositions tested in this figure are composition Pembrolizumab (CMP-004) and CMP-005. The modified IL-2 polypeptides tested in this figure are Proleukin and Composition CMP-002.

FIG. 4B shows an analogous plot for CMP-034 and parent anti-PD-L1 antibody Durvalumab.

### Example 4—FcRn Receptor Binding of Immunoconjugates

The ability of antibodies and corresponding immunoconjugates to bind to the human FcRn receptor was determined by alphaLISA as described below.

The interaction of the unmodified and of conjugated anti-PD1 antibodies with the human neonatal Fc receptor (FcRn) at pH 6 was measured using the AlphaLISA Human FcRn Binding Kit (AL3095C) from Perkin Elmer (Schwerzenbach, Switzerland). The AlphaLISA detection of FcRn and IgG binding uses IgG coated AlphaLISA® acceptor beads to interact with biotinylated human FcRn captured on Streptavidin-coated donor beads. When reference IgG binds to FcRn, donor and acceptor beads come into proximity enabling the transfer of singlet oxygen that trigger a cascade of energy transfer reactions in the acceptor beads, resulting in a sharp peak of light emission at 615 nm. Addition of a free IgG antibodies into the alphaLISA mixture creates a competition for the binding of FcRn to the reference antibody resulting in a loss of signal.

Briefly, test molecules were measured in serial dilutions starting at 5 uM down to 64 pM and incubated with AlphaLISA reaction mixture consisting of 800 nM of recombinant biotinylated human FcRn, 40 μg/ml of human IgG conjugated Acceptor beads, and 40 μg/ml of Streptavidin coated Donor beads in pH 6 MES buffer. After 90 min at 23° C. in the dark, AlphaLISA signal was measured on an EnSpire plate reader (Excitation at 680 nm, Emission at 615 nm) from Perkin Elmer (Schwerzenbach, Switzerland).

FcRn binding of other antibodies (e.g., anti-CD20, anti-PD-L1, anti-TNFα, etc.) was measured by analogous methods.

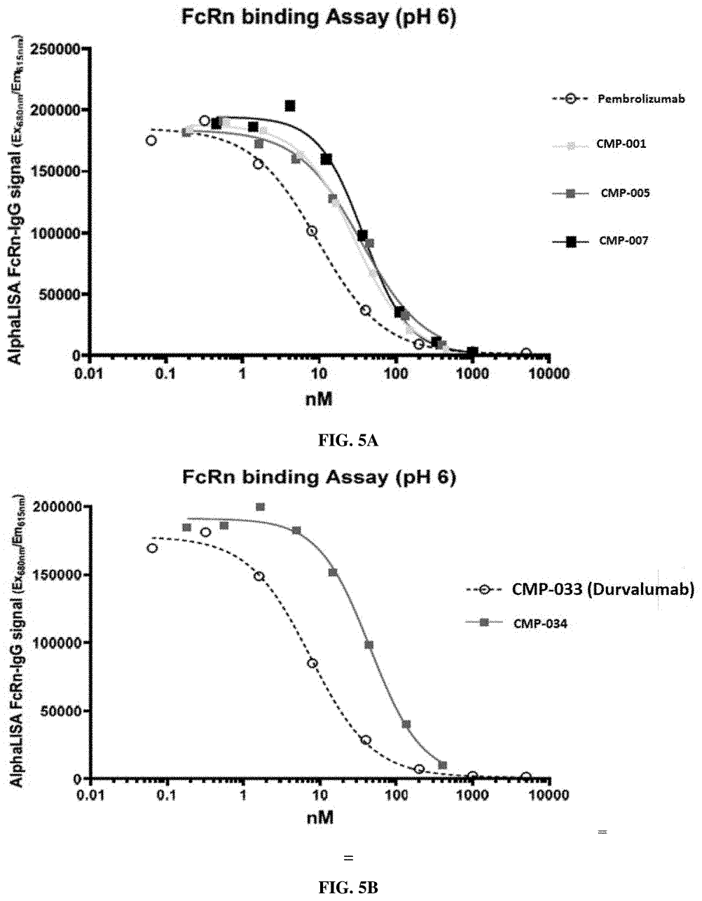

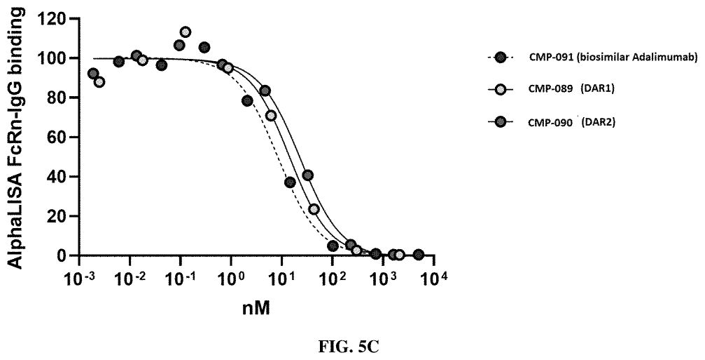

FIG. 5A shows plots measuring ability of the unmodified and of conjugated anti-PD1 antibodies to bind to human neonatal Fc receptor (FcRn) at pH 6, with the figure showing mean AlphaLISA FcRn-IgG signal on the y-axis and dosage of the unmodified and of conjugated anti-PD1 antibodies on the x-axis. The compositions in this figure are Pembrolizumab (CMP-004) and CMP-005, CMP-007, and CMP-001.

FIG. 5B shows plots measuring ability of unmodified and of conjugated anti-PD-L1 antibodies to bind to human FcRn at pH 6, with the figure showing mean AlphaLISA FcRn-IgG signal on the y-axis and dosage of the unmodified and of conjugated anti-PD-L1 antibodies on the x-axis. The constructs shown in this figure are CMP-033 and CMP-034.

FIG. 5C shows plots analogous to FIGS. 5A and 5B, but for unconjugated and conjugated anti-TNFa antibodies. The constructs shown in this figure are CMP-091 (unmodified antibody), CMP-089 (DAR1), and CMP-090 (DAR2).

The Table 12 below provides FcRn binding for a variety of immunoconjugates and parent antibodies provided herein.

TABLE 12
FcRn Binding

| | | Antigen | | | | |
|---|---|---|---|---|---|---|
| | | bound by | IL-2 | | Conjugation | KD |
| Composition | Antibody | antibody | polypeptide | DAR | site | (nM) |
| CMP-004 | Pembrolizumab | PD-1 | — | 0 | — | 7.31 |
| CMP-001 | Pembrolizumab | PD-1 | CMP-003 | 1 | K248 | 26.31 |
| CMP-005 | Pembrolizumab | PD-1 | CMP-003 | 1.5 | K248 | 27.80 |
| CMP-007 | Pembrolizumab | PD-1 | CMP-003 | 2 | K248 | 85.80 |
| CMP-103 | Pembrolizumab | PD-1 | CMP-003 | 1 | K288 | 18.56 |
| CMP-025 | Pembrolizumab | PD-1 | CMP-003 | 2 | K288 | 48.04 |
| CMP-012 | Pembrolizumab | PD-1 | CMP-010 | 1 | K248 | NT |
| CMP-018 | Pembrolizumab | PD-1 | CMP-010 | 2 | K248 | NT |
| CMP-013 | J43 | PD-1 | — | 0 | — | 5966 |
| CMP-014 | J43 | PD-1 | CMP-003 | 1 | K248 | NT |
| CMP-019 | J43 | PD-1 | CMP-003 | 2 | K248 | NT |
| CMP-017 | J43 | PD-1 | CMP-010 | 1 | K248 | NT |
| CMP-101 | J43 | PD-1 | CMP-010 | 2 | K248 | NT |
| CMP-024 | Humanized J43 | PD-1 | CMP-003 | 1 | K248 | NT |
| CMP-105 | LZM-009 | PD-1 | — | 0 | — | 15.27 |
| CMP-016 | LZM-009 | PD-1 | CMP-003 | 1 | K248 | 25.27 |
| CMP-027 | LZM-009 | PD-1 | CMP-003 | 2 | K248 | NT |
| CMP-102 | LZM-009 | PD-1 | CMP-003 | 1 | K288 | 10.69 |
| CMP-028 | LZM-009 | PD-1 | CMP-003 | 1.9 | K288 | 27.11 |
| CMP-032 | Biosimilar | CD20 | | 0 | | 4.034 |
| | rituximab | | | | | |
| CMP-030 | Biosimilar | CD20 | CMP-003 | 1 | K248 | 21.99 |
| | rituximab | | | | | |
| CMP-033 | durvalumab | PD-L1 | — | — | — | NT |
| CMP-034 | durvalumab | PD-L1 | CMP-003 | 1.6 | K248 | NT |
| (CMP-034) | | | | | | |
| CMP-039 | pembrolizumab | PD-1 | CMP-095 | 1 | K248 | NT |
| CMP-040 | pembrolizumab | PD-1 | CMP-095 | 2 | K248 | NT |
| CMP-041 | LMZ-009 | PD-1 | CMP-095 | 1 | K248 | 21.67 |
| CMP-096 | LMZ-009 | PD-1 | CMP-095 | 2 | K248 | NT |
| CMP-089 | Biosimilar | TNFα | CMP-086 | 1 | K248 | See FIG. |
| | Adalimumab | | | | | 5C. |
| CMP-090 | Biosimilar | TNFα | CMP-086 | 2 | K248 | See FIG. |
| | Adalimumab | | | | | 5C |
| CMP-092 | Biosimilar | TNFα | CMP-086 | 1 | K248 | NT |
| | Infliximab | | | | | |
| CMP-093 | Biosimilar | TNFα | CMP-086 | 2 | K248 | NT |
| | Infliximab | | | | | |

NT = not tested. Values in above table are the combined results of different experiments run on different days.

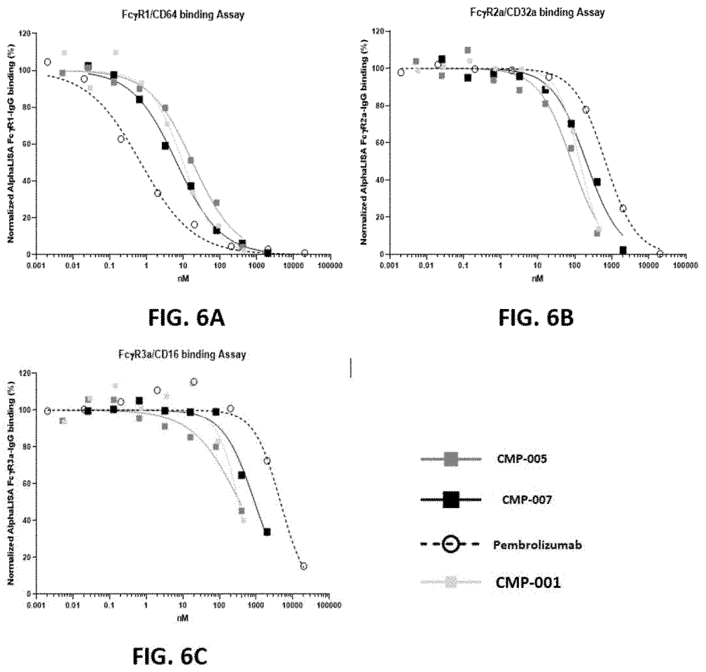

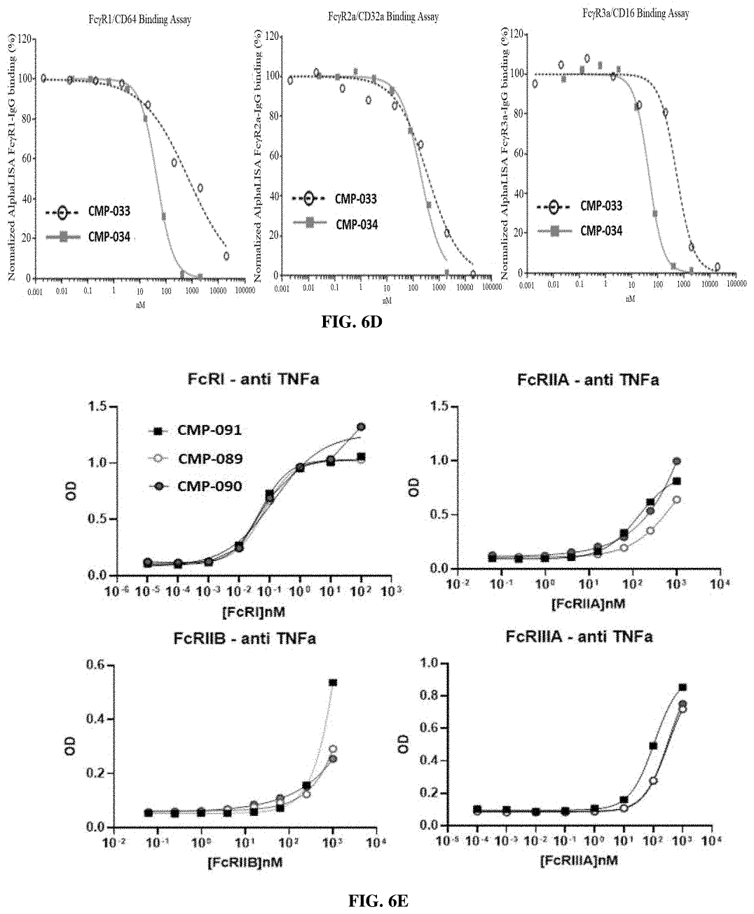

### Example 5: Human FcRg Binding Assays of Immunoconjugates (FIGS. 6A-E)

The interaction of the unmodified and of conjugated antibodies with human Fc gamma receptors I (FcγRI/CD64), with human Fc gamma receptors II (FcγRIIa/CD32), and with low affinity human Fc gamma receptors III FcγR3a/CD16 V158 were measured using the AlphaLISA® Human FcγR1/CD64 (AL3081C), FcγRIIa/CD32a 167H (AL3086C), and FcγRIII/CD16 176P/F158 (AL3471HV) binding Kits respectively (Perkin Elmer, Schwerzenbach, Switzerland).

The AlphaLISA detection of Fc gamma Receptors and IgG binding uses human IgG Fc region coated AlphaLISA® acceptor beads to interact with biotinylated human FcγRI, FcγRIIa, or FcγRIIIa captured on Streptavidin-coated donor beads. When reference IgG binds to Fc gamma receptors, donor and acceptor beads come into proximity enabling the transfer of singlet oxygen that triggers a cascade of energy transfer reactions in the acceptor beads, resulting in a sharp peak of light emission at 615 nm. Addition of a free IgG antibodies into the alphaLISA mixture creates a competition for the binding of Fc gamma Receptors to the reference IgG Fc region resulting in a loss of signal.

Briefly, test molecules were measured in serial dilutions starting at 5 uM down to 4 pM and incubated with AlphaLISA® reaction mixture consisting of 40 ug/ml of human IgG Fc conjugated Acceptor beads, and 40 ug/ml of Streptavidin coated Donor beads and of recombinant biotinylated human FcγRI (200 nM), FcγRIIa (120 nM), or FcγRIIIa (8 nM). After 90 min at 23° C. in the dark, AlphaLISA® signal was measured on an EnSpire plate reader (Excitation at 680 nm, Emission at 615 nm) from Perkin Elmer (Schwerzenbach, Switzerland).

FIG. 6A shows plots measuring ability of the unmodified and of conjugated anti-PD1 antibodies to bind to human Fc gamma receptor I (CD64), with the figure showing mean AlphaLISA® FcγRI-IgG signal on the y-axis and dosage of the unmodified and of conjugated anti-PD1 antibodies on the x-axis. The compositions tested in this figure are Pembrolizumab (CMP-004) and CMP-005, CMP-007, CMP-001.

FIG. 6B shows plots measuring ability of the unmodified and of conjugated anti-PD1 antibodies to bind to human Fc gamma receptor IIa (CD32a), with the figure showing mean AlphaLISA® FcγRIIa-IgG signal on the y-axis and dosage of the unmodified and of conjugated anti-PD1 antibodies on the x-axis. The compositions tested in this figure are Pembrolizumab (CMP-004), CMP-005, CMP-007, and CMP-001.

FIG. 6C shows plots measuring ability of the unmodified and of conjugated anti-PD1 antibodies to bind to human Fc gamma receptor IIIa (CD16), with the figure showing mean AlphaLISA® FcγRIIIa-IgG signal on the y-axis and dosage of the unmodified and of conjugated anti-PD1 antibodies on the x-axis. The compositions tested in this figure are Pembrolizumab CMP-005, CMP-007, and CMP-001.

FIG. 6D shows plots measuring ability of the unmodified and of conjugated anti-PD-L1 antibodies to bind to human Fc gamma receptors CD64 (left), CD32a (center), and CD16 (right), with each plot showing mean AlphaLISA® signal on the y axis and dosage of the unmodified and of conjugated anti-PD-L1 antibodies on the x-axis. The constructs tested in this figure are CMP-033 (Durvalumab) and CMP-034.

FIG. 6E shows plots measuring ability of the unmodified and of conjugated anti-TNFα antibodies to bind to human Fc gamma receptors FcRI (top left), FcRIIa (top right), FcRIIb (bottom left), and FcRIIIa (bottom right), with each plot showing mean AlphaLISA® signal on the y axis and dosage of the unmodified and of conjugated anti-TNFα antibodies on the x-axis. The constructs tested in this figure are CMP-091 (biosimilar adalimumab), CMP-089, and CMP-090.

The ability of a variety of immunoconjugates provided herein and corresponding parent antibodies to bind the tested Fcg receptors is shown in Table 13 below. The values represent the affinity constant (k_{d}) as measured according the above or analogous assays.

TABLE 13
Fc gamma recptor binding

| | | Antigen | | | | | | | |
|---|---|---|---|---|---|---|---|---|---|
| | | bond | Conju- | | | FcgRI | FcgRIIa | FcgRIIb | FcgRIIIa |
| Composi- | | by | gated | | Conjuga- | CD64 | CD32a | CD32b | CD16 |
| tion | Antibody | antibody | protein | DAR | tion site | (nM) | (nM) | (nM) | (nM) |
| CMP-004 | Pembrolizumab | PD-1 | — | 0 | — | 0.488 | 160.2 | 209.3 | 289 |
| CMP-001 | Pembrolizumab | PD-1 | CMP- | 1 | K248 | 1.265 | 313.7 | 744.5 | 339.5 |
| | | | 003 | | | | | | |
| CMP-005 | Pembrolizumab | PD-1 | CMP- | 1.5 | K248 | NT | NT | NT | NT |
| | | | 003 | | | | | | |
| CMP-007 | Pembrolizumab | PD-1 | CMP- | 2 | K248 | 18.57 | 314.2 | 780.2 | 390 |
| | | | 003 | | | | | | |
| CMP-103 | Pembrolizumab | PD-1 | CMP- | 1 | K288 | 1.261 | 488.9 | 2082.3 | 609.05 |
| | | | 003 | | | | | | |
| CMP-025 | Pembrolizumab | PD-1 | CMP- | 2 | K288 | 6.128 | 767.9 | 5084.6 | 1055.5 |
| | | | 003 | | | | | | |
| CMP-012 | Pembrolizumab | PD-1 | CMP- | 1 | K248 | NT | NT | NT | NT |
| | | | 010 | | | | | | |
| CMP-018 | Pembrolizumab | PD-1 | CMP- | 2 | K248 | NT | NT | NT | NT |
| | | | 010 | | | | | | |
| CMP-013 | J43 | PD-1 | — | 0 | — | 0.105 | 128.1 | 349.6 | 193.1 |
| CMP-014 | J43 | PD-1 | CMP- | 1 | K248 | 0.13 | 1298 | 245.8 | 404.8 |
| | | | 003 | | | | | | |
| CMP-019 | J43 | PD-1 | CMP- | 2 | K248 | 0.107 | 1270 | 737.4 | 549.4 |
| | | | 003 | | | | | | |
| CMP-017 | J43 | PD-1 | CMP- | 1 | K248 | NT | NT | NT | NT |
| | | | 010 | | | | | | |
| CMP-101 | J43 | PD-1 | CMP- | 2 | K248 | NT | NT | NT | NT |
| | | | 010 | | | | | | |
| CMP-024 | Humanized J43 | PD-1 | CMP- | 1 | K248 | NT | NT | NT | NT |
| | | | 003 | | | | | | |
| CMP-105 | LZM-009 | PD-1 | — | 0 | — | 0.248 | 904.6 | 543.6 | 921.5 |
| CMP-016 | LZM-009 | PD-1 | CMP- | 1 | K248 | 3.81 | 1142 | 4709 | 481.1 |
| | | | 003 | | | | | | |
| CMP-027 | LZM-009 | PD-1 | CMP- | 2 | K248 | NT | NT | NT | NT |
| | | | 003 | | | | | | |
| CMP-102 | LZM-009 | PD-1 | CMP- | 1 | K288 | 2.298 | 1179 | 6683 | 272.5 |
| | | | 003 | | | | | | |
| CMP-028 | LZM-009 | PD-1 | CMP- | 1.9 | K288 | 11.99 | 1873 | 3341 | 5407 |
| | | | 003 | | | | | | |
| CMP-032 | generic | CD20 | — | — | — | 0.2332 | 739.7 | 879.9 | 133.3 |
| | rituximab | | | | | | | | |
| CMP-030 | generic | CD20 | CMP- | 1 | K248 | .3007 | 685.8 | 995.9 | 339.4 |
| | rituximab | | 003 | | | | | | |
| CMP-033 | durvalumab | PD-L1 | | | | See | See | NT | See |
| | | | | | | FIG. | FIG. | | FIG. |
| | | | | | | 6D | 6D | | 6D |
| CMP-034 | durvalumab | PD-L1 | CMP- | 1.6 | K248 | See | See | NT | See |
| (CMP-034) | | | 003 | | | FIG. | FIG. | | FIG. |
| | | | | | | 6D | 6D | | 6D |
| CMP-039 | pembrolizumab | PD-1 | CMP- | 1 | K248 | NT | NT | NT | NT |
| | | | 095 | | | | | | |
| CMP-040 | pembrolizumab | PD-1 | CMP- | 2 | K248 | NT | NT | NT | NT |
| | | | 095 | | | | | | |
| CMP-041 | LMZ-009 | PD-1 | CMP- | 1 | K248 | NT | NT | NT | NT |
| | | | 095 | | | | | | |
| CMP-096 | LMZ-009 | PD-1 | CMP- | 2 | K248 | NT | NT | NT | NT |
| | | | 095 | | | | | | |
| | Biosimilar | TNFa | — | — | — | See | See | See | See |
| | adalimumab | | | | | FIG. | FIG. | FIG. | FIG. |
| | | | | | | 6E | 6E | 6E | 6E a |
| CMP-089 | Biosimilar | TNFα | CMP- | 1 | K248 | See | See | See | See |
| | adalimumab | | 086 | | | FIG. | FIG. | FIG. | FIG. |
| | | | | | | 6E | 6E | 6E | 6E |
| CMP-090 | Biosimilar | TNFα | CMP- | 2 | K248 | See | See | See | See |
| | adalimuma | | 086 | | | FIG. | FIG. | FIG. | FIG. |
| | | | | | | 6E | 6E | 6E | 6E |
| | Biosimilar | TNFa | — | — | — | NT | NT | NT | NT |
| | Infliximab | | | | | | | | |
| CMP-092 | Biosimilar | TNFα | CMP- | 1 | K248 | NT | NT | NT | NT |
| | Infliximab | | 086 | | | | | | |
| CMP-093 | Biosimilar | TNFα | CMP- | 2 | K248 | NT | NT | NT | NT |
| | Infliximab | | 086 | | | | | | |

NT = not tested. Value in above table 13 are the combined results of different experiments run on different days.

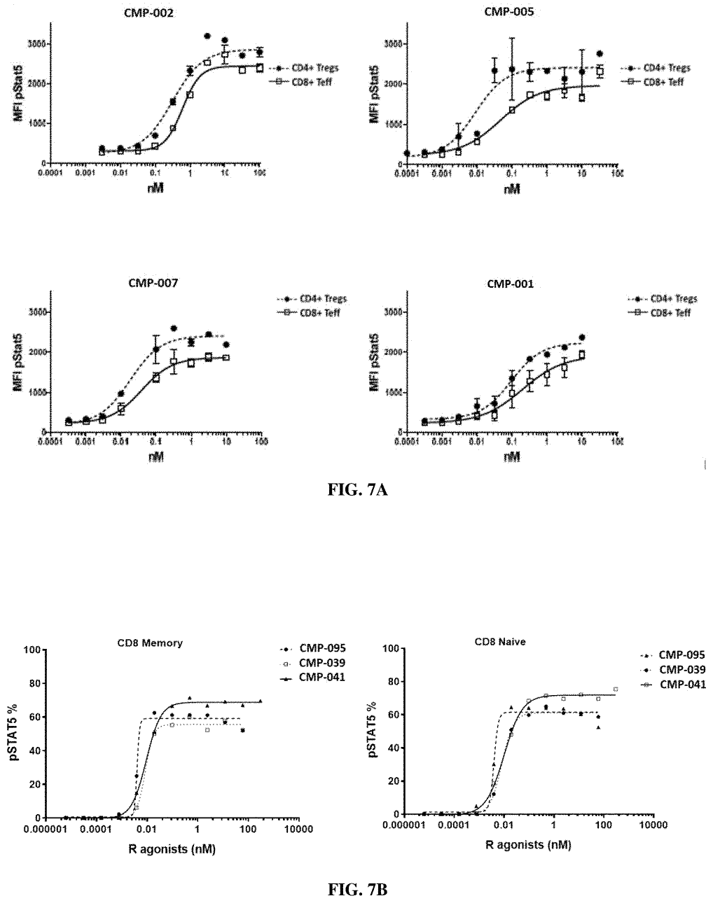

### Example 6: IL2 pStat5 Activation (FIGS. 7-9)

An experiment was performed to determine the effect of various IL-2 polypeptides on human T-cell populations. Primary pan T-cells (CD4+, CD8+ and Tregs Tcells) were obtained from healthy donor buffy coat by peripheral blood mononuclear cell (PBMC) purification using ficoll gradient centrifugation followed by negative isolation with magnetic beads and then cryopreserved until use. Pan T-cells were thawed, allowed them to recover overnight in T-cell medium (RPMI 10% FCS, 1% Glutamin, 1% NEAA, 25 μM βMeoH, 1% NaPyrovate) and after two washing steps with PBS cells were resuspended in PBS. When indicated, cells are pre-incubated during 20 min at 37° C./with 100 nM of unconjugated anti-PD1 antibody Pembrolizumab (CMP-004). Cells were then distributed at 200'000 cells per well and stimulated with 3.16-fold serial dilutions of modified IL-2 polypeptides unconjugated and conjugated to anti-PD1 antibody with a starting concentration of 316 nM down to 3 pM, for 40 min at 37° C./5% CO₂. After incubation, cells were fixed and permeabilized using the Transcription Factor Phospho Buffer kit followed by a surface and intracellular immunostaining for CD4, CD8, CD25, FoxP3, CD45RA and pStat5 to enable cell subsets identification and measure of levels of Stat5 (signal transducer and activator of transcription 5) phosphorylation. The FACS (fluorescence activated cell sorting) measurement was done either with a NovoCyte or a Quanteon Flow Cytometer from Acea.

pStat5 MFI (medium fluorescence intensity) signal for the following T-cell subsets were plotted against concentrations of wild type or of modified IL-2 polypeptides. Half maximal effective concentration (EC₅₀) was calculated based on a variable slope, four parameter analysis using GraphPad PRISM software.

Gating Strategy for T-Cell Subsets Identification

| | |
|---|---|
| T-Reg | CD4+, CD2^{Hi}, FoxP3+ |
| CD8 Teff | CD8+ |
| Naïve CD8 Teff | CD8+, CD45RA+ |
| Memory CD8 | CD8+, CD45RA− |
| Teff | |
| CD4 conv | CD4+, FoxP3− |

TABLE 14
EC₅₀ values of the STAT5 phosphorylation assay in Human primary pan T-cells

| Composition | | | IL-2 | | Conjugation | EC₅₀ | EC₅₀ | CDS/Treg |
|---|---|---|---|---|---|---|---|---|
| Proleukin | Antibody | Linker? | polypeptide | DAR | site | Tregs | CD8 | ratio |
| CMP-003 | — | — | CMP-003 | — | — | 0.385 | 0.818 | 2.1 |
| CMP-010 | — | — | CMP-010 | — | — | 0.386 | 0.941 | 2.4 |
| CMP-001 | Pembrolizumab | | CMP-003 | 1 | K248 | 0.411 | 2.287 | 5.6 |
| CMP-005 | Pembrolizumab | | CMP-003 | 1.5 | K248 | 0.228 | 1.013 | 4.4 |
| CMP-007 | Pembrolizumab | | CMP-003 | 2 | K248 | 0.154 | 0.833 | 5.4 |
| CMP-103 | Pembrolizumab | | CMP-003 | 1 | K288 | 0.081 | 0.996 | 12.3 |
| CMP-025 | Pembrolizumab | | CMP-003 | 2 | K288 | NT | NT | NT |
| CMP-012 | Pembrolizumab | | CMP-010 | 1 | K248 | 7.775 | 10.72 | 1.8 |
| CMP-018 | Pembrolizumab | | CMP-010 | 2 | K248 | 0.229 | 2.211 | 9.7 |
| CMP-014 | J43 | | CMP-003 | 1 | K248 | 3.153 | 3.966 | 1.3 |
| CMP-019 | J43 | | CMP-003 | 2 | K248 | NT | NT | NT |
| CMP-017 | J43 | | CMP-010 | 1 | K248 | 6.705 | 7.902 | 1.2 |
| CMP-101 | J43 | | CMP-010 | 2 | K248 | NT | NT | NT |
| CMP-024 | Humanized J43 | | CMP-003 | 1 | K248 | NT | NT | NT |
| CMP-016 | LZM-009 | | CMP-003 | 1 | K248 | 0.098 | 0.864 | 8.8 |
| CMP-027 | LZM-009 | | CMP-003 | 2 | K248 | NT | NT | NT |
| CMP-102 | LZM-009 | | CMP-003 | 1 | K288 | NT | NT | NT |
| CMP-028 | LZM-009 | | CMP-003 | 1.9 | K288 | NT | NT | NT |
| CMP-030 | generic | CD20 | CMP-003 | 1 | K248 | 1.8 | NT | NT |
| | rituximab | | | | | | | |
| CMP-034 | durvalumab | PD-L1 | CMP-003 | 1.6 | K248 | NT | NT | NT |
| (CMP-034) | | | | | | | | |
| CMP-095 | | | CMP-095 | | | See | See | See |
| | | | | | | FIG. | FIG. | FIG. 7B |
| | | | | | | 7B | 7B | |
| CMP-039 | pembrolizumab | PD-1 | CMP-095 | 1 | K248 | See | See | See |
| | | | | | | FIG. | FIG. | FIG. 7B |
| | | | | | | 7B | 7B | |
| CMP-040 | pembrolizumab | PD-1 | CMP-095 | 2 | K248 | NT | NT | NT |
| CMP-041 | LMZ-009 | PD-1 | CMP-095 | 1 | K248 | NT | NT | NT |
| CMP-096 | LMZ-009 | PD-1 | CMP-095 | 2 | K248 | NT | NT | NT |
| CMP-086 | — | — | CMP-086 | — | — | 2.03 | 4095.72 | |
| CMP-089 | Biosimilar | TNFα | CMP-086 | 1 | K248 | 14.77 | 474.68 | |
| | adalimumab | | | | | | | |
| CMP-090 | Biosimilar | TNFα | CMP-086 | 2 | K248 | 0.66 | 720.27 | |
| | adalimumab | | | | | | | |
| CMP-092 | infliximab | TNFα | CMP-086 | 1 | K248 | NT | NT | NT |
| CMP-093 | infliximab | TNF | CMP-086 | 2 | K248 | NT | NT | NT |

NT: Not Tested. The data provided in the above table 14 was generated from different experiments ran on different days.

FIG. 7A shows plots measuring the effect of the modified IL-2 polypeptides unconjugated and conjugated to the anti-PD1 antibody on the inducement of T_{eff} and T_{reg} cells in an in vitro sample of human T-cells, with the figure showing mean fluorescence intensity for phosphorylated signal transducer and activator of transcription 5 (pSTAT5) on the y-axis and dosage of modified IL-2 polypeptide and immunocytokines on the x-axis. The modified IL-2 polypeptide tested in this figure is CMP-002. The immunocytokines tested in this figure are CMP-005, CMP-007, CMP-001.

FIG. 7B shows plots measuring the effect of synthetic IL-7 (CMP-095), and IL-7/anti-PD1 antibody conjugates (CMP-039, CMP-041) on STAT5 phosphorylation in CD8 naïve and CD8 Memory cells.

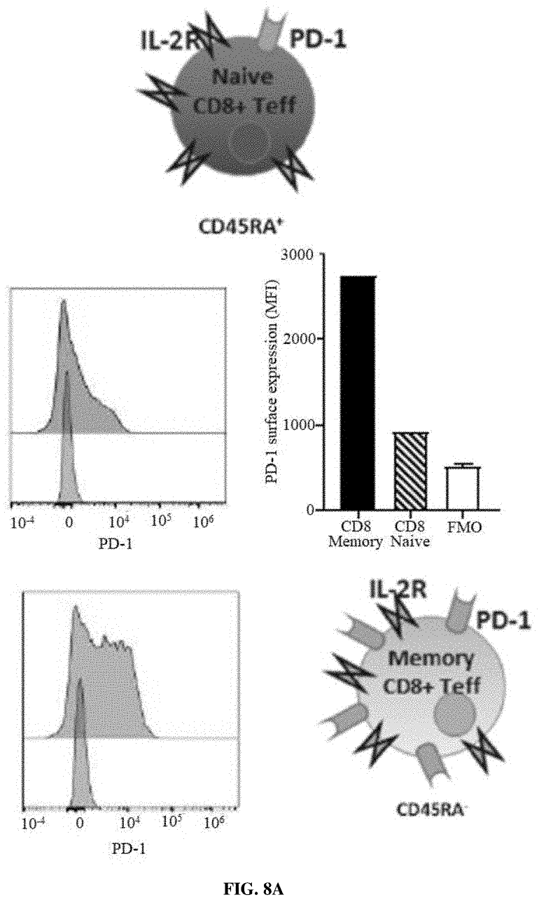

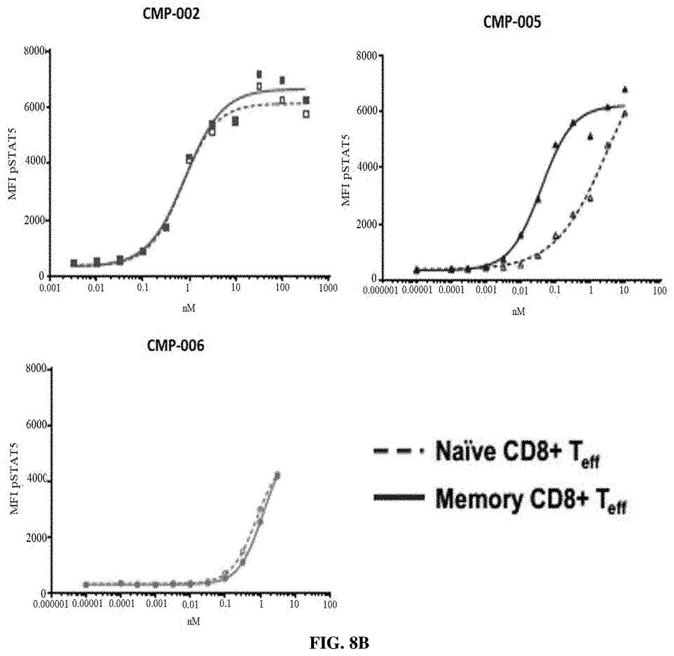

FIG. 8A shows plots measuring the level of surface expression of PD-1/CD279 on resting memory (CD45RA−) and naïve (CD45RA+) CD8+ T_{eff} cells freshly isolate from peripheral blood of healthy donors.

FIG. 8B shows plots measuring the effect of the modified IL-2 polypeptides unconjugated and conjugated to the anti-PD1 antibody on the inducement of resting CD8+ T_{eff} cells in an in vitro sample of human T-cells in the presence or absence of excess amounts of unconjugated anti-PD1 antibody, with the figure showing mean fluorescence intensity for phosphorylated signal transducer and activator of transcription 5 (pSTAT5) on the y-axis and dosage of modified IL-2 polypeptide and immunocytokines on the x-axis. The modified IL-2 polypeptide tested in this figure is CMP-002 and the immunocytokines tested in this figure are CMP-005, and CMP-006 as a control.

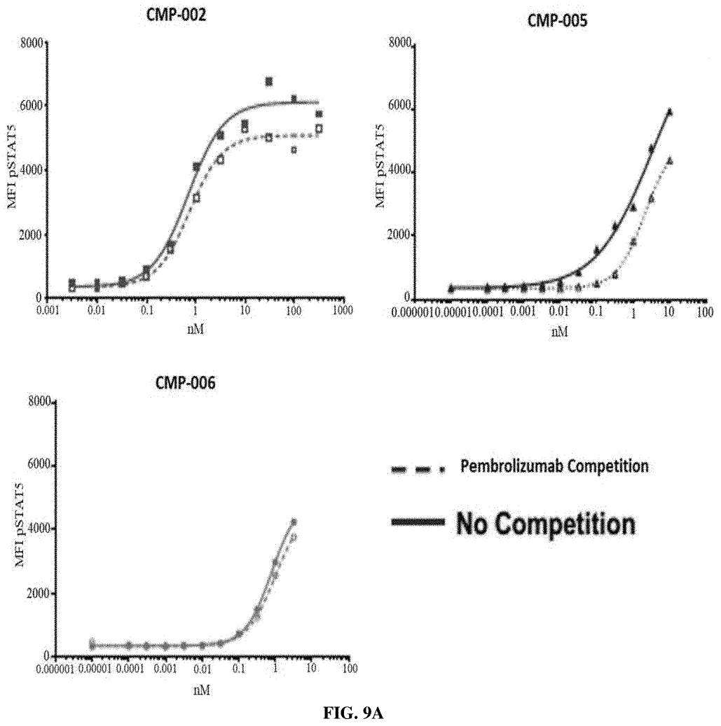

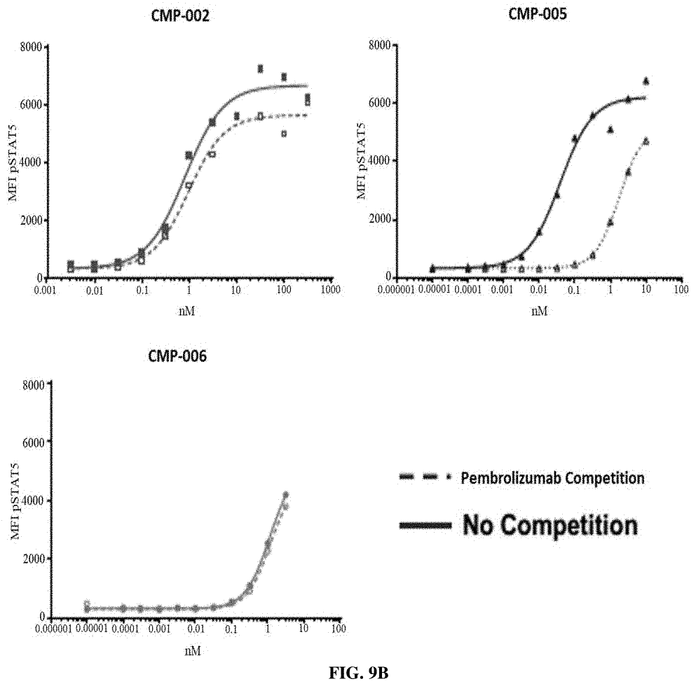

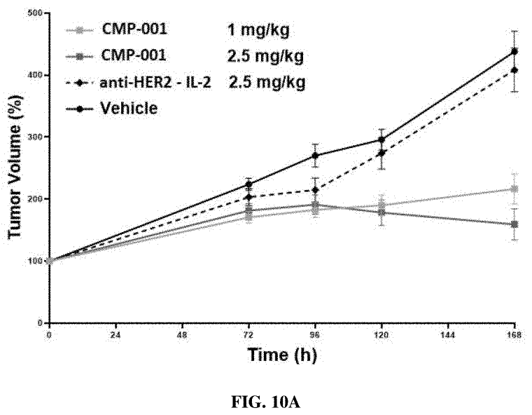

FIG. 9A shows plots measuring the effect of the modified IL-2 polypeptides unconjugated and conjugated to the anti-PD1 antibody on the inducement of resting naïve (CD45RA+) CD8+ T_{eff} cells in an in vitro sample of human T-cells in the presence or absence of excess amounts of unconjugated anti-PD1 antibody CMP-004, with the figure showing mean fluorescence intensity for phosphorylated signal transducer and activator of transcription 5 (pSTAT5) on the y-axis and dosage of modified IL-2 polypeptide and immunocytokines on the x-axis. The modified IL-2 polypeptide tested in this figure is CMP-002 and the immunocytokines tested in this figure are CMP-005 and CMP-006 as a control.

FIG. 9B shows plots measuring the effect of the modified IL-2 polypeptides unconjugated and conjugated to the anti-PD1 antibody on the inducement of resting memory (CD45RA−) CD8+ T_{eff} cells in an in vitro sample of human T-cells in the presence or absence of excess amounts of unconjugated anti-PD1 antibody CMP-004 (Pembrolizumab), with the figure showing mean fluorescence intensity for phosphorylated signal transducer and activator of transcription 5 (pSTAT5) on the y-axis and dosage of modified IL-2 polypeptide and immunocytokines on the x-axis. The modified IL-2 polypeptide tested in this figure is CMP-002 and the immunocytokines tested in this figure are CMP-005 and CMP-006 as a control.

### Example 7: Immunoconjugates Display In Vivo Activities Associated with Both the Antibody and the Conjugated Protein

The various immunoconjugates provided herein were assessed in the in vivo experiments provided below which demonstrate the activity of one or both components of the conjugate, or the synergy of the both components in combination as a conjugate.

### Example 7A—In Vivo Efficacy Study of Anti-PD-1 Antibody/IL-2 Conjugate

An in vivo PK/PD study was performed in mice. Naïve, 6-8 weeks old, BALB/c-hPD1 female mice (GemPharmatech Co, Ltd, Nanjing, China) were inoculated subcutaneously at the left flank with wild type CT26 tumor cells (3×10⁵) in 0.1 mL of PBS for tumor development. The animals were randomized (using an Excel-based randomization software performing stratified randomization based upon tumor volumes), and treatment started when the average tumor volume reached approximately 186 mm³. Animals treated with Composition A received a single 10 mL/kg bolus intravenous (i.v.) injection of 1, and 2.5 mg/kg of PD-1 antibody conjugated with modified IL-2 polypeptide. Animals treated with control Her2-targeted immunocytokine Composition O (Trastuzumab antibody conjugated to IL-2 polypeptide) received a single 10 mL/kg bolus intravenous (i.v.) injection of 2.5 mg/kg of anti-Her2 antibody conjugated with modified IL-2 polypeptide. After inoculation, the animals were checked daily for morbidity and mortality. At the time, animals were checked for effects on tumor growth and normal behavior such as mobility, food and water consumption, body weight gain/loss (body weights were measured twice weekly), eye/hair matting and any other abnormal effect. Tumor sizes were measured three times a week in two dimensions using a caliper, and the volume was expressed in mm³ using the formula: V=0.5 a×b² where a and b are the long and short diameters of the tumor, respectively. Death and observed clinical signs were recorded on the basis of the numbers of animals within each subset.

Pharmacokinetic study included 9 time points (5 min, 1 h, 6 h, 12 h, 24 h, 72 h, 96 h, 120 h, 168 h) with 3 mice sampled per time points. At indicated time points, blood samples were collected in the presence of EDTA either via tail vein sampling or via cardiac puncture (end-point). In addition, 72 h, 96 h, 120 h, 168 h after injection 3 mice per group were sacrificed and tumor samples were collected.

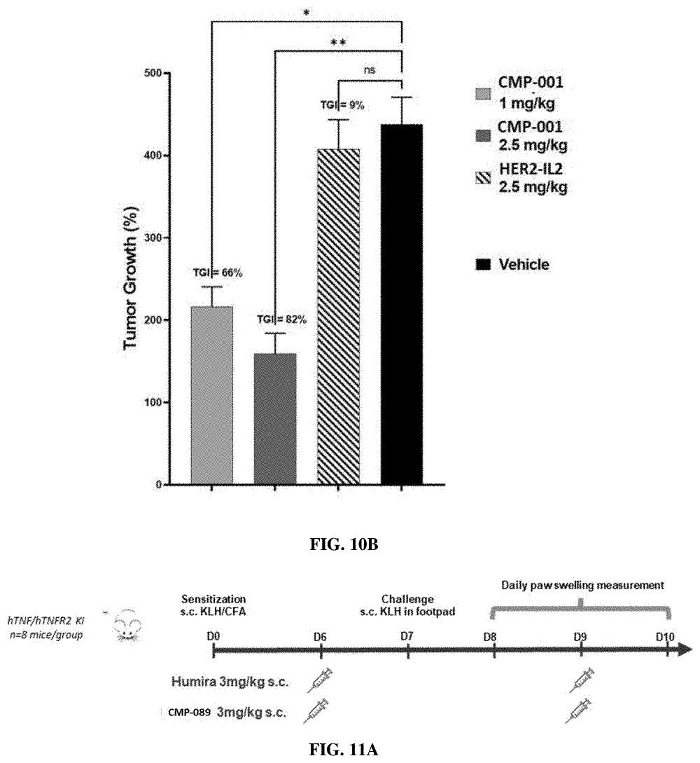

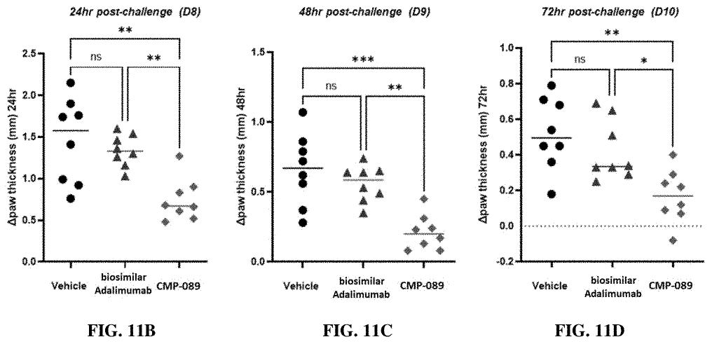

FIG. 10A shows a plot describing the effect of PD-1 targeted and untargeted immunocytokines on the growth of CT26 syngeneic colon carcinoma tumors in hPD1 humanized BALB/c mice. The immunocytokine tested in this figure is Composition A tested as a single agent at 1, and 2.5 mg/kg after a single injection schedule. Control Her2-targeted immunocytokine Composition O (Trastuzumab antibody conjugated to IL-2 polypeptide) was also tested at 2.5 mg/kg. (mean±SEM).

FIG. 10B shows a bar chart describing the effect PD-1 targeted and untargeted immunocytokines on the growth of CT26 syngeneic colon carcinoma tumors in hPD1 humanized BALB/c mice 7 days after treatment. The immunocytokine tested in this figure is Composition A tested as a single agent at 1, and 2.5 mg/kg after a single injection schedule. Control Her2-targeted immunocytokine Composition O (Trastuzumab antibody conjugated to IL-2 polypeptide) was also tested at 2.5 mg/kg. (mean±SEM; ** one-way ANOVA P-value<0.001).

### Example 7B: CMP-089 Suppresses KLH-Induced Delayed Type Hypersensitivity

The ability of CMP-089 to suppress antigen-driven ear inflammation was tested in a mouse delayed type hypersensitivity (DTH) model. DTH represents a local T effector recall response to a previously encountered antigen. Here, mice are first sensitized to keyhole limpet hemocyanin (KLH) by immunization s.c. with KLH and then rechallenged several days later with an intradermal injection of the same antigen into the ear resulting in local tissue inflammation and swelling.

To demonstrate that CMP-089 can suppress antigen-driven ear inflammation in this model (a function observed for the IL-2 syntenin CMP-086 conjugated to a 30 kDa PEG group (data not shown)), when conjugated to biosimilar Adalimumab), hTNF/hTNFR2 KI mice (12-13 weeks old) were randomly allocated to experimental groups (n=8/group). On Day 0, animals were administered with an emulsion of 100 μg KLH in CFA by s.c. injection at the two flanks near the base of the tail. Vehicle, Humira™ or CMP-089 were administered at 3 mg/kg s.c. on days 6 and 9. Following baseline measurements of paw thickness (right and left paw) on day 7, all animals were challenged with an intra-dermal injection of 20 μg/20 μl KLH in saline in the left footpad, while the right footpad was injected subcutaneously with 20 μl saline to serve as control. Paw thickness measurements of left and right paws were performed 24 h (d8), 48 h (d9) and 72 h (d10) post-challenge. A schematic of the experimental protocol is shown in FIG. 11A. CMP-089 significantly suppressed paw inflammation at all time points compared to vehicle, whereas biosimilar Adalimumab had no significant effect (see FIG. 11B for change in paw thickness data at 24 hours post challenge, FIG. 11C for change in paw thickness data at 48 hours post challenge, and FIG. 11D for change in paw thickness data at 72 hours post challenge), thus demonstrating that the IL-2 syntenin was functional in vivo in suppressing DTH when conjugated to Humira™.

### Example 7C—Anti-Tumor Efficacy of PD1:IL-7 Immunocytokines—In Vivo Tumor Growth Inhibition

An experiment was performed as described below in order to assess pharmacokinetic (PK) and pharmacodynamics (PD) properties of the immunocytokines provided herein (e.g., CMP-041), as well as to assess anti-tumor efficacy.

Transgenic BALBc-hPD1 mice (BALB/cJGpt-Pdcd1em1Cin(hPDCD1)/Gpt), which are genetically modified with knock-in of human PD-1 as sold by GemPharmatech (Cat #T002726) were implanted with the syngeneic CT26 tumor model treated with various dose combinations of immunocytokines with flow cytometric and relative tumor volume readouts.

Six groups of seven mice were used in the treatment study according to the Table 15 below.

TABLE 15
Study Design

| | Test | No of | Dosage | Dosing | Dosing |
|---|---|---|---|---|---|
| Group | Article | animals | (mg/kg) | Route | Frequency |
| G1 | Vehicle | 7 | — | sc | QWx2 |
| G2 | Livzon PD1 | 7 | 10 | sc | QWx2 |
| | AB | | | | |
| G3 | Livzon | 7 | 1 | sc | QWx2 |
| | PD1:IL7wt | | | | |
| | (CMP-041) | | | | |
| G4 | Livzon | 7 | 3 | sc | QWx2 |
| | PD1:IL7wt | | | | |
| | (CMP-041) | | | | |
| G5 | Livzon | 7 | 10 | sc | QWx2 |
| | PD1:IL7wt | | | | |
| | (CMP-041) | | | | |

QWx2 dosing = once weekly dosing, with two total doses administered.

For flow cytometry and cytokine analysis of blood samples, blood were treated with dipotassium ethylenediaminetetraacetic acid (K2-EDTA). Samples used for cytokine analysis were centrifuged twice to ensure removal of all cell debris; 5 minutes at 300 g before being transferred into fresh tubes and centrifuged again for 5 minutes at 2000 g.

For analysis of tumor samples, the tumor was first weighed, with samples for PK analysis and cytokine measurements snap frozen for analysis. Plasma intended for use in cytokine analysis was centrifuged twice to ensure removal of all cell debris; 5 minutes at 300 g before being transferred into fresh tubes and centrifuged again for 5 minutes at 2000 g Fresh samples were used for flow cytometry.

Flow cytometry was conducted using two panels (A or B)_of antibodies for cellular markers as follows.

Panel A (where the m prefix indicates murine specific antibodies): mCD3epsilon-FITC (BD 553062), mCD45-AF700 (Biolegend 103127), mCD44-APC/Cy7 (Biolegend 103028), mCD4-eF450 (ThermoFisher 48-0042-82), mCD69-BV 605 (Biolegend 104530), mCD8-BV650 (Biolegend 100742), mCD25-PE (BD 553866), mCD127-PE-Cy7 (Biolegend 121120), mKI67-PerCp/Cy5.5 (Biolegend 652423), mFoxP3-APC (eBioscience 17-5772-B2), Live/dead Aqua Zombie (Biolegend 423102).

Panel B (where the m prefix indicates murine specific antibodies): mCD3epsilon-FITC (BD 553062), mLy6-PerCp/Cy5.5 (Biolegend 127615), mCD45-AF700 (Biolegend 103127), mCD11b-APC/Cy7, mF4/80-BV421 (Biolegend 123137), mLy6-BV605 (BD 563011), mCD206-APC (Thermo Fisher 17-2061-82), Live/dead Aqua Zombie (Biolegend 423102).

Body weight measurements of mice over the course of the study were measured and for each group, weights of the animals remained relatively constant, indicating each construct was well tolerated at the doses administered.

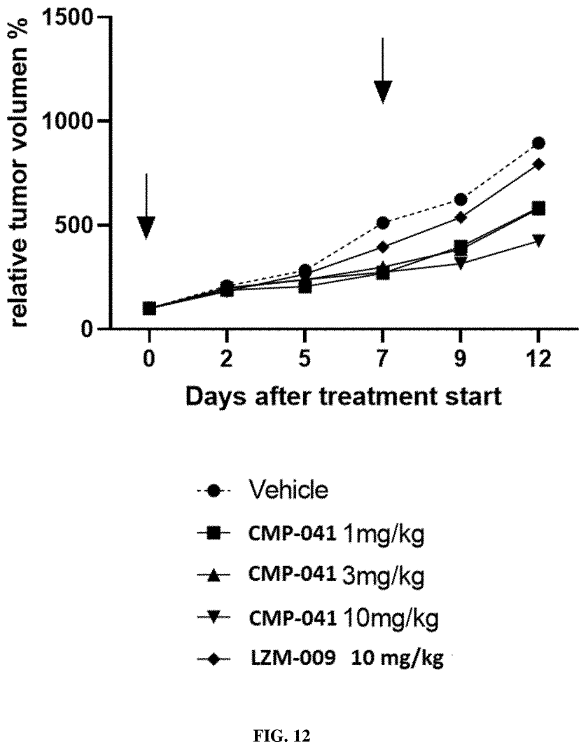

Relative tumor volume for the various groups during the course of the study is shown in FIG. 12, which shows a comparison of vehicle, LZM-009, and three doses of CMP-041 (1 mg/kg, 3 mg/kg, and 10 mg/kg once weekly for two weeks). CMP-041 was observed to display dose dependent efficacy in reducing tumor volume and greater efficacy than unmodified antibody alone, even at a lower dose.

### Example 7D: Tumor Growth Inhibition Study with Anti-CD20 Antibody Conjugates in C57BL/6 Mice

In order to assess anti-tumor effects of CMP-030 immunocytokines, an in vivo study was performed in C57BL/6 mice bearing EL4-hCD20 SQ tumors according to the parameters provided below.

Cell culture: The EL4 murine thymic lymphoma cell line was purchased from ATCC. Cells are maintained in vitro as monolayer culture in DMEM supplemented with 10% heat inactivated FBS at 37° C. in a humidified atmosphere of 5% CO2. B-hCD20 EL4 cells that were genetically modified to overexpress human CD20 coding sequence in EL4 cells are provided by Biocytogen Pharmaceuticals (Beijing) Co., Ltd. To test in vivo efficacy of CD20 immunocytokines, C57BL/6 mice were subcutaneously injected with B-hCD20 EL4 tumor cells (2×105) in 0.1 mL PBS in the right hind flank for tumor development. 48 tumor-bearing animals were randomly enrolled into six study groups when the mean tumor size reaches approximately 50-100 mm3. Each group tested consisted of 8 mice. The groups were as follows: G1—Vehicle; G2—biosimilar Rituximab administered twice weekly at 10 mg/kg; G3—CMP-030 administered once weekly at 1.25 mg/kg; G4—CMP-030 administered once weekly at 2.5 mg/kg; G5—CMP-030 administered twice weekly at 1.25 mg/kg; and G6—CMP-030 administered once weekly at 5 mg/kg. The dosing in G5 and G6 was not well tolerated and the study discontinued for those groups. All dosing was performed via intraperitoneal administration.

Body weight of mice from each group were measured at various time points post administration (Day 0, Day 2, Day 3, Day 7 (second administration for biosimilar Rituximab, and Day 10)). Mice administered CMP-030 showed an initial modest drop in body weight (˜10% at Day 3) after the first dose which appeared to recover after several days (by Day 7).

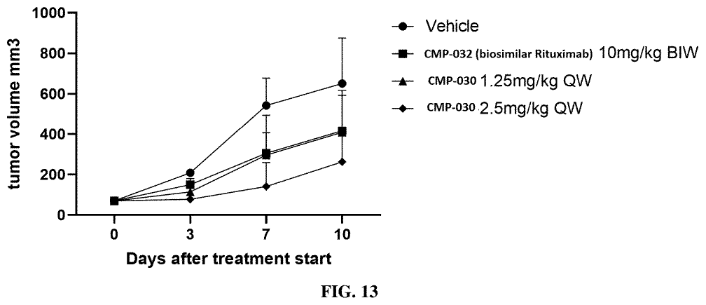

FIG. 13 shows tumor volume for the indicated groups at various time points post-administration. The once weekly dosing of CMP-030 at 1.25 mg/kg performed comparable to twice weekly biosimilar rituximab at 10 mg/kg. Notably, the 2.5 mg/kg of CMP-030 once weekly group showed superior tumor growth inhibition in this model at all time points. This data support a synergistic effect of the conjugate composition.

### Example 8—Synthesis of IL-7

### General Methods of IL-7 Synthesis

General strategy: Synthetic IL-7 polypeptides are synthesized by ligating individual peptide segments prepared by solid phase peptide synthesis (SPPS). Briefly, peptide segments (Seg1, Seg2, Seg3 and Seg4) were prepared using SPPS, and any desired modification to the amino acid sequence of wild-type IL-7 (SEQ ID NO: 186) was incorporated during the synthesis. After purification of the individual fragments, IL-7-Seg1 and IL-7-Seg2 were ligated together, as well as IL-7-Seg3 and IL-7-Seg4. The resulting IL-7-Seg12 and IL-7-Seg34 were purified and ligated together to afford IL-7-Seg1234 with cysteines protected with Acm groups (IL-7-Seg1234-Acm). The Acm groups of IL-7-Seg1234-Acm were then universally deprotected and purified to afford synthetic IL-7 linear protein. The resulting synthetic IL-7 linear proteins were then rearranged and folded. Individual peptides are synthesized on an automated peptide synthesizer using the methods described below.

Materials and solvents: Fmoc-amino acids with suitable side chain protecting groups for Fmoc-SPPS, resins polyethylene glycol derivatives used for peptide functionalization and reagents were commercially available and were used without further purification. HPLC grade CH₃CN from was used for analytical and preparative RP-HPLC purification.

Loading of protected ketoacid derivatives (segment 1-3) on amine-based resin: 5 g of Rink-amide MBHA or ChemMatrix resin (1.8 mmol scale) was swollen in DMF for 30 min. Fmoc-deprotection was performed by treating the resin twice with 20% piperidine in DMF (v/v) at r.t. for 10 min. followed by several washes with DMF. Fmoc-AA-protected-α-ketoacid (1.8 mmol, 1.00 equiv.) was dissolved in 20 mL DMF and pre-activated with HATU (650 mg, 1.71 mmol, 0.95 equiv.) and DIPEA (396 μL, 3.6 mmol, 2.00 equiv.). The reaction mixture was added to the swollen resin. It was let to react for 6 h at r.t. under gentle agitation. The resin was rinsed thoroughly with DMF. Capping of unreacted amines on the resin was performed by addition of a solution of acetic anhydride (1.17 mL) and DIPEA (2.34 mL) in DMF (20 mL). It was let to react at r.t. for 15 min under gentle agitation. The resin was rinsed thoroughly with DCM followed by diethyl ether and dried. The loading of the resin was measured (0.25 mmol/g) following the method described in M. Gude, et al. (2003) Lett. Pept. Sci., 9, 203.

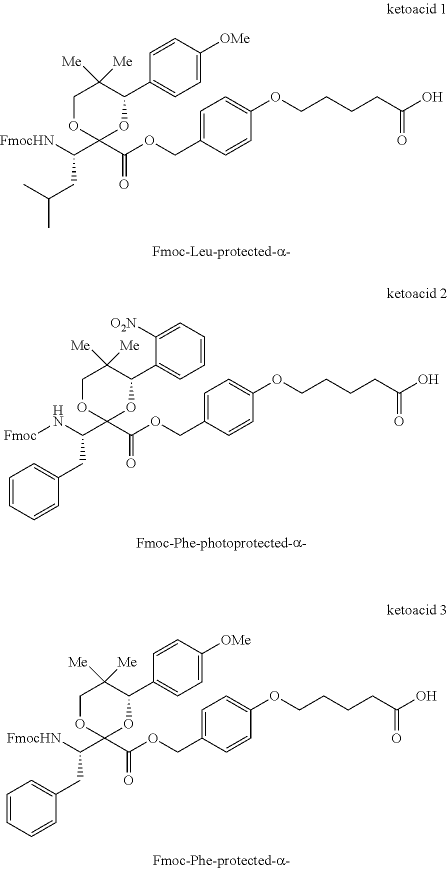

### Protected Ketoacid Used

Solid-phase peptide synthesis (SPPS): The peptide segments were synthesized on an automated peptide synthesizer using Fmoc-SPPS chemistry. The following Fmoc-amino acids with side-chain protecting groups were used: Fmoc-Ala-OH, Fmoc-Arg(Pbf)-OH, Fmoc-Asn(Trt)-OH, Fmoc-Asp(OtBu)-OH, Fmoc-Cys(Acm)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Nle-OH, Fmoc-Phe-OH, Fmoc-Pro-OH, Fmoc-Ser(tBu)-OH, Fmoc-Thr(tBu)-OH, Fmoc-Tyr (tBu), Fmoc-Trp(Boc)-OH, Fmoc-Val-OH and Fmoc or Boc-Opr-OH (Opr=5-(S)-oxaproline). Fmoc-pseudoproline dipeptides were incorporated in the synthesis if necessary. Fmoc deprotection reactions were performed with 20% piperidine in DMF or NMP containing 0.1 M Cl-HOBt (2×2 min). Coupling reactions were performed with Fmoc-amino acid (3.0-8.0 equiv to resin substitution), HCTU or HATU (2.9-8 equiv) as coupling reagents and DIPEA or NMM (6-16 equiv) in DMF or NMP at room temperature. The solution containing the reagents was added to the resin and allowed to react for 15 min, 30 min, or 2 h depending on the amino acid. Double coupling reactions were performed as needed. Unreacted free amines were capped using 20% acetic anhydride in DMF or NMP and 0.8 M NMM in DMF or NMP.

Resin cleavage and side chain deprotection. Once the peptide synthesis was completed, the peptides were cleaved from the resin using a cleavage cocktail at room temperature for 2 h. The resin was filtered off, and the filtrate was concentrated and treated with cold diethyl ether, triturated and centrifuged. The ether layer was carefully decanted, the residue was suspended again in diethyl ether, triturated and centrifuged. Ether washings were repeated twice. The resulting crude peptide was dried under vacuum and stored at −20° C. An aliquot of the solid obtained was solubilized in 1:1 CH₃CN/H₂O with 0.1% TFA (v/v) and analyzed by analytical RP-HPLC using C18 column (3.6 μm, 150×4.6 mm) at 60° C. The molecular weight of the product was identified using MALDI-TOF or LC-MS.

Purification of the peptides: Peptide segments, ligated peptides and linear proteins were purified by RP-HPLC. Different gradients were applied for the different peptides. The mobile phase was MilliQ-H₂O with 0.1% TFA (v/v) (Buffer A) and HPLC grade CH₃CN with 0.1% TFA (v/v) (Buffer B). Preparative HPLC was performed on a (50×250 mm) or on a C18 column (50×250 mm) at a flow rate of 40 mL/min at 40° C. or 60° C.

Purification: The peptide fragments purification was performed on standard preparative HPLC instruments. Preparative HPLC was performed on C18 column (5 μm, 110 Å, 50×250 mm) at a flow rate of 40 mL/min on C18 column (5 μm, 110 Å, 20×250 mm) or C4 column (5 μm, 300 Å, 20.0×250 mm) at a flow rate of 10 m/min. For both columns, room temperature, 40° C., or 60° C. were used during the purification. The mobile phase was MilliQ-H₂O with 0.1% TFA (v/v) (Buffer A) and HPLC grade CH₃CN with 0.1% TFA (v/v) (Buffer B).

Characterization of the peptides: Peptides and proteins were characterized by high resolution Fourier-transform mass spectrometry (FTMS) using a SolariX (9.4 T magnet) spectrometer (Bruker, Billerica, USA) equipped with a dual ESI/MALDI-FTICR source, using 4-hydroxy-α-cyanocinnamic acid (HCCA) as matrix. Peptides segments, ligated peptides and linear proteins were analyzed by RP-HPLC using analytical HPLC instruments using standard C4 column (3.6 μm, 150×4.6 mm) at room temperature or standard C18 column (3.6 μm, 150×4.6 mm) with a flow rate of 1 mL/min at 50° C. The peptide fragments were analyzed using a gradient of 20% B to 95% B in 12 min (Method A), 10% B to 85% B in 12 min (Method B) or 10% B to 95% B in 12 min (Method C).

### Preparation of a Synthetic IL-7 Polypeptide of SEQ ID NO: 187.

### Segment 1: IL-7(1-34)-Leu-α-Ketoacid

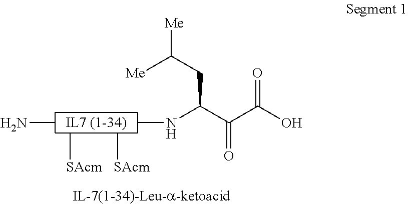

Segment 1 was synthesized on a 0.2 mmol scale on Rink Amide MBHA resin pre-loaded with Fmoc-Leu-protected-α-ketoacid (description in the general methods) (0.8 g) with a substitution capacity of ˜0.25 mmol/g.

Automated Fmoc-SPPS of Segment 1: The peptide elongation cycles including amino acid coupling, capping and Fmoc deprotection were performed as described in the general methods.

After the peptide elongation, the resin was washed with DCM and dried under vacuum. The mass of the dried peptidyl resin was 1.6 g. The peptide was cleaved from the resin using a mixture of 95:2.5:2.5 TFA:DODT:H₂O (10 mL/g resin) at room temperature for 2.0 h. The compound was precipitated as described in the general methods. 702 mg of crude peptide were obtained.

Purification of crude Segment 1 was performed by preparative HPLC using a C18 column (5 μm, 110 Å, 250×50 mm) at a flow rate of 40 m/min at 60° C. with a gradient of 30 to 80% B in 25 min. The fractions containing the purified product were pooled and lyophilized to obtain Segment 1 as a white solid in 94% purity. The isolated yield based on the resin loading was 17% (135 mg). MS (ESI): C₁₇₁H₂₈₁N₄₃O₆₃S₂; Average isotope calculated 1337.9940 Da [M+H]³⁺; found: 1337.9933 Da [M+H]³⁺. Retention time (analytical Method A): 5.66 min.

### Segment 2: Opr-IL-7(37-74)-Phe-Photoprotected-α-Ketoacid

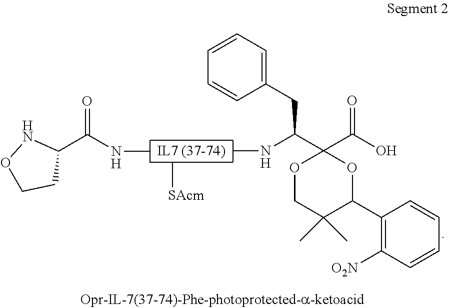

Segment 2 was synthesized on a 0.2 mmol scale on Rink Amide MBHA resin pre-loaded with Fmoc-Phe-photoprotected-α-ketoacid (description in the general methods) with a substitution capacity of 0.25 mmol/g.

The peptide elongation cycles including amino acid coupling, capping and Fmoc deprotection were performed as described in the general methods.

After the peptide elongation, the resin was washed with DCM and diethyl ether and dried under vacuum. The mass of the dried peptidyl resin was 2.2 g. The peptide was cleaved from the resin using a mixture of 95:2.5:2.5 TFA/DODT/H₂O (15 mL/g resin) at room temperature for 2.0 h. The compound was precipitated as described in the general methods. 1.2 g of crude peptide were obtained.

Purification of crude Segment 2 was performed by preparative HPLC using a C18 column (5 μm, 110 Å, 250×50 mm) at a flow rate of 40 m/min at 40° C. using CH₃CN/H₂O with a gradient of 10 to 60% B in 30 min. The fractions containing the purified product were pooled and lyophilized to obtain Segment 2 as a white solid in 97% purity. The isolated yield based on the resin loading was 20% (203 mg). RMS (ESI): C₂₃₄H₃₅₂N₆₅O₆₂S; m/z calculated: 5098.6114 Da [M+H]⁺; found: 5098.6026 Da [M+H]⁺. Retention time (analytical Method A): 6.30 min.

### Segment 3: Fmoc-Opr-IL-7(77-112)-Leu-α-Ketoacid

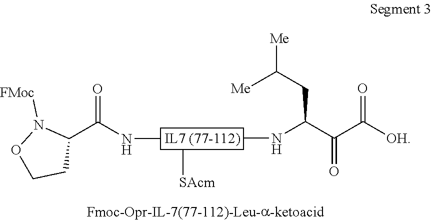

Segment 3 was synthesized on a 0.1 mmol scale on Rink Amide resin pre-loaded with Fmoc-Leu-protected-α-ketoacid (description in the general methods) with a substitution capacity of ˜0.29 mmol/g. 345 mg of resin was swollen in DMF for 15 min.

Automated Fmoc-SPPS of Segment 3: The peptide elongation cycles including amino acid coupling, capping and Fmoc deprotection were performed as described in the general methods.

After the peptide elongation, the resin was washed with DCM and dried under vacuum. The mass of the dried peptidyl resin was 0.94 g. The peptide was cleaved from the resin using a mixture of 95:2.5:2.5 TFA:DODT:H₂O (10 mL/g resin) at room temperature for 2.0 h. The compound was precipitated as described in the general methods. 473 mg of crude peptide were obtained.

Purification of crude Segment 3 was performed by preparative HPLC using a C18 column (5 μm, 110 Å, 50×250 mm) at a flow rate of 40 m/min at 40° C. with a gradient of 10 to 50% B in 40 min. The fractions containing the purified product were pooled and lyophilized to obtain Segment 3 as a white solid in 98% purity. The isolated yield based on the resin loading was 25% (107 mg). HRMS (ESI): C₁₉₃H₃₁₅N₅₁O₅₇S; Average isotope calculated 4294.0030 Da [M+H]; found: 4293.2962 Da. Retention time (analytical Method B): 6.29 min.

### Segment 4: Opr-IL-7(115-152)

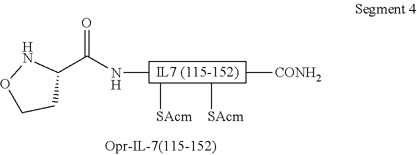

Segment 4 was synthesized on a 0.1 mmol scale on Rink Amide MBHA resin with a substitution capacity of ˜0.34 mmol/g. 294 mg of resin was swollen in DMF for 15 min. Automated Fmoc-SPPS of Segment 4: The peptide elongation cycles including amino acid coupling, capping and Fmoc deprotection were performed as described in the general methods.

The resin was washed with DCM and dried under vacuum. The mass of the dried peptidyl resin was 725 mg. The peptide was cleaved from the resin using a mixture of 92.5:2.5:2.5:2.5 TFA:TIPS:DODT:H₂O (10 mL/g resin) at room temperature for 2.0 h. The compound was precipitated as described in the general methods. 145 mg of crude peptide were obtained.

Purification of crude Segment 4 was performed by preparative HPLC using a Gemini NX-C18 110 Å column (5 μm, 50×250 mm) at a flow rate of 40 mL/min at 40° C. with a gradient of 10 to 50% B in 40 min. The fractions containing the purified product were pooled and lyophilized to obtain Segment 4 as a white solid in 98% purity. The isolated yield based on the resin loading was 8% (40 mg). HRMS (ESI): C₂₁₅H₃₆₁N₆₁O₆₀S₂; Average isotope calculated 4823.6568 Da [M]; found: 4823.6542 Da. Retention time (analytical Method B): 6.15 min.

### Segment 12: IL-7-Seg12 Preparation

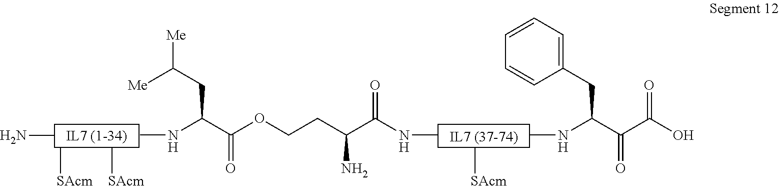

Segment1 (17.5 mg; 4.36 μmol; 1.1 equiv) ketoacid and Segment2 (20 mg; 3.92 μmol; 1.0 equiv) were dissolved in 15 mM DMSO:H₂O (9.5:0.5) containing 0.1 M oxalic acid (241 μL). A very homogeneous liquid solution was obtained. The ligation vial was protected from light and the mixture was heated overnight at 60° C. After completion of the ligation, the mixture was diluted with 1:1 CH₃CN:H₂O with 0.1% TFA (v/v) (4 mL), and the mixture was irradiated at a wavelength of 365 nm for 1.5 h to allow photodeprotection of the C-terminal ketoacid. The reaction mixture was further diluted with 1:1 CH₃CN/H₂O (q.s. 10 mL) with TFA (0.1%, v/v).

The diluted mixture was filtered and injected into preparative HPLC. Crude ligated peptide was purified by preparative HPLC using a C18 column (5 μm, 50×250 mm) at a flow rate of 40 mL/min at 60° C., with a 2-step gradient: 10 to 40% B in 5 min, then 40 to 70% Bin 30 min. The fractions containing the purified product were pooled and lyophilized to obtain Segment 12 as a white solid in 98% purity. The isolated yield was 38% (13.1 mg). MS (ESI): C₃₉₃H₆₁₉N₁₀₇O₁₂₀S₃; m/z calculated: 8858.4917 Da [M+H]⁺; found: 8858.4928 Da [M+H]⁺. Retention time (analytical Method B): 5.41 min.

### Segment 34: IL-7-Seg34 Preparation

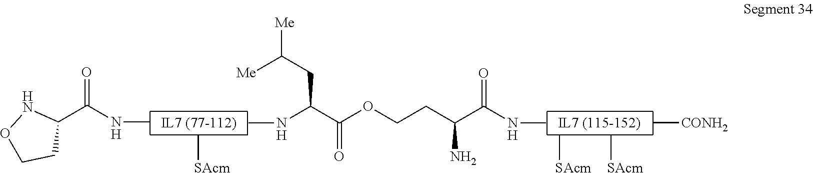

Peptide ketoacid Segment 3 (55.0 mg; 12.8 μmol; 1.2 equiv) and hydroxylamine peptide Segment 4 (51.5 mg; 10.6 μmol; 1.0 equiv) were dissolved in 9:1 DMSO/H₂O containing 0.1 M oxalic acid (530 μL). A very homogeneous liquid solution was obtained. It was let to react The reaction was heated overnight at 60° C. Upon completion of the ligation reaction, the mixture was diluted with DMSO (1060 μL). Fmoc deprotection was performed initiated by adding diethylamine (80 μL, 5%, v/v) at room temperature for 15 min. A second portion of diethylamine (80 μL) in DMSO (1590 μL) was added to the reaction mixture, and the resulting mixture was reacted that was stirred at room temperature for another 15 min.

Trifluoroacetic acid (160 μL) was added in order to neutralize the reaction mixture. A very homogeneous and colorless liquid solution was obtained. The resulting mixture was further diluted with 1:1 CH₃CN/H₂O (q.s. 15 mL) with TFA (0.1%, v/v). The diluted mixture was filtered and purified by preparative HPLC using a C18 column (5 μm, 250×50 mm) at a flow rate of 40 mL/min at 40° C. with a gradient of 10% to 50% B in 40 min. The fractions containing the purified product were pooled and lyophilized to obtain Segment 34 as a white solid in 92% purity. The isolated yield was 51.5 mg (55%). HRMS (ESI): C₃₉₂H₆₆₆N₁₁₂O₁₁₃S₃; Average isotope calculated 8851.9104 Da [M]; found: 8851.8897 Da. Retention time (analytical Method B): 6.08 min.

Preparation of SEQ ID NO: 187-Seg1234 with Acm (SEQ ID NO: 187)

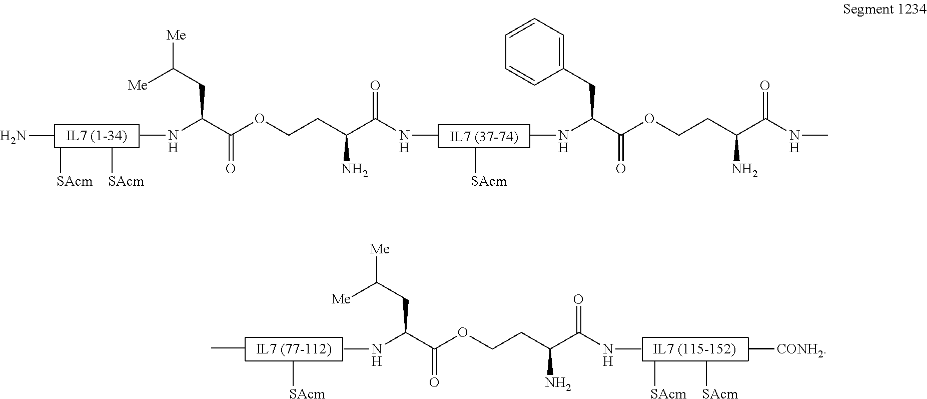

Peptide ketoacid Segment12 (17.4 mg; 1.96 μmol; 1.2 equiv) and hydroxylamine peptide Segment13 (14.5 mg; 1.64 μmol; 1.0 equiv) were dissolved in DMSO:H₂O (9.5:0.5) containing 0.1 M oxalic acid (110 μL, 15 mM peptide concentration). A homogeneous liquid solution was obtained, and the solution was heated overnight at 60° C.

After completion of the ligation the mixture was diluted with 1:1 H₂O/CH₃CN (q.s. 8 mL) containing TFA (0.1%, v/v). The diluted mixture was filtered and injected into preparative HPLC. Crude ligated peptide was purified by preparative HPLC using a C18 column (5 μm, 250×50 mm) at a flow rate of 40 m/min at 60° C. using with a gradient of 30 to 80% B in 30 min. The fractions containing the purified product were pooled and lyophilized to obtain SEQ ID NO: 187 (tri-depsipeptide) with Acm as a white solid in 99% purity. The isolated yield was 28% (8 mg). HRMS (ESI): C₇₈₄H₁₂₈₅N₂₁₉O₂₃₁S₆; Average isotope calculated 17666.4121 Da [M]; found: 17666.4233 Da. Retention time (analytical Method C): 5.33 min.

### Acm Deprotection for the Preparation of SEQ ID NO: 187-Linear Protein: IL-7-Linear Protein (SEQ ID NO: 187)

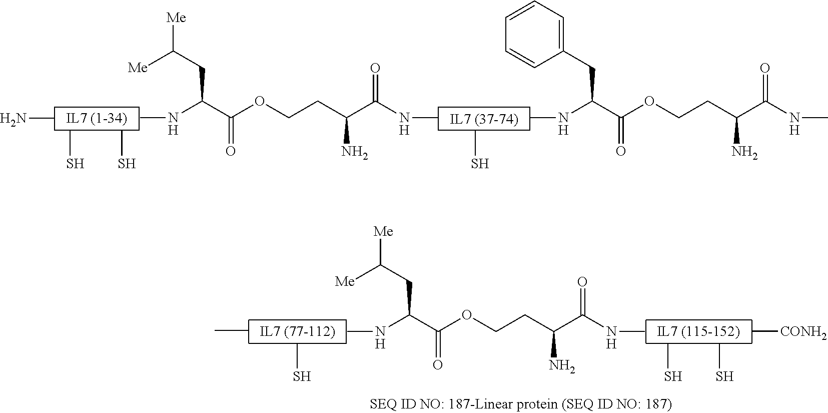

SEQ ID NO: 187 (5.8 mg; 0.33 μmol) was dissolved in AcOH:H₂O (1:1) (1.3 mL, 0.25 mM protein concentration) and silver acetate (13 mg, 1%, m/v) was added to the solution. The mixture was shaken for 2.5 h at 50° C. protected from light.

After completion of reaction, the sample was diluted with 1:1 CH₃CN:H₂O with 0.1% TFA (v/v). The sample was purified by preparative HPLC on a C18 column (5 μm, 110 Å, 250×20 mm) at a flow rate of 10 mL/min at room temperature using CH₃CN/H₂O with 0.1% TFA (v/v) as mobile phase, with a two-step gradient: 10 to 30% CH₃CN in 5 min and 30 to 95% CH₃CN in 20 min. The fractions containing the purified product were pooled and lyophilized to obtain 2.8 mg SEQ ID NO: 187-Linear protein as a white powder in 98% purity. (49% yield for Acm deprotection and purification steps). MS (ESI): C₇₆₆H₁₂₅₅N₂₁₃O₂₂₅S₆; m/z calculated: 17240.1893 Da [M+H]; found: 17240.1636 Da [M+H].

### SEQ ID NO: 187-Folded Protein: Rearrangement and Folding of IL-7 Linear Protein.

2.3 mg (0.133 μmol) of the linear IL-7 protein were dissolved in 7.5 mL of 50 mM Tris buffer containing 6 M GnHCl, 50 mM NaCl, 1 mM EDTA and 2 mM CysHCl (18 μM protein concentration), which was adjusted to pH 8.0 by adding a solution of 6 M aqueous HCl. The mixture was gently shaken at rt for 2 h. The rearrangement was monitored by analytical reverse phase HPLC.

The solution with the rearranged protein was cooled to 4° C. and diluted (×3) with 15 mL of 50 mM Tris buffer containing 50 mM NaCl and 0.1 M Arg, which was adjusted to pH 8.0 by adding a solution of 6 M aqueous HCl. The folding was allowed to proceed for 48 h at 4° C. The folding was monitored according to the rearrangement monitoring conditions.

After completion of folding reaction as ascertained by HPLC, the sample was acidified with TFA to pH=3, and purified by preparative HPLC using a C4 column (5 μm, 20×250 mm) at a flow rate of 10 mL/min at rt with a gradient of 30 to 85% B in 50 min. The fractions containing the purified product were pooled and lyophilized to obtain 0.8 mg of the folded IL-7 polypeptide (35% yield) as a white powder. The purity and identity of the pure folded protein was further confirmed by analytical HPLC and ESI/MS. MS (ESI): C₇₆₆H₁₂₄₉N₂₁₃O₂₂₅S₆; m/z calculated: 17235.0 Da [M+H]⁺; found: 17235.0 Da [M+H]⁺. FIG. 3B shows characterization data of folded SEQ ID NO: 187 IL-7 protein.

N-Terminal Modified Synthetic IL-7 (SEQ ID NO: 187 with Azide Conjugation Handle in N-Terminus (CMP-095)).

The method for synthesizing IL-7 according to the above protocol is modified in order to prepare construct SEQ ID No: 187 with azide conjugation handle in N-terminus (Composition AA). Composition AA differs from the IL-7 polypeptide of SEQ ID NO: 187 prepared above (i.e., SEQ ID NO: 187) in that CMP-095 contains a modified N-terminal amine having a structure

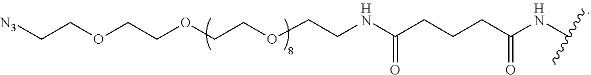

This version is prepared analogously to the IL-7 of SEQ ID NO: 187 in example 2A above with the following modification performed after final Fmoc deprotection of the N-terminal residue. Manual coupling reaction is performed at r.t. for 2 h by addition of glutaric anhydride (CAS RN 108-55-4, 114.10 mg, 5 equiv.) and DIPEA (242 μL, 7 equiv.) in DMF to the resin. Secondly, coupling with commercially available O-(2-Aminoethyl)-O′-(2-azidoethyl) nonaethylene glycol (Compound 2, 421 mg, 4 equiv) in DMF is performed at r.t. for 3 hours by addition of DIPEA (276 μL, 8 equiv) and HATU (300.4 mg, 3.95 equiv) in DMF to the resin.

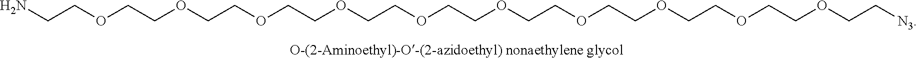 [SMILES: [N-]=[N+]=NCCOCCOCCOCCOCCOCCOCCOCCOCCOCCOCCN]

The resin is then washed and cleaved as per the normal protocol, and the modified N-terminal fragment is used in the remaining ligation steps as described supra.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A conjugate comprising: (a) an antibody or an antigen binding fragment; (b) a recombinant protein or a synthetic protein of from about 50 to about 500 amino acid residues in length; and (c) one or more linker(s) connecting the antibody or the antigen binding fragment to the synthetic protein or the recombinant protein, wherein the one or more linker(s) comprises a chemical polymer.

2. The conjugate of claim 1, wherein the one or more linker(s) is/are attached to the antibody or the antigen binding fragment at a pre-selected site of the antibody or antigen binding fragment.

3. The conjugate of claim 1, wherein the one or more linker(s) is/are attached to the recombinant protein or the synthetic protein at a pre-selected site of the recombinant protein or the synthetic protein.

4. (canceled)

5. The conjugate of claim 1, wherein the one or more linker(s) is/are attached to the recombinant protein or the synthetic protein, the antibody, or the antigen binding fragment at (a) non-terminal amino acid(s).

6-8. (canceled)

9. The conjugate of claim 1, wherein the chemical polymer comprises poly(alkylene oxide), polysaccharide, poly(vinyl pyrrolidone), poly(vinyl alcohol), polyoxazoline, poly(acryloylmorpholine), or a combination thereof.

10. (canceled)

11. The conjugate of claim 1, wherein the conjugate comprises the synthetic protein.

12. The conjugate of claim 11, wherein the synthetic protein comprises from about 50 to about 300 amino acid residues, from about 50 to about 250 amino acid residues, from about 50 to about 200 amino acid residues, from about 75 to about 300 amino acid residues, from about 75 to about 250 amino acid residues, from about 75 to about 200 amino acid residues, from about 100 to about 300 amino acid residues, from about 100 to about 250 amino acid residues, or from about 100 to about 200 amino acid residues.

13. The conjugate of claim 11, wherein the synthetic protein comprises an amino acid residue that contains a protein conjugation handle.

14. The conjugate of claim 13, wherein the amino acid residue that contains the protein conjugation handle is incorporated during synthesis of the synthetic protein.

15-16. (canceled)

17. The conjugate of claim 13, wherein at least part of the one or more linker(s) is/are formed by a conjugation reaction between the protein conjugation handle and an antibody conjugation handle attached to the antibody or the antigen binding fragment.

18-20. (canceled)

21. The conjugate of claim 1, wherein the antibody or the antigen binding fragment comprises an IgG.

22. The conjugate of claim 22, wherein the IgG comprises an IgG1, an IgG2a, an IgG4, or is derived therefrom.

23-24. (canceled)

25. The conjugate of claim 1, wherein the one or more linker(s) is/are connected to the antibody or the antigen binding fragment at an amino acid residue in a Fc region of the antibody or the antigen binding fragment.

26-28. (canceled)

29. The conjugate of claim 1, wherein linker is attached to the antibody at an Fc region at a position of a K246 amino acid residue, a K248 amino acid residue, a K288 amino acid residue, a K317 amino acid residue, or a combination thereof (Eu numbering).

30. The conjugate of claim 29, wherein the second point of attachment is at the K248 amino acid residue.

31. The conjugate of claim 1, wherein the antibody or the antigen binding fragment selectively binds to a cancer antigen, an immune cell target molecule, a self-antigen, or a combination thereof.

32-34. (canceled)

35. The conjugate of claim 11, wherein the synthetic protein comprises a therapeutic protein.

36. The conjugate of claim 11, wherein the synthetic protein comprises a cytokine.

37. The conjugate of claim 36, wherein the cytokine comprises a modified interleukin 2 (IL2), a modified IL7, a modified IL18, or a combination thereof.

38. The conjugate of claim 37, wherein the cytokine comprises the modified IL2.

39-133. (canceled)
